(19) United States
(12) Patent Application Publication
(10) Pub. No.: US 2025/0114452 A1
(43) Pub. Date: Apr. 10, 2025

(54) LAT ACTIVATING CHIMERIC ANTIGEN RECEPTOR T CELLS AND METHODS OF USE THEREOF

(71) Applicant: The Regents of the University of Colorado, A Body Corporate, Denver, CO (US)

(72) Inventors: Mark KOHLER, Denver, CO (US); Catherine DANIS, Denver, CO (US); Terry J. FRY, Bethesda, MD (US); Lillie LEACH, Denver, CO (US)

(21) Appl. No.: 18/294,650

(22) PCT Filed: Aug. 4, 2022
(86) PCT No.: PCT/US2022/039487

§ 371 Date: Feb. 2, 2024

Related U.S. Application Data
(60) Provisional application No. 63/321,549, filed on Mar. 18, 2022.
(60) Provisional application No. 63/229,344, filed on Aug. 4, 2021.

(51) Int. Cl. A61K 40/11; A61K 40/31; A61K 40/42; A61P 35/02; C07K 14/705; C07K 14/725; C07K 16/28; C12N 5/0783

(52) U.S. Cl. CPC A61K 40/11; A61K 40/31; A61K 40/4211; A61K 40/4212; A61P 35/02; C07K 14/7051; C07K 14/70517; C07K 16/2803; C12N 5/0636; A61K 2239/48; C07K 2317/622; C07K 2319/02; C07K 2319/03; C12N 2510/00

(57) ABSTRACT

The disclosure describes T cells that express chimeric antigen receptors (CARs), as well as pharmaceutical compositions comprising T cells and methods of making and using such T cells. Particularly, this disclosure describes T cells expressing a first CAR that binds to a first antigen and a second CAR comprising a LAT intracellular signaling domain that binds to a second antigen, and methods of use in treating cancers, such as solid tumors and hematological malignancies.

### RELATED APPLICATIONS

This application claims priority to, and the benefit of, U.S. Provisional Patent Application No. 63/321,549, filed on Mar. 18, 2022, and U.S. Provisional Patent Application No. 63/229,344, filed on Aug. 4, 2021, each of which is incorporated herein by reference in its entirety.

### GOVERNMENT SUPPORT

This invention was made with government support under Grant No. K12CA086913-20 awarded by the National Institutes of Health. The government has certain rights in the invention.

### FIELD OF INVENTION

The present invention relates generally to the fields of molecular biology, immunology, oncology and medicine. More particularly, it concerns immune cells expressing chimeric antigen receptors, such as chimeric antigen receptors that bind to a target protein.

### BACKGROUND OF THE INVENTION

Over the past decade, Chimeric Antigen Receptor (CAR) T cell therapy has demonstrated remarkable efficacy against B-lineage leukemias, lymphomas and multiple myeloma and held promise for the treatment of all malignancies which are otherwise incurable with conventional therapies. Across multiple clinical trials, CAR T cells targeting the CD19 antigen have induced complete remission in 70-90% of patients with multiply-relapsed and/or refractory acute lymphoblastic leukemia (ALL). This remarkable upfront success does not, however, translate to long term remissions for many patients, as longitudinal studies have demonstrated that less than 50% of CAR T cell treated patients remain in remission beyond 1 year after therapy due to post-CAR relapses. Post-CAR relapses present a clinical challenge as conventional chemotherapy, antibody-based therapies (blinatumomab and inotuzumab) and retreatment with the same CAR T cells have been found to infrequently be capable of reinducing patients into remissions, the majority of which were short-lived.

CD19-directed CAR T cell therapy for relapse and/or refractory B-lineage lymphomas has demonstrated similar results, with Objective Response Rates (ORR) of 52-82%, and 40-54% of patients achieving a Complete Response (CR), yet disease recurrence and/or progression after CAR T cell therapy remains common with less than 40% of patients remaining progression-free 1 year later. Consistent with the experience in leukemia, there are no established therapies which are effective for lymphoma patients whose disease relapsed and/or progressed after CAR T cells and reinfusion of the same CAR T cells has been largely ineffective.

Relapses after CAR therapy occur through a variety of mechanisms. In B cell leukemias treated with CD19-directed CAR T cells, upfront treatment failures and relapses in which the leukemia continues to express the CD19 antigen are highly correlated to low levels of CAR T cell expansion and a short duration of CAR T cell persistence in the patient, and it is generally held that improving CAR T cell expansion and persistence would improve outcomes by preventing relapses of antigen-positive leukemias. Another major mechanism of relapse after CAR T cell therapy is the modulation of the targeted antigen on the malignant cells as a means of escaping CAR T cell detection. In B cell leukemias, this has been mostly observed as the emergence of CD19-negative leukemia cells upon relapse. Similarly, decreased surface expression of the CD19 antigen on B-lineage lymphomas has been implicated in refractoriness to and relapse after treatment with CD19-directed CAR T cells. In either antigen-loss or down-modulation current CAR T cell therapies directed at CD19 are ineffective, an outcome which has been generalizable to other CAR-targeted antigens beyond CD19.

To overcome antigen-modulated relapses in leukemia/lymphoma CARs have been developed to target alternative antigens. CD22-directed CAR T cells have demonstrated the ability to induce remissions in 70-80% of patients with ALL, including patients with CD19-negative relapses after immunotherapy. Unfortunately, relapse after CD22-directed CAR T cell therapy was frequently observed in patients, due largely to down-regulation of the CD22 antigen. Currently, CD22 CAR T cell therapy is being used to bridge patients to a consolidative hematopoietic stem cell transplant (HSCT), however the long-term outcomes of this strategy are not yet known and many patients may be ineligible due to significant co-morbidities, prior HSCT(s) or a lack of a suitable donor. Thus, the clinical utility of CD22-directed CAR T cells is limited by the inability to target malignant cells expressing low-levels of antigen, similar to CD19 CAR T cell experience in lymphoma and likely representing a fundamental problem for any therapy targeting an antigen using T cells (or other immune effector cells) expressing a 2^{nd} generation CAR.

Thus, there is a need in the art for alternative approaches for generating genetically engineered immune cells (e.g. T cells) that are useful as therapeutics. There exists a need for new strategies to mitigate relapse after CAR T cell therapy to improve patient outcomes by enhancing the persistence and antigen-sensitivity of CAR T cells, and to improve the clinical efficacy of CAR T cell therapy against a variety of antigens and malignancies. The present invention addresses these unmet needs in the art.

### SUMMARY OF INVENTION

The present disclosure provides genetically modified immune cells comprising: a) a first chimeric antigen receptor (CAR) comprising an antigen recognition domain that binds to a first antigen, a transmembrane domain and an intracellular signaling domain; b) a second CAR comprising an antigen recognition domain that binds to an antigen, a transmembrane domain and a Linker for Activation of T cell (LAT) intracellular signaling domain.

In some aspects, the first antigen and the second antigen are different. In some aspects, first antigen and the second antigen are the same.

In some aspects, the intracellular signaling domain of the first CAR comprises a CD3zeta intracellular signaling domain. In some aspects, the CD3zeta intracellular signaling domain comprises the amino acid sequence of SEQ ID NO: 24 or SEQ ID NO. 25, preferably wherein the CD3zeta intracellular signaling domain comprises the amino acid sequence of SEQ ID NO: 24.

In some aspects, the intracellular signaling domain of the first CAR further comprises at least one additional intracellular signaling domains selected from the group consisting of a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8a intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain and any combination thereof. In some aspects, the at least one additional intracellular signaling domain is a 4-1BB intracellular signaling domain comprising the amino acid sequence of SEQ ID NO: 17.

In some aspects, the LAT intracellular signaling domain of the second CAR comprises the amino acid sequence of any one of SEQ ID NOs: 26-34, preferably wherein the LAT intracellular signaling domain of the second CAR comprises the amino acid sequence of SEQ ID NO: 27.

In some aspects, the LAT intracellular signaling domain of the second CAR comprises the amino acid sequence of SEQ ID NO: 26 having a substitution of arginine for the lysine (K25R) at position 25 of SEQ ID NO: 26, a substitution of glutamic acid for the glycine at position 133 (G133E) of SEQ ID NO: 26, a substitution of arginine for the lysine at position 206 (K206R) of SEQ ID No 26, or any combination of the preceding substitutions.

In some aspects, the LAT intracellular signaling domain of the second CAR comprises the amino acid sequence of SEQ ID NO: 32 having a substitution of arginine for the lysine (K25R) at position 25 of SEQ ID NO: 32, a substitution of glutamic acid for the glycine at position 104 (G104E) of SEQ ID NO: 32, a substitution of arginine for the lysine at position 177 (K177R) of SEQ ID No. 32, or any combination of the preceding substitutions.

In some aspects, the LAT intracellular signaling domain of the second CAR comprises the amino acid sequence of SEQ ID NO: 33 having a substitution of arginine for the lysine (K25R) at position 25 of SEQ ID NO: 33, a substitution of glutamic acid for the glycine at position 103 (G103E) of SEQ ID NO: 33, a substitution of arginine for the lysine at position 176 (K176R) of SEQ ID No: 33, or any combination of the preceding substitutions.

In some aspects, the LAT intracellular signaling domain of the second CAR comprises the amino acid sequence of SEQ ID NO: 34 having a substitution of arginine for the lysine (K25R) at position 25 of SEQ ID NO: 34, a substitution of glutamic acid for the glycine at position 132 (G132E) of SEQ ID NO: 34, a substitution of arginine for the lysine at position 205 (K205R) of SEQ ID No. 34, or any combination of the preceding substitutions.

In some aspects, the transmembrane domain of the first CAR and/or the second CAR is derived from a transmembrane domain selected from the group consisting of a CD8a transmembrane domain, a CD28 transmembrane domain, a CD3z transmembrane domain, a CD4 transmembrane domain, a 4-1BB transmembrane domain, a OX40 transmembrane domain, a ICOS transmembrane domain, a PD-1 transmembrane domain, a LAG-3 transmembrane domain, a 2B4 transmembrane domain, a BTLA transmembrane domain and any combination thereof. In some aspects, the transmembrane domain of the first CAR is derived from a CD8alpha transmembrane domain comprising the amino acid sequence of SEQ ID NO: 13. In some aspects, the transmembrane domain of the second CAR is derived from a CD28 transmembrane domain comprising the amino acid sequence of SEQ ID NO: 14.

In some aspects, the antigen recognition domain of the first CAR and/or the antigen recognition domain of the second CAR is an antibody, an antibody fragment, a single chain antibody, a single domain antibody, an scFv, a VH or a VHH or antigen binding fragment thereof.

In some aspects, the antigen recognition domain of the first CAR and the antigen recognition domain of the second CAR further comprises a leader domain selected from the group consisting of a CD8alpha leader domain. In some aspects, the leader domain is a CD8alpha leader domain comprising the amino acid sequence of SEQ ID NO: 1 or SEQ ID NO: 2.

In some aspects, the first antigen and the second antigen are tumor associated antigens. In some aspects, a tumor associated antigen is selected from a group consisting of CD19, CD22, CD20, CD138, BCMA, CD33, CD123, FLT, CLL, CD56, CD34, CD117, CD14, CD133, CD44v6, CD47, CD64, CD96, CD97, CD99, CD45, CD9, Muc1, Lewis-Y, IL1RAP, FR-beta, CD5, CD7, CD38, CD30, B7-H3, HER2, CD44v6, CEA, c-Met, EGFRVIII, Epcam, EphA2, FR-alpha, GD2, GPC3, IL13R-alpha2, IL11R-alpha, L1-CAM, mesothelin, MUC1, MUC16, NKGD2 and PSCA. In some aspects, the first antigen is CD22. In some aspects, the second antigen is CD19.

In some aspects, the immune cell is a T-cell, a Natural Killer (NK) cell, a Natural Killer (NK)-like cell, a Cytokine Induced Killer (CIK) cell, a hematopoietic progenitor cell, a peripheral blood (PB) derived T cell or an umbilical cord blood (UCB) derived T-cell. In some aspects, the immune cell is a T-cell. In some aspects, the immune cell is an iPS-derived immune cell.

In some aspects, the first CAR comprises an amino acid sequence of SEQ ID NO: 69, SEQ ID NO: 102, SEQ ID NO: 306, or SEQ ID NO: 309. In some aspects, the second CAR comprises an amino acid sequence of SEQ ID NO: 71, SEQ ID NO: 100, SEQ ID NO: 206, or SEQ ID NO: 300-308.

In some aspects, the genetically modified immune cell comprises a first CAR comprising the amino he amino acid sequence of SEQ ID NO: 102 and a second CAR comprising SEQ ID NO: 100. In some aspects, the genetically modified immune cell comprises a first CAR comprising the amino acid sequence of SEQ ID NO: 102 and a second CAR comprising the amino acid sequence of SEQ ID NO: 306. In some aspects, the genetically modified immune cell comprises a first CAR comprising the amino acid sequence of SEQ ID NO: 309 and a second CAR comprising the amino acid sequence of SEQ ID NO: 100.

The present disclosure provides a composition comprising genetically modified immune cells of the present disclosure and a pharmaceutically acceptable carrier.

The present disclosure provides a composition comprising a population of cells, wherein the plurality of cells of the population comprises the genetically modified immune cells of the present disclosure. In some aspects, the plurality of the cells of the population comprises at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the genetically modified immune cells of the present disclosure.

The present disclosure provides polynucleotides encoding the first CAR and the second CAR of the present disclosure. In some aspects, a nucleic acid sequence encoding a self-cleaving peptide sequence is located in between the nucleic acid sequence encoding the first CAR and the nucleic acid sequence encoding the second CAR. In some aspects, the self-cleaving peptide sequence comprises the amino acid sequence of SEQ ID NO: 79. In some aspects, the first CAR and the second CAR encoded on a single vector. In some aspects, the vector is a viral vector, a lentivirus vector, a non-viral vector or a transposon. In some aspects, the vector is a bicistronic lentiviral vector.

The present disclosure provides a method of producing a population of genetically modified immune cells, comprising: a) introducing into a plurality of immune cells a composition comprising the polynucleotide sequence of the present disclosure, thereby generating a population of genetically modified immune cells; b) culturing the population of genetically modified immune cells under conditions suitable for integration of the polynucleotide sequence; c) expanding and/or selecting at least one cell from the population of genetically modified immune cells that expresses the first CAR and the second CAR on the cell surface.

The present disclosure provides a method of treating cancer in a subject in need thereof comprising administrating a composition of the present disclosure. In some aspects, the administration of a composition comprising a modified immune cell comprising first CAR and the second CAR increases the immune response against a target cell in comparison to the administration of a composition comprising a modified immune cell comprising a first CAR alone. In some aspects, the increased immune response at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater than a composition comprising a modified immune cell comprising a first CAR alone. In some aspects, the cancer is a solid tumor, a B cell malignancy, a myeloid malignancy, a T-cell malignancy, acute lymphoblastic leukemia, acute lymphoblastic lymphoma, Non-Hodgkin lymphoma, Hodgkin's lymphoma, chronic lymphocytic leukemia, multiple myeloma, acute myeloid leukemia, myelodysplastic syndrome, myeloproliferative neoplasms, chronic myeloid leukemia, T lymphoblastic leukemia, T lymphoblastic lymphoma or Anaplastic Large Cell Leukemia. In some aspects, the cancer has a low cell surface expression of the first antigen and/or a low cell surface expression of the second antigen.

### BRIEF DESCRIPTION OF THE DRAWINGS

FIGS. 1A-D show that antigen density impacts CAR T cell efficacy and signaling through LAT. FIG. 1A are images showing NSG mice inoculated with NALM6 expressing no, low- or WT-levels of CD22. Mice were treated with CD22 CAR T cells generated from a healthy donor 5 days later. Leukemia progression was followed by bioluminescent imaging. FIGS. 1B and 1C are western blots showing Jurkat cells stably expressing CD22 CAR that were stimulated with NALM6 cells expressing No, Low- or WT-levels of CD22 for 2, 5 or 10 min. Western blot analysis was performed on lysate and probed for phospho- and total ZAP70 (FIG. 1B) and LAT (FIG. 1C). FIG. 1D is a histogram depicting CD22 CAR T cells that were co-incubated with NALM6 cells expressing No, Low-, WT- or High-levels of CD22 antigen for 15 min. Cells were fixed and permeabilized and phospho-ERK was evaluated by flow cytometry.

FIGS. 2A-B show the design of exemplary bicistronic LAT-CAR and ALA-CAR constructs disclosed herein. FIG. 2A is a schematic of a standard 2nd Generation (Gen) (2G) CD22 CAR. FIG. 2B is a schematic of an exemplary bicistronic LAT-CAR or ALA-CAR comprising a first CAR (e.g. 2G CD22 CAR) expressed with a second CAR (e.g. “LAT-CAR” or “ALA-CAR” such as a CD19-directed CAR incorporating the LAT intracellular domain that will amplify the CAR response to low antigen).

FIGS. 2C-F show that bicistronic LAT-CAR increases antigen sensitivity of CD22 CAR. FIG. 2C are whole-body bioluminescent images of NSG mice inoculated with 10⁶ CD22-Low NALM6 and treated with 3×10⁶ or 2.5×10⁶ standard 2G CD22 CAR T (CD22 CART) cells or bicistronic LAT-CAR T cells (ALA-CART) or untreated (No Tx) and followed by BLI twice weekly. FIG. 2D is a line graph showing the quantification of the BLI imaging shown in FIG. 2C. FIG. 2E is a graph showing the survival of the mice cohorts treated in FIG. 2C. FIG. 2F is a series of graphs showing the analysis of bone marrow samples obtained from the surviving mice treated with bicistronic LAT-CAR T cells in FIG. 2C and demonstrates the continued persistence of bicistronic LAT-CAR T cells 50 days after initial treatment.

FIG. 3 is a graph showing that 2G-CAR T cells have reduced in vitro leukemia killing against CD22-low NALM6. CD22 2G-CAR T cells were generated from healthy donor T cells and co-incubated for 6 days with GFP+ NALM6 cells expressing WT-(upright triangles) or Low-(upside down triangles) levels of CD22 antigen at an E:T of 1:1. Leukemia cell killing was monitored over time by flow cytometry. Leukemia cell counts were normalized to counting beads in the co-culture and are depicted on the y-axis. Days in co-culture are depicted on the x-axis.

FIG. 4 is a series of flow cytometry histograms showing post-transduction enrichment of CAR-positive T cells. T cells from a healthy donor were activated and transduced with lentivirus containing the bicistronic CD22/19 LAT-CAR construct. Two days later, surface expression of CAR was determined by staining cells with fluorescently-labeled CD22-Fc and CD19-Fc (top). CAR+ cells were positively selected using Miltenyi beads and T cells were expanded for 4 more days. At the end of expansion, T cells were stained again for surface CAR expression (bottom) demonstrating enrichment of CAR+ cells for downstream experiments.

FIG. 5 is a series of graphs showing surface co-expression of the first and second CAR of the bicistronic CAR of the present disclosure as measured by flow cytometry (top panels) and relative intensity of surface expression of the first CAR (ALA-CART-CD22BBz) of the present disclosure relative to a standard 2^{nd} generation CAR (2G CD22 BBz) (bottom panel).

FIG. 6 is a series of graphs showing surface expression of CAR constructs utilizing different transmembrane domains in the second CAR (e.g. LAT CAR) of the bicistronic CAR of the present disclosure. Use of the LAT transmembrane domain in the LAT-CAR resulted in minimal expression of the presently disclosed bicistronic CAR on the surface of T cells from 3 healthy donors (top), whereas the incorporation of a transmembrane domain derived from the CD28 molecule into the second CAR (e.g. LAT CAR) of the presently disclosed bicistronic CAR construct resulted in efficient surface expression of the LAT CAR in the T cells of the same healthy donors (bottom).

FIG. 7 is a series of western blot images and graphs showing the increased expression of LAT and increased activating phosphorylation of LAT (p-LAT225) in cells transduced with the bicistronic CAR constructs of the present disclosure (“ALA-CART” or “22×19 ALACART”), in response to normal (+) or low (Low) levels of CD22 on leukemia cells, relative to cells transduced with a 2G CD22Bz (“22Bz”).

FIG. 8 is a series of western blot images and graphs showing the expression levels of total Phospholipase C-gamma (PLCg) and the enhanced activation of PLCg by phosphorylation (p-PLCg) in cells transduced with the bicistronic CAR constructs of the present disclosure (“22×19 LAT” or “22×19 ALACART”) in response to normal (+) or low (Low) levels of CD22 on leukemia cells, relative to cells transduced with a 2G CD22Bz (“22Bz”).

FIG. 9 is a graph showing leukemia-killing by CAR T cells as the ratio of leukemia cells to CAR cells in cultures comprising NALM6 leukemia cells expressing various combinations of CD19 and CD22 antigens (DN—double negative, 19-, 22-, WT or 22 Low) and bicistronic CAR T cells of the present disclosure (22×19LAT) or a CD22 CAR control.

FIG. 10 is a series of graphs showing hIL-2 concentration and hIFNg concentration in cultures (as measured by ELISA) comprising NALM6 leukemia cells co-cultured with the bicistronic CAR T cells of the present disclosure (22×19LAT).

FIG. 11A is a series of images showing whole-body bioluminescent imaging (BLI) analysis in mice bearing leukemia expressing wild type levels of CD22, subsequently treated with the bicistronic CAR constructs of the present disclosure (ALA-CART) compared to mice treated with a standard 2^{nd} generation CAR (CD22 CART) and mice undergoing no treatment (No Tx).

FIG. 11B is a graph showing the quantification of the bioluminescent imaging (BLI) analysis in mice treated with the bicistronic CAR constructs of the present disclosure (22×19 ALACART) or the second generation CAR constructs (CD22BBz CAR) in FIG. 11A.

FIG. 11C are flow cytometry plots and a graph showing analysis of bone marrow samples taken from mice treated with standard 2^{nd} generation CARs compared to mice treated with exemplary bicistronic CAR constructs of the present disclosure 50 days after CAR T cell infusion. These data demonstrate enhanced persistence of the presently disclosed bicistronic CAR T cells (ALA-CART) relative to standard second generation CAR T cells (CD22 CART).

FIG. 12A-12D is a series of charts, flow cytometry plots and graphs showing the increased in vivo persistence of the disclosed bicistronic CAR (“22×19 LAT” or “22×19ALA-CART”). FIG. 12A is a series of graphs showing flow cytometric analysis of bone marrow samples taken from mice treated with standard 2^{nd} generation CAR T cells (“22SA”) versus those treated with the bicistronic CAR T cells (“22×19LAT”) of the present disclosure. These data demonstrate enhanced persistence of the presently disclosed CAR T cells (“22×19LAT”) is primarily driven by persistence of CD4+ CAR T cells (top panels) relative to CD8+ CAR T cells (bottom panels). FIG. 12B is a series of flow cytometry histograms showing decreased expression of the exhaustion marker, CD39, on the surface of the bicistronic CAR T cells of the present disclosure (“22×19ALACART”) relative to standard second generation CD22 CAR T cells (“22BBz”) at 50 days after CAR T cell infusion. FIG. 12C is a series of flow cytometry plots and summarizing graphs showing the analysis of various T cell populations in samples obtained from mice treated with the bicistronic CAR T cells of the present disclosure (“22×19 ALA-CART”) versus mice treated with the standard second generation CD22 CAR T cells (“22SA”) 50 days after CAR T cell infusion. These data demonstrate an increased proportion of the CAR T cells of the present disclosure having a central memory (CM) phenotype that has been correlated with long-term persistence. FIG. 12D is a series of flow cytometry plots, histograms and summarizing graphs showing the analysis of IL-7 Receptor-alpha (IL7RA) expression on CAR T cells obtained from mice treated with the bicistronic CAR T cells of the present disclosure (“22×19ALACART” or “22×19LAT”) versus mice treated with the standard second generation CAR22 CAR T cells (“22BBz”). These results demonstrate increased expression of the IL7RA on CD4 T cells with an Effector Memory (EM) and Effector Memory-expressing CD45RA (T-EMRA) subpopulations in bicistronic CAR T cells versus second generation CD CAR T cells, suggesting enhanced ability for long-term persistence of these cells.

FIGS. 13A-13B is a series of imaging data and graphs showing an exemplary bicistronic LAT-CAR (ALA-CART) is effective against each targeted antigen. FIG. 13A is a series of images showing bioluminescent imaging (BLI) analysis in mice inoculated with leukemia expressing both antigens targeted by the bicistronic CAR constructs of the present disclosure (WT NALM6 CD19+/CD22+) or inoculated with leukemia expressing one or the other antigen targeted by the CAR of the present disclosure (CD19-NALM6 (CD22+) or CD22-NALM6 (CD19+)). Leukemia-bearing mice were treated with the bicistronic CAR T cells of the present disclosure (ALA-CART) versus the standard second-generation CAR T cells (CD22 CART) versus no treatment (No Tx). Leukemia was eradicated by the bicistronic CAR T cell of the present disclosure regardless of which antigen(s) were present on the leukemia FIG. 13B is a graph showing the percentage of CAR T cells in bone marrow samples obtained from mice treated with the bicistronic CAR T cells of the present disclosure after complete leukemia clearance, demonstrating the persistence of the bicistronic CAR T cell from the present disclosure in response to leukemia expressing both (WT) or either (CD19-, CD22-) targeted antigens.

FIGS. 14A-14C are a series of flow cytometry histograms and graphs showing phosphorylation of signaling molecules in exemplary bicistronic CAR T cells of the present disclosure (22×19LAT) or second generation CD22 CAR T cells (22BBz) co-cultured with NALM6 leukemia cells express no (DN), both (WT) or one or the other (19-, 22-) of the targeted antigens. FIG. 14A shows ERK (p-ERK) expression. FIG. 14B shows p38 (p-p38) expression. FIG. 14C shows PLCg (p-PLCg) expression.

FIG. 15 shows images and graphs of the quantified bioluminescent imaging (BLI) analysis in mice inoculated with CD22-low leukemia and treated with the bicistronic CAR constructs of the present disclosure designed to solely target the CD22 antigen (SAff/SAff-LAT, SAff/HiAff-LAT, HiAff/SAff-LAT, HiAff/HiAff-LAT) versus mice treated with standard CD22 CAR T cells (22SAff (SEQ ID NO: 69)). Various combinations of antigen-binding domains (scFv's) were tested utilizing a standard affinity (SAff) and a high-affinity (HiAff) scFv on either the first, second or both CARs of the presently disclosed construct. Of these various combinations, the use of the high-affinity scFv on both CARs (HiAff/HiAff) demonstrated the best clearance of CD22-low leukemia.

FIG. 16 shows images and graphs of quantified bioluminescent imaging (BLI) analysis of mice inoculated with leukemia expressing normal (NALM6 WT) or low (NALM6 22low) levels of the CD22 antigen followed by treatment with the bicistronic CAR constructs of the present disclosure utilizing the high-affinity scFv at both positions (“HiAff/HiAff LAT” or “22ALACART4”) versus mice treated with the standard second generation CD22 CAR (22SAff) versus mice treated with untransduced T cells (Mock). These data demonstrate the ability of the HiAff/HiAffLAT version of the present disclosure to eradicate CD22-low leukemia while only targeting the CD22 antigen.

FIGS. 17A-17D show a series of graphs showing the flow cytometric analysis of the phenotypes of CAR cells of the present disclosure at the completion of manufacturing relative to the phenotypes of standard second generation CD22 CAR T cells (22BBz). Various versions of the present disclosure analyzed in this figure include CAR T cells targeting CD22 only with the standard-affinity scFv on both CARs (22ALACART1), CAR T cells targeting CD22 only with the standard-affinity scFv on the first CAR and the high-affinity scFv on the second CAR (22ALACART2), CAR T cells targeting CD22 only with the high-affinity scFv on the first CAR and the standard-affinity scFv on the second CAR (22ALACART3), CAR T cells targeting CD22 only with the high-affinity scFv on both CARs (22ALACART4), CAR T cells targeting CD22 and CD19 with the standard-affinity CD22 scFv on the first CAR and a CD19-targeting scFv on the second CAR (22×19ALACART). Phenotypic analysis of T cells subsets, including T stem cell memory (Tsem), central memory (Tem), effector memory (Tem) and effector memory re-expressing CD45RA (temra) were analyzed in CD4 (FIG. 17A) and CD8 (FIG. 17C) CAR T cells. IL-7 Receptor-alpha (IL7RA) surface expression was also evaluated on CD4 (FIG. 17B) and CD8 (FIG. 17D) CAR T cells. These data demonstrate that transduction of T cells with the presently disclosed bicistronic CAR construct yielded CAR T cell products composed of a higher percentage of Tscm cells than the standard second generation CAR, regardless of the combination of scFvs used. Similarly, IL7RA expression was uniformly higher in all configurations of the presently disclosed bicistronic CAR T cells relative to the IL7RA expression of standard CD22 CAR T cells.

FIG. 18 is a series of graphs showing the flow cytometric analysis of the expression of CD39, a marker associated with T cell exhaustion, on T cells transduced with the various configurations of the presently disclosed bicistronic CAR (22-ALA-CART) (SAff/SAff-LAT, SAff (SA)/HiAff-LAT, HiAff/SAff (SA)-LAT, HiAff/HiAff-LAT) versus expression on T cells transduced with the standard 2^{nd} generation CD22 CAR T (22SA). Analysis of T cells was subdivided into analysis of CD4+CAR (“CAR4”) (top) and CD8+CAR (“CAR8”) (bottom) CAR T cells. Expression of the CD39 exhaustion marker was lower on T cells transduced with any of the configurations of the presently disclosed bicistronic CAR than on T cells transduced with the standard 2^{nd} generation CD22 CAR.

FIG. 19 is a series of whole-body bioluminescent images depicting leukemia progression and in vivo activity of exemplary bicistronic LAT-CAR T cells (19ALA-CART) in mice compared to standard 2^{nd} generation CD19 CAR T cells (CD19BBz) and non-transduced T cell (Mock) controls in mice Images were taken between 1 day (D−1) and 14 days (D14) after T cell injection, as indicated. Bioluminescent activity is indicated by color (Radiance).

FIG. 20 is a series of whole-body bioluminescent images depicting leukemia progression and in vivo potency of an exemplary bicistronic LAT-CAR T cells (19ALA-CART) in mice engrafted with CD19-high NALM6 cells compared to a standard 2^{nd} generation CD19 CAR T cells (CD19BBz) and non-transduced T cell (Mock) controls. Images were taken between 1 day (D−1) and 42 days (D42) after T cell injection, as indicated. Bioluminescent activity is indicated by color (Radiance).

FIG. 21 is a graph of CAR T cell-mediated killing of CD22-low leukemia cells after overnight co-culture with exemplary bicistronic 22ALA-CAR T cell variants (LAT-WT (SEQ ID NO: 26), LAT-K52R (SEQ ID NO: 27), LAT-233R (SEQ ID NO: 28), LAT-K52R+K233R (SEQ ID NO: 29)) variants compared to control T cells (Mock) at multiple ratios. The ratio of effector CAR T cells to target leukemia cells (E:T Ratio) is depicted on the x-axis. Cell killing is indicated on the y-axis as specific lysis (%).

FIGS. 22A-22B are a series of graphs showing CAR T cell-mediated killing of CD22-low leukemia cells after overnight co-culture with exemplary 22ALA-CART variants (LAT-K52R (SEQ ID NO: 27), LAT-K52R+G160E (SEQ ID NO: 30), LAT-K52R+K233R (SEQ ID NO: 29), LAT-K52R+K233R+G160E (SEQ ID NO: 31)) compared to control T cells (Mock) at multiple ratios. The ratio of effector CAR T cells to target leukemia cells (E:T Ratio) is depicted on the x-axis. Cell killing is indicated on the y-axis as specific lysis (%). FIG. 22A shows cell killing by LAT-CARs with mutations at the ubiquitination site K52 with (LAT-K52R+G160E, LAT-K52R+K233R+G160E) or without the PLC-activating mutation G160E (LAT-K52R).

FIG. 22B shows cell killing by LAT-CARs with mutations at the ubiquitination sites K52 and K233 with (LAT-K52R+G160E) or without the PLC-activating mutation G160E (LAT-K52R+K233R).

FIG. 23A-23B are a series of graphs showing the function of the bicistronic LAT-CAR T cells (ALA-CART) relative to the standard 2^{nd} generation CD22 CAR T cells FIG. 23A are graphs of the quantification of the cytokines IL-2 and Interferon-gamma (IFNg) produced by either the bicistronic ALA-CART cells (22×19ALACART) or standard 2^{nd} generation CD22 CAR T cells (22BBz) after overnight co-culture with CD22-low NALM6 cells or CD22 (−) NALM6 cells. FIG. 23B is a graph showing the specific lysis of CD22-low NALM6 cells and CD22 (−) NALM6 cells by either bicistronic ALA-CART cells (22×19ALACART) or standard 2^{nd} generation CD22 CAR T cells (22BBz) after overnight co-culture at various E:T ratios. **** indicates a statistical significance with a p value of <0.0001.

FIG. 24A-24C show a series of whole-body bioluminescent images and graphs depicting the in vivo persistence of the disclosed CAR targeting NALM6 through recognition of the CD22 antigen only. FIG. 24A shows bioluminescent images of mice engrafted with WT NALM6 leukemia and treated with the disclosed bicistronic LAT-CAR T cells solely targeting CD22 (22ALA-CART) versus mice treated with standard 2^{nd} generation CD22 CAR T cells (22BBz) versus mice treated with untransduced (Mock) T cells. FIG. 24B are a series of graphs showing the quantification of persistent bicistronic CAR T cells (22ALACART4) or 2^{nd} generation CD22 CAR T cells (22BBz) in the bone marrow of mice 40 days after initial treatment, demonstrating enhanced in vivo persistence of the disclosed bicistronic CAR (22ALACART4). FIG. 24C are a series of graphs showing the quantification of the differentiation states (CM, EM and TEMRA) of persistent bicistronic CAR T cells and 2^{nd} generation CD22 CAR T cells from FIG. 24B, demonstrating increased percentages of the disclosed CAR with a memory phenotype.

FIGS. 25A-25B are a series of graphs showing phenotypes of exemplary CAR cells of the present disclosure at the completion of manufacturing compared to standard CD22 CAR T cells (22BBz). FIG. 25A are a series of pie charts showing the phenotypic analysis of T cells subsets, including T stem cell memory (TSCM), central memory (TCM), effector memory (TEM) and effector memory re-expressing CD45RA (TEMRA) in the presently disclosed bicistronic CAR T cells (22ALA-CART) compared to standard 2^{nd} generation CAR T cells (22BBz). FIG. 25B are a series of graphs showing the percentage of T cells (CD4+CAR (“CAR4”) or CD8+ CAR (“CAR8”)) with a TSCM phenotype from 3 different T cell donors after manufacturing the disclosed CAR (22ALA-CART) and the standard 2^{nd} generation CAR (22BBz).

### DETAILED DESCRIPTION OF THE INVENTION

The present invention generally provides cells, including immune cells (e.g., T cells, B cells, Natural Killer (NK) cells, monocytes, macrophages or artificially generated cells with immune effector function) derived from a patient, a healthy donor, a differentiated stem cell (including but not limited to induced pluripotent stem cells (iPSC), embryonic stem cells, hematopoietic and/or other tissue specific stem cells) or a non-human source, which are genetically modified to express a first antigen recognizing receptor (e.g., chimeric antigen receptor (CAR)) that binds to a first antigen along with a second antigen recognizing receptor (e.g., CAR) comprising the intracellular signaling domain of the Linker for Activation of T cell (LAT) that binds to a second antigen, and methods of use thereof for the treatment of cancer, infection, autoimmunity, alloimmunity, lymphoproliferative disease, pathologic immune dysregulation and other pathologies where an increase in an antigen-specific immune response is desired or for the facilitation of solid organ or hematopoietic stem cell transplantation. The first CAR and the second CAR may recognize an identical epitope or different epitopes on the same antigen, or epitopes found on two distinct antigens. Immune cell (e.g. T cell) activation is mediated by engagement of either the first CAR to its cognate antigen (e.g., CD22) or the second CAR comprising a LAT intracellular domain to its cognate antigen (e.g., CD19) with signal amplification leading to enhanced persistence, antigen-sensitivity and efficacy occurring when both the first and second CARs are simultaneously engaged to their respective cognate (e.g., CD22 and CD19).

CARs, which are at times referred to as artificial T cell receptors, chimeric T cell receptors (cTCR), T-bodies or chimeric immunoreceptors, are engineered receptors now well known in the art. They are used primarily to transform immune effector cells, in particular T cells, to provide those cells with a desired antigen specificity and effector response. Adoptive cell therapies using CAR-T cells are particularly under investigation in the field of cancer therapy. In these therapies, T cells are removed from a patient, donor or are derive from a stem cell source and engineered to express CARs specific to the antigens found in a particular form of cancer. The CAR-T cells, which can then recognize and kill the cancer cells, are reintroduced into the patient whereupon the CAR T cells undergo proliferative expansion, elimination of target antigen-positive cells and, in a minority of patients, transition to a long-lasting, persistent population with retained anti-tumor effector activity.

First generation CARs provide a TCR-like signal from an Immunoreceptor Tyrosine-based Activation Motif (ITAM) containing intracellular signaling domain, most commonly derived from the CD3 zeta (CD3z) molecule, and thereby elicit tumoricidal functions. However, the engagement of CD3z-chain fusion receptors may not suffice to elicit substantial IL-2 secretion and/or T cell proliferation in the absence of a concomitant co-stimulatory signal. In physiological T cell responses, optimal lymphocyte activation requires the engagement of one or more co-stimulatory receptors such as CD28 or 4-1BB. In the setting of suboptimal activation elicited by first generation CARs, T cell activity in vivo is often transient and incapable of controlling the malignancy.

Second (2^{nd}) generation CARs have been constructed to transduce a functional antigen-dependent co-stimulatory signal in human primary T cells in addition to antigen-dependent TCR-like signal, permitting T cell proliferation in addition to tumoricidal activity. Second generation CARs most commonly provide co-stimulation using co-stimulatory domains (synonymously, co-stimulatory signaling regions) derived from CD28 or 4-1BB. The combined delivery of co-stimulation plus a CD3 zeta signal renders 2^{nd} generation CARs superior in terms of function as compared to their first generation counterparts (CD3z signal alone). An example of a 2^{nd} generation CAR is found in U.S. Pat. No. 7,446,190, incorporated herein by reference.

Third (3rd) generation CARs have also been prepared. These combine multiple co-stimulatory domains (synonymously, co-stimulatory signaling regions) with a TCR-like signaling domain in cis, such as CD28+4-1BB+CD3z or CD28+OX40+CD3z, to further augment potency. In the 3rd generation CARs, the co-stimulatory domains are aligned in series in the CAR endodomain and are generally placed upstream of CD3z or its equivalent. In general, however, the results achieved with these third generation CARs have been disappointing, showing only a marginal improvement over 2nd generation configurations, with some 3rd generation CARs being inferior to 2nd generation configurations.

This present invention is the first to utilize a first CAR (i.e. a 1st generation, a 2nd generation or a 3rd generation CAR) in conjunction with a second CAR having the intracellular signaling domain of LAT as a means of amplifying CAR signaling and enhancing persistence and antigen-sensitivity. Unlike the first CAR, the second CAR lacks a TCR-like signaling region such as CD3z. These T cells genetically engineered to express the dual CAR system demonstrate superior activity and persistence as compared to 1st generation CAR-T cells, 2nd generation CAR-T cells, and 3rd generation CAR-T cells. Thus, the present invention overcomes problems associated with current technologies by providing antigen-specific immune cells (e.g T cells) for immunotherapy, such as for the treatment of immune-related diseases, including cancer, autoimmune disorders and infection.

The invention is based, at least in part, on the discovery that low levels of antigen resulted in diminished Linker of T cell Activation (LAT) utilization downstream of the CAR. LAT is a scaffolding protein which acts as a key component of the signalosome and has been shown to amplify signals generated by antigen receptors in T cells by increasing cytokine release after receptor activation. The incorporation of a second, LAT-containing chimeric antigen receptor leads to significantly higher levels of LAT activation upon antigen stimulation than a second generation CAR by itself.

The invention is based, at least in part, on the discovery that the simultaneous engagement of two antigens co-expressed by a tumor cell by a first co-stimulatory and ITAM-containing receptor and a second LAT-containing antigen recognizing receptor is useful for activating and stimulating an immunoreactive cell. In particular, the reactivity against cells expressing either antigen alone may be diminished relative to responses to cells expressing both antigens due to a lack of cooperative signaling, yet productive T cell activation can occur against target cells expressing even low levels of either targeted antigen. However, T cell activation in the presence of both antigens is greater than the T cell activation with either CAR alone. Thus, this approach augments the T cell reactivity against tumors expressing low levels of tumor associated antigens.

The sensitivity of CARs for their cognate antigen greatly impacts patient outcomes of those who received CAR T therapy. Multiple sub-clones of the pre-B ALL cell line, NALM6, expressing variable amounts of the CD22 antigen were generated and when the level of CD22 falls below 1500-2000 molecules per cell there is a significant decrease in CAR T cell cytokine production, cytotoxicity, effector differentiation, persistence and in vivo efficacy. The impact of antigen density on CAR T cell function is not unique to CD22 CAR T cells, as CAR T cells against CD19, CD20, HER2, ALK and B7-H3 have all been shown to have decreased activity against antigen-low targets. Furthermore, recent clinical observations have associated low levels of CD19 antigen with treatment failure and/or relapse in patients undergoing CD19-directed CAR T cell therapy for diffuse large B cell lymphoma.

While the impact of low antigen-sensitivity of CAR T cells has been described, the mechanism underlying it has not yet been elucidated. A high sensitivity to low levels of antigen is a hallmark of conventional T cells activated through their endogenous T cell receptor (TCR), with evidence of T cell activation occurring in response to fewer than 10 antigen-MHC complexes/cell and full effector responses to fewer than 200 antigen-MHC complexes/cell. The sensitivity of the TCR is due, in part, to the formation of a highly organized immune synapse and subsequently, the formation of the signalosome around a conglomerate of LAT molecules in which the signal transduction machinery of the T cell localizes at the site of antigen binding to amplify proximal signaling events and activate multiple divergent downstream signaling pathways. CARs, conversely, do not form well-organized immune synapses in which to concentrate the necessary components of the signalosome to the site of receptor-activation within a cell. The disorganization of the CAR immune synapse and subsequent inefficient assembly and utilization of the signalosome leads to suboptimal signaling within the T cell, impairing the T cell response to low levels of antigen and diminishing higher-level T cell functions, such as the establishment of a long-lived population of persistent CAR T cells in vivo.

The inability of CAR T cells to target low-levels of antigen is immediately of clinical importance, as this is the major mechanism for relapse in patients treated with CD22 CART cells, which is the most proven therapeutic option for patients with CD19-negative leukemia after immunotherapy. Similarly, evidence is mounting that low levels of CD19 antigen are associated with increased risk of primary treatment failure and relapse in patients with Diffuse Large B cell Lymphoma. Clinical studies of B Cell Maturation Antigen (BCMA)-directed CAR T cell have suggested that upfront efficacy of the CAR T cells is diminished in patients with multiple myeloma expressing low levels of the targeted BCMA antigen. Furthermore, reduced expression of BCMA has been commonly observed upon disease progression and/or relapse after CAR T cell therapy, further emphasizing the clinical importance of enabling CAR T cells to efficiently target antigen low malignant cells.

Accordingly the present invention provides a novel approach to addressing the shortcoming of current CAR T cell therapy by improving the ability of the T cells to recognize tumor cell that express low levels of antigen, and by increasing CAR T cell persistence, thereby improving clinical patient outcomes.

While the immune cells of the present disclosure may be targeted to any combination of antigens, exemplary antigens for the CARs disclosed herein include but are not limited to CD22 and CD19 In particular aspects, the immune cells are dually targeted to an antigen combination including but not limited to CD19 and CD20, CD20 and CD22, CD19 and CD79a, CD22 and CD79a, CD20 and CD79a, CD19 and CD79b, CD22 and CD79b, CD20 and CD79b, CD19 and CD5, CD138 and BCMA, CD38 and BCMA, CD19 and BCMA, CD19 and CD138, CD19 and GPRC5D, BCMA and GPRC5D, CD138 and GPRC5D, CD38 and GPRC5D, CD5 and CD7, CD5 and TCR alpha or beta chain, CD7 and TCR alpha or beta chain, CD5 and CD38, CD7 and CD38, CD30 and ALK, CD33 and FLT3, CD33 and CD123, CD33 and CLEC1A, CD33 and CD56, CD33 and CD34, CD33 and CD117, CD33 and CD14, CD33 and CD133, CD33 and CD44v6, CD33 and CD47, CD33 and CD64, CD33 and CD96, CD33 and CD97, CD33 and CD99, CD33 and CD16, CD33 and CD45, CD33 and CD9, CD33 and Muc1, CD33 and Lewis-Y, CD33 and IL1-RAP, CD33 and FR-beta, CD33 and ROR1, CD123 and FLT3, CD123 and CLEC1A, CD123 and CD56, CD123 and CD34, CD123 and CD117, CD123 and CD14, CD123 and CD133, CD123 and CD44v6, CD123 and CD47, CD123 and CD64, CD123 and CD96, CD123 and CD97, CD123 and CD99, CD123 and CD16, CD123 and CD45, CD123 and CD9, CD123 and Muc1, CD123 and Lewis-Y, CD123 and IL1-RAP, CD123 and FR-beta, CD123 and ROR1, FLT3 and CLEC1A, FLT3 and CD56, FLT3 and CD34, FLT3 and CD117, FLT3 and CD14, FLT3 and CD133, FLT3 and CD44v6, FLT3 and CD47, FLT3 and CD64, FLT3 and CD96, FLT3 and CD97, FLT3 and CD99, FLT3 and CD16, FLT3 and CD45, FLT3 and CD9, FLT3 and Much, FLT3 and Lewis. Y, FLT3 and IL1-RAP, FLT3 and FR-beta, FLT3 and ROR1, CLEC1A and CD56, CLEC1A and CD34, CLEC1A and CD117, CLEC1A and CD14, CLEC1A and CD133, CLEC1A and CD44v6, CLEC1A and CD47, CLEC1A and CD64, CLEC1A and CD96, CLEC1A and CD97, CLEC1A and CD99, CLEC1A and CD16, CLEC1A and CD45, CLEC1A and CD9, CLEC1A and Muc1, CLEC1A and Lewis-Y, CLEC1A and IL1-RAP, CLEC1A and FR-beta, CLEC1A and ROR1, CD56 and CD34, CD56 and CD117, CD56 and CD14, CD56 and CD133, CD56 and CD44v6, CD56 and CD47, CD56 and CD64, CD56 and CD96, CD56 and CD97, CD56 and CD99, CD56 and CD16, CD56 and CD45, CD56 and CD9, CD56 and Muc1, CD56 and Lewis-Y, CD56 and IL1-RAP, CD56 and FR-beta, CD56 and ROR1, CD34 and CD117, CD34 and CD14, CD34 and CD133, CD34 and CD44v6, CD34 and CD47, CD34 and CD64, CD34 and CD96, CD34 and CD97, CD34 and CD99, CD34 and CD16, CD34 and CD45, CD34 and CD9, CD34 and Muc1, CD34 and Lewis-Y, CD34 and IL1-RAP, CD34 and FR-beta, CD34 and ROR1, CD117 and CD14, CD117 and CD133, CD117 and CD44v6, CD117 and CD47, CD117 and CD64, CD117 and CD96, CD117 and CD97, CD117 and CD99, CD117 and CD16, CD117 and CD45, CD117 and CD9, CD117 and Muc1, CD117 and Lewis-Y, CD117 and IL1-RAP, CD117 and FR-beta, CD117 and ROR1, CD14 and CD133, CD14 and CD44v6, CD14 and CD47, CD14 and CD64, CD14 and CD96, CD14 and CD97, CD14 and CD99, CD14 and CD16, CD14 and CD45, CD14 and CD9, CD14 and Much, CD14 and Lewis-Y, CD14 and IL1-RAP, CD14 and FR-beta, CD14 and ROR1, CD133 and CD44v6, CD133 and CD47, CD133 and CD64, CD133 and CD96, CD133 and CD97, CD133 and CD99, CD133 and CD16, CD133 and CD45, CD133 and CD9, CD133 and Much, CD133 and Lewis-Y, CD133 and IL1-RAP, CD133 and FR-beta, CD133 and ROR1, CD44V6 and CD47, CD44V6 and CD64, CD44V6 and CD96, CD44V6 and CD97, CD44V6 and CD99, CD44V6 and CD16, CD44V6 and CD45, CD44V6 and CD9, CD44V6 and Muc1, CD44V6 and Lewis-Y, CD44V6 and IL1-RAP, CD44V6 and FR-beta, CD44V6 and ROR1, CD47 and CD64, CD47 and CD96, CD47 and CD97, CD47 and CD99, CD47 and CD16, CD47 and CD45, CD47 and CD9, CD47 and Muc1, CD47 and Lewis-Y, CD47 and IL1-RAP, CD47 and FR-beta, CD47 and ROR1, CD64 and CD96, CD64 and CD97, CD64 and CD99, CD64 and CD16, CD64 and CD45, CD64 and CD9, CD64 and Muc1, CD64 and Lewis-Y, CD64 and IL1-RAP, CD64 and FR-beta, CD64 and ROR1, CD96 and CD97, CD96 and CD99, CD96 and CD16, CD96 and CD45, CD96 and CD9, CD96 and Muc1, CD96 and Lewis-Y, CD96 and IL1-RAP, CD96 and FR-beta, CD96 and ROR1, CD97 and CD99, CD97 and CD16, CD97 and CD45, CD97 and CD9, CD97 and Muc1, CD97 and Lewis-Y, CD97 and IL1-RAP, CD97 and FR-beta, CD97 and ROR1, CD99 and CD16, CD99 and CD45, CD99 and CD9, CD99 and Muc1, CD99 and Lewis-Y, CD99 and IL1-RAP, CD99 and FR-beta, CD99 and ROR1, CD16 and CD45, CD16 and CD9, CD16 and Muc1, CD16 and Lewis-Y, CD16 and IL1-RAP, CD16 and FR-beta, CD16 and ROR1, CD45 and CD9, CD45 and Much, CD45 and Lewis-Y, CD45 and IL1-RAP, CD45 and FR-beta, CD45 and ROR1, CD9 and Muc1, CD9 and Lewis-Y, CD9 and IL1-RAP, CD9 and FR-beta, CD9 and ROR1, MUC1 and Lewis-Y, MUC1 and IL1-RAP, MUC1 and FR-beta, MUC1 and ROR1, Lewis-Y and IL1-RAP, Lewis-Y and FR-beta, Lewis-Y and ROR1, IL1-RAP and FR-beta, IL1-RAP and ROR1, FR-beta and ROR1, B7-H3 and HER2, B7-H3 and CD44v6, B7-H3 and CEA, B7-H3 and CD133, B7-H3 and c-Met, B7-H3 and EGFRvIII, B7-H3 and EPCAM, B7-H3 and EPHA2, B7-H3 and FR-alpha, B7-H3 and GD2, B7-H3 and GPC3, B7-H3 and IL-13R-alpha2, B7-H3 and IL-11R-alpha, B7-H3 and L1-CAM, B7-H3 and Mesothelin, B7-H3 and MUC1, B7-H3 and MUC16, B7-H3 and IL1-RAP, B7-H3 and CD99, B7-H3 and PSCA, B7-H3 and PSMA, B7-H3 and ROR1, B7-H3 and ALK, HER2 and CD44v6, HER2 and CEA, HER2 and CD133, HER2 and c-Met, HER2 and EGFRvIII, HER2 and EPCAM, HER2 and EPHA2, HER2 and FR-alpha, HER2 and GD2, HER2 and GPC3, HER2 and IL-13R-alpha2, HER2 and IL-11R-alpha, HER2 and L1-CAM, HER2 and Mesothelin, HER2 and MUC1, HER2 and MUC16, HER2 and IL1-RAP, HER2 and CD99, HER2 and PSCA, HER2 and PSMA, HER2 and ROR1, HER2 and ALK, CD44v6 and CEA, CD44v6 and CD133, CD44v6 and c-Met, CD44v6 and EGFRvIII, CD44v6 and EPCAM, CD44v6 and EPHA2, CD44v6 and FR-alpha, CD44v6 and GD2, CD44v6 and GPC3, CD44v6 and IL-13R-alpha2, CD44v6 and IL-11R-alpha, CD44v6 and L1-CAM, CD44v6 and Mesotheli, CD44v6 and MUC1, CD44v6 and MUC16, CD44v6 and IL1-RAP, CD44v6 and CD99, CD44v6 and PSCA, CD44v6 and PSMA, CD44v6 and ROR1, CD44v6 and ALK, CEA and CD133, CEA and c-Met, CEA and EGFRVIII, CEA and EPCAM, CEA and EPHA2, CEA and FR-alpha, CEA and GD2, CEA and GPC3, CEA and IL-13R-alpha2, CEA and IL-11R-alpha, CEA and L1-CAM, CEA and Mesothelin, CEA and MUC1, CEA and MUC16, CEA and IL1-RAP, CEA and CD99, CEA and PSCA, CEA and PSMA, CEA and ROR1, CEA and ALK, CD133 and c-Met, CD133 and EGFRvIII, CD133 and EPCAM, CD133 and EPHA2, CD133 and FR-alpha, CD133 and GD2, CD133 and GPC3, CD133 and IL-13R-alpha2, CD133 and IL-11R-alpha, CD133 and L1-CAM, CD133 and Mesothelin, CD133 and MUC1, CD133 and MUC16, CD133 and IL1-RAP, CD133 and CD99, CD133 and PSCA, CD133 and PSMA, CD133 and ROR1, CD133 and ALK, c-Met and EGFRvIII, c-Met and EPCAM, c-Met and EPHA2, c-Met and FR-alpha, c-Met and GD2, c-Met and GPC3, c-Met and IL-13R-alpha2, c-Met and IL-11R-alpha, c-Met and L1-CAM, c-Met and Mesothelin, c-Met and MUC1, c-Met and MUC16, c-Met and IL1-RAP, c-Met and CD99, c-Met and PSCA, c-Met and PSMA, c-Met and ROR1, c-Met and ALK, EGFRvIII and EPCAM, EGFRvIII and EPHA2, EGFRvIII and FR-alpha, EGFRvIII and GD2, EGFRvIII and GPC3, EGFRVIII and IL-13R-alpha2, EGFRvIII and IL-11R-alpha, EGFRvIII and L1-CAM, EGFRvIII and Mesothelin, EGFRvIII and MUC1, EGFRvIII and MUC16, EGFRVIII and IL1-RAP, EGFRvIII and CD99, EGFRvIII and PSCA, EGFRvIII and PSMA, EGFRvIII and ROR1, EGFRvIII and ALK, EPCAM and EPHA2, EPCAM and FR-alpha, EPCAM and GD2, EPCAM and GPC3, EPCAM and IL-13R-alpha2, EPCAM and IL-11R-alpha, EPCAM and L1-CAM, EPCAM and Mesothelin, EPCAM and MUC1, EPCAM and MUC16, EPCAM and IL1-RAP, EPCAM and CD99, EPCAM and PSCA, EPCAM and PSMA, EPCAM and ROR1, EPCAM and ALK, EPHA2 and FR-alpha, EPHA2 and GD2, EPHA2 and GPC3, EPHA2 and IL-13R-alpha2, EPHA2 and IL-11R-alpha, EPHA2 and L1-CAM, EPHA2 and Mesothelin, EPHA2 and MUC1, EPHA2 and MUC16, EPHA2 and IL1-RAP, EPHA2 and CD99, EPHA2 and PSCA, EPHA2 and PSMA, EPHA2 and ROR1, EPHA2 and ALK, FR-alpha and GD2, FR-alpha and GPC3, FR-alpha and IL-13R-alpha2, FR-alpha and IL-11R-alpha, FR-alpha and L1-CAM, FR-alpha and Mesothelin, FR-alpha and MUC1, FR-alpha and MUC16, FR-alpha and IL1-RAP, FR-alpha and CD99, FR-alpha and PSCA, FR-alpha and PSMA, FR-alpha and ROR1, FR-alpha and ALK, GD2 and GPC3, GD2 and IL-13R-alpha2, GD2 and IL-11R-alpha, GD2 and L1-CAM, GD2 and Mesothelin, GD2 and MUC1, GD2 and MUC16, GD2 and IL1-RAP, GD2 and CD99, GD2 and PSCA, GD2 and PSMA, GD2 and ROR1, GD2 and ALK, GPC3 and IL-13R-alpha2, GPC3 and IL-11R-alpha, GPC3 and L1-CAM, GPC3 and Mesothelin, GPC3 and MUC1, GPC3 and MUC16, GPC3 and IL1-RAP, GPC3 and CD99, GPC3 and PSCA, GPC3 and PSMA, GPC3 and ROR1, GPC3 and ALK, IL-13R-alpha2 and IL-11R-alpha, IL-13R-alpha2 and L1-CAM, IL-13R-alpha2 and Mesothelin, IL-13R-alpha2 and MUC1, IL-13R-alpha2 and MUC16, IL-13R-alpha2 and IL1-RAP, IL-13R-alpha2 and CD99, IL-13R-alpha2 and PSCA, IL-13R-alpha2 and PSMA, IL-13R-alpha2 and ROR1, IL-13R-alpha2 and ALK, IL-11R-alpha and L1-CAM, IL-11R-alpha and Mesothelin, IL-11R-alpha and MUC1, IL-11R-alpha and MUC16, IL-11R-alpha and IL1-RAP, IL-11R-alpha and CD99, IL-11R-alpha and PSCA, IL-11R-alpha and PSMA, IL-11R-alpha and ROR1, IL-11R-alpha and ALK, L1-CAM and Mesothelin, L1-CAM and MUC1, L1-CAM and MUC16, L1-CAM and IL1-RAP, L1-CAM and CD99, L1-CAM and PSCA, L1-CAM and PSMA, L1-CAM and ROR1, L1-CAM and ALK, Mesothelin and MUC1, Mesothelin and MUC16, Mesothelin and IL1-RAP, Mesothelin and CD99, Mesothelin and PSCA, Mesothelin and PSMA, Mesothelin and ROR1, Mesothelin and ALK, MUC1 and MUC16, MUC1 and IL1-RAP, MUC1 and CD99, MUC1 and PSCA, MUC1 and PSMA, MUC1 and ROR1, MUC1 and ALK, MUC16 and IL1-RAP, MUC16 and CD99, MUC16 and PSCA, MUC16 and PSMA, MUC16 and ROR1, MUC16 and ALK, IL1-RAP and CD99, IL1-RAP and PSCA, IL1-RAP and PSMA, IL1-RAP and ROR1, IL1-RAP and ALK, CD99 and PSCA, CD99 and PSMA, CD99 and ROR1, CD99 and ALK, PSCA and PSMA, PSCA and ROR1, PSCA and ALK, PSMA and ROR1, PSMA and ALK, ROR1 and ALK. In any of the preceding antigen combinations, either the first CAR or the second CAR (e.g. the first co-stimulatory and ITAM-containing CAR and the second LAT-containing antigen recognizing CAR) can be specific for either of the antigens in the combination. In a non-limiting example, for the CD20 and CD22 antigen combination, the first CAR (co-stimulatory and ITAM-containing CAR) can be specific for CD20 and the second CAR (LAT-containing antigen recognizing CAR) can be specific for CD22, or the first CAR (co-stimulatory and ITAM-containing CAR) can be specific for CD22 and the second CAR (LAT-containing antigen recognizing CAR) can be specific for CD20.

In addition, the expression of two CARs provides the T cells increased specificity by limiting the off-target toxicity of the cells, such that a signal is only provided to the T cells to kill when the cells contact both antigens expressed on a tumor, as well as enhanced in vivo proliferation and persistence. Thus, normal cells that express only one antigen may not be targeted by the T cells of the disclosure.

Genetic reprogramming of immune cells, such as NK cells and T cells, for adoptive cancer immunotherapy has clinically relevant applications and benefits such as 1) increased ability to recognize tumor cells expressing low levels of antigen 2) increased cell persistence and proliferation. Accordingly, the present disclosure also provides methods for treating immune-related disorders, such as cancer, comprising adoptive cell immunotherapy with any of the engineered immune cells provided herein.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “about” is used to indicate that a value includes the inherent variation of error for the device, the method being employed to determine the value, or the variation that exists among the study subjects.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. For example, in some embodiments, if a full-length polypeptide binds a given ligand, a portion of that full-length polypeptide also binds to the same ligand.

The terms “protein” and “polypeptide” are used interchangeably herein.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either in vitro or in vivo.

A “plasmid,” a common type of a vector, is an extra-chromosomal DNA molecule separate from the chromosomal DNA that is capable of replicating independently of the chromosomal DNA In certain cases, it is circular and double-stranded.

An “origin of replication” (“ori”) or “replication origin” is a DNA sequence, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences, however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

A “gene,” “polynucleotide,” “coding region,” “sequence,” “segment,” “fragment,” or “transgene” that “encodes” a particular protein, is a section of a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to a RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an functional effector element) are connected in such a way as to permit transcription of the nucleic acid molecule. “Operably linked” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of molecules that are not found in a single polypeptide in nature.

| The term “homology” refers to the percent of identity between the nucleic acid residues of two polynucleotides or the amino acid residues of two polypeptides. The correspondence between one sequence and another can be determined by techniques known in the art. For example, homology can be determined by a direct comparison of the sequence information between two polypeptides by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two polynucleotide (e.g., DNA), or two polypeptide, sequences are “substantially homologous” to each other when at least about 80%, at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs, “iPSCs” or “iPS cells”.

An “embryonic stem (ES) cell” is an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult, including germ cells (e.g., sperm and eggs).

“Induced pluripotent stem cells” (iPSes, iPSCs or iPS cells) are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T-cells, B cells, NK cells) (see e.g., Doulatov et al., 2012; Notta et al., 2015).

A “multilymphoid progenitor” (MLP) is defined to describe any progenitor that gives rise to all lymphoid lineages (B, T, and NK cells), but that may or may not have other (myeloid) potentials (Doulatov et al., 2010) and is CD45RA⁺/CD10+/CD7⁺. Any B, T, and NK progenitor can be referred to as an MLP. A “common myeloid progenitor” (CMP) refers to CD45RA⁺/CD135⁺/CD10⁺/CD7⁺ cells that can give rise to granulocytes, monocytes, megakaryocytes and erythrocytes.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

“Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

As used herein, the term “subject” or “subject in need thereof refers to a mammal, preferably a human being, male or female at any age that is in need of a therapeutic intervention, a cell transplantation or a tissue transplantation. Typically, the subject is in need of therapeutic intervention, cell or tissue transplantation (also referred to herein as recipient) due to a disorder or a pathological or undesired condition, state, or syndrome, or a physical, morphological or physiological abnormality which is amenable to treatment via therapeutic intervention, cell or tissue transplantation.

As used herein, a “disruption” or “alteration” in reference to a gene refers to a homologous recombination event with a nucleic acid molecule (e.g., an endogenous gene sequence) which results in elimination or reduction of expression of one or more gene products encoded by the subject gene in a cell, compared to the level of expression of the gene product in the absence of the disruption. Exemplary gene products include mRNA and protein products encoded by the subject gene. Alteration in some cases is transient or reversible and in other cases is permanent. Alteration in some cases is of a functional or full-length protein or mRNA, despite the fact that a truncated or nonfunctional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of or associated with the gene, such as at the DNA level. Exemplary methods for gene alteration include gene silencing, knockdown, knockout, and/or gene alteration techniques, such as gene editing. Examples of gene editing methods include CRISPR/Cas systems, meganuclease systems, Zinc Finger Protein (ZFP) and Zinc Finger Nuclease (ZFN) systems and/or transcription activator-like protein (TAL), transcription activator-like effector protein (TALE) or TALE nuclease protein (TALEN) systems. Examples of gene alteration also include antisense technology, such as RNAi, siRNA, shRNA, and/or ribozymes, which generally result in transient reduction of expression, as well as gene editing techniques which result in targeted gene inactivation or alteration, e.g., by induction of breaks and/or homologous recombination. Examples include insertions, mutations, and deletions. The alterations typically result in the repression and/or complete absence of expression of a normal or “wild-type” product encoded by the gene. Exemplary of such gene alterations are insertions, frameshift and missense mutations, deletions, substitutions, knock-in, and knock-out of the gene or part of the gene, including deletions of the entire gene. Such alterations can occur in the coding region, e.g., in one or more exons, resulting in the inability to produce a full-length product, functional product, or any product, such as by insertion of a stop codon. Such alterations may also occur by alterations in the promoter or enhancer or other region affecting activation of transcription, so as to prevent transcription of the gene. Gene alterations include gene targeting, including targeted gene inactivation by homologous recombination.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a NK cell, B cell, or a T cell, or innate immune cell to a stimulus. In one embodiment, the response is specific for a particular antigen (an “antigen-specific response”).

As used herein, the term “antigen” is a molecule capable of being bound by an antibody, T-cell receptor, Chimeric Antigen Receptor and or engineered immune receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

An “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies bind to the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

The term “Graft-Versus-Host Disease (GVHD)” refers to a common and serious complication of bone marrow or other tissue transplantation wherein there is a reaction of donated immunologically competent lymphocytes against a transplant recipient's own tissue. GVHD is a possible complication of any transplant that uses or contains stem cells from either a related or an unrelated donor. In some embodiments, the GVHD is chronic GVHD (cGVHD).

A “parameter of an immune response” is any particular measurable aspect of an immune response, including, but not limited to, cytokine secretion (IFN-γ, etc.), chemokine secretion, altered migration or cell accumulation, immunoglobulin production, dendritic cell maturation, regulatory activity, number of immune cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of 3H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or treatment of a disease or condition refers to executing a protocol or treatment plan, which may include administering one or more drugs to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols or treatment plans that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis or recurrence. Treatment of cancer may also refer to prolonging survival of a subject with cancer.

“Antigen recognition moiety” or “antigen recognition domain” refers to a molecule or portion of a molecule that specifically binds to an antigen. In one embodiment, the antigen recognition moiety is an antibody, antibody like molecule or fragment thereof and the antigen is a tumor antigen.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHL CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” may be for example proteins that are members of the Ig-superfamily which are able to selectively bind a partner.

The terms “fragment of an antibody,” “antibody fragment,”, “functional fragment of an antibody,” and “antigen-binding portion” are used interchangeably herein to mean one or more fragments or portions of an antibody that retain the ability to specifically bind to an antigen (see, generally, Holliger et al. (2005) Nat. Biotech. 23 (9): 1126-29). The antibody fragment desirably comprises, for example, one or more CDRs, the variable region (or portions thereof), the constant region (or portions thereof), or combinations thereof.

Examples of antibody fragments include, but are not limited to, (i) a Fab fragment, which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al. (1988), Science 242:423-6; Huston et al. (1988) Proc. Natl. Acad. Sci. USA 85:5879-83; and Osbourn et al. (1998) Nat. Biotechnol. 16:778-81) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

A “chimeric antigen receptor” is also known as an artificial cell receptor, a chimeric cell receptor, or a chimeric immunoreceptor. Chimeric antigen receptors (CARs) are engineered receptors, which graft a selected specificity onto an immune effector cell. CARs typically have an extracellular domain (ectodomain), which comprises an antigen-binding domain and a stalk region, a transmembrane domain and an intracellular (endodomain) domain.

A “stalk region”, which encompasses the terms “spacer region” or “hinge domain” or “hinge”, is used to link the antigen-binding domain to the transmembrane domain. As used herein, the term “stalk region” generally means any oligonucleotide or polypeptide that functions to link the transmembrane domain to, either the extracellular domain or, the cytoplasmic domain in the polypeptide chain of a CAR. In embodiments, it is flexible enough to allow the antigen-binding domain to orient in different directions to facilitate antigen recognition.

The term “functional portion,” when used in reference to a CAR, refers to any part or fragment of a CAR described herein, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example, about 10%, 25%, 30%, 50%, 68%, 80%, 90%, 95%, or more, of the parent CAR.

The term “functional variant,” as used herein, refers to a polypeptide, or a protein having substantial or significant sequence identity or similarity to the reference polypeptide, and retains the biological activity of the reference polypeptide of which it is a variant. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 70% identical, about 75% identical, about 80% identical, about 85% identical, about 90% identical, about 95% identical, or about 99% identical to the nucleic acid sequence encoding the parent CAR.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “T cell” refers to T lymphocytes, and includes, but is not limited to, γ/δ T cells, α/β T cells, NK T cells, CD4⁺ T cells and CD8⁺ T cells. CD4⁺ T cells include THO, Tₕ1 and TH2 cells, as well as regulatory T cells (Treg). There are at least three types of regulatory T cells: CD4⁺ CD25⁺ T_{reg}, CD25 T_{H}3 T_{reg}, and CD25 T_{R} 1 T_{reg}. “Cytotoxic T cell” refers to a T cell that can kill another cell. The majority of cytotoxic T cells are CD8⁺ MHC class I-restricted T cells, however some cytotoxic T cells are CD4⁺. In preferred embodiments, the T cell of the present disclosure is CD4⁺ or CD8⁺.

The activation state of a T cell defines whether the T cell is “resting” (i.e, in the Gₒ phase of the cell cycle) or “activated” to proliferate after an appropriate stimulus such as the recognition of its specific antigen, or by stimulation with OKT3 antibody, PHA or PMA, etc. The “phenotype” of the T cell (e.g., naive, central memory, effector memory, lytic effectors, help effectors (THI and TH2 cells), and regulatory effectors), describes the function the cell exerts when activated. A healthy donor has T cells of each of these phenotypes, and which are predominately in the resting state. A naive T cell will proliferate upon activation, and then differentiate into a memory T cell or an effector T cell. It can then assume the resting state again, until it gets activated the next time, to exert its new function and may change its phenotype again. An effector T cell will divide upon activation and antigen-specific effector function.

“Natural killer T cells” (NKT cells) not to be confused with natural killer cells of the innate immune system) bridge the adaptive immune system with the innate immune system. Unlike conventional T cells that recognize peptide antigens presented by major histocompatibility complex (WIC) molecules, NKT cells recognize glycolipid antigen presented by a molecule called CD1d. Once activated, these cells can perform functions ascribed to both Th and Tc cells (i.e., cytokine production and release of cytolytic/cell killing molecules). They are also able to recognize and eliminate some tumor cells and cells infected with herpes viruses.

“Natural killer cells” (“NK cells”) are a type of cytotoxic lymphocyte of the innate immune system. In some instances, NK cells provide a first line defense against viral infections and/or tumor formation. NK cells can detect MHC presented on infected or cancerous cells, triggering cytokine release, and subsequently induce lysis and apoptosis. NK cells can further detect stressed cells in the absence of antibodies and/or MHC, thereby allowing a rapid immune response.

“Tumor antigen” as used herein refers to any antigenic substance produced, expressed or overexpressed in tumor cells. It may, for example, trigger an immune response in the host.

Alternatively, for purposes of this disclosure, tumor antigens may be proteins that are expressed by both healthy and tumor cells but because they identify a certain tumor type, are a suitable therapeutic target. In one embodiment, the tumor antigen is CD22. In one embodiment, the tumor antigen is CD19.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B cells, endothelial cells, activated T cells, and dendritic cells; or other molecules, naturally occurring or synthetic, such as purified MHC Class I molecules complexed to 2-microglobulin.

The term “culturing” refers to the in vitro maintenance, differentiation, and/or propagation of cells in suitable media By “enriched” is meant a composition comprising cells present in a greater percentage of total cells than is found in the tissues where they are present in an organism.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

### II. IMMUNE CELLS

Certain embodiments of the present disclosure concern immune cells which express a chimeric antigen receptor (CAR). The immune cells may be T cells (e.g., regulatory T cells, CD4⁺ T cells, CD8⁺ T cells, or gamma-delta T cells), NK cells, invariant NK cells, NKT cells, stem cells (e.g., mesenchymal stem cells (MSCs) or induced pluripotent stem (iPSC) cells). In some embodiments, the cells are monocytes or granulocytes, e.g., myeloid cells, macrophages, neutrophils, dendritic cells, mast cells, eosinophils, and/or basophils. Also provided herein are methods of producing and engineering the immune cells and methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing. In some embodiments, the immune cells are isolated from blood, such as peripheral blood or cord blood. In some embodiments, immune cells isolated from cord blood have enhanced immunomodulation capacity, such as measured by CD4-positive or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood. The population of immune cells can be derived from induced pluripotent stem cells (iPSCs) and/or any other stem cell known in the art. In some aspects, the iPSCS and/or stem cells used to derive the population of immune cells can be obtained from a subject in need of therapy or suffering from a disease associate with reduced immune cell activity, thus these IPSCs and/or stem cells will be autologous to the subject in need of therapy. Alternatively, the iPSCs and/or stem cells can be obtained from a donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells are may or may not be human leukocyte antigen (HLA)-compatible. To be rendered subject-compatible, allogeneic cells can be treated to reduce immunogenicity.

### 1. T Cells

T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. There are six types of T-cells, namely: Helper T-cells (e.g CD4+ cells), Cytotoxic T-cells (also known as TC, cytotoxic T lymphocyte, CTL, T-killer cell, cytolytic T cell, CD8+ T-cells or killer T cell), Memory T-cells ((i) stem memory TSCM cells, like naive cells, are CD45RO−, CCR7+, CD45RA+, CD62L+ (L-selectin), CD27+, CD28+ and IL-7Ra+, but they also express large amounts of CD95, IL-2R, CXCR3, and LFA-1, and show numerous functional attributes distinctive of memory cells); (ii) central memory TCM cells express L-selectin and the CCR7, they secrete IL-2, but not IFNg or IL-4, and (iii) effector memory TEM cells, however, do not express L-selectin or CCR7 but produce effector cytokines like IFNg and IL-4), Regulatory T-cells (Tregs, suppressor T cells, or CD4+CD25+ regulatory T cells), Natural Killer T-cells (NKT) and Gamma Delta T-cells.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

### 2. Genetically Engineered Antigen Receptors

The immune cells of the disclosure (e.g., autologous or allogeneic T cells (e.g., regulatory T cells, CD4⁺ T cells, CD8⁺ T cells, or gamma-delta T cells), NK cells, invariant NK cells, NKT cells, stem cells (e.g., MSCs or iPS cells) can be genetically engineered to express antigen receptors such as engineered CARs and/or TCRs. For example, the host cells (e.g, autologous or allogeneic T cells) are modified to express a CAR having antigenic specificity for a cancer antigen. In particular embodiments, T cells are engineered to express a CAR. The T cells may be further engineered to express a TCR. Multiple CARs and/or TCRs, such as to different antigens, may be added to a single cell type, such as T cells.

Suitable methods of modification are known in the art. See, for instance, Sambrook and Ausubel, supra. For example, the cells may be transduced to express a TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen receptors, and genetically engineered products of such nucleic acids. In some embodiments, the nucleic acids are heterologous. In some embodiments, the nucleic acids are not naturally occurring, such as a nucleic acid not found in nature (e.g., chimeric).

In some embodiments, the CAR contains an extracellular antigen-recognition domain that specifically binds to an antigen (e.g., a tumor antigen or a pathogen antigen). In some embodiments, the antigen is a protein expressed on the surface of cells (e.g., cancerous cells).

Exemplary engineered antigen receptors, including CARs and recombinant TCRs, as well as methods for engineering and introducing the receptors into cells, include those described, for example, in PCT Publication Nos. WO 2000/14257, WO 2013126726, WO 2012/129514, WO 2014/031687, WO 2013/166321, WO 2013/071154, and WO 2013/123061, U.S. Patent Application Publication Nos. US 2002/131960, US 2013/287748, and US 2013/0149337; and U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479,118; International Patent Application Publication No.: WO 2014/055668 A1, and European Patent Application Publication No. EP2537416; and/or those described by Sadelain et al., 2013; Davila et al., 2013; Turtle et al., 2012; Wu et al., 2012.

### 3. Chimeric Antigen Receptors

In some aspects, the present disclosure provides a population of genetically modified immune cells (e.g. T cells) engineered to express a first chimeric antigen receptor (CAR) and/or a polynucleotide encoding a CAR, wherein the CAR comprises (a) an antigen recognition domain that specifically binds to a first antigen (e.g. CD22); a transmembrane domain; and an intracellular signaling domain and (b) a second chimeric antigen receptor (CAR) and/or a polynucleotide encoding a CAR, wherein the second CAR comprises (a) an antigen recognition domain that specifically binds to an antigen, wherein the antigen may differ from the antigen to which the first CAR binds (e.g. CD22 and CD19) or may be the same antigen to which the first CAR binds (e.g. CD22 and CD22); a transmembrane domain; and a LAT intracellular signaling domain. In some embodiments, the intracellular domain of the first CAR comprises one or more (e.g, one, two, three, or more) co-stimulatory domains.

In some embodiments, the genetically engineered cells include additional CARs, including activating or stimulatory CARs, co-stimulatory CARs (see, e.g., PCT Publ. No. WO 2014/055668), and/or inhibitory CARs (iCARs, see, e.g., Fedorov et al., 2013). The CARs generally include an extracellular antigen (or ligand) recognition domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone. For example, once an antigen is recognized by the extracellular antigen recognition domain, the intracellular signaling components transmit an activation signal to the T cell that induces the T cell to destroy a targeted tumor cell.

### A. Antigen Recognition Domains

In some embodiments, the antigen recognition domain of the CARs described herein may recognize an epitope comprising the shared space between one or more antigens. In some embodiments, the antigen recognition domain comprises complementary determining regions (CDRs) of a monoclonal antibody, variable regions of a monoclonal antibody, an scFv, a VH, a VHH, a single domain antibody (e.g., a camelid single domain antibody), an antibody mimetic and/or antigen binding fragments thereof. In some embodiments, the specificity of the antigen recognition domain is derived from a protein or peptide (e.g., a ligand in a receptor-ligand pair) that specifically binds to another protein or peptide (e.g., a receptor in a receptor-ligand pair). In some embodiments, the antigen recognition domain comprises an aptamer, a T cell receptor (TCR)-like antibody, or a single chain TCR (scTCR). Almost any moiety that binds a given target (e.g., tumor associated antigen (TAA)) with sufficient affinity can be used as an antigen recognition domain. The arrangement of the antigen recognition domain could be multimeric, such as a diabody or multimers. In some embodiments, the multimers can be formed by cross pairing of the variable portion of the light and heavy chains into a diabody.

In some embodiments, the antigen recognition domain of the CARs described herein comprises an antibody mimetic. The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen Exemplary antibody mimetics include, but are not limited to, an affibody, an afflilin, an affimer, an affitin, an alphabody, an anticalin, an avimer (also known as avidity multimer), a DARPin (Designed Ankyrin Repeat Protein), a Fynomer, a Kunitz domain peptide, a monobody and a centyrin.

In some embodiments, the first CAR provided herein comprise a single chain variable fragments (scFv) derived from monoclonal antibodies specific for tumor associated antigen (e.g., CD22), a hinge domain, a transmembrane domain, and an ITAM-containing intracellular signaling domain (e.g. CD3ζ). Such molecules result in the transmission of an ITAM-mediated signal in response to recognition by the scFv of its target. In some embodiments, the first CAR further comprises an additional intracellular signaling domain (“costimulatory domain”).

In some embodiments, the second CAR provided herein comprises a single chain variable fragments (scFv) derived from monoclonal antibodies specific for tumor associated antigen (e.g., CD19), a hinge domain, a transmembrane domain, and a LAT intracellular signaling domain. Such molecules result in the transmission of a LAT signal in response to recognition by the scFv of its target and amplify the signal from the first CAR.

Nucleic acids encoding any of the CARs described herein are also provided. Nucleic acids encoding the CAR may be humanized. In some embodiments, the nucleic acid encoding a CAR provided herein is codon-optimized for expression in human cells. In some embodiments, the disclosure provides a full-length CAR cDNA or coding region.

In some embodiments, the antigen recognition domain of a CAR provided herein comprises a fragment of the VH and VL chains of a single-chain variable fragment (scFv) that specifically bind CD22. Accordingly, the antigen recognition domain of a CAR provided herein can comprise any scFv known in the art to specifically bind CD22.

In some embodiments, the antigen recognition domain of a CAR provided herein comprises a fragment of the VH and VL chains of a single-chain variable fragment (scFv) that specifically bind CD19 such as those described in U.S. Patent Appl. Publ. Nos. 2020/0246382, PCT Appl. Publ. Nos. WO 2020223445 and WO 2020123691, each of which is incorporated herein by reference in its entirety. Accordingly, the antigen recognition domain of a CAR provided herein can comprise any scFv known in the art to specifically bind CD19.

In some embodiments, the antigen recognition domain of the CAR described herein binds (e.g. specifically binds) to the antigens described in Table 1. The antigen specific CAR, when expressed on the cell surface, redirects the specificity of immune cells (e.g. T cells) to the respective antigen.

TABLE 1
Exemplary Targets of Antigen Recognition Domains

| | | |
|---|---|---|
| | UniProt | NCBI |
| Protein Name | ID | Accession No. |

B cell malignancies

| | | | |
|---|---|---|---|
| CD19 | B-lymphocyte antigen CD19; | P15391 | NM_001178098 |
| | Cluster of Differentiation 19; B- | | |
| | Lymphocyte Surface Antigen | | |
| | B4; T-Cell Surface Antigen | | |
| | Leu-12; CVID3 | | |
| CD22 | Cluster of Differentiation 22 | P20273 | NM_001185099 |
| CD20 | B-lymphocyte antigen CD20; | P11836 | NM_021950 |
| | B-lymphocyte cell-surface | | NM_152866 |
| | antigen B1; CD20 antigen; | | NM_152867 |
| | CD20 receptor; leukocyte | | |
| | surface antigen Leu-16; | | |
| | membrane-spanning 4-domains, | | |
| | subfamily A, member 1 | | |
| CD138 | syndecan-1; CD138 antigen; | P18827 | NM_001006946 |
| | heparan sulfate proteoglycan | | NM_002997 |
| | fibroblast growth factor receptor; | | |
| | syndecan proteoglycan 1; SDC; | | |
| | CD138; SYND1; syndecan | | |
| BCMA | Tumor necrosis factor receptor | Q02223 | NM_001192 |
| | superfamily member 17 | | |
| | (TNFRSF17); B cell maturation | | |
| | antigen; B-cell maturation factor; | | |
| | B-cell maturation protein; BCM; | | |
| | BCMA; CD269; TNFRSF13A | | |
| CD10 | | | |
| CD5 | T-cell surface glycoprotein CD5; | P06127 | NM_001346456 |
| | CD5 antigen (p56-62); | | NM_014207 |
| | epididymis secretory | | |
| | sperm binding protein; | | |
| | lymphocyte antigen T1/Leu-1; | | |
| | T1; LEU1; CD5 molecule | | |
| CD79a | | | |
| CD79b | | | |

Myeloid Malignancies

| | | | |
|---|---|---|---|
| CD33 | myeloid cell surface antigen | P20138 | NM_001082618 |
| | CD33; CD33 antigen (gp67); | | NM_001177608 |
| | CD33 molecule transcript; gp67; | | NM_001772 |
| | sialic acid-binding Ig-like lectin | | |
| | 3; p67; SIGLEC3; SIGLEC-3 | | |
| CD123 | interleukin-3 receptor subunit | P26951 | NM_001267713 |
| | alpha; CD123 antigen; IL-3 | | NM_002183 |
| | receptor subunit alpha; IL-3R | | |
| | subunit alpha; IL-3R-alpha; IL- | | |
| | 3RA; interleukin 3 receptor, | | |
| | alpha (low affinity); IL3R; | | |
| | CD123; IL3RX; IL3RY; | | |
| | IL3RAY; hIL-3Ra | | |
| FLT3 | receptor-type tyrosine-protein | P36888 | NM_004119 |
| | kinase FLT3; CD135 antigen; FL | | |
| | cytokine receptor; fetal liver | | |
| | kinase 2; fms related tyrosine | | |
| | kinase 3; fms-like tyrosine | | |
| | kinase 3; growth factor | | |
| | receptor tyrosine kinase type | | |
| | III; stem cell tyrosine kinase 1; | | |
| | FLK2; STK1; CD135; FLK-2 | | |
| CLEC1A | C-type lectin domain family 1 | Q8NC01 | NM_001297748 |
| | member A; C-type lectin-like | | NM_001297749 |
| | receptor-1; CLEC1; CLEC-1 | | NM_001297750 |
| | | | NM_001297751 |
| | | | NM_016511 |
| CD56 | neural cell adhesion molecule 1; | P13591 | NM_000615 |
| | antigen recognized by | | NM_001076682 |
| | monoclonal antibody 5.1H11; | | NM_001242607 |
| | neural cell adhesion | | NM_001242608 |
| | molecule, NCAM; CD56; | | NM_001386289 |
| | NCAM; MSK39 | | NM_001386290 |
| | | | NM_001386291 |
| | | | NM_001386292 |
| | | | NM_181351 |
| CD34 | hematopoietic progenitor cell | P28906 | NM_001025109 |
| | antigen CD34; CD34 antigen; | | NM_001773 |
| | CD34 molecule | | |
| CD117 | KIT proto-oncogene, receptor | P10721 | NM_000222 |
| | tyrosine kinase; mast/stem cell | | NM_001093772 |
| | growth factor receptor Kit; c-Kit | | NM_001385284 |
| | protooncogene; p145 c-kit; | | NM_001385285 |
| | piebald trait protein; proto- | | NM_001385286 |
| | oncogene c-Kit; proto- | | NM_001385288 |
| | oncogene tyrosine-protein | | NM_001385290 |
| | kinase Kit; soluble KIT variant | | NM_001385292 |
| | 1; tyrosine-protein kinase Kit; | | |
| | v-kit Hardy-Zuckerman 4 | | |
| | feline sarcoma viral oncogene | | |
| | homolog; v-kit Hardy- | | |
| | Zuckerman 4 feline sarcoma | | |
| | viral oncogene-like protein; | | |
| | PBT; SCFR; C-Kit; CD117; | | |
| | MASTC | | |
| CD14 | CD14 molecule; monocyte | P08571 | NM_000591 |
| | differentiation antigen CD14; | | NM_001040021 |
| | myeloid cell-specific leucine- | | NM_001174104 |
| | rich glycoprotein; Cluster of | | NM_001174105 |
| | Differentiation 14 | | |
| CD133 | prominin-1; antigen AC133; | O43490 | NM_001145847 |
| | hProminin; hematopoietic stem | | NM_001145848 |
| | cell antigen; prominin-like | | NM_001145849 |
| | protein 1; RP41; AC133; | | NM_001145850 |
| | CD133; MCDR2; STGD4; | | NM_001145851 |
| | CORD12; PROML1; | | NM_001145852 |
| | MSTP061 | | NM_001371406 |
| | | | NM_001371407 |
| | | | NM_NM_ |
| | | | 006017_ |
| | | | 001371408 |
| CD44v6 | CD44 molecule variant 6; | P16070-6 | NM_001202555 |
| | CD44 antigen variant 6; | | |
| | CD44 molecule isoform 6; | | |
| CD47 | leukocyte surface antigen | Q08722 | NM_001382306 |
| | CD47; CD47 antigen | | NM_001777 |
| | (Rh-related antigen, | | NM_198793 |
| | integrin-associated | | |
| | signal transducer); CD47 | | |
| | glycoprotein; Rh-related | | |
| | antigen; antigen identified | | |
| | by monoclonal antibody | | |
| | 1D8; antigenic surface | | |
| | determinant protein OA3; | | |
| | integrin associated protein; | | |
| | integrin-associated signal | | |
| | transducer; CD47 molecule | | |
| CD64 | high affinity immunoglobulin | P12314 | NM_000566 |
| | gamma Fc receptor I; Fc | | NM_001378804 |
| | fragment of IgG, high | | NM_001378805 |
| | affinity Ia, receptor (CD64); | | NM_001378806 |
| | Fc fragment of IgG, high | | NM_001378807 |
| | affinity Ia, receptor for | | NM_001378808 |
| | (CD64), Fc gamma receptor | | NM_001378809 |
| | Ia; Fc-gamma RI; Fc- | | NM_001378810 |
| | gamma receptor I A1; IgG Fc | | NM_001378811 |
| | receptor I; fc-gamma RIA; | | |
| | fcgammaRIa; Fc fragment | | |
| | of IgG receptor Ia; CD64; | | |
| | FCRI; CD64A; IGFR1 | | |
| CD96 | T-cell surface protein tactile; | P40200 | NM_001318889 |
| | T cell activation, increased late | | NM_005816 |
| | expression; cell surface antigen | | NM_198196 |
| | CD96; t cell-activated | | |
| | increased late expression | | |
| | protein; TACTILE; | | |
| | CD96 molecule | | |
| CD97 | adhesion G protein-coupled | P48960 | NM_001025160 |
| | receptor E5; CD97 molecule; | | NM_001784 |
| | leukocyte antigen CD97; seven | | NM_078481 |
| | transmembrane helix receptor; | | |
| | seven-span transmembrane | | |
| | protein; seven-transmembrane, | | |
| | heterodimeric receptor | | |
| | associated with inflammation; | | |
| | adhesion G protein-coupled | | |
| | receptor E5; TM7LN1 | | |
| CD99 | CD99 antigen; E2 antigen; | P14209 | NM_001122898 |
| | MIC2 (monoclonal antibody | | NM_001321367 |
| | 12E7); T-cell surface | | NM_001321368 |
| | glycoprotein E2; antigen | | NM_001321369 |
| | identified by monoclonal | | NM_001321370 |
| | 12E7, Y homolog; antigen | | NM_002414 |
| | identified by monoclonal | | |
| | antibodies 12E7, F21 and | | |
| | O13; cell surface antigen | | |
| | 12E7; cell surface antigen | | |
| | HBA-71; cell surface | | |
| | antigen O13; surface antigen | | |
| | MIC2; CD99 molecule (Xg | | |
| | blood group); HBA71; | | |
| | MIC2X; MIC2Y; MSK5X | | |
| CD16 | low affinity immunoglobulin | P08637 | NM_000569 |
| | gamma Fc region receptor III- | | NM_001127592 |
| | A; CD16a antigen; Fc fragment | | NM_001127593 |
| | of IgG, low affinity III, | | NM_001127595 |
| | receptor for (CD16); Fc | | NM_001127596 |
| | fragment of IgG, low | | NM_001329120 |
| | affinity IIIa, receptor (CD16a); | | NM_001329122 |
| | Fc gamma receptor III-A; | | NM_001386450 |
| | Fc-gamma RIII-alpha; | | |
| | Fc-gamma receptor III- | | |
| | 2 (CD 16); Fc-gamma | | |
| | receptor IIIb (CD16); | | |
| | FegammaRIIIA; igG Fc | | |
| | receptor III-2; immuno- | | |
| | globulin G Fc receptor | | |
| | III; low affinity immuno- | | |
| | globulin gamma receptor | | |
| | III-a Fc fragment; neutrophil- | | |
| | specific antigen NA; CD16; | | |
| | FCG3; CD16A; FCGR3; | | |
| | IGFR3; IMD20; FCR-10; | | |
| | FCRIII; FCGRIII; | | |
| | FCRIIIA; Fc fragment of | | |
| | IgG receptor IIIa | | |
| CD45 | receptor-type tyrosine-protein | P08575 | NM_001267798 |
| | phosphatase C; CD45 antigen; | | NM_002838 |
| | T200 glycoprotein; T200 | | NM_080921 |
| | leukocyte common antigen; | | |
| | protein tyrosine phosphatase, | | |
| | receptor type, c polypeptide; | | |
| | protein tyrosine phosphatase | | |
| | receptor type C; PTPRC; | | |
| | LCA; LY5; B220; CD45; | | |
| | L-CA; T200; CD45R; GP180 | | |
| CD9 | CD9 antigen; 5H9 antigen; | P21926 | NM_001330312 |
| | BA-2/p24 antigen; CD9 | | NM_001769 |
| | antigen (p24); antigen CD9; | | |
| | cell growth-inhibiting gene | | |
| | 2 protein; leukocyte antigen | | |
| | MIC3; motility related protein- | | |
| | 1; tetraspanin-29; MIC3; MRP- | | |
| | 1; BTCC-1; DRAP-27; | | |
| | TSPAN29; TSPAN-29; CD9 | | |
| | molecule | | |
| Muc1 | mucin-1; H23 antigen; breast | P15941 | NM_001018016 |
| | carcinoma-associated antigen | | NM_001018017 |
| | DF3; cancer antigen 15-3; | | NM_001044390 |
| | carcinoma-associated mucin; | | NM_001044391 |
| | episialin; krebs von den | | NM_001044392 |
| | Lungen-6; mucin 1, trans- | | NM_001044393 |
| | membrane; peanut-reactive | | NM_001204285 |
| | urinary mucin, polymorphic | | NM_001204286 |
| | epithelial mucin; tumor | | NM_001204287 |
| | associated epithelial mucin; | | NM_001204288 |
| | tumor-associated | | NM_001204289 |
| | epithelial membrane antigen; | | NM_001204290 |
| | EMA; MCD; PEM; PUM; | | NM_001204291 |
| | KL-6; MAM6; MCKD; | | NM_001204292 |
| | PEMT; CD227; H23AG; | | NM_001204293 |
| | MCKD1; MUC-1; | | NM_001204294 |
| | ADMCKD; ADTKD2; | | NM_001204295 |
| | ADMCKD1; CA 15-3; | | NM_001204296 |
| | MUC-1/X; MUC1/ZD; | | NM_001204297 |
| | MUC-1/SEC | | NM_001371720 |
| | | | NM_002456 |
| Lewis-Y | | | |
| IL1RAP | interleukin-1 receptor accessory | Q9NPH3 | NM_001167928 |
| | protein; IL-1 receptor accessory | | NM_001167929 |
| | protein; interleukin-1 receptor 3; | | NM_001167930 |
| | interleukin-1 receptor accessory | | NM_001167931 |
| | protein beta; IL1R3; C3orf13; | | NM_001364879 |
| | IL-IRAcP; IL1RAP | | NM_001364880 |
| | | | NM_001364881 |
| | | | NM_002182 |
| | | | NM_134470 |
| FR-beta | folate receptor beta; folate | P14207 | NM_000803 |
| | receptor 2 (fetal); folate | | NM_001113534 |
| | receptor alpha; folate | | NM_001113535 |
| | receptor, fetal/placental; | | NM_001113536 |
| | folate-binding protein, fetal/ | | |
| | placental; placental folate- | | |
| | binding protein; FBP; FOLR1; | | |
| | FR-P3; FRbeta; FR-BETA; | | |
| | BETA-HFR; FBP/PL-1; | | |
| | FOLR2 | | |

T cell malignancies

| | | | |
|---|---|---|---|
| CD5 | T-cell surface glycoprotein | P06127 | NM_001346456 |
| | CDS; CD5 antigen (p56-62); | | NM_014207 |
| | epididymis secretory sperm | | |
| | binding protein; lymphocyte | | |
| | antigen T1/Leu-1; T1; | | |
| | LEU1; CDS molecule | | |
| CD7 | T-cell antigen CD7; CD7 | P09564 | NM_006137 |
| | antigen (p41); T-cell leukemia | | |
| | antigen; T-cell surface | | |
| | antigen Leu-9; p41 | | |
| | protein; GP40; TP41; Tp40; | | |
| | LEU-9; CD7 molecule | | |
| CD38 | ADP-ribosyl cyclase/cyclic | P28907 | NM_001775 |
| | ADP-ribose hydrolase 1; | | |
| | 2′-phospho-ADP-ribosyl | | |
| | cyclase; 2′-phospho-cyclic- | | |
| | ADP-ribose transferase; | | |
| | ADP-ribosyl cyclase 1; CD38 | | |
| | antigen (p45); NAD(+) | | |
| | nucleosidase; cluster of | | |
| | differentiation 38; cyclic ADP- | | |
| | ribose hydrolase 1; ecto- | | |
| | nicotinamide adenine | | |
| | dinucleotide | | |
| | glycohydrolase; ADPRC1; | | |
| | ADPRC1; CD38 molecule | | |
| CD30 | tumor necrosis factor receptor | P28908 | NM_001243 |
| | superfamily member 8; CD30L | | NM_001281430 |
| | receptor; Ki-1 antigen; cytokine | | |
| | receptor CD30; lymphocyte | | |
| | activation antigen CD30; CD30; | | |
| | Ki-1; D1S166E; TNFRSF8 | | |

Solid Tumors

| | | | |
|---|---|---|---|
| B7-H3 | CD276 antigen; B7 homolog 3; | Q5ZPR3 | NM_001024736 |
| | costimulatory molecule; B7H3; | | NM_001329628 |
| | B7-H3; B7RP-2; 4Ig-B7-H3; | | NM_001329629 |
| | CD276 molecule | | NM_025240 |
| HER2 | receptor tyrosine-protein kinase | P04626 | NR_110535.2 |
| | erbB-2; c-erb B2/neu protein; | | NM_001382782.1 |
| | herstatin; human epidermal | | NM_001289936.2 |
| | growth factor receptor 2; | | NM_001005862.3 |
| | metastatic lymph | | NM_001289938.2 |
| | node gene 19 protein; | | XM_024450643.1 |
| | neuro/glioblastoma derived | | XM_024450642.1 |
| | oncogene homolog; | | XM_024450641.1 |
| | neuroblastoma/glioblastoma | | NM_001382783.1 |
| | derived oncogene homolog; | | NM_001382787.1 |
| | proto-oncogene Neu; | | NM_001382784.1 |
| | proto-oncogene c-ErbB-2; | | NM_001382786.1 |
| | tyrosine kinase-type cell | | NM_001382789.1 |
| | surface receptor HER2; | | NM_001382788.1 |
| | v-erb-b2 avian | | NM_001382785.1 |
| | erythroblastic leukemia viral | | NM_004448.4 |
| | oncogene homolog 2; v-erb- | | NM_001289937.2 |
| | b2 avian erythroblastic | | NM_001382796.1 |
| | leukemia viral | | NM_001382798.1 |
| | oncoprotein 2; v-erb-b2 | | NM_001382800.1 |
| | erythroblastic leukemia viral | | NM_001382797.1 |
| | oncogene homolog 2; | | NM_001382805.1 |
| | neuro/glioblastoma derived | | NM_001382792.1 |
| | oncogene homolog; NEU; | | NM_001382793.1 |
| | NGL; HER2; TKR1; CD340; | | NM_001382803.1 |
| | HER-2; VSCN2; MLN 19; | | NM_001382794.1 |
| | HER-2/neu; erb- | | NM_001382795.1 |
| | b2 receptor tyrosine kinase 2; | | NM_001382801.1 |
| | ERBB2 | | NM_001382790.1 |
| | | | NM_001382806.1 |
| | | | NM_001382802.1 |
| | | | NM_001382799.1 |
| | | | NM_001382791.1 |
| | | | NM_001382804.1 |
| CD44v6 | CD44 molecule variant 6; | P16070-6 | NM_001202555 |
| | CD44 antigen variant 6; CD44 | | |
| | molecule isoform 6; | | |
| CEA | carcinoembryonic antigen- | P06731 | NM_004363.6 |
| | related cell adhesion | | NM_001291484.3 |
| | molecule 5; carcinoembryonic | | XM_017026145.2 |
| | antigen related | | XM_017026146.2 |
| | cell adhesion molecule 5; | | XM_011526322.2 |
| | meconium antigen 100; CEA; | | NM_001308398.2 |
| | CD66e; CEA cell adhesion | | |
| | molecule 5; CEACAM5 | | |
| CD133 | prominin-1; antigen AC133; | O43490 | NM_001145847 |
| | hProminin; hematopoietic stem | | NM_001145848 |
| | cell antigen; prominin- | | NM_001145849 |
| | like protein 1; RP41; AC133; | | NM_001145850 |
| | CD133; MCDR2; STGD4; | | NM_001145851 |
| | CORD12; PROML1; | | NM_001145852 |
| | MSTP061 | | NM_001371406 |
| | | | NM_001371407 |
| | | | NM NM_ |
| | | | 006017_ |
| | | | 001371408 |
| c-Met | hepatocyte growth factor | P08581 | XR_001744772.1 |
| | receptor; HGF receptor; | | NM_001127500.3 |
| | HGF/SF receptor; SF | | NM_000245.4 |
| | receptor; proto-oncogene | | NM_001324402.2 |
| | c-Met; scatter factor receptor; | | NM_001324401.3 |
| | tyrosine-protein kinase | | XM_011516223.1 |
| | Met; HGFR; AUTS9; | | XM_006715990.2 |
| | RCCP2; c-Met; DFNB97; | | NM_005228.5 |
| | MET proto-oncogene, | | NM_001346899.2 |
| | receptor tyrosine kinase; | | NM_001346941.2 |
| | MET | | NM_001346898.2 |
| | | | NM_001346897.2 |
| | | | NM_201284.2 |
| | | | NM_201282.2 |
| | | | NM_201283.2 |
| | | | NM_001346900.2 |
| EGFRvIII | epidermal growth factor | P00533 | |
| | receptor; avian erythroblastic | | |
| | leukemia viral (v-erb-b) | | |
| | oncogene homolog; cell | | |
| | growth inhibiting protein 40; | | |
| | cell proliferation-inducing | | |
| | protein 61; epidermal growth | | |
| | factor receptor tyrosine | | |
| | kinase domain; erb-b2 | | |
| | receptor tyrosine kinase 1; | | |
| | proto-oncogene c-ErbB-1; | | |
| | receptor tyrosine-protein | | |
| | kinase erbB-1; ERBB; | | |
| | ERRP; HER1; mENA; | | |
| | ERBB1; PIG61; NISBD2; | | |
| | epidermal growth factor | | |
| | receptor; EGFR; EGFRvIII | | |
| Epcam | epithelial cell adhesion | P16422 | NM_002354 |
| | molecule; adenocarcinoma- | | |
| | associated antigen; cell | | |
| | surface glycoprotein Trop-1; | | |
| | epithelial glycoprotein 314; | | |
| | human epithelial glycoprotein- | | |
| | 2; major gastrointestinal tumor- | | |
| | associated protein GA733-2; | | |
| | membrane component, | | |
| | chromosome 4, surface marker | | |
| | (35 kD glycoprotein); | | |
| | trophoblast cell surface | | |
| | antigen 1; tumor-associated | | |
| | calcium signal transducer | | |
| | 1; ESA; KSA; M4S1; MK-1; | | |
| | DIAR5; EGP-2; EGP40; | | |
| | KS1/4; MIC18; TROP1; | | |
| | EGP314; HNPCC8; | | |
| | TACSTD1; EPCAM | | |
| EphA2 | ephrin type-A receptor 2; | P29317 | NM_001329090 |
| | epithelial cell receptor protein | | NM_004431 |
| | tyrosine kinase; soluble | | |
| | EPHA2 variant 1; tyrosine- | | |
| | protein kinase receptor | | |
| | ECK; ECK; CTPA; ARCC2; | | |
| | CTPP1; CTRCT6; EPH | | |
| | receptor A2; EPHA2 | | |
| FR- | folate receptor alpha; FR- | P15328 | NM_016724.3 |
| alpha | alpha; KB cells FBP; adult | | NM_016725.3 |
| | folate-binding protein; | | NM_000802.3 |
| | folate binding protein; | | NM_016729.3 |
| | folate receptor 1 (adult); folate | | |
| | receptor, adult; ovarian tumor- | | |
| | associated antigen MOv18; | | |
| | FBP; FOLR; NCFTD; | | |
| | FRalpha; FOLR1 | | |
| GD2 | | | |
| GPC3 | glypican-3; glypican | P51654 | NM_001164619.2 |
| | proteoglycan 3; heparan | | NM_001164618.2 |
| | sulphate proteoglycan; | | NM_004484.4 |
| | intestinal protein OCI-5; | | NM_001164617.2 |
| | secreted glypican-3; SGB; | | XM_017029413.2 |
| | DGSX; MXR7; SDYS; | | |
| | SGBS; OCI-5; SGBS1; | | |
| | GTR2-2; GPC3; glypican 3 | | |
| IL-13R- | interleukin-13 receptor subunit | Q14627 | NM_000640 |
| alpha2 | alpha-2; IL-13 receptor subunit | | |
| | alpha-2; IL-13R subunit alpha- | | |
| | 2; IL-13R-alpha-2; IL-13RA2; | | |
| | cancer/testis antigen 19; | | |
| | interleukin 13 binding protein; | | |
| | interleukin 13 receptor alpha | | |
| | 2 chain; interleukin 13 | | |
| | receptor, alpha 2; CT19; IL- | | |
| | 13R; IL13BP; CD213A2; | | |
| | IL13RA2 | | |
| IL-11R- | interleukin-11 receptor subunit | Q14626 | NM_001142784 |
| alpha | alpha; IL-11 receptor subunit | | |
| | alpha; IL-11R subunit | | |
| | alpha; interleukin 11 | | |
| | receptor, alpha; interleukin-11 | | |
| | receptor alpha chain; | | |
| | CRSDA; IL11RA | | |

In some embodiments, the antigen recognition domain of the CAR described herein binds (e.g. specifically binds) to at least one of L1-CAM, Mesothelin, MUC1, MUC16, NKGD2, PSCA, PSMA, ROR1 and ALK. The antigen specific CAR, when expressed on the cell surface, redirects the specificity of immune cells (e.g. T cells) to the respective antigen.

In some embodiments, the antigen recognition domain of a CAR described herein binds (e.g., specifically binds) to CD22. The CD22-specific CAR, when expressed on the cell surface, redirects the specificity of T cells to human CD22 (see, e.g., Accession Nos. NM_001185099; NM_001185100; NM_001185101; NM_001278417 and NP_001172028; NP_001172029; NP_001172030; NP_001265346; NP_001762).

In some embodiments, the antigen recognition domain of a CAR described herein binds (e.g., specifically binds) to CD19. The CD19-specific CAR, when expressed on the cell surface, redirects the specificity of T cells to human CD19 (see, e.g., Accession Nos. NM_001178098; NM_001770; NM_001385732 and NP_001171569; NP_001761).

### i) Antigen Recognition Domains Comprising an Anti-CD22 Antibody or Fragment Thereof

In some embodiments, the antigen recognition domain of a CAR provided herein comprises an antibody or an antigen-binding fragment thereof. In some embodiments, the antigen recognition domain of a CAR provided herein comprises a single chain antibody fragment (scFv) comprising a light chain variable domain (VL) and heavy chain variable domain (VH) of a monoclonal anti-CD22 antibody. Optionally, the VH and VL may be joined by a flexible linker, such as a glycine-serine linker or a Whitlow linker. In some embodiments, the antigen binding moiety may comprise VH and VL that are directionally linked, for example, from N to C terminus, VH-linker-VL or VL-linker-VH.

In some embodiments, the antigen recognition domain of a CAR provided herein comprises an scFv whose affinity for CD22 has been optimized to induce cytotoxicity of tumor cells that produce high levels or normal levels of CD22. In some embodiments, the antigen recognition domain of a CAR provided herein comprises an scFv whose affinity for CD22 has been optimized to induce cytotoxicity of tumor cells that produce low levels of CD22.

Exemplary anti-CD22 scFvs from which antigen recognition domains for use in a CAR described herein may be derived include, but are not limited to, m971 and immunologically active and/or antigen-binding fragments thereof. Thus, in some embodiments, the antigen recognition domain of a CAR provided herein comprises a VH and VL derived from any one of the anti-CD22 antibody m971. In some embodiments, the antigen recognition domain of a CAR provided herein comprises a VH and VL separated by a linker.

The amino acid sequences of the VH (and corresponding CDRH1, CDRH2, and CDRH3) and VL (and corresponding CDRL1, CDRL2, and CDRL3) of the High-Affinity m971 and Low-Affinity m971 are provided below. The affinity of the “standard affinity” m971 is about KD=3.1 nM. The affinity of the “High Affinity” m971 is about KD=18 pM (Ramakrishna et al, Clin Cancer Res, 2019. PMID: 31110075.)

High Affinity m971 full length-amino acid sequence:
(SEQ ID NO: 208)
MALPVTALLLPLALLLHAARPQVQLQQSGPGMVKPSQTLSLTCAISGDSVSSNSVAWN
WIRQSPSRGLEWLGRTYYRSTWYNDYAVSMKSRITINPDTNKNQFSLQLNSVTPEDTAV
YYCAREVTGDLEDAFDIWGQGTMVTVSSGGGGSGGGGSGGGGSDIQMIQSPSSLSASV
GDRVTITCRASQTIWSYLNWYRQRPGEAPNLLIYAASSLQSGVPSRFSGRGSGTDFTLTIS
SLQAEDFATYYCQQSYSIPQTFGQGTKLEIK
High Affinity m971-VH-amino acid:
(SEQ ID NO: 209)
MALPVTALLLPLALLLHAARPQVQLQQSGPGMVKPSQTLSLTCAISGDSVSSNSVAWN
WIRQSPSRGLEWLGRTYYRSTWYNDYAVSMKSRITINPDTNKNQFSLQLNSVTPEDTAV
YYCAREVTGDLEDAFDIWGQGTMVTVSS
High Affinity m971-VL-amino acid:
(SEQ ID NO: 210)
DIQMIQSPSSLSASVGDRVTITCRASQTIWSYLNWYRQRPGEAPNLLIYAASSLQSGVPSR
FSGRGSGTDFTLTISSLQAEDFATYYCQQSYSIPQTFGQGTKLEIK
High Affinity m971 linker:
(SEQ ID NO: 211)
GGGGSGGGGSGGGGS
High Affinity-M971-CDRH1:
(SEQ ID NO: 212)
GDSVSSNSVA
High Affinity-M971-CDRH2:
(SEQ ID NO: 213)
TYYRSTWYN
High Affinity-M971-CDRH3:
(SEQ ID NO: 86)
AREVTGDLEDAFDI
High Affinity-M971-CDRL1:
(SEQ ID NO: 87)
QTIWSY
High Affinity-M971-CDRL2:
(SEQ ID NO: 88)
AAS
High Affinity-M971-CDRL3:
(SEQ ID NO: 89)
QQSYSIPQT
High Affinity m971 full length-nucleic acid:
(SEQ ID NO: 214)
ATGGCTCTGCCTGTGACAGCTCTGCTGCTGCCTCTGGCCCTGCTGCTCCATGCTGCTA
GACCTCAGGTGCAGCTCCAGCAGTCTGGCCCAGGAATGGTCAAGCCTAGCCAGACC
CTGAGCCTGACCTGCGCCATCAGCGGCGACAGCGTGTCCTCTAACAGCGTCGCCTGG
AACTGGATCAGACAGAGCCCCAGCAGAGGCCTGGAATGGCTGGGCCGGACCTACTA
CCGGTCCACGTGGTACAACGACTACGCCGTGTCCATGAAGTCCCGGATCACCATCAA
CCCCGACACCAACAAGAACCAGTTCTCCCTGCAGCTGAACAGCGTGACCCCTGAGG
ACACCGCCGTGTACTACTGCGCCAGAGAAGTGACCGGCGACCTGGAAGATGCCTTC
GACATCTGGGGCCAGGGCACCATGGTCACCGTGTCTAGCGGAGGCGGCGGAAGCGG
TGGAGGCGGTAGCGGCGGTGGCGGTTCCGACATCCAGATGATCCAGAGCCCTAGCT
CCCTGAGCGCCAGCGTGGGCGACAGAGTGACCATCACCTGTCGGGCCAGCCAGACC
ATCTGGTCCTACCTGAATTGGTATCGGCAGCGGCCAGGCGAGGCCCCTAACCTGCTG
ATCTATGCCGCCAGCAGCCTGCAGAGCGGCGTGCCAAGCAGATTCTCTGGCAGAGG
CTCCGGCACCGACTTCACCCTGACAATCAGTTCCCTGCAGGCCGAGGACTTCGCCAC
CTACTACTGCCAGCAGTCCTACAGCATCCCTCAGACCTTCGGCCAGGGGACCAAGCT
GGAAATCAAG
Standard Affinity m971 full length-amino acid
(SEQ ID NO: 310)
ASATMALPVTALLLPLALLLHAARPQVQLQQSGPGLVKPSQTLSLTCAISGDSVSSNSAA
WNWIRQSPSRGLEWLGRTYYRSKWYNDYAVSVKSRITINPDTSKNQFSLQLNSVTPEDT
AVYYCAREVTGDLEDAFDIWGQGTMVTVSSGGGGSDIQMTQSPSSLSASVGDRVTITCR
ASQTIWSYLNWYQQRPGKAPNLLIYAASSLQSGVPSRFSGRGSGTDFTLTISSLQAEDFA
TYYCQQSYSIPQTFGQGTKLEIK
Standard Affinity m971 linker:
(SEQ ID NO: 215)
GGGGS
Standard Affinity m971 scFV-nucleic acid
(SEQ ID NO: 216)
CTCGAGATGGCTCTGCCTGTGACAGCTCTGCTGCTGCCTCTGGCCCTGCTGCTCCATG
CTGCTAGACCTCAGGTGCAGCTCCAGCAGTCTGGCCCAGGACTGGTCAAGCCTAGCC
AGACCCTGAGCCTGACCTGCGCCATCAGCGGCGACAGCGTGTCCTCTAACAGCGCC
GCCTGGAACTGGATCAGACAGAGCCCCAGCAGAGGCCTGGAATGGCTGGGCCGGAC
CTACTACCGGTCCAAGTGGTACAACGACTACGCCGTGTCCGTGAAGTCCCGGATCAC
CATCAACCCCGACACCAGCAAGAACCAGTTCTCCCTGCAGCTGAACAGCGTGACCC
CTGAGGACACCGCCGTGTACTACTGCGCCAGAGAAGTGACCGGCGACCTGGAAGAT
GCCTTCGACATCTGGGGCCAGGGCACCATGGTCACCGTGTCTAGCGGAGGCGGCGG
AAGCGACATCCAGATGACCCAGAGCCCTAGCTCCCTGAGCGCCAGCGTGGGCGACA
GAGTGACCATCACCTGTCGGGCCAGCCAGACCATCTGGTCCTACCTGAATTGGTATC
AGCAGCGGCCAGGCAAGGCCCCTAACCTGCTGATCTATGCCGCCAGCAGCCTGCAG
AGCGGCGTGCCAAGCAGATTCTCTGGCAGAGGCTCCGGCACCGACTTCACCCTGAC
AATCAGTTCCCTGCAGGCCGAGGACTTCGCCACCTACTACTGCCAGCAGTCCTACAG
CATCCCTCAGACCTTCGGCCAGGGGACCAAGCTGGAAATCAAGACTAGT

In some embodiments, the antigen recognition domain of a CAR described herein comprises complementarity determining regions (CDRs) and/or a heavy chain variable domain (VH) and a light chain variable domain (VL) derived from the anti-CD22 antibody m971. The m971 antibody comprises a VH comprising the amino acid sequence of SEQ ID NO: 82 and a VL comprising the amino acid sequence of SEQ ID NO: 83. The amino acid sequences of the VH (and corresponding CDRH1, CDRH2, and CDRH3) and VL (and corresponding CDRL1, CDRL2, and CDRL3) of m971 are provided below:

M971-VH:
(SEQ ID NO: 82)
QVQLQQSGPGLVKPSQTLSLTCAISGDSVSSNSAAWNWIRQSPSRGLEW
LGRTYYRSKWYNDYAVSVKSRITINPDTSKNQFSLQLNSVTPEDTAVYY
CAREVTGDLEDAFDIWGQGTMVTVSS
M971-VL:
(SEQ ID NO: 83)
DIQMTQSPSSLSASVGDRVTITCRASQTIWSYLNWYQQRPGKAPNLLIY
AASSLQSGVPSRFSGRGSGTDFTLTISSLQAEDFATYYCQQSYSIPQTF
GQGTKLEIK
M971-CDRH1:
(SEQ ID NO: 84)
GDSVSSNSAA
M971-CDRH2:
(SEQ ID NO: 85)
TYYRSKWYN
M971-CDRH3:
(SEQ ID NO: 86)
AREVTGDLEDAFDI
M971-CDRL1:
(SEQ ID NO: 87)
QTIWSY
M971-CDRL2:
(SEQ ID NO: 88)
AAS
M971-CDRL3:
(SEQ ID NO: 89)
QQSYSIPQT

In some embodiments, the antigen recognition domain of a CAR described herein comprises an scFv comprising a VH and a VL, wherein the VH comprises a CDRH1 of SEQ ID NO: 84, a CDRH2 of SEQ ID NO: 85, and a CDRH3 of SEQ ID NO: 86, and the VL comprises a CDRL1 of SEQ ID NO: 87, a CDRL2 of SEQ ID NO: 88, and a CDRL3 of SEQ ID NO: 89. In some embodiments, the antigen recognition domain of a CAR described herein comprising a VH and a VL, wherein the VH comprises the amino acid sequence of SEQ ID NO: 82, and the VL comprises the amino acid sequence of SEQ ID NO: 83.

The antigen recognition domain of the CARs provided herein may include CDRs and/or VH and VL derived from an anti-CD22 antibody (or antigen binding fragment thereof). Anti-CD22 antibodies of the disclosure can comprise any one of the partial light chain sequences known in the art and/or any one of partial heavy chain sequences known in the art. In some embodiments, the antigen recognition domain of a CAR described herein comprises an scFv comprising a VH and a VL, wherein the VH comprises the amino acid sequence of a VH from an anti-CD22 antibody known in the art, and the VL comprises the amino acid sequence of the corresponding VL known in the art.

In some embodiments, the antigen recognition domain of a CAR described herein comprises an scFv comprising a VH and a VL, wherein the VH comprises a CDRH1, a CDRH2, and a CDRH3 each comprising the amino acid sequence of a CDRH1, a CDRH2, and a CDRH3 of an anti-CD22 antibody known in the art, and wherein and the VL comprises a CDRL1, a CDRL2, and a CDRL3 each comprising the amino acid sequence of a CDRL1, a CDRL2, and a CDRL3 of the same anti-CD22 antibody known in the art. Determination of CDR regions is well within the skill of the art. It is understood that in some embodiments, CDRs can be a combination of the Kabat and Chothia CDR (also termed “combined CRs” or “extended CDRs”).

In some embodiments, the CDRs are the Kabat CDRs. In other embodiments, the CDRs are the Chothia CDRs. In other embodiments, the CDRs are IMGT CDRs. In other words, in embodiments with more than one CDR, the CDRs may be any of Kabat, Chothia, IMGT combination CDRs, or combinations thereof.

### ii) Antigen Recognition Domains Comprising an Anti-CD19 Antibody or Fragment Thereof

In some embodiments, the antigen recognition domain of a CAR provided herein comprises an scFv whose affinity for CD19 has been optimized to induce cytotoxicity of tumor cells that produce high levels or normal levels of CD19. In some embodiments, the antigen recognition domain of a CAR provided herein comprises an scFv whose affinity for CD19 has been optimized to induce cytotoxicity of tumor cells that produce low levels of CD19. Illustrative examples of such affinity tuning are provided in Caruso et al. (2015) Cancer Res. 75:3505-18 and Liu et al. (2015) Cancer Res. 75:3596-607.

In some embodiments, the antigen recognition domain of a CAR provided herein comprises an antibody or an antigen-binding fragment thereof. In some embodiments, the antigen recognition domain of a CAR provided herein comprises a single chain antibody fragment (scFv) comprising a light chain variable domain (VL) and heavy chain variable domain (VH) of a monoclonal anti-CD19 antibody. Optionally, the VH and VL may be joined by a flexible linker, such as a glycine-serine linker or a Whitlow linker. In some embodiments, the scFv is humanized. In some embodiments, the antigen binding moiety may comprise VH and VL that are directionally linked, for example, from N to C terminus, VH-linker-VL or VL-linker-VH.

In some embodiments, the antigen recognition domain of a CAR provided herein comprises an scFv whose affinity for CD19 has been optimized to induce cytotoxicity of tumor cells that produce high levels or normal levels of CD19. In some embodiments, the antigen recognition domain of a CAR provided herein comprises an scFv whose affinity for CD19 has been optimized to induce cytotoxicity of tumor cells that produce low levels of CD19.

In some embodiments, the antigen recognition domain of a CAR provided herein comprises an amino acid sequence that is at least 90%, at least 91%, at least 92%, at least 93%, at least 94%, at least 95%, at least 96%, at least 97%, at least 98%, at least 99% or at least 100% identical to the amino acid sequence of SEQ ID NOs: 90.

In some embodiments, the antigen recognition domain of a CAR provided herein comprises an amino acid sequence that is at least 90%, at least 91%, at least 92%, at least 93%, at least 94%, at least 95%, at least 96%, at least 97%, at least 98%, at least 99% or at least 100% identical to the amino acid sequence of any one of SEQ ID NO: 91.

Exemplary anti-CD19 scFvs from which antigen recognition domains for use in a CAR described herein may be derived include, but are not limited to, FMC63 and immunologically active and/or antigen-binding fragments thereof. Thus, in some embodiments, the antigen recognition domain of a CAR provided herein comprises a VH and VL derived from any one of the anti-CD19 antibodies FMC63.

Exemplary anti-CD19 scFvs from which antigen recognition domains for use in a CAR described herein may be derived include, but are not limited to, inebilizumab (MEDI-551), MDX-1342, tafasitamab, obexelimab, B4 (Merck), hA19 (immunomedics), and immunologically active and/or antigen-binding fragments thereof. Thus, in some embodiments, the antigen recognition domain of a CAR provided herein comprises a VH and VL derived from any one of these anti-CD19 antibodies.

In some embodiments, the antigen recognition domain of a CAR described herein comprises complementarity determining regions (CDRs) and/or a heavy chain variable domain (VH) and a light chain variable domain (VL) derived from the anti-CD19 antibody FMC63. The FMC63 antibody comprises a VH comprising the amino acid sequence of SEQ ID NO: 92 and a VL comprising the amino acid sequence of SEQ ID NO: 93. The amino acid sequences of the VH (and corresponding CDRH1, CDRH2, and CDRH3) and VL (and corresponding CDRL1, CDRL2, and CDRL3) of FMC63 are provided below:

FMC63-VH:
(SEQ ID NO: 92)
EVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTSVTV
FMC63-VL:
(SEQ ID NO: 93)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEIT
FMC63-CDRH1:
(SEQ ID NO: 94)
GVSLPDYG
FMC63-CDRH2:
(SEQ ID NO: 95)
IWGSETT
FMC63-CDRH3:
(SEQ ID NO: 96)
AKHYYYGGSYAMDY
FMC63-CDRL1:
(SEQ ID NO: 97)
QDISKY
FMC63-CDRL2:
(SEQ ID NO: 98)
HTS
FMC63-CDRL3:
(SEQ ID NO: 99)
QQGNTLPYT

In some embodiments, the antigen recognition domain of a CAR described herein comprises an scFv comprising a VH and a VL, wherein the VH comprises a CDRH1 of SEQ ID NO: 94, a CDRH2 of SEQ ID NO: 95, and a CDRH3 of SEQ ID NO: 96, and the VL comprises a CDRL1 of SEQ ID NO: 97, a CDRL2 of SEQ ID NO: 98, and a CDRL3 of SEQ ID NO: 99. In some embodiments, the antigen recognition domain of a CAR described herein comprising a VH and a VL, wherein the VH comprises the amino acid sequence of SEQ ID NO: 92, and the VL comprises the amino acid sequence of SEQ ID NO: 93.

The antigen recognition domain of the CARs provided herein may include CDRs and/or VH and VL derived from an anti-CD19 antibody (or antigen binding fragment thereof) Anti-CD19 antibodies of the disclosure can comprise any one of the partial light chain sequences known in the art and/or any one of partial heavy chain sequences known in the art. In some embodiments, the antigen recognition domain of a CAR described herein comprises an scFv comprising a VH and a VL, wherein the VH comprises the amino acid sequence of a VH from an anti-CD19 antibody known in the art, and the VL comprises the amino acid sequence of the corresponding VL from an anti-CD19 antibody known in the art.

In some embodiments, the antigen recognition domain of a CAR described herein comprises an scFv comprising a VH and a VL, wherein the VH comprises a CDRH1, a CDRH2, and a CDRH3 each comprising the amino acid sequence of a CDRH1, a CDRH2, and a CDRH3 of an anti-CD19 antibody known in the art, and wherein and the VL comprises a CDRL1, a CDRL2, and a CDRL3 each comprising the amino acid sequence of a CDRL1, a CDRL2, and a CDRL3 of the same anti-CD19 antibody known in the art. Determination of CDR regions is well within the skill of the art. It is understood that in some embodiments, CDRs can be a combination of the Kabat and Chothia CDR (also termed “combined CRs” or “extended CDRs”).

In some embodiments, the CDRs are the Kabat CDRs. In other embodiments, the CDRs are the Chothia CDRs. In other embodiments, the CDRs are IMGT CDRs. In other words, in embodiments with more than one CDR, the CDRs may be any of Kabat, Chothia, IMGT combination CDRs, or combinations thereof.

### B. Signal Peptides

In some embodiments, any of the CARs provided herein comprises a signal peptide (also known as a signal peptide, signal sequence, signal peptide sequence, leader peptide, and leader peptide sequence). In some embodiments, the antigen recognition domain of the CAR described herein comprises a signal peptide or a leader peptide sequence. Exemplary signal sequences include but are not limited to a CD8α signal sequence or an IgG signal sequence. In some embodiments, the CAR described herein does not comprise a signal peptide. In some embodiments, the T cell or populations of T cells provided herein comprise a CAR comprising a signal peptide. In some embodiments, the T cell or populations of T cell provided herein comprise a CAR that does not comprise a signal peptide.

In some embodiments, the CAR (e.g., the antigen recognition domain of the CAR) may comprise a human CD8α signal sequence comprising an amino acid sequence having at least 90%, 91%, 92%, 93%, 94%, 95%, 96%, 97%, 98%, 99% or 100% identity with the amino acid sequence of SEQ ID NO: 1.

In some embodiments, the CAR (e.g., the antigen recognition domain of the CAR) may comprise a human CD8α signal sequence comprising an amino acid sequence having at least 90%, 91%, 92%, 93%, 94%, 95%, 96%, 97%, 98%, 99% or 100% identity with the amino acid sequence of SEQ ID NO: 2.

In some embodiments, the CAR (e.g., the antigen recognition domain of the CAR) may comprise a human IgG signal sequence comprising an amino acid sequence having at least 90%, 91%, 92%, 93%, 94%, 95%, 96%, 97%, 98%, 99% or 100% identity with the amino acid sequence of SEQ ID NO: 3.

In some embodiments, the CAR (e.g., the antigen recognition domain of the CAR) may comprise a human IgG signal sequence comprising an amino acid sequence having at least 90%, 91%, 92%, 93%, 94%, 95%, 96%, 97%, 98%, 99% or 100% identity with the amino acid sequence of SEQ ID NO: 4.

### C. Hinge Domains

In some embodiments, a hinge domain (also known as a spacer region or a stalk region) is located between the antigen recognition domain and the transmembrane domain of the CAR. In particular, stalk regions are used to provide more flexibility and accessibility for the extracellular antigen recognition domain. In some embodiments, a hinge domain may comprise up to about 300 amino acids. In some embodiments, the hinge comprises about 10 to about 100 amino acids in length. In some embodiments, the hinge comprises about 25 to about 50 amino acids in length. In some embodiments, the hinge domain establishes an optimal effector-target inter membrane distance. In some embodiments, the hinge domain provides flexibility for antigen recognition domain to bind the target antigen. Any protein that is stable and/or dimerizes can serve this purpose.

A hinge domain may be derived from all or part of naturally occurring molecules, such as from all or part of the extracellular region of CD8, CD8α, CD4, CD28, 4-1BB, or IgG (in particular, the hinge domain of an IgG, for example from IgG1, IgG2 or IgG4), or from all or part of an antibody heavy-chain constant region. Alternatively, the hinge domain may be a synthetic sequence that corresponds to a naturally occurring hinge sequence, or may be an entirely synthetic hinge sequence. In some embodiments, it corresponds to Fc domains of a human immunoglobulin, e.g., either the CH2 or CH3 domain. In some embodiments, the CH2 and CH3 hinge domain of a human immunoglobulin that has been modified to improve dimerization. In some embodiments, the hinge is a hinge portion of an immunoglobulin. In some embodiments, the hinge domain comprises a CH3 region of a human immunoglobulin. In some embodiments, the hinge domain comprises a CH2 and CH3 region of a human immunoglobulin. In some embodiments, the CH2 region comprises a human IgG1, IgG2 or IgG4 immunoglobulin CH2 region.

In some embodiments, the hinge domain is a part of human CD8α chain (e.g., NP_001139345.1). In some embodiments, the hinge domain of CARs described herein comprises a subsequence of CD8α, CD28, or the constant region of an immunoglobulin (e.g. IgG1, IgG2, IgG3, IgG4) either in wild-type form or mutated to avoid Fc-receptor binding in particular the hinge domain of any of an CD8α, or a CD28. In some embodiments, the stalk region comprises a human CD8α hinge, or a human CD28 hinge.

In some embodiments, the hinge may comprise or consist of a human CD8g. hinge domain comprising an amino acid sequence having at least 90%, 91%, 92%, 93%, 94%, 95%, 96%, 97%, 98%, 99% or 100% identity with the amino acid sequence of SEQ ID NO: 5.

In some embodiments, the hinge may comprise or consist of a human CD8α hinge domain comprising an amino acid sequence having at least 90%, 91%, 92%, 93%, 94%, 95%, 96%, 97%, 98%, 99% or 100% identity with the amino acid sequence of SEQ ID NO: 6.

In some embodiments, the hinge may comprise or consist of a human CD28 hinge domain comprising an amino acid sequence having at least 90%, 91%, 92%, 93%, 94%, 95%, 96%, 97%, 98%, 99% or 100% identity with the amino acid sequence of SEQ ID NO: 7.

In some embodiments, the hinge may comprise or consist of a human CD28 hinge domain comprising an amino acid sequence having at least 90%, 91%, 92%, 93%, 94%, 95%, 96%, 97%, 98%, 99% or 100% identity with the amino acid sequence of SEQ ID NO: 8.

### D. Transmembrane Domains

Suitable transmembrane domains for a CAR disclosed herein have the ability to (a) be expressed at the surface of a cell, which is in some embodiments an immune cell such as, for example a T cell, and/or (b) interact with the ligand-binding domain and intracellular signaling domain for directing cellular response of an immune cell against a predefined target cell. The transmembrane domain can be derived either from a natural or from a synthetic source. The transmembrane domain can be derived from any membrane-bound or transmembrane protein. As non-limiting examples, the transmembrane domains can include the transmembrane region(s) of alpha, beta, delta, or gamma of the T-cell receptor; or a transmembrane region from CD8, CD8α, CD8 beta, CD28, CD3-epsilon, CD3-delta, CD3-gamma, CD3z, CD4, 4-1BB, OX40, ICOS, PD-1, LAG-3, 2B4 or BTLA transmembrane domain or a portion of any of the foregoing or a combination of any of the foregoing. In some embodiments, the transmembrane domain comprises a CD8α transmembrane domain. In some embodiments, the transmembrane domain comprises a CD28 transmembrane domain.

Alternatively, the transmembrane domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the intracellular domain of a CAR. In some embodiments, the linker is a glycine-serine linker.

In some embodiments, the transmembrane domain of a CAR provided herein may comprise or consist of a human CD8α transmembrane domain comprising an amino acid sequence having at least 90%, 91%, 92%, 93%, 94%, 95%, 96%, 97%, 98%, 99% or 100% identity with the amino acid sequence of SEQ ID NO: 13.

In some embodiments, the transmembrane domain of a CAR provided herein may comprise or consist of a human CD28 transmembrane domain comprising an amino acid sequence having at least 90%, 91%, 92%, 93%, 94%, 95%, 96%, 97%, 98%, 99% or 100% identity with the amino acid sequence of SEQ ID NO: 14.

### E. Costimulatory Domains

The intracellular domain of a CAR provided herein may comprise one or more costimulatory domains. Exemplary costimulatory domains include, but are not limited to a 4-1BB (CD137), CD28, CD97, CD11a-CD18, CD2, ICOS, CD27, CD154, CD8α, OX40 (CD134), ZAP40, CD30, GITR, HVEM, DAP10, DAP12, MyD88, 2B4 costimulatory domain, or a fragment thereof, or a combination thereof. In some instances, a first CAR described herein comprises one or more, or two or more of costimulatory domains selected from a 4-1BB (CD137), CD28, CD97, CD11a-CD18, CD2, ICOS, CD27, CD154, CD8α, OX40 (CD134), ZAP40, CD30, GITR, HVEM, DAP10, DAP12, MyD88, 2B4 costimulatory domain, or a fragment thereof, or a combination thereof. In some embodiments, a CAR described herein comprises a CD28 costimulatory domain or a fragment thereof. In some embodiments, a CAR described herein comprises a 4-1BB (CD137) costimulatory domain or a fragment thereof.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 15.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 16.

In some embodiments, the costimulatory domain of a CAR provided herein may comprise or consist of a human 4-1BB costimulatory domain comprising an amino acid sequence having at least 90%, 91%, 92%, 93%, 94%, 95%, 96%, 97%, 98%, 99% or 100% identity with the amino acid sequence of SEQ ID NO: 17.

### F. Activation Domain

In some embodiments, the activation domain of a CAR disclosed herein is responsible for activation of at least one of the normal effector functions of the immune cell (e.g. T cell) in which the CAR is expressed. The terms “intracellular signaling domain” or “intracellular domain” are used interchangeably and refer to a domain that comprises a co-stimulatory domain and/or an activation domain. The term “effector function” refers to a specialized function of a cell. Effector function of a T-cell, for example, may be cytolytic activity or helper activity including the secretion of cytokines. The term “activation domain” refers to the portion of a protein which transduces the effector function signal and directs the cell to perform a specialized function. While usually an entire activation domain can be employed, in many cases it is not necessary to use the entire chain. To the extent that a truncated portion of the activation domain is used, such truncated portion may be used in place of the intact chain as long as it transduces the effector function signal. The term activation domain is thus meant to include any truncated portion of the activation domain sufficient to transduce the effector function signal. In some embodiments, the activation domain further comprises a signaling domain for T-cell activation. In some instances, the signaling domain for T-cell activation comprises an intracellular domain derived from CD3ζ (CD3zeta; CD3z) or an intracellular domain derived from LAT In some embodiments, the CAR described herein comprises at least one (e.g., one, two, three, or more) activation domains selected from a CD3ζ or LAT activation domain, or a portion of any of the foregoing. In some embodiments, the CAR described herein has an activation domain comprising a domain derived from CD3ζ (CD3zeta; CD3z). In some embodiments, the CAR described herein has an activation domain comprising a domain derived from LAT.

In some embodiments, the activation domain of a CAR described herein may comprise or consist of a CD3zeta activation domain (e.g., a human CD3zeta activation domain) comprising an amino acid sequence having at least 90%, 91%, 92%, 93%, 94%, 95%, 96%, 97%, 98%, 99% or 100% identity with the amino acid sequence of SEQ ID NO: 24.

In some embodiments, the activation domain of a CAR described herein may comprise or consist of a CD3zeta activation domain (e.g., a human CD3zeta activation domain) comprising an amino acid sequence having at least 90%, 91%, 92%, 93%, 94%, 95%, 96%, 97%, 98%, 99% or 100% identity with the amino acid sequence of SEQ ID NO: 25.

In some embodiments, the CD3zeta activation domain comprises a mutation in an ITAM domain. Examples of mutations in ITAM domains of CD3zeta are provided in Feucht et al., Nat Med. 2019; 25 (1): 82-88. In some embodiments, each of the two tyrosine residues in one or more of ITAM1, ITAM2, or ITAM3 domains of the CD3zeta activation domain are point-mutated to a phenylalanine residue. In some embodiments, the CD3zeta activation domain comprises a deletion of one or more of the ITAM1, ITAM2, or ITAM3 domains.

In some embodiments, the activation domain of a CAR described herein may comprise or consist of a LAT activation domain (e.g., a human LAT activation domain) comprising an amino acid sequence having at least 90%, 91%, 92%, 93%, 94%, 95%, 96%, 97%, 98%, 99% or 100% identity with the amino acid sequence of any one of SEQ ID NOs: 26-34.

In some embodiments, the LAT activation domain comprises a mutation in a ubiquitination site.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 27.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 28

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 29.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 30.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 31.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 26 having a substitution of arginine for the lysine (K25R) at position 25 of SEQ ID NO: 26, a substitution of glutamic acid for the glycine at position 133 (G133E) of SEQ ID NO: 26, a substitution of arginine for the lysine at position 206 (K206R) of SEQ ID NO: 26, or any combination of the preceding substitutions.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 32 having a substitution of arginine for the lysine (K25R) at position 25 of SEQ ID NO: 32, a substitution of glutamic acid for the glycine at position 104 (G104E) of SEQ ID NO: 32, a substitution of arginine for the lysine at position 177 (K177R) of SEQ ID NO: 32, or any combination of the preceding substitutions.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 33 having a substitution of arginine for the lysine (K25R) at position 25 of SEQ ID NO: 33, a substitution of glutamic acid for the glycine at position 103 (G103E) of SEQ ID NO: 33, a substitution of arginine for the lysine at position 176 (K176R) of SEQ ID NO: 33, or any combination of the preceding substitutions.

In some embodiments, the activation domain of a CAR provided herein may comprise or consist of a LAT intracellular domain comprising an amino acid sequence having at least 90%, 91%, 92%, 93%, 94%, 95%, 96%, 97%, 98%, 99% or 100% identity with the amino acid sequence of SEQ ID NO: 34 having a substitution of arginine for the lysine (K25R) at position 25 of SEQ ID NO: 34, a substitution of glutamic acid for the glycine at position 132 (G132E) of SEQ ID NO. 34, a substitution of arginine for the lysine at position 205 (K205R) of SEQ ID NO: 34, or any combination of the preceding substitutions.

Included in the scope of the invention are nucleic acid sequences that encode functional portions of the CAR described herein. Functional portions encompass, for example, those parts of a CAR that retain the ability to recognize target cells, or detect, treat, or prevent a disease, to a similar extent, the same extent, or to a higher extent, as the parent CAR.

In embodiments, the CARs described herein contain additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

The term “functional variant,” as used herein in reference to a CAR, refers to a CAR, a polypeptide, or a protein having substantial or significant sequence identity or similarity to the CAR encoded by a nucleic acid sequence, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99% identical to the nucleic acid sequence encoding the parent CAR.

A CAR described herein include (including functional portions and functional variants thereof) glycosylated, amidated, carboxylated, phosphorylated, esterified, N-acylated, cyclized via, e.g., a disulfide bridge, or converted into an acid addition salt and/or optionally dimerized or polymerized.

Table 8 provides exemplary amino acid sequences of the domains which can be used in the CARs described herein. In some embodiments, a CAR provided herein comprises one or more domains described in Table 8, or a fragment or portion thereof.

TABLE 8
Exemplary Amino Acid Sequences of CAR Domains

| | | |
|---|---|---|
| | | SEQ ID |
| Exemplary CAR domains | Amino Acid Sequence | NO: |

SIGNAL PEPTIDE

| | | |
|---|---|---|
| human CD8alpha signal | ASATMALPVTALLLPLALLIHAARP | 1 |
| sequence | | |
| human CD8alpha signal | MALPVTALLLPLALLLHAARP | 2 |
| sequence | | |
| human IgG heavy chain signal | GSMEFGLSWLFLVAILKGVQCSR | 3 |
| sequence | | |
| human IgG heavy chain signal | MEFGLSWLFLVAILKGVQCSR | 4 |
| sequence | | |

HINGES

| | | |
|---|---|---|
| human CD8alpha hinge | LETTTPAPRPPTPAPTIASQPLSLRPEACRPAAGGAVHTRG | 5 |
| domain | LDFACD | |
| human CD8alpha hinge | TTTPAPRPPTPAPTIASQPLSLRPEACRPAAGGAVHTRGLD | |
| domain | FACD | |
| human CD28 hinge domain | SRIEVMYPPPYLDNEKSNGTIIHVKGKHLCPSPLFPGPSKP | 7 |
| human CD28 hinge domain | IEVMYPPPYLDNEKSNGTIIHVKGKHLCPSPLFPGPSKP | 8 |
| human IgG1 hinge domain | EPKSCDKTHTCPPCPAPELLGGPSVFLFPPKPKDTLMISRT | 9 |
| | PEVTCVVVDVSHEDPEVKFNWYVDGVEVHNAKTKPREEQYN | |
| | STYRVVSVLTVLHQDWLNGKEYKCKVSNKALPAPIEKTISK | |
| | AKGQPREPQVYTIPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| human IgG1 hinge domain | EPKSCDKTHTCP | 10 |
| human IgG4 hinge domain | ESKYGPPCPSCPAPEFLGGPSVFLFPPKPKDTLMISRTPEV | 11 |
| | TCVVVDVSQEDPEVQFNWYVDGVEVHNAKTKPREEQFNSTY | |
| | RVVSVLTVLHQDWLNGKEYKCKVSNKGLPSSIEKTISKAKG | |
| | QPREPQVYTLPPSQEEMTKNQVSLTCLVKGFYPSDIAVEWE | |
| | SNGQPENNYKTTPPVLDSDGSFFLYSRLTVDKSRWQEGNVF | |
| | SCSVMHEALHNHYTQKSLSLSLGK | |
| human IgG4 hinge domain | ESKYGPPCPSCP | 12 |

TRANSMEMBRANE

| | | |
|---|---|---|
| human CD8alpha | IYIWAPLAGTCGVLLLSLVITLYC | 13 |
| transmembrane domain | | |
| human CD28 transmembrane | FWVLVVVGGVLACYSLLVTVAFIIFWV | 14 |
| domain | | |

COSTIMULATORY DOMAINS

| | | |
|---|---|---|
| human CD28 costimulatory | RSKRSRGGHSDYMNMTPRRPGPTRKHYQPYAPPRDFA | 15 |
| domain | AYRS | |
| human CD28 costimulatory | RSKRSRLLHSDYMNMTPRRPGPTRKHYQPYAPPRDEA | 16 |
| domain | AYRS | |
| human 4-1BB costimulatory | KRGRKKLLYIFKQPFMRPVQTTQEEDGCSCRFPEEEE | 17 |
| domain | GGCEL | |
| human DAP10 costimulatory | LCARPRRSPAQEDGKVYINMPGRG | 18 |
| domain | | |
| human DAP12 costimulatory | YFLGRLVPRGRGAAEAATRKQRITETESPYQELQGQR | 19 |
| domain | SDVYSDINTQRPYYK | |
| human 2B4 costimulatory | WRRKRKEKQSETSPKEFLTIYEDVKDLKTRRNHEQEQ | 20 |
| domain | TFPGGGSTIYSMIQSQSSAPTSQEPAYTLYSLIQPSR | |
| | KSGSRKRNHSPSENSTIYEVIGKSQPKAQNPARLSRK | |
| | ELENFDVYS | |
| human OX40 costimulatory | ALYLLRRDQRLPPDAHKPPGGGSERTPIQEEQADAHS | 21 |
| domain | TLAKI | |
| human CD27 costimulatory | HQRRKYRSNKGESPVEPAEPCHYSCPREEEGSTIPIQ | 22 |
| domain | EDYRKPEPACSP | |
| human CD27 costimulatory | QRRKYRSNKGESPVEPAEPCHYSCPREEEGSTIPIQE | 23 |
| domain | DYRKPEPACSP | |

ACTIVATION DOMAINS

| | | |
|---|---|---|
| human CD3zeta intracellular | DIRVKFSRSADAPAYQQGQNQLYNELNLGRREEYDVL | 24 |
| signaling domain | DKRRGRDPEMGGKPRRKNPQEGLYNELQKDKMAEAYS | |
| | EIGMKGERRRGKGHDGLYQGLSTATKDTYDALHMQAL | |
| | PPR | |
| human CD3zeta intracellular | RVKFSRSADAPAYQQGQNQLYNELNLGRREEYDVLDK | 25 |
| signaling domain | RRGRDPEMGGKPRRKNPQEGLYNELQKDKMAEAYSEI | |
| | GMKGERRRGKGHDGLYQGLSTATKDTYDALHMQALPP | |
| | R | |
| human LAT intracellular | HCHRLPGSYDSTSSDSLYPRGIQFKRPHTVAPWPPAY | 26 |
| signaling domain (“LAT- | PPVTSYPPLSQPDLLPIPRSPQPLGGSHRTPSSRRDS | |
| WT”) | DGANSVASYENEGASGIRGAQAGWGVWGPSWTRLTPV | |
| | SLPPEPACEDADEDEDDYHNPGYLVVLPDSTPATSTA | |
| | APSAPALSTPGIRDSAFSMESIDDYVNVPESGESAEA | |
| | SLDGSREYVNVSQELHPGAAKTEPAALSSQEAEEVEE | |
| | EGAPDYENLQELN | |
| human LAT intracellular | HCHRLPGSYDSTSSDSLYPRGIQFRRPHTVAPWPPAY | 27 |
| signaling domain (“K52R” | PPVTSYPPLSQPDLLPIPRSPQPIGGSHRTPSSRRDS | |
| or “LAT-K52R”) | DGANSVASYENEGASGIRGAQAGWGVWGPSWIRLTPV | |
| | SLPPEPACEDADEDEDDYHNPGYLVVLPDSTPATSTA | |
| | APSAPALSTPGIRDSAFSMESIDDYVNVPESGESAEA | |
| | SLDGSREYVNVSQELHPGAAKTEPAALSSQEAEEVEE | |
| | EGAPDYENLQELN | |
| human LAT intracellular | HCHRLPGSYDSTSSDSLYPRGIQFKRPHTVAPWPPAY | 28 |
| signaling domain (“K233R” | PPVTSYPPLSQPDLLPIPRSPQPLGGSHRTPSSRRDS | |
| or “LAT-K233R”) | DGANSVASYENEGASGIRGAQAGWGVWGPSWIRLTPV | |
| | SLPPEPACEDADEDEDDYHNPGYLVVLPDSTPATSTA | |
| | APSAPALSTPGIRDSAFSMESIDDYVNVPESGESAEA | |
| | SLDGSREYVNVSQELHPGAARTEPAALSSQEAEEVEE | |
| | EGAPDYENLQELN | |
| human LAT intracellular | HCHRLPGSYDSTSSDSLYPRGIQFRRPHTVAPWPPAY | 29 |
| signaling domain (“K52R + | PPVTSYPPLSQPDLLPIPRSPQPLGGSHRTPSSRRDS | |
| K233R” or “LAT- | DGANSVASYENEGASGIRGAQAGWGVWGPSWIRLTPV | |
| K52R + K233R”) | SIPPEPACEDADEDEDDYHNPGYLVVLPDSTPATSTA | |
| | APSAPALSTPGIRDSAFSMESIDDYVNVPESGESAEA | |
| | SLDGSREYVNVSQELHPGAARTEPAALSSQEAEEVEE | |
| | EGAPDYENLQELN | |
| human LAT intracellular | HCHRLPGSYDSTSSDSLYPRGIQFRRPHTVAPWPPAY | 30 |
| signaling domain | PPVTSYPPLSQPDLLPIPRSPQPLGGSHRTPSSRRDS | |
| (“K52R + G160E” or “LAT- | DGANSVASYENEGASGIRGAQAGWGVWGPSWTRLTPV | |
| K52R + G160E”) | SLPPEPACEDADEDEDDYHNPEYLVVLPDSTPATSTA | |
| | APSAPALSTPGIRDSAFSMESIDDYVNVPESGESAEA | |
| | SLDGSREYVNVSQELHPGAAKTEPAALSSQEAEEVEE | |
| | EGAPDYENLQELN | |
| human LAT intracellular | HCHRLPGSYDSTSSDSLYPRGIQFRRPHTVAPWPPAY | 31 |
| signaling domain | PPVTSYPPLSQPDLLPIPRSPQPLGGSHRTPSSRRDS | |
| (“K52R + K233R + G160E” or | DGANSVASYENEGASGIRGAQAGWGVWGPSWIRLTPV | |
| “LAT- | SLPPEPACEDADEDEDDYHNPEYLVVLPDSTPATSTA | |
| K52R + K233R + G160E”) | APSAPALSTPGIRDSAFSMESIDDYVNVPESGESAEA | |
| | SLDGSREYVNVSQELHPGAARTEPAALSSQEAEEVEE | |
| | EGAPDYENLQELN | |
| human LAT intracellular | HCHRLPGSYDSTSSDSLYPRGIQFKRPHTVAPWPPAY | 32 |
| signalling domain alternative | PPVTSYPPLSQPDLLPIPRSPQPLGGSHRTPSSRRDS | |
| isoform | DGANSVASYENEEPACEDADEDEDDYHNPGYLVVLPD | |
| | STPATSTAAPSAPALSTPGIRDSAFSMESIDDYVNVP | |
| | ESGESAEASLDGSREYVNVSQELHPGAAKTEPAALSS | |
| | QEAEEVEEEGAPDYENLQELN | |
| human LAT intracellular | HCHRLPGSYDSTSSDSLYPRGIQEKRPHTVAPWPPAY | 33 |
| signalling domain alternative | PPVTSYPPLSQPDLLPIPSPQPLGGSHRTPSSRRDSD | |
| isoform | GANSVASYENEEPACEDADEDEDDYHNPGYLVVLPDS | |
| | TPATSTAAPSAPALSTPGIRDSAFSMESIDDYVNVPE | |
| | SGESAEASLDGSREYVNVSQELHPGAAKTEPAALSSQ | |
| | EAEEVEEEGAPDYENLQELN | |
| human LAT intracellular | HCHRLPGSYDSTSSDSLYPRGIQFKRPHTVAPWPPAY | 34 |
| signalling domain alternative | PPVTSYPPLSQPDLLPIPSPQPLGGSHRTPSSRRDSD | |
| isoform | GANSVASYENEGASGIRGAQAGWGVWGPSWIRLTPVS | |
| | LPPEPACEDADEDEDDYHNPGYLVVLPDSTPATSTAA | |
| | PSAPALSTPGIRDSAFSMESIDDYVNVPESGESAEAS | |
| | LDGSREYVNVSQELHPGAAKTEPAALSSQEAEEVEEE | |
| | GAPDYENLQELN | |

Table 9 provides exemplary nucleic acid sequences of the domains which can be used to encode the CARs described herein. In some embodiments, a nucleic acid sequence encoding a CAR provided herein comprises one or more sequences described in Table 9, or a fragment or portion thereof.

TABLE 9
Exemplary Nucleic Acid Sequences of CAR Domains

| | | |
|---|---|---|
| | | SEQ ID |
| Exemplary CAR domains | Nucleic Acid Sequence | NO: |

SIGNAL PEPTIDE

| | | |
|---|---|---|
| human CD8alpha signal | GCTAGCGCCACCATGGCTCTGCCTGTGACAGCTCTGC | 35 |
| sequence | TGCTGCCTCTGGCCCTGCTGCTCCATGCTGCTAGACC | |
| | T | |
| human CD8alpha signal | ATGGCTCTGCCTGTGACAGCTCTGCTGCTGCCTCTGG | 36 |
| sequence | CCCTGCTGCTCCATGCTGCTAGACCT | |
| human IgG heavy chain signal | GGATCCATGGAGTTTGGCCTGAGCTGGCTGTTCCTGG | 37 |
| sequence | TGGCCATCCTCAAGGGCGTGCAGTGCTCCAGG | |
| human IgG heavy chain signal | ATGGAGTTTGGCCTGAGCTGGCTGTTCCTGGTGGCCA | 38 |
| sequence | TCCTCAAGGGCGTGCAGTGCTCCAGG | |

HINGES

| | | |
|---|---|---|
| human CD8alpha hinge | CTCGAGACCACCACCCCCGCCCCTAGGCCTCCCACACCTGC | 39 |
| domain | CCCCACAATCGCCTCCCAGCCTCTCAGCCTGAGGCCTGAAG | |
| | CTTGCAGGCCCGCTGCCGGAGGAGCTGTCCATACCAGGGGA | |
| | CTCGACTTCGCCTGCGAC | |
| human CDSalpha hinge | ACCACCACCCCCGCCCCTAGGCCTCCCACACCTGCCCCCAC | 40 |
| domain | AATCGCCTCCCAGCCTCTCAGCCTGAGGCCTGAAGCTTGCA | |
| | GGCCCGCTGCCGGAGGAGCTGTCCATACCAGGGGACTCGAC | |
| | TTCGCCTGCGAC | |
| human CD28 hinge domain | TCTAGAATCGAAGTGATGTACCCTCCACCTTACCTGGACAA | 41 |
| | CGAGAAGTCCAACGGCACCATCATCCACGTGAAGGGCAAGC | |
| | ACCTGTGTCCTTCTCCACTGTTCCCCGGACCTAGCAAGCCT | |
| human CD28 hinge domain | ATCGAAGTGATGTACCCTCCACCTTACCTGGACAACGAGAA | 42 |
| | GTCCAACGGCACCATCATCCACGTGAAGGGCAAGCACCTGT | |
| | GTCCTTCTCCACTGTTCCCCGGACCTAGCAAGCCT | |
| human IgG1 hinge domain | GAGCCCAAGAGCTGCGACAAGACCCACACCTGCCCCCCCTG | 43 |
| | CCCCGCCCCCGAGCTGCTGGGCGGCCCCAGCGTGTTCCTGT | |
| | TCCCCCCCAAGCCCAAGGACACCCTGATGATCAGCCGGACC | |
| | CCCGAGGTGACCTGCGTGGTGGTGGACGTGAGCCACGAGGA | |
| | CCCCGAGGTGAAGTTCAACTGGTACGTGGACGGCGTGGAGG | |
| | TGCACAACGCCAAGACCAAGCCCCGGGAGGAGCAGTACAAC | |
| | AGCACCTACCGGGTGGTGAGCGTGCTGACCGTGCTGCACCA | |
| | GGACTGGCTGAACGGCAAGGAGTACAAGTGCAAGGTGAGCA | |
| | ACAAGGCCCTGCCCGCCCCCATCGAGAAGACCATCAGCAAG | |
| | GCCAAGGGCCAGCCCCGGGAGCCCCAGGTGTACACCCTGCC | |
| | CCCCAGCCGGGACGAGCTGACCAAGAACCAGGTGAGCCTGA | |
| | CCTGCCTGGTGAAGGGCTTCTACCCCAGCGACATCGCCGTG | |
| | GAGTGGGAGAGCAACGGCCAGCCCGAGAACAACTACAAGAC | |
| | CACCCCCCCCGTGCTGGACAGCGACGGCAGCTTCTTCCTGT | |
| | ACAGCAAGCTGACCGTGGACAAGAGCCGGTGGCAGCAGGGC | |
| | AACGTGTTCAGCTGCAGCGTGATGCACGAGGCCCTGCACAA | |
| | CCACTACACCCAGAAGAGCCTGAGCCTGAGCCCCGGCAAG | |
| Human IgG1 hinge domain | GAGCCCAAGAGCTGCGACAAGACCCACACCTGCCCC | 44 |
| human IgG4 hinge domain | GAGAGCAAGTACGGCCCCCCCTGCCCCAGCTGCCCCGCCCC | 45 |
| | CGAGTTCCTGGGCGGCCCCAGCGTGTTCCTGTTCCCCCCCA | |
| | AGCCCAAGGACACCCTGATGATCAGCCGGACCCCCGAGGTG | |
| | ACCTGCGTGGTGGTGGACGTGAGCCAGGAGGACCCCGAGGT | |
| | GCAGTTCAACTGGTACGTGGACGGCGTGGAGGTGCACAACG | |
| | CCAAGACCAAGCCCCGGGAGGAGCAGTTCAACAGCACCTAC | |
| | CGGGTGGTGAGCGTGCTGACCGTGCTGCACCAGGACTGGCT | |
| | GAACGGCAAGGAGTACAAGTGCAAGGTGAGCAACAAGGGCC | |
| | TGCCCAGCAGCATCGAGAAGACCATCAGCAAGGCCAAGGGC | |
| | CAGCCCCGGGAGCCCCAGGTGTACACCCTGCCCCCCAGCCA | |
| | GGAGGAGATGACCAAGAACCAGGTGAGCCTGACCTGCCTGG | |
| | TGAAGGGCTTCTACCCCAGCGACATCGCCGTGGAGTGGGAG | |
| | AGCAACGGCCAGCCCGAGAACAACTACAAGACCACCCCCCC | |
| | CGTGCTGGACAGCGACGGCAGCTTCTTCCTGTACAGCCGGC | |
| | TGACCGTGGACAAGAGCCGGTGGCAGGAGGGCAACGTGTTC | |
| | AGCTGCAGCGTGATGCACGAGGCCCTGCACAACCACTACAC | |
| | CCAGAAGAGCCTGAGCCTGAGCCTGGGCAAG | |
| human IgG4 hinge domain | GAGAGCAAGTACGGCCCCCCCTGCCCC | 46 |

TRANSMEMBRANE

| | | |
|---|---|---|
| human CD8alpha | ATTTACATTTGGGCCCCTCTGGCTGGAACCTGCGGAG | 47 |
| transmembrane domain | TCCTGCTGCTGTCCCTGGTGATCACACTGTACTGT | |
| human CD28 transmembrane | TTCTGGGTGCTCGTTGTTGTTGGCGGCGTGCTGGCCT | 48 |
| domain | GTTACAGCCTGCTGGTTACCGTGGCCTTCATCATCTT | |
| | TTGGGTG | |

COSTIMULATORY DOMAINS

| | | |
|---|---|---|
| human CD28 costimulatory | CGAAGCAAGCGGAGCCGGGGAGGACACAGCGACTACA | 49 |
| domain | TGAACATGACCCCTCGGAGGCCAGGCCCCACCAGAAA | |
| | GCACTACCAGCCCTACGCCCCTCCCCGGGACTTTGCC | |
| | GCCTATCGGAGC | |
| human CD28 costimulatory | CGAAGCAAGCGGAGCCGGCTGCTGCACAGCGACTACA | 50 |
| domain | TGAACATGACCCCTCGGAGGCCAGGCCCCACCAGAAA | |
| | GCACTACCAGCCCTACGCCCCTCCCCGGGACTTTGCC | |
| | GCCTATCGGAGC | |
| human 4-1BB costimulatory | AAGAGGGGCAGAAAGAAGCTGCTCTACATCTTCAAGC | 51 |
| domain | AGCCCTTTTATGAGACCCGTGCAGACAACCCAGGAGGA | |
| | AGACGGATGCAGCTGCAGGTTCCCTGAGGAGGAGGAG | |
| | GGCGGCTGCGAACTG | |
| human DAP10 costimulatory | CTGTGCGCCCGGCCCCGGCGGAGCCCCGCCCAGGAGG | 52 |
| domain | ACGGCAAGGTGTACATCAACATGCCCGGCCGGGGC | |
| human DAP12 costimulatory | TACTTCCTGGGCCGGCTGGTGCCCCGGGGCCGGGGCG | 53 |
| domain | CCGCCGAGGCCGCCACCCGGAAGCAGCGGATCACCGA | |
| | GACCGAGAGCCCCTACCAGGAGCTGCAGGGCCAGCGG | |
| | AGCGACGTGTACAGCGACCTGAACACCCAGCGGCCCT | |
| | ACTACAAG | |
| human 2B4 costimulatory | TGGCGGCGGAAGCGGAAGGAGAAGCAGAGCGAGACCA | 54 |
| domain | GCCCCAAGGAGTTCCTGACCATCTACGAGGACGTGAA | |
| | GGACCTGAAGACCCGGCGGAACCACGAGCAGGAGCAG | |
| | ACCTTCCCCGGCGGCGGCAGCACCATCTACAGCATGA | |
| | TCCAGAGCCAGAGCAGCGCCCCCACCAGCCAGGAGCC | |
| | CGCCTACACCCTGTACAGCCTGATCCAGCCCAGCCGG | |
| | AAGAGCGGCAGCCGGAAGCGGAACCACAGCCCCAGCT | |
| | TCAACAGCACCATCTACGAGGTGATCGGCAAGAGCCA | |
| | GCCCAAGGCCCAGAACCCCGCCCGGCTGAGCCGGAAG | |
| | GAGCTGGAGAACTTCGACGTGTACAGC | |
| human OX40 costimulatory | GCCCTGTACCTGCTGCGGCGGGACCAGCGGCTGCCCC | 55 |
| domain | CCGACGCCCACAAGCCCCCCGGCGGCGGCAGCTTCCG | |
| | GACCCCCATCCAGGAGGAGCAGGCCGACGCCCACAGC | |
| | ACCCTGGCCAAGATC | |
| human CD27 costimulatory | CACCAGCGGCGGAAGTACCGGAGCAACAAGGGCGAGA | 56 |
| domain | GCCCCGTGGAGCCCGCCGAGCCCTGCCACTACAGCTG | |
| | CCCCCGGGAGGAGGAGGGCAGCACCATCCCCATCCAG | |
| | GAGGACTACCGGAAGCCCGAGCCCGCCTGCAGCCCC | |
| human CD27 costimulatory | CAGCGGCGGAAGTACCGGAGCAACAAGGGCGAGAGCC | 57 |
| domain | CCGTGGAGCCCGCCGAGCCCTGCCACTACAGCTGCCC | |
| | CCGGGAGGAGGAGGGCAGCACCATCCCCATCCAGGAG | |
| | GACTACCGGAAGCCCGAGCCCGCCTGCAGCCCC | |

ACTIVATION DOMAINS

| | | |
|---|---|---|
| human CD3zeta intracellular | GATATCAGGGTGAAGTTCAGCAGGAGCGCCGACGCCC | 58 |
| signaling domain | CCGCTTATCAACAGGGCCAGAACCAGCTGTACAACGA | |
| | GCTGAACCTCGGCAGAAGAGAGGAGTATGACGTGCTG | |
| | GACAAGAGGAGGGGCAGGGACCCTGAGATGGGCGGCA | |
| | AGCCTAGAAGAAAGAACCCCCAGGAAGGCCTCTACAA | |
| | CGAACTGCAGAAGGACAAGATGGCCGAGGCCTACAGC | |
| | GAGATCGGCATGAAAGGCGAGAGAAGGAGGGGAAAGG | |
| | GACATGACGGCCTGTACCAGGGACTCTCCACAGCCAC | |
| | CAAGGACACCTACGATGCCCTGCACATGCAGGCTCTG | |
| | CCCCCTAGA | |
| human CD3zeta intracellular | AGGGTGAAGTTCAGCAGGAGCGCCGACGCCCCCGCTT | 59 |
| signaling domain | ATCAACAGGGCCAGAACCAGCTGTACAACGAGCTGAA | |
| | CCTCGGCAGAAGAGAGGAGTATGACGTGCTGGACAAG | |
| | AGGAGGGGCAGGGACCCTGAGATGGGGGGCAAGCCTA | |
| | GAAGAAAGAACCCCCAGGAAGGCCTCTACAACGAACT | |
| | GCAGAAGGACAAGATGGCCGAGGCCTACAGCGAGATC | |
| | GGCATGAAAGGCGAGAGAAGGAGGGGAAAGGGACATG | |
| | ACGGCCTGTACCAGGGACTCTCCACAGCCACCAAGGA | |
| | CACCTACGATGCCCTGCACATGCAGGCTCTGCCCCCT | |
| | AGA | |
| human LAT intracellular | CACTGCCACAGACTGCCCGGCAGCTACGATAGCACCA | 60 |
| signaling domain | GCAGCGATTCTCTGTACCCCAGAGGCATCCAGTTCAG | |
| | ACGGCCTCATACAGTGGCTCCCTGGCCTCCTGCTTAC | |
| | CCTCCTGTGACAAGCTACCCACCTCTGAGCCAGCCTG | |
| | ACCTGCTGCCTATTCCTAGAAGCCCTCAGCCTCTCGG | |
| | CGGCAGCCATAGAACACCTAGCAGCAGAAGAGATAGC | |
| | GACGGCGCCAATAGCGTGGCCAGCTACGAAAATGAAG | |
| | GCGCCTCTGGCATTAGAGGCGCCCAAGCTGGATGGGG | |
| | AGTTTGGGGACCTAGCTGGACAAGACTGACCCCTGTG | |
| | TCTCTGCCTCCTGAACCTGCCTGCGAAGATGCCGACG | |
| | AGGACGAGGATGACTATCACAACCCTGGCTACCTGGT | |
| | GGTGCTGCCTGATAGCACACCAGCCACATCTACAGCC | |
| | GCTCCTAGTGCTCCTGCTCTGAGCACACCTGGCATCA | |
| | GAGACAGCGCCTTCAGCATGGAATCCATCGACGACTA | |
| | CGTGAACGTGCCCGAGTCTGGCGAATCTGCCGAAGCC | |
| | TCTCTTGACGGCAGCCGCGAGTATGTGAACGTGTCCC | |
| | AAGAACTGCATCCCGGCGCTGCCAAAACAGAACCTGC | |
| | TGCTCTGTCTAGCCAAGAGGCCGAGGAAGTGGAAGAA | |
| | GAAGGCGCCCCTGACTACGAGAACCTGCAAGAGCTGA | |
| | AC | |
| human LAT intracellular | CACTGCCACAGACTGCCCGGCAGCTACGATAGCACCA | 61 |
| signaling domain | GCAGCGATTCTCTGTACCCCAGAGGCATCCAGTTCAA | |
| | ACGGCCTCATACAGTGGCTCCCTGGCCTCCTGCTTAC | |
| | CCTCCTGTGACAAGCTACCCACCTCTGAGCCAGCCTG | |
| | ACCTGCTGCCTATTCCTAGAAGCCCTCAGCCTCTCGG | |
| | CGGCAGCCATAGAACACCTAGCAGCAGAAGAGATAGC | |
| | GACGGCGCCAATAGCGTGGCCAGCTACGAAAATGAAG | |
| | GCGCCTCTGGCATTAGAGGCGCCCAAGCTGGATGGGG | |
| | AGTTTGGGGACCTAGCTGGACAAGACTGACCCCTGTG | |
| | TCTCTGCCTCCTGAACCTGCCTGCGAAGATGCCGACG | |
| | AGGACGAGGATGACTATCACAACCCTGGCTACCTGGT | |
| | GGTGCTGCCTGATAGCACACCAGCCACATCTACAGCC | |
| | GCTCCTAGTGCTCCTGCTCTGAGCACACCTGGCATCA | |
| | GAGACAGCGCCTTCAGCATGGAATCCATCGACGACTA | |
| | CGTGAACGTGCCCGAGTCTGGCGAATCTGCCGAAGCC | |
| | TCTCTTGACGGCAGCCGCGAGTATGTGAACGTGTCCC | |
| | AAGAACTGCATCCCGGCGCTGCCAAAACAGAACCTGC | |
| | TGCTCTGTCTAGCCAAGAGGCCGAGGAAGTGGAAGAA | |
| | GAAGGCGCCCCTGACTACGAGAACCTGCAAGAGCTGA | |
| | AC | |
| human LAT intracellular | CACTGCCACAGACTGCCCGGCAGCTACGATAGCACCA | 62 |
| signaling domain | GCAGCGATTCTCTGTACCCCAGAGGCATCCAGTTCAG | |
| | ACGGCCTCATACAGTGGCTCCCTGGCCTCCTGCTTAC | |
| | CCTCCTGTGACAAGCTACCCACCTCTGAGCCAGCCTG | |
| | ACCTGCTGCCTATTCCTAGAAGCCCTCAGCCTCTCGG | |
| | CGGCAGCCATAGAACACCTAGCAGCAGAAGAGATAGC | |
| | GACGGCGCCAATAGCGTGGCCAGCTACGAAAATGAAG | |
| | GCGCCTCTGGCATTAGAGGCGCCCAAGCTGGATGGGG | |
| | AGTTTGGGGACCTAGCTGGACAAGACTGACCCCTGTG | |
| | TCTCTGCCTCCTGAACCTGCCTGCGAAGATGCCGACG | |
| | AGGACGAGGATGACTATCACAACCCTGGCTACCTGGT | |
| | GGTGCTGCCTGATAGCACACCAGCCACATCTACAGCC | |
| | GCTCCTAGTGCTCCTGCTCTGAGCACACCTGGCATCA | |
| | GAGACAGCGCCTTCAGCATGGAATCCATCGACGACTA | |
| | CGTGAACGTGCCCGAGTCTGGCGAATCTGCCGAAGCC | |
| | TCTCTTGACGGCAGCCGCGAGTATGTGAACGTGTCCC | |
| | AAGAACTGCATCCCGGCGCTGCCAGAACAGAACCTGC | |
| | TGCTCTGTCTAGCCAAGAGGCCGAGGAAGTGGAAGAA | |
| | GAAGGCGCCCCTGACTACGAGAACCTGCAAGAGCTGA | |
| | AC | |
| human LAT intracellular | CACTGCCACAGACTGCCCGGCAGCTACGATAGCACCA | 63 |
| signaling domain | GCAGCGATTCTCTGTACCCCAGAGGCATCCAGTTCAA | |
| | ACGGCCTCATACAGTGGCTCCCTGGCCTCCTGCTTAC | |
| | CCTCCTGTGACAAGCTACCCACCTCTGAGCCAGCCTG | |
| | ACCTGCTGCCTATTCCTAGAAGCCCTCAGCCTCTCGG | |
| | CGGCAGCCATAGAACACCTAGCAGCAGAAGAGATAGC | |
| | GACGGCGCCAATAGCGTGGCCAGCTACGAAAATGAAG | |
| | GCGCCTCTGGCATTAGAGGCGCCCAAGCTGGATGGGG | |
| | AGTTTGGGGACCTAGCTGGACAAGACTGACCCCTGTG | |
| | TCTCTGCCTCCTGAACCTGCCTGCGAAGATGCCGACG | |
| | AGGACGAGGATGACTATCACAACCCTGGCTACCTGGT | |
| | GGTGCTGCCTGATAGCACACCAGCCACATCTACAGCC | |
| | GCTCCTAGTGCTCCTGCTCTGAGCACACCTGGCATCA | |
| | GAGACAGCGCCTTCAGCATGGAATCCATCGACGACTA | |
| | CGTGAACGTGCCCGAGTCTGGCGAATCTGCCGAAGCC | |
| | TCTCTTGACGGCAGCCGCGAGTATGTGAACGTGTCCC | |
| | AAGAACTGCATCCCGGCGCTGCCAGAACAGAACCTGC | |
| | TGCTCTGTCTAGCCAAGAGGCCGAGGAAGTGGAAGAA | |
| | GAAGGCGCCCCTGACTACGAGAACCTGCAAGAGCTGA | |
| | AC | |
| human LAT intracellular | CACTGCCACAGACTGCCCGGCAGCTACGATAGCACCA | 64 |
| signaling domain | GCAGCGATTCTCTGTACCCCAGAGGCATCCAGTTCAG | |
| | ACGGCCTCATACAGTGGCTCCCTGGCCTCCTGCTTAC | |
| | CCTCCTGTGACAAGCTACCCACCTCTGAGCCAGCCTG | |
| | ACCTGCTGCCTATTCCTAGAAGCCCTCAGCCTCTCGG | |
| | CGGCAGCCATAGAACACCTAGCAGCAGAAGAGATAGC | |
| | GACGGCGCCAATAGCGTGGCCAGCTACGAAAATGAAG | |
| | GCGCCTCTGGCATTAGAGGCGCCCAAGCTGGATGGGG | |
| | AGTTTGGGGACCTAGCTGGACAAGACTGACCCCTGTG | |
| | TCTCTGCCTCCTGAACCTGCCTGCGAAGATGCCGACG | |
| | AGGACGAGGATGACTATCACAACCCTGAGTACCTGGT | |
| | GGTGCTGCCTGATAGCACACCAGCCACATCTACAGCC | |
| | GCTCCTAGTGCTCCTGCTCTGAGCACACCTGGCATCA | |
| | GAGACAGCGCCTTCAGCATGGAATCCATCGACGACTA | |
| | CGTGAACGTGCCCGAGTCTGGCGAATCTGCCGAAGCC | |
| | TCTCTTGACGGCAGCCGCGAGTATGTGAACGTGTCCC | |
| | AAGAACTGCATCCCGGCGCTGCCAAAACAGAACCTGC | |
| | TGCTCTGTCTAGCCAAGAGGCCGAGGAAGTGGAAGAA | |
| | GAAGGCGCCCCTGACTACGAGAACCTGCAAGAGCTGA | |
| | AC | |
| human LAT intracellular | CACTGCCACAGACTGCCCGGCAGCTACGATAGCACCA | 65 |
| signaling domain | GCAGCGATTCTCTGTACCCCAGAGGCATCCAGTTCAG | |
| | ACGGCCTCATACAGTGGCTCCCTGGCCTCCTGCTTAC | |
| | CCTCCTGTGACAAGCTACCCACCTCTGAGCCAGCCTG | |
| | ACCTGCTGCCTATTCCTAGAAGCCCTCAGCCTCTCGG | |
| | CGGCAGCCATAGAACACCTAGCAGCAGAAGAGATAGC | |
| | GACGGCGCCAATAGCGTGGCCAGCTACGAAAATGAAG | |
| | GCGCCTCTGGCATTAGAGGCGCCCAAGCTGGATGGGG | |
| | AGTTTGGGGACCTAGCTGGACAAGACTGACCCCTGTG | |
| | TCTCTGCCTCCTGAACCTGCCTGCGAAGATGCCGACG | |
| | AGGACGAGGATGACTATCACAACCCTGAGTACCTGGT | |
| | GGTGCTGCCTGATAGCACACCAGCCACATCTACAGCC | |
| | GCTCCTAGTGCTCCTGCTCTGAGCACACCTGGCATCA | |
| | GAGACAGCGCCTTCAGCATGGAATCCATCGACGACTA | |
| | CGTGAACGTGCCCGAGTCTGGCGAATCTGCCGAAGCC | |
| | TCTCTTGACGGCAGCCGCGAGTATGTGAACGTGTCCC | |
| | AAGAACTGCATCCCGGCGCTGCCAGAACAGAACCTGC | |
| | TGCTCTGTCTAGCCAAGAGGCCGAGGAAGTGGAAGAA | |
| | GAAGGCGCCCCTGACTACGAGAACCTGCAAGAGCTGA | |
| | AC | |
| human LAT intracellular | CACTGCCACCGGCTGCCCGGCAGCTACGACAGCACCA | 66 |
| signalling domain alternative | GCAGCGACAGCCTGTACCCCCGGGGCATCCAGTTCAA | |
| isoform | GCGGCCCCACACCGTGGCCCCCTGGCCCCCCGCCTAC | |
| | CCCCCCGTGACCAGCTACCCCCCCCTGAGCCAGCCCG | |
| | ACCTGCTGCCCATCCCCCGGAGCCCCCAGCCCCTGGG | |
| | CGGCAGCCACCGGACCCCCAGCAGCCGGCGGGACAGC | |
| | GACGGCGCCAACAGCGTGGCCAGCTACGAGAACGAGG | |
| | AGCCCGCCTGCGAGGACGCCGACGAGGACGAGGACGA | |
| | CTACCACAACCCCGGCTACCTGGTGGTGCTGCCCGAC | |
| | AGCACCCCCGCCACCAGCACCGCCGCCCCCAGCGCCC | |
| | CCGCCCTGAGCACCCCCGGCATCCGGGACAGCGCCTT | |
| | CAGCATGGAGAGCATCGACGACTACGTGAACGTGCCC | |
| | GAGAGCGGCGAGAGCGCCGAGGCCAGCCTGGACGGCA | |
| | GCCGGGAGTACGTGAACGTGAGCCAGGAGCTGCACCC | |
| | CGGCGCCGCCAAGACCGAGCCCGCCGCCCTGAGCAGC | |
| | CAGGAGGCCGAGGAGGTGGAGGAGGAGGGCGCCCCCG | |
| | ACTACGAGAACCTGCAGGAGCTGAAC | |
| human LAT intracellular | CACTGCCACCGGCTGCCCGGCAGCTACGACAGCACCA | 67 |
| signalling domain alternative | GCAGCGACAGCCTGTACCCCCGGGGCATCCAGTTCAA | |
| isoform | GCGGCCCCACACCGTGGCCCCCTGGCCCCCCGCCTAC | |
| | CCCCCCGTGACCAGCTACCCCCCCCTGAGCCAGCCCG | |
| | ACCTGCTGCCCATCCCCAGCCCCCAGCCCCTGGGCGG | |
| | CAGCCACCGGACCCCCAGCAGCCGGGGGGACAGCGAC | |
| | GGCGCCAACAGCGTGGCCAGCTACGAGAACGAGGAGC | |
| | CCGCCTGCGAGGACGCCGACGAGGACGAGGACGACTA | |
| | CCACAACCCCGGCTACCTGGTGGTGCTGCCCGACAGC | |
| | ACCCCCGCCACCAGCACCGCCGCCCCCAGCGCCCCCG | |
| | CCCTGAGCACCCCCGGCATCCGGGACAGCGCCTTCAG | |
| | CATGGAGAGCATCGACGACTACGTGAACGTGCCCGAG | |
| | AGCGGCGAGAGCGCCGAGGCCAGCCTGGACGGCAGCC | |
| | GGGAGTACGTGAACGTGAGCCAGGAGCTGCACCCCGG | |
| | CGCCGCCAAGACCGAGCCCGCCGCCCTGAGCAGCCAG | |
| | GAGGCCGAGGAGGTGGAGGAGGAGGGCGCCCCCGACT | |
| | ACGAGAACCTGCAGGAGCTGAAC | |
| human LAT intracellular | CACTGCCACCGGCTGCCCGGCAGCTACGACAGCACCA | 68 |
| signalling domain alternative | GCAGCGACAGCCTGTACCCCCGGGGCATCCAGTTCAA | |
| isoform | GCGGCCCCACACCGTGGCCCCCTGGCCCCCCGCCTAC | |
| | CCCCCCGTGACCAGCTACCCCCCCCTGAGCCAGCCCG | |
| | ACCTGCTGCCCATCCCCAGCCCCCAGCCCCTGGGGGG | |
| | CAGCCACCGGACCCCCAGCAGCCGGCGGGACAGCGAC | |
| | GGCGCCAACAGCGTGGCCAGCTACGAGAACGAGGGCG | |
| | CCAGCGGCATCCGGGGCGCCCAGGCCGGCTGGGGCGT | |
| | GTGGGGCCCCAGCTGGACCCGGCTGACCCCCGTGAGC | |
| | CTGCCCCCCGAGCCCGCCTGCGAGGACGCCGACGAGG | |
| | ACGAGGACGACTACCACAACCCCGGCTACCTGGTGGT | |
| | GCTGCCCGACAGCACCCCCGCCACCAGCACCGCCGCC | |
| | CCCAGCGCCCCCGCCCTGAGCACCCCCGGCATCCGGG | |
| | ACAGCGCCTTCAGCATGGAGAGCATCGACGACTACGT | |
| | GAACGTGCCCGAGAGCGGCGAGAGCGCCGAGGCCAGC | |
| | CTGGACGGCAGCCGGGAGTACGTGAACGTGAGCCAGG | |
| | AGCTGCACCCCGGCGCCGCCAAGACCGAGCCCGCCGC | |
| | CCTGAGCAGCCAGGAGGCCGAGGAGGTGGAGGAGGAG | |
| | GGCGCCCCCGACTACGAGAACCTGCAGGAGCTGAAC | |

### G. Exemplary CAR Constructs

### i) Anti-CD22 CAR Constructs

Disclosed herein are CARs that specifically bind to CD22. In some embodiments, the CAR comprises an antigen recognition domain that specifically binds human CD22, a hinge domain comprising or consisting of a CD8 hinge domain, a transmembrane domain comprising or consisting of a CD8α transmembrane domain; a costimulatory domain comprising or consisting of a 4-1BB costimulatory domain; and an intracellular signaling domain comprising or consisting of a CD3zeta activation domain. Also disclosed herein are nucleic acid sequences encoding said CARs. In some embodiments, a T cell or population of T cells described herein is genetically modified to express at least one of the exemplary anti-CD22 CAR constructs described herein.

An exemplary anti-CD22 CAR, (“CAR1”, “CD22 CAR”, “2nd generation CAR”, “2^{nd} generation CD22 CAR”, “2G CD22 CAR”, “CD22 CART”, “CD22BBz CAR”, “CD22BBz” “2nd Gen CD22BBz”, “CD222-2nd Gen CAR”, “22BBz”, “22SA”, “22SAff” or “2G CAR”) amino acid sequence is shown below. (CD8α signal peptide, CD22 scFv (m971), CD8α hinge, CD8α transmembrane domain, 4-1BB signaling domain, CD3z signaling domain)

(SEQ ID NO: 69)
ASATMALPVTALLLPLALLLHAARPQVQLQQSGPGLVKPSQTLSLTCAISGDSVSSNSAAWNWI
RQSPSRGLEWLGRTYYRSKWYNDYAVSVKSRITINPDTSKNQFSLQLNSVTPEDTAVYYCAREV
TGDLEDAFDIWGQGTMVTVSSGGGGSDIQMTQSPSSLSASVGDRVTITCRASQTIWSYLNWYQQ
RPGKAPNLLIYAASSLQSGVPSRFSGRGSGTDFTLTISSLQAEDFATYYCQQSYSIPQTFGQGT
KLEIKLETTTPAPRPPTPAPTIASQPLSLRPEACRPAAGGAVHTRGLDFACDIYIWAPLAGTCG
VILLSLVITLYCKRGRKKLLYIFKQPFMRPVQTTQEEDGCSCRFPEEEEGGCELDIRVKFSRSA
DAPAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 69

An exemplary anti-CD22 CAR (“CAR1”, “CD22 CAR”, “2nd generation CAR”, “2nd generation CD22 CAR”, “2G CD22 CAR”, “CD22 CART”, “CD22BBz CAR”, “CD22BBz” “2nd Gen CD22BBz”, “CD222-2nd Gen CAR”, “22BBz”, “22SA”, “22SAff” or “2G CAR”) amino acid sequence is shown below. (CD8α signal peptide, CD22 scFv (m971), CD8α hinge, CD8α transmembrane domain, 4-1BB signaling domain, CD3z signaling domain)

(SEQ ID NO: 102)
MALPVTALLLPLALLLHAARPQVQLQQSGPGLVKPSQTLSLTCAISGDSVSSNSAAWNWIRQSP
SRGLEWLGRTYYRSKWYNDYAVSVKSRITINPDTSKNQFSLQLNSVTPEDTAVYYCAREVTGDL
EDAFDIWGQGTMVTVSSGGGGSDIQMTQSPSSLSASVGDRVTITCRASQTIWSYLNWYQQRPGK
APNLLIYAASSLQSGVPSRFSGRGSGTDFTLTISSLQAEDFATYYCQQSYSIPQTFGQGTKLEI
KLETTTPAPRPPTPAPTIASQPLSLRPEACRPAAGGAVHTRGLDFACDIYIWAPLAGTCGVLLL
SLVITLYCKRGRKKLLYIFKQPFMRPVQTTQEEDGCSCRFPEEEEGGCELDIRVKFSRSADAPA
YQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 102.

An exemplary anti-CD22 CAR, “CAR1”, “CD22 CAR”, “2nd generation CAR” or “2G CAR” polynucleotide sequence is shown below. (CD8α signal peptide, CD22 scFv (m971), CD8α hinge, CD5α transmembrane domain, 4-1BB signaling domain, CD3z signaling domain)

(SEQ ID NO: 70)
GCTAGCGCCACCATGGCTCTGCCTGTGACAGCTCTGCTGCTGCCTCTGGCCCTGCTGCTCCATG
CTGCTAGACCTCAGGTGCAGCTCCAGCAGTCTGGCCCAGGACTGGTCAAGCCTAGCCAGACCCT
GAGCCTGACCTGCGCCATCAGCGGCGACAGCGTGTCCTCTAACAGCGCCGCCTGGAACTGGATC
AGACAGAGCCCCAGCAGAGGCCTGGAATGGCTGGGCCGGACCTACTACCGGTCCAAGTGGTACA
ACGACTACGCCGTGTCCGTGAAGTCCCGGATCACCATCAACCCCGACACCAGCAAGAACCAGTT
CTCCCTGCAGCTGAACAGCGTGACCCCTGAGGACACCGCCGTGTACTACTGCGCCAGAGAAGTG
ACCGGCGACCTGGAAGATGCCTTCGACATCTGGGGCCAGGGCACCATGGTCACCGTGTCTAGCG
GAGGCGGCGGAAGCGACATCCAGATGACCCAGAGCCCTAGCTCCCTGAGCGCCAGCGTGGGCGA
CAGAGTGACCATCACCTGTCGGGCCAGCCAGACCATCTGGTCCTACCTGAATTGGTATCAGCAG
CGGCCAGGCAAGGCCCCTAACCTGCTGATCTATGCCGCCAGCAGCCTGCAGAGCGGCGTGCCAA
GCAGATTCTCTGGCAGAGGCTCCGGCACCGACTTCACCCTGACAATCAGTTCCCTGCAGGCCGA
GGACTTCGCCACCTACTACTGCCAGCAGTCCTACAGCATCCCTCAGACCTTCGGCCAGGGGACC
AAGCTGGAAATCAAGCTCGAGACCACCACCCCCGCCCCTAGGCCTCCCACACCTGCCCCCACAA
TCGCCTCCCAGCCTCTCAGCCTGAGGCCTGAAGCTTGCAGGCCCGCTGCCGGAGGAGCTGTCCA
TACCAGGGGACTCGACTTCGCCTGCGACATTTACATTTGGGCCCCTCTGGCTGGAACCTGCGGA
GTCCTGCTGCTGTCCCTGGTGATCACACTGTACTGTAAGAGGGGCAGAAAGAAGCTGCTCTACA
TCTTCAAGCAGCCCTTTATGAGACCCGTGCAGACAACCCAGGAGGAAGACGGATGCAGCTGCAG
GTTCCCTGAGGAGGAGGAGGGCGGCTGCGAACTGGATATCAGGGTGAAGTTCAGCAGGAGCGCC
GACGCCCCCGCTTATCAACAGGGCCAGAACCAGCTGTACAACGAGCTGAACCTCGGCAGAAGAG
AGGAGTATGACGTGCTGGACAAGAGGAGGGGCAGGGACCCTGAGATGGGCGGCAAGCCTAGAAG
AAAGAACCCCCAGGAAGGCCTCTACAACGAACTGCAGAAGGACAAGATGGCCGAGGCCTACAGC
GAGATCGGCATGAAAGGCGAGAGAAGGAGGGGAAAGGGACATGACGGCCTGTACCAGGGACTCT
CCACAGCCACCAAGGACACCTACGATGCCCTGCACATGCAGGCTCTGCCCCCTAGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 70.

An exemplary anti-CD22 CAR, “CAR1”, “CD22 CAR”, “2nd generation CAR” or “2G CAR” polynucleotide sequence is shown below. (CD8α signal peptide, CD22 scFv (m971), CD8α hinge, CD8α transmembrane domain, 4-1BB signaling domain, CD3z signaling domain)

(SEQ ID NO: 103)
ATGGCTCTGCCTGTGACAGCTCTGCTGCTGCCTCTGGCCCTGCTGCTCCATGCTGCTAGACCTC
AGGTGCAGCTCCAGCAGTCTGGCCCAGGACTGGTCAAGCCTAGCCAGACCCTGAGCCTGACCTG
CGCCATCAGCGGCGACAGCGTGTCCTCTAACAGCGCCGCCTGGAACTGGATCAGACAGAGCCCC
AGCAGAGGCCTGGAATGGCTGGGCCGGACCTACTACCGGTCCAAGTGGTACAACGACTACGCCG
TGTCCGTGAAGTCCCGGATCACCATCAACCCCGACACCAGCAAGAACCAGTTCTCCCTGCAGCT
GAACAGCGTGACCCCTGAGGACACCGCCGTGTACTACTGCGCCAGAGAAGTGACCGGCGACCTG
GAAGATGCCTTCGACATCTGGGGCCAGGGCACCATGGTCACCGTGTCTAGCGGAGGCGGCGGAA
GCGACATCCAGATGACCCAGAGCCCTAGCTCCCTGAGCGCCAGCGTGGGCGACAGAGTGACCAT
CACCTGTCGGGCCAGCCAGACCATCTGGTCCTACCTGAATTGGTATCAGCAGCGGCCAGGCAAG
GCCCCTAACCTGCTGATCTATGCCGCCAGCAGCCTGCAGAGCGGCGTGCCAAGCAGATTCTCTG
GCAGAGGCTCCGGCACCGACTTCACCCTGACAATCAGTTCCCTGCAGGCCGAGGACTTCGCCAC
CTACTACTGCCAGCAGTCCTACAGCATCCCTCAGACCTTCGGCCAGGGGACCAAGCTGGAAATC
AAGCTCGAGACCACCACCCCCGCCCCTAGGCCTCCCACACCTGCCCCCACAATCGCCTCCCAGC
CTCTCAGCCTGAGGCCTGAAGCTTGCAGGCCCGCTGCCGGAGGAGCTGTCCATACCAGGGGACT
CGACTTCGCCTGCGACATTTACATTTGGGCCCCTCTGGCTGGAACCTGCGGAGTCCTGCTGCTG
TCCCTGGTGATCACACTGTACTGTAAGAGGGGCAGAAAGAAGCTGCTCTACATCTTCAAGCAGC
CCTTTATGAGACCCGTGCAGACAACCCAGGAGGAAGACGGATGCAGCTGCAGGTTCCCTGAGGA
GGAGGAGGGCGGCTGCGAACTGGATATCAGGGTGAAGTTCAGCAGGAGCGCCGACGCCCCCGCT
TATCAACAGGGCCAGAACCAGCTGTACAACGAGCTGAACCTCGGCAGAAGAGAGGAGTATGACG
TGCTGGACAAGAGGAGGGGCAGGGACCCTGAGATGGGCGGCAAGCCTAGAAGAAAGAACCCCCA
GGAAGGCCTCTACAACGAACTGCAGAAGGACAAGATGGCCGAGGCCTACAGCGAGATCGGCATG
AAAGGCGAGAGAAGGAGGGGAAAGGGACATGACGGCCTGTACCAGGGACTCTCCACAGCCACCA
AGGACACCTACGATGCCCTGCACATGCAGGCTCTGCCCCCTAGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 103.

An exemplary bicistronic anti-CD22 CAR and anti-CD22 CAR “CAR1-linker-CAR2” or “LAT-CAR” or “22ALA-CART” with wild type LAT domain amino acid sequence is shown below (CAR1; Furin/P2A linker; CAR2)

(SEQ ID NO: 217)
MALPVTALLLPLALLLHAARPQVQLQQSGPGMVKPSQTLSLTCAISGDSVSSNSVAWNWIRQS
PSRGLEWLGRTYYRSTWYNDYAVSMKSRITINPDTNKNQFSLQLNSVTPEDTAVYYCAREVTG
DLEDAFDIWGQGTMVTVSSGGGGSGGGGSGGGGSDIQMIQSPSSLSASVGDRVTITCRASQTI
WSYLNWYRQRPGEAPNLLIYAASSLQSGVPSRFSGRGSGTDFTLTISSLQAEDFATYYCQQSYSI
PQTFGQGTKLEIKLETTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELD
IRVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRRKRRGSG
TPDPWGSGATNFSLLKQAGDVEENPGPGSMALPVTALLLPLALLLHAARPDYKDDD
DKQVQLQQSGPGMVKPSQTLSLTCAISGDSVSSNSVAWNWIRQSPSRGLEWLGRTYYR
STWYNDYAVSMKSRITINPDTNKNQFSLQLNSVTPEDTAVYYCAREVTGDLEDAFDIW
GQGTMVTVSSGGGGSGGGGSGGGGSDIQMIQSPSSLSASVGDRVTITCRASQTIWSYLN
WYRQRPGEAPNLLIYAASSLQSGVPSRFSGRGSGTDFTLTISSLQAEDFATYYCQQSYSIP
QTFGQGTKLEIKSRIEVMYPPPYLDNEKSNGTIIHVKGKHLCPSPLFPGPSKPFWVLVVV
GGVLACYSLLVTVAFIIFWVHCHRLPGSYDSTSSDSLYPRGIQFKRPHTVAPWPPAYPPV
TSYPPLSQPDLLPIPRSPQPLGGSHRTPSSRRDSDGANSVASYENEGASGIRGAQAGWGV
WGPSWTRLTPVSLPPEPACEDADEDEDDYHNPGYLVVLPDSTPATSTAAPSAPALSTPGI
RDSAFSMESIDDYVNVPESGESAEASLDGSREYVNVSQELHPGAAKTEPAALSSQEAEE
VEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 217.

An exemplary bicistronic anti-CD22 CAR and anti-CD22 CAR “CAR1-linker-CAR2” or “LAT-CAR” or “22ALA-CART” with K52R mutation in the LAT domain amino acid sequence is shown below (CAR1; Furin/P2A linker; CAR2, K52R)

(SEQ ID NO: 218)
MALPVTALLLPLALLLHAARPQVQLQQSGPGMVKPSQTLSLTCAISGDSVSSNSVAWNWIRQS
PSRGLEWLGRTYYRSTWYNDYAVSMKSRITINPDTNKNQFSLQLNSVTPEDTAVYYCAREVTG
DLEDAFDIWGQGTMVTVSSGGGGSGGGGSGGGGSDIQMIQSPSSLSASVGDRVTITCRASQTI
WSYLNWYRQRPGEAPNLLIYAASSLQSGVPSRFSGRGSGTDFTLTISSLQAEDFATYYCQQSYSI
PQTFGQGTKLEIKLETTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELD
IRVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRRKRRGSG
TPDPWGSGATNFSLLKQAGDVEENPGPGSMALPVTALLLPLALLLHAARPDYKDDD
DKQVQLQQSGPGMVKPSQTLSLTCAISGDSVSSNSVAWNWIRQSPSRGLEWLGRTYYR
STWYNDYAVSMKSRITINPDTNKNQFSLQLNSVTPEDTAVYYCAREVTGDLEDAFDIW
GQGTMVTVSSGGGGSGGGGSGGGGSDIQMIQSPSSLSASVGDRVTITCRASQTIWSYLN
WYRQRPGEAPNLLIYAASSLQSGVPSRFSGRGSGTDFTLTISSLQAEDFATYYCQQSYSIP
QTFGQGTKLEIKSRIEVMYPPPYLDNEKSNGTIIHVKGKHLCPSPLFPGPSKPFWVLVVV
GGVLACYSLLVTVAFIIFWVHCHRLPGSYDSTSSDSLYPRGIQFRRPHTVAPWPPAYPPV
TSYPPLSQPDLLPIPRSPQPLGGSHRTPSSRRDSDGANSVASYENEGASGIRGAQAGWGV
WGPSWTRLTPVSLPPEPACEDADEDEDDYHNPGYLVVLPDSTPATSTAAPSAPALSTPGI
RDSAFSMESIDDYVNVPESGESAEASLDGSREYVNVSQELHPGAAKTEPAALSSQEAEE
VEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 218.

An exemplary bicistronic anti-CD22 CAR and anti-CD22 CAR “CAR1-linker-CAR2” or “LAT-CAR” or “22ALA-CART” with K233R mutation in the LAT domain amino acid sequence is shown below (CAR1; Furin/P2A linker; CAR2, K233R)

(SEQ ID NO: 219)
MALPVTALLLPLALLLHAARPQVQLQQSGPGMVKPSQTLSLTCAISGDSVSSNSVAWNWIRQS
PSRGLEWLGRTYYRSTWYNDYAVSMKSRITINPDTNKNQFSLQLNSVTPEDTAVYYCAREVTG
DLEDAFDIWGQGTMVTVSSGGGGSGGGGSGGGGSDIQMIQSPSSLSASVGDRVTITCRASQTI
WSYLNWYRQRPGEAPNLLIYAASSLQSGVPSRFSGRGSGTDFTLTISSLQAEDFATYYCQQSYSI
PQTFGQGTKLEIKLETTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELD
IRVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRRKRRGSG
TPDPWGSGATNFSLLKQAGDVEENPGPGSMALPVTALLLPLALLLHAARPDYKDDD
DKQVQLQQSGPGMVKPSQTLSLTCAISGDSVSSNSVAWNWIRQSPSRGLEWLGRTYYR
STWYNDYAVSMKSRITINPDTNKNQFSLQLNSVTPEDTAVYYCAREVTGDLEDAFDIW
GQGTMVTVSSGGGGSGGGGSGGGGSDIQMIQSPSSLSASVGDRVTITCRASQTIWSYLN
WYRQRPGEAPNLLIYAASSLQSGVPSRFSGRGSGTDFTLTISSLQAEDFATYYCQQSYSIP
QTFGQGTKLEIKSRIEVMYPPPYLDNEKSNGTIIHVKGKHLCPSPLFPGPSKPFWVLVVV
GGVLACYSLLVTVAFIIFWVHCHRLPGSYDSTSSDSLYPRGIQFKRPHTVAPWPPAYPPV
TSYPPLSQPDLLPIPRSPQPLGGSHRTPSSRRDSDGANSVASYENEGASGIRGAQAGWGV
WGPSWTRLTPVSLPPEPACEDADEDEDDYHNPGYLVVLPDSTPATSTAAPSAPALSTPGI
RDSAFSMESIDDYVNVPESGESAEASLDGSREYVNVSQELHPGAARTEPAALSSQEAEE
VEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 219.

An exemplary bicistronic anti-CD22 CAR and anti-CD22 CAR “CAR1-linker-CAR2” or “LAT-CAR” or “22ALA-CART” with K52R+K233R mutations in the LAT domain amino acid sequence is shown below (CAR1; Furin/P2A linker; CAR2, K52R, K233R)

(SEQ ID NO: 220)
MALPVTALLLPLALLLHAARPQVQLQQSGPGMVKPSQTLSLTCAISGDSVSSNSVAWNWIRQS
PSRGLEWLGRTYYRSTWYNDYAVSMKSRITINPDTNKNQFSLQLNSVTPEDTAVYYCAREVTG
DLEDAFDIWGQGTMVTVSSGGGGSGGGGSGGGGSDIQMIQSPSSLSASVGDRVTITCRASQTI
WSYLNWYRQRPGEAPNLLIYAASSLQSGVPSRFSGRGSGTDFTLTISSLQAEDFATYYCQQSYSI
PQTFGQGTKLEIKLETTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELD
IRVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRRKRRGSG
TPDPWGSGATNFSLLKQAGDVEENPGPGSMALPVTALLLPLALLLHAARPDYKDDD
DKQVQLQQSGPGMVKPSQTLSLTCAISGDSVSSNSVAWNWIRQSPSRGLEWLGRTYYR
STWYNDYAVSMKSRITINPDTNKNQFSLQLNSVTPEDTAVYYCAREVTGDLEDAFDIW
GQGTMVTVSSGGGGSGGGGSGGGGSDIQMIQSPSSLSASVGDRVTITCRASQTIWSYLN
WYRQRPGEAPNLLIYAASSLQSGVPSRFSGRGSGTDFTLTISSLQAEDFATYYCQQSYSIP
QTFGQGTKLEIKSRIEVMYPPPYLDNEKSNGTIIHVKGKHLCPSPLFPGPSKPFWVLVVV
GGVLACYSLLVTVAFIIFWVHCHRLPGSYDSTSSDSLYPRGIQFRRPHTVAPWPPAYPPV
TSYPPLSQPDLLPIPRSPQPLGGSHRTPSSRRDSDGANSVASYENEGASGIRGAQAGWGV
WGPSWTRLTPVSLPPEPACEDADEDEDDYHNPGYLVVLPDSTPATSTAAPSAPALSTPGI
RDSAFSMESIDDYVNVPESGESAEASLDGSREYVNVSQELHPGAARTEPAALSSQEAEE
VEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 220.

An exemplary bicistronic anti-CD22 CAR and anti-CD22 CAR “CAR1-linker-CAR2” or “LAT-CAR” or “22ALA-CART” with K52R+G160E mutations in the LAT domain amino acid sequence is shown below (CAR1; Furin/P2A linker; CAR2, K52R, G160E)

(SEQ ID NO: 221)
MALPVTALLLPLALLLHAARPQVQLQQSGPGMVKPSQTLSLTCAISGDSVSSNSVAWNWIRQS
PSRGLEWLGRTYYRSTWYNDYAVSMKSRITINPDTNKNQFSLQLNSVTPEDTAVYYCAREVTG
DLEDAFDIWGQGTMVTVSSGGGGSGGGGGGGGSDIQMIQSPSSLSASVGDRVTITCRASQTI
WSYLNWYRQRPGEAPNLLIYAASSLQSGVPSRFSGRGSGTDFTLTISSLQAEDFATYYCQQSYSI
PQTFGQGTKLEIKLETTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELD
IRVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRRKRRGSG
TPDPWGSGATNFSLLKQAGDVEENPGPGSMALPVTALLLPLALLLHAARPDYKDDD
DKQVQLQQSGPGMVKPSQTLSLTCAISGDSVSSNSVAWNWIRQSPSRGLEWLGRTYYR
STWYNDYAVSMKSRITINPDTNKNQFSLQLNSVTPEDTAVYYCAREVTGDLEDAFDIW
GQGTMVTVSSGGGGSGGGGSGGGGSDIQMIQSPSSLSASVGDRVTITCRASQTIWSYLN
WYRQRPGEAPNLLIYAASSLQSGVPSRFSGRGSGTDFTLTISSLQAEDFATYYCQQSYSIP
QTFGQGTKLEIKSRIEVMYPPPYLDNEKSNGTIIHVKGKHLCPSPLFPGPSKPFWVLVVV
GGVLACYSLLVTVAFIIFWVHCHRLPGSYDSTSSDSLYPRGIQFRRPHTVAPWPPAYPPV
TSYPPLSQPDLLPIPRSPQPLGGSHRTPSSRRDSDGANSVASYENEGASGIRGAQAGWGV
WGPSWTRLTPVSLPPEPACEDADEDEDDYHNPEYLVVLPDSTPATSTAAPSAPALSTPGI
RDSAFSMESIDDYVNVPESGESAEASLDGSREYVNVSQELHPGAAKTEPAALSSQEAEE
VEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 221.

An exemplary bicistronic anti-CD22 CAR and anti-CD22 CAR “CAR1-linker-CAR2” or “LAT-CAR” or “22ALA-CART” with K52R+K233R+G160E mutations in the LAT domain amino acid sequence is shown below (CAR1; Furin/P2A linker; CAR2, K52R, K233R, G160E)

(SEQ ID NO: 222)
MALPVTALLLPLALLLHAARPQVQLQQSGPGMVKPSQTLSLTCAISGDSVSSNSVAWNWIRQS
PSRGLEWLGRTYYRSTWYNDYAVSMKSRITINPDTNKNQFSLQLNSVTPEDTAVYYCAREVTG
DLEDAFDIWGQGTMVTVSSGGGGSGGGGSGGGGSDIQMIQSPSSLSASVGDRVTITCRASQTI
WSYLNWYRQRPGEAPNLLIYAASSLQSGVPSRFSGRGSGTDFTLTISSLQAEDFATYYCQQSYSI
PQTFGQGTKLEIKLETTTPAPRPPTPAPTIASQPLSLRPEACRPAAGGAVHTRGLDFACDIYIW
APLAGTCGVLLLSLVITLYCKRGRKKLLYIFKQPFMRPVQTTQEEDGCSCRFPEEEEGGCELD
IRVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPRRKRRGSG
TPDPWGSGATNFSLLKQAGDVEENPGPGSMALPVTALLLPLALLLHAARPDYKDDD
DKQVQLQQSGPGMVKPSQTLSLTCAISGDSVSSNSVAWNWIRQSPSRGLEWLGRTYYR
STWYNDYAVSMKSRITINPDTNKNQFSLQLNSVTPEDTAVYYCAREVTGDLEDAFDIW
GQGTMVTVSSGGGGSGGGGSGGGGSDIQMIQSPSSLSASVGDRVTITCRASQTIWSYLN
WYRQRPGEAPNLLIYAASSLQSGVPSRFSGRGSGTDFTLTISSLQAEDFATYYCQQSYSIP
QTFGQGTKLEIKSRIEVMYPPPYLDNEKSNGTIIHVKGKHLCPSPLFPGPSKPFWVLVVV
GGVLACYSLLVTVAFIIFWVHCHRLPGSYDSTSSDSLYPRGIQFRRPHTVAPWPPAYPPV
TSYPPLSQPDLLPIPRSPQPLGGSHRTPSSRRDSDGANSVASYENEGASGIRGAQAGWGV
WGPSWTRLTPVSLPPEPACEDADEDEDDYHNPEYLVVLPDSTPATSTAAPSAPALSTPGI
RDSAFSMESIDDYVNVPESGESAEASLDGSREYVNVSQELHPGAARTEPAALSSQEAEE
VEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 222.

### ii) Exemplary Anti-CD19 CAR Constructs

Disclosed herein are CARs that specifically bind to CD19. In some embodiments, the CAR comprises an antigen recognition domain that specifically binds human CD19, a hinge domain comprising or consisting of a CD28 hinge domain, a transmembrane domain comprising or consisting of a CD28 transmembrane domain; and an intracellular signaling domain comprising or consisting of a LAT intracellular signaling domain. Also disclosed herein are nucleic acid sequences encoding said CARs. In some embodiments, a T cell or population of T cells described herein is genetically modified to express at least one of the exemplary anti-CD19 CAR constructs described herein.

An exemplary bicistronic anti-CD19 CAR and anti-CD19 CAR “CAR1-linker-CAR2” or “LAT-CAR” or “19ALA-CART” amino acid sequence is shown below (CAR1; Furin/P2A linker; CAR2)

(SEQ ID NO: 223)
GSMEFGLSWLELVAILKGVQCSRDIQMTQTTSSLSASLGDRVTISCRASQDISKYLNWYQQKPD
GTVKLLIYHTSRLHSGVPSRESGSGSGTDYSLTISNLEQEDIATYFCQQGNTLPYTFGGGTKLEI
TGSTSGSGKPGSGEGSTKGEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYYCAKHYYYGGSYAMD
YWGQGTSVTVLETTTPAPRPPTPAPTIASQPLSLRPEACRPAAGGAVHTRGLDFACDIYIWAPL
AGTCGVLLLSLVITLYCKRGRKKLLYIFKQPFMRPVQTTQEEDGCSCRFPEEEEGGCELDIRV
KFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPRRKRRGSGTP
DPWGSGATNFSLLKQAGDVEENPGPGSMEFGLSWLFLVAILKGVQCSRDYKDDDDK
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEITGSTSGSGKPGSGEG
STKGEVKLQESGPGLVAPSQSLSVTCTVSGVSLPDYGVSWIRQPPRKGLEWLGVIWGSE
TTYYNSALKSRLTIIKDNSKSQVFLKMNSLQTDDTAIYYCAKHYYYGGSYAMDYWGQG
TSVTVSRIEVMYPPPYLDNEKSNGTIIHVKGKHLCPSPLFPGPSKPFWVLVVVGGVLACY
SLLVTVAFIFWVHCHRLPGSYDSTSSDSLYPRGIQFRRPHTVAPWPPAYPPVTSYPPLSQ
PDLLPIPRSPQPLGGSHRTPSSRRDSDGANSVASYENEGASGIRGAQAGWGVWGPSWTR
LTPVSLPPEPACEDADEDEDDYHNPGYLVVLPDSTPATSTAAPSAPALSTPGIRDSAFSM
ESIDDYVNVPESGESAEASLDGSREYVNVSQELHPGAAKTEPAALSSQEAEEVEEEGAP
DYENLQELN

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 223.

An exemplary bicistronic anti-CD19 CAR and anti-CD19 CAR “CAR1-linker-CAR2” or “LAT-CAR” or “19ALA-CART” polynucleotide sequence is shown below. (CAR1; furin/P2A linker; CAR2).

(SEQ ID NO: 224)
ATGGAGTTCGGATTATCTTGGTTATTTTTAGTAGCGATTTTGAAAGGAGTCCAATGTAGTCG
AGAACAAAAACTCATCTCAGAAGAGGATCTGGATATTCAAATGACACAAACTACCTCTTCTT
TATCTGCGAGTTTGGGAGATCGAGTTACTATAAGTTGCCGGGCTAGTCAGGATATTAGTAA
GTATCTCAATTGGTATCAACAAAAGCCGGATGGGACAGTCAAATTATTAATTTATCATACAT
CTCGATTACACAGTGGAGTACCAAGTCGGTTCAGTGGGTCTGGTAGCGGCACGGATTATT
CTTTGACTATATCTAATCTTGAGCAAGAAGATATAGCTACCTACTTTTGTCAGCAAGGTAAT
ACCTTGCCATACACGTTTGGAGGGGGGACCAAACTGGAGATTACAGGTAGTACGAGTGGT
TCTGGTAAGCCCGGCAGCGGAGAAGGTTCTACTAAAGGAGAGGTTAAATTACAAGAGTCT
GGCCCAGGCTTAGTGGCCCCTTCTCAATCTTTGTCTGTTACATGCACGGTCTCTGGGGTAT
CTTTACCAGACTATGGGGTATCTTGGATACGGCAACCCCCACGAAAAGGGCTCGAATGGT
TGGGAGTAATCTGGGGTTCTGAAACTACATATTACAATTCTGCGTTAAAATCTCGATTGACA
ATCATAAAAGATAATTCTAAGAGTCAAGTGTTCTTAAAAATGAACTCTTTGCAAACAGATGAT
ACTGCAATTTATTATTGTGCAAAACATTATTACTACGGAGGGAGTTATGCAATGGATTATTG
GGGGCAAGGGACTTCTGTCACCGTACTCGAGACCACCACCCCCGCCCCTAGGCCTCCCA
CACCTGCCCCCACAATCGCCTCCCAGCCTCTCAGCCTGAGGCCTGAAGCTTGCAGGCCC
GCTGCCGGAGGAGCTGTCCATACCAGGGGACTCGACTTCGCCTGCGACATTTACATTTGG
GCCCCTCTGGCTGGAACCTGCGGAGTCCTGCTGCTGTCCCTGGTGATCACACTGTACTGT
AAGAGGGGCAGAAAGAAGCTGCTCTACATCTTCAAGCAGCCCTTTATGAGACCCGTGCAG
ACAACCCAGGAGGAAGACGGATGCAGCTGCAGGTTCCCTGAGGAGGAGGAGGGCGGCT
GCGAACTGGATATCAGGGTGAAGTTCAGCAGGAGCGCCGACGCCCCCGCTTATCAACAG
GGCCAGAACCAGCTGTACAACGAGCTGAACCTCGGCAGAAGAGAGGAGTATGACGTGCT
GGACAAGAGGAGGGGCAGGGACCCTGAGATGGGCGGCAAGCCTAGAAGAAAGAACCCC
CAGGAAGGCCTCTACAACGAACTGCAGAAGGACAAGATGGCCGAGGCCTACAGCGAGAT
CGGCATGAAAGGCGAGAGAAGGAGGGGAAAGGGACATGACGGCCTGTACCAGGGACTC
TCCACAGCCACCAAGGACACCTACGATGCCCTGCACATGCAGGCTCTGCCCCCTAGAAG
GAAGAGAAGAGGCTCTGGTACCCCCGATCCTTGGGGAAGCGGCGCTACCAACTTCTC
CCTGCTCAAGCAGGCTGGCGATGTGGAGGAGAACCCCGGCCCCGGATCCATGGAGTT
TGGCCTGAGCTGGCTGTTCCTGGTGGCCATCCTCAAGGGCGTGCAGTGCTCCAGGGA
CTACAAAGACGATGACGACAAGGACATCCAGATGACCCAGACCACAAGCAGCCTGA
GCGCTTCCCTCGGCGACAGGGTGACCATCTCCTGTAGAGCCTCCCAAGACATCTCCA
AGTACCTGAACTGGTATCAGCAGAAACCCGACGGCACCGTGAAGCTGCTGATCTAC
CACACCAGCAGGCTGCATTCCGGCGTGCCCTCCAGATTTTCCGGCAGCGGCTCTGGT
ACCGACTACAGCCTCACCATCAGCAACTTAGAACAGGAGGACATCGCCACATATTT
CTGCCAACAGGGAAACACACTCCCCTATACCTTCGGCGGCGGCACAAAGTTAGAAA
TCACCGGCTCCACATCCGGCAGCGGAAAACCTGGTTCTGGCGAGGGCAGCACCAAG
GGCGAAGTGAAGCTGCAGGAAAGCGGACCTGGACTGGTCGCTCCCAGCCAGAGCCT
CAGCGTGACCTGTACAGTGAGCGGCGTGAGCCTGCCTGATTACGGCGTGAGCTGGA
TTAGACAGCCTCCCAGGAAGGGCTTAGAATGGCTCGGCGTGATTTGGGGCAGCGAG
ACAACCTACTATAACAGCGCCCTGAAGAGCAGGCTCACCATTATCAAGGACAACAG
CAAATCCCAGGTCTTCCTGAAGATGAACAGCCTCCAGACCGACGACACCGCCATCT
ACTACTGCGCCAAGCACTACTATTATGGCGGCTCCTACGCCATGGACTACTGGGGCC
AGGGCACCAGCGTGACAGTGTCTAGAATCGAAGTGATGTACCCTCCACCTTACCTGG
ACAACGAGAAGTCCAACGGCACCATCATCCACGTGAAGGGCAAGCACCTGTGTCCT
TCTCCACTGTTCCCCGGACCTAGCAAGCCTTTCTGGGTGCTCGTTGTTGTTGGCGGCG
TGCTGGCCTGTTACAGCCTGCTGGTTACCGTGGCCTTCATCATCTTTTGGGTGCACTG
CCACAGACTGCCCGGCAGCTACGATAGCACCAGCAGCGATTCTCTGTACCCCAGAG
GCATCCAGTTCAGACGGCCTCATACAGTGGCTCCCTGGCCTCCTGCTTACCCTCCTGT
GACAAGCTACCCACCTCTGAGCCAGCCTGACCTGCTGCCTATTCCTAGAAGCCCTCA
GCCTCTCGGCGGCAGCCATAGAACACCTAGCAGCAGAAGAGATAGCGACGGCGCCA
ATAGCGTGGCCAGCTACGAAAATGAAGGCGCCTCTGGCATTAGAGGCGCCCAAGCT
GGATGGGGAGTTTGGGGACCTAGCTGGACAAGACTGACCCCTGTGTCTCTGCCTCCT
GAACCTGCCTGCGAAGATGCCGACGAGGACGAGGATGACTATCACAACCCTGGCTA
CCTGGTGGTGCTGCCTGATAGCACACCAGCCACATCTACAGCCGCTCCTAGTGCTCC
TGCTCTGAGCACACCTGGCATCAGAGACAGCGCCTTCAGCATGGAATCCATCGACG
ACTACGTGAACGTGCCCGAGTCTGGCGAATCTGCCGAAGCCTCTCTTGACGGCAGCC
GCGAGTATGTGAACGTGTCCCAAGAACTGCATCCCGGCGCTGCCAAAACAGAACCT
GCTGCTCTGTCTAGCCAAGAGGCCGAGGAAGTGGAAGAAGAAGGCGCCCCTGACTA
CGAGAACCTGCAAGAGCTGAACTGA

In some embodiments, the anti-CD19 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 224.

An exemplary anti-CD19 CAR, “CAR1”, “CD19BBz”, “2^{nd} generation CD19 CAR” or “2G CD19 CAR” amino acid sequence is shown below. (CD8α signal peptide, CD19 scFv (FMC63), CD8α hinge, CD8α transmembrane domain, 4-1BB signaling domain, CD3z signaling domain

(SEQ ID NO: 225)
GSMEFGLSWLFLVAILKGVQCSRDIQMTQTTSSLSASLGDRVTISCRASQDISKYLNWY
QQKPDGTVKLLIYHTSRLHSGVPSRFSGSGSGTDYSLTISNLEQEDIATYFCQQGNT
LPYTFGGGTKLEITGSTSGSGKPGSGEGSTKGEVKLQESGPGLVAPSQSLSVTCTVS
GVSLPDYGVSWIRQPPRKGLEWLGVIWGSETTYYNSALKSRLTIIKDNSKSQVFLK
MNSLQTDDTAIYYCAKHYYYGGSYAMDYWGQGTSVTVLETTTPAPRPPTPAPTIASQ
PLSLRPEACRPAAGGAVHTRGLDFACDIYIWAPLAGTCGVLLLSLVITLYCKRGRKKLL
YIFKQPFMRPVQTTQEEDGCSCRFPEEEEGGCELDIRVKFSRSADAPAYQQGQNQLYN
ELNLGRREEYDVLDKRRGRDPEMGGKPRRKNPQEGLYNELQKDKMAEAYSEIGMKGERRR
GKGHDGLYQGLSTATKDTYDALHMQALPPR

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 225.

An exemplary anti-CD19 CAR “CAR2” or “CD19 CAR” amino acid sequence is shown below. (IgG signal peptide, CD19 scFv (FMC63), CD28 hinge, CD28 transmembrane domain LAT signaling domain (with K52R mutation)

(SEQ ID NO: 71)
GSMEFGLSWLFLVAILKGVQCSRDIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGTKLE
ITGSTSGSGKPGSGEGSTKGEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYYCAKHYYYGGSYAMD
YWGQGTSVTVSRIEVMYPPPYLDNEKSNGTIIHVKGKHLCPSPLFPGPSKPFWVLVVVGGVLAC
YSLLVTVAFIIFWVHCHRLPGSYDSTSSDSLYPRGIQFRRPHTVAPWPPAYPPVTSYPPLSQPD
ILPIPRSPQPLGGSHRTPSSRRDSDGANSVASYENEGASGIRGAQAGWGVWGPSWIRLTPVSLP
PEPACEDADEDEDDYHNPGYLVVLPDSTPATSTAAPSAPALSTPGIRDSAFSMESIDDYVNVPE
SGESAEASLDGSREYVNVSQELHPGAAKTEPAALSSQEAEEVEEEGAPDYENLQELN

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 71.

An exemplary anti-CD19 CAR “CAR2” or “CD19 CAR” amino acid sequence is shown below. (IgG signal peptide, CD19 scFv (FMC63), CD28 hinge, CD28 transmembrane domain, LAT signaling domain (with K52R mutation)

(SEQ ID NO: 100)
MEFGLSWLFLVAILKGVQCSRDIQMTQTTSSLSASLGDRVTISCRASQDISKYLNWYQQKPDGT
VKLLIYHTSRLHSGVPSRFSGSGSGTDYSLTISNLEQEDIATYFCQQGNTLPYTFGGGTKLEIT
GSTSGSGKPGSGEGSTKGEVKLQESGPGLVAPSQSLSVTCTVSGVSLPDYGVSWIRQPPRKGLE
WLGVIWGSETTYYNSALKSRLTIIKDNSKSQVFLKMNSLQTDDTAIYYCAKHYYYGGSYAMDYW
GQGTSVTVSRIEVMYPPPYLDNEKSNGTIIHVKGKHLCPSPLFPGPSKPFWVLVVVGGVLACYS
LLVTVAFIIFWVHCHRLPGSYDSTSSDSLYPRGIQFRRPHTVAPWPPAYPPVTSYPPLSQPDLL
PIPRSPQPLGGSHRTPSSRRDSDGANSVASYENEGASGIRGAQAGWGVWGPSWIRLTPVSIPPE
PACEDADEDEDDYHNPGYLVVLPDSTPATSTAAPSAPALSTPGIRDSAFSMESIDDYVNVPESG
ESAEASLDGSREYVNVSQELHPGAAKTEPAALSSQEAEEVEEEGAPDYENLQELN

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 100.

An exemplary anti-CD19 CAR “CAR2” or “CD19 CAR” polynucleotide sequence is shown below. (IgG signal peptide, CD19 scFv (FMC63), CD28 hinge, CD28 transmembrane domain, LAT signaling domain (with K52R mutation)

(SEQ ID NO: 72)
GGATCCATGGAGTTTGGCCTGAGCTGGCTGTTCCTGGTGGCCATCCTCAAGGGCGTGCAGTGCT
CCAGGGACATCCAGATGACCCAGACCACAAGCAGCCTGAGCGCTTCCCTCGGCGACAGGGTGAC
CATCTCCTGTAGAGCCTCCCAAGACATCTCCAAGTACCTGAACTGGTACCAGCAGAAACCCGAC
GGCACCGTGAAGCTGCTGATCTACCACACCAGCAGGCTGCATTCCGGCGTGCCCTCCAGATTTT
CCGGCAGCGGCTCTGGTACCGACTACAGCCTCACCATCAGCAACTTAGAACAGGAGGACATCGC
CACATATTTCTGCCAACAGGGAAACACACTCCCCTATACCTTCGGCGGCGGCACAAAGTTAGAA
ATCACCGGCTCCACATCCGGCAGCGGAAAACCTGGTTCTGGCGAGGGCAGCACCAAGGGCGAAG
TGAAGCTGCAGGAAAGCGGACCTGGACTGGTCGCTCCCAGCCAGAGCCTCAGCGTGACCTGTAC
AGTGAGCGGCGTGAGCCTGCCTGATTACGGCGTGAGCTGGATTAGACAGCCTCCCAGGAAGGGC
TTAGAATGGCTCGGCGTGATTTGGGGCAGCGAGACAACCTACTATAACAGCGCCCTGAAGAGCA
GGCTCACCATTATCAAGGACAACAGCAAATCCCAGGTCTTCCTGAAGATGAACAGCCTCCAGAC
CGACGACACCGCCATCTACTACTGCGCCAAGCACTACTATTATGGCGGCTCCTACGCCATGGAC
TACTGGGGCCAGGGCACCAGCGTGACAGTGTCTAGAATCGAAGTGATGTACCCTCCACCTTACC
TGGACAACGAGAAGTCCAACGGCACCATCATCCACGTGAAGGGCAAGCACCTGTGTCCTTCTCC
ACTGTTCCCCGGACCTAGCAAGCCTTTCTGGGTGCTCGTTGTTGTTGGCGGCGTGCTGGCCTGT
TACAGCCTGCTGGTTACCGTGGCCTTCATCATCTTTTGGGTGCACTGCCACAGACTGCCCGGCA
GCTACGATAGCACCAGCAGCGATTCTCTGTACCCCAGAGGCATCCAGTTCAGACGGCCTCATAC
AGTGGCTCCCTGGCCTCCTGCTTACCCTCCTGTGACAAGCTACCCACCTCTGAGCCAGCCTGAC
CTGCTGCCTATTCCTAGAAGCCCTCAGCCTCTCGGCGGCAGCCATAGAACACCTAGCAGCAGAA
GAGATAGCGACGGCGCCAATAGCGTGGCCAGCTACGAAAATGAAGGCGCCTCTGGCATTAGAGG
CGCCCAAGCTGGATGGGGAGTTTGGGGACCTAGCTGGACAAGACTGACCCCTGTGTCTCTGCCT
CCTGAACCTGCCTGCGAAGATGCCGACGAGGACGAGGATGACTATCACAACCCTGGCTACCTGG
TGGTGCTGCCTGATAGCACACCAGCCACATCTACAGCCGCTCCTAGTGCTCCTGCTCTGAGCAC
ACCTGGCATCAGAGACAGCGCCTTCAGCATGGAATCCATCGACGACTACGTGAACGTGCCCGAG
TCTGGCGAATCTGCCGAAGCCTCTCTTGACGGCAGCCGCGAGTATGTGAACGTGTCCCAAGAAC
TGCATCCCGGCGCTGCCAAAACAGAACCTGCTGCTCTGTCTAGCCAAGAGGCCGAGGAAGTGGA
AGAAGAAGGCGCCCCTGACTACGAGAACCTGCAAGAGCTGAACTGATGAGTCGAC

In some embodiments, the anti-CD19 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 72.

An exemplary anti-CD19 CAR “CAR2” or “CD19 CAR” polynucleotide sequence is shown below. (IgG signal peptide, CD19 scFv (FMC63), CD28 hinge, CD28 transmembrane domain, LAT signaling domain (with K52R mutation)

(SEQ ID NO: 101)
ATGGAGTTTGGCCTGAGCTGGCTGTTCCTGGTGGCCATCCTCAAGGGCGTGCAGTGCTCCAGGG
ACATCCAGATGACCCAGACCACAAGCAGCCTGAGCGCTTCCCTCGGCGACAGGGTGACCATCTC
CTGTAGAGCCTCCCAAGACATCTCCAAGTACCTGAACTGGTACCAGCAGAAACCCGACGGCACC
GTGAAGCTGCTGATCTACCACACCAGCAGGCTGCATTCCGGCGTGCCCTCCAGATTTTCCGGCA
GCGGCTCTGGTACCGACTACAGCCTCACCATCAGCAACTTAGAACAGGAGGACATCGCCACATA
TTTCTGCCAACAGGGAAACACACTCCCCTATACCTTCGGCGGCGGCACAAAGTTAGAAATCACC
GGCTCCACATCCGGCAGCGGAAAACCTGGTTCTGGCGAGGGCAGCACCAAGGGCGAAGTGAAGC
TGCAGGAAAGCGGACCTGGACTGGTCGCTCCCAGCCAGAGCCTCAGCGTGACCTGTACAGTGAG
CGGCGTGAGCCTGCCTGATTACGGCGTGAGCTGGATTAGACAGCCTCCCAGGAAGGGCTTAGAA
TGGCTCGGCGTGATTTGGGGCAGCGAGACAACCTACTATAACAGCGCCCTGAAGAGCAGGCTCA
CCATTATCAAGGACAACAGCAAATCCCAGGTCTTCCTGAAGATGAACAGCCTCCAGACCGACGA
CACCGCCATCTACTACTGCGCCAAGCACTACTATTATGGCGGCTCCTACGCCATGGACTACTGG
GGCCAGGGCACCAGCGTGACAGTGTCTAGAATCGAAGTGATGTACCCTCCACCTTACCTGGACA
ACGAGAAGTCCAACGGCACCATCATCCACGTGAAGGGCAAGCACCTGTGTCCTTCTCCACTGTT
CCCCGGACCTAGCAAGCCTTTCTGGGTGCTCGTTGTTGTTGGCGGCGTGCTGGCCTGTTACAGC
CTGCTGGTTACCGTGGCCTTCATCATCTTTTGGGTGCACTGCCACAGACTGCCCGGCAGCTACG
ATAGCACCAGCAGCGATTCTCTGTACCCCAGAGGCATCCAGTTCAGACGGCCTCATACAGTGGC
TCCCTGGCCTCCTGCTTACCCTCCTGTGACAAGCTACCCACCTCTGAGCCAGCCTGACCTGCTG
CCTATTCCTAGAAGCCCTCAGCCTCTCGGCGGCAGCCATAGAACACCTAGCAGCAGAAGAGATA
GCGACGGCGCCAATAGCGTGGCCAGCTACGAAAATGAAGGCGCCTCTGGCATTAGAGGCGCCCA
AGCTGGATGGGGAGTTTGGGGACCTAGCTGGACAAGACTGACCCCTGTGTCTCTGCCTCCTGAA
CCTGCCTGCGAAGATGCCGACGAGGACGAGGATGACTATCACAACCCTGGCTACCTGGTGGTGC
TGCCTGATAGCACACCAGCCACATCTACAGCCGCTCCTAGTGCTCCTGCTCTGAGCACACCTGG
CATCAGAGACAGCGCCTTCAGCATGGAATCCATCGACGACTACGTGAACGTGCCCGAGTCTGGC
GAATCTGCCGAAGCCTCTCTTGACGGCAGCCGCGAGTATGTGAACGTGTCCCAAGAACTGCATC
CCGGCGCTGCCAAAACAGAACCTGCTGCTCTGTCTAGCCAAGAGGCCGAGGAAGTGGAAGAAGA
AGGCGCCCCTGACTACGAGAACCTGCAAGAGCTGAACTGATGAGTCGAC

In some embodiments, the anti-CD19 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 101.

### A. Other Exemplary First CARs of the Disclosure

### An Exemplary Anti-CD19 CAR

(SEQ ID NO: 309)
GSMEFGLSWLFLVAILKGVQCSRDIQMTQTTSSLSASLGDRVTISCRASQDISKYLNWY
QQKPDGTVKLLIYHTSRLHSGVPSRFSGSGSGTDYSLTISNLEQEDIATYFCQQGNTLPYTFGG
GTKLEITGSTSGSGKPGSGEGSTKGEVKLQESGPGLVAPSQSLSVTCTVSGVSLPDYGVSWIRQ
PPRKGLEWLGVIWGSETTYYNSALKSRLTIIKDNSKSQVFLKMNSLQTDDTAIYYCAKHYYYGG
SYAMDYWGQGTSVTVLETTTPAPRPPTPAPTIASQPLSLRPEACRPAAGGAVHTRGLDFACDI
YIWAPLAGTCGVLLLSLVITLYCKRGRKKLLYIFKQPFMRPVQTTQEEDGCSCRFPEEEEGGC
ELDIRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 309.

### B. Other Exemplary Second CARs

### An Exemplary Anti-CD22-LAT CAR

(SEQ ID NO: 300)
GSMALPVTALLLPLALLLHAARPDYKDDDDKQVQLQQSGPGMVKPSQTLSLTC
AISGDSVSSNSVAWNWIRQSPSRGLEWLGRTYYRSTWYNDYAVSMKSRITINPDTNKN
QFSLQLNSVTPEDTAVYYCAREVTGDLEDAFDIWGQGTMVTVSSGGGGSGGGGSGGG
GSDIQMIQSPSSLSASVGDRVTITCRASQTIWSYLNWYRQRPGEAPNLLIYAASSLQSGVP
SRFSGRGSGTDFTLTISSLQAEDFATYYCQQSYSIPQTFGQGTKLEIKSRIEVMYPPPYLD
NEKSNGTIIHVKGKHLCPSPLFPGPSKPFWVLVVVGGVLACYSLLVTVAFIIFWVHCHRL
PGSYDSTSSDSLYPRGIQFKRPHTVAPWPPAYPPVTSYPPLSQPDLLPIPRSPQPLGGSHRT
PSSRRDSDGANSVASYENEGASGIRGAQAGWGVWGPSWTRLTPVSLPPEPACEDADED
EDDYHNPGYLVVLPDSTPATSTAAPSAPALSTPGIRDSAFSMESIDDYVNVPESGESAEA
SLDGSREYVNVSQELHPGAAKTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 300.

### An Exemplary Anti-CD22-LAT-K52R CAR

(SEQ ID NO: 301)
GSMALPVTALLLPLALLLHAARPDYKDDDDKQVQLQQSGPGMVKPSQTLSLTC
AISGDSVSSNSVAWNWIRQSPSRGLEWLGRTYYRSTWYNDYAVSMKSRITINPDTNKN
QFSLQLNSVTPEDTAVYYCAREVTGDLEDAFDIWGQGTMVTVSSGGGGSGGGGSGGG
GSDIQMIQSPSSLSASVGDRVTITCRASQTIWSYLNWYRQRPGEAPNLLIYAASSLQSGVP
SRFSGRGSGTDFTLTISSLQAEDFATYYCQQSYSIPQTFGQGTKLEIKSRIEVMYPPPYLD
NEKSNGTIIHVKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIPRSPQPLGGSHRT
PSSRRDSDGANSVASYENEGASGIRGAQAGWGVWGPSWTRLTPVSLPPEPACEDADED
EDDYHNPGYLVVLPDSTPATSTAAPSAPALSTPGIRDSAFSMESIDDYVNVPESGESAEA
SLDGSREYVNVSQELHPGAAKTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 301.

### An Exemplary Anti-CD22-LAT-K233R CAR

(SEQ ID NO: 302)
GSMALPVTALLLPLALLLHAARPDYKDDDDKQVQLQQSGPGMVKPSQTLSLTC
AISGDSVSSNSVAWNWIRQSPSRGLEWLGRTYYRSTWYNDYAVSMKSRITINPDTNKN
QFSLQLNSVTPEDTAVYYCAREVTGDLEDAFDIWGQGTMVTVSSGGGGSGGGGSGGG
GSDIQMIQSPSSLSASVGDRVTITCRASQTIWSYLNWYRQRPGEAPNLLIYAASSLQSGVP
SRFSGRGSGTDFTLTISSLQAEDFATYYCQQSYSIPQTFGQGTKLEIKSRIEVMYPPPYLD
NEKSNGTIIHVKGKHLCPSPLFPGPSKPFWVLVVVGGVLACYSLLVTVAFIIFWVHCHRL
PGSYDSTSSDSLYPRGIQFKRPHTVAPWPPAYPPVTSYPPLSQPDLLPIPRSPQPLGGSHRT
PSSRRDSDGANSVASYENEGASGIRGAQAGWGVWGPSWTRLTPVSLPPEPACEDADED
EDDYHNPGYLVVLPDSTPATSTAAPSAPALSTPGIRDSAFSMESIDDYVNVPESGESAEA
SLDGSREYVNVSQELHPGAARTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 302.

### An Exemplary Anti-CD22-LAT-K52R-K233R CAR

(SEQ ID NO: 303)
GSMALPVTALLLPLALLLHAARPDYKDDDDKQVQLQQSGPGMVKPSQTLSLTC
AISGDSVSSNSVAWNWIRQSPSRGLEWLGRTYYRSTWYNDYAVSMKSRITINPDTNKN
QFSLQLNSVTPEDTAVYYCAREVTGDLEDAFDIWGQGTMVTVSSGGGGSGGGGSGGG
GSDIQMIQSPSSLSASVGDRVTITCRASQTIWSYLNWYRQRPGEAPNLLIYAASSLQSGVP
SRESGRGSGTDFTLTISSLQAEDFATYYCQQSYSIPQTFGQGTKLEIKSRIEVMYPPPYLD
NEKSNGTIIHVKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIPRSPQPLGGSHRT
PSSRRDSDGANSVASYENEGASGIRGAQAGWGVWGPSWTRLTPVSLPPEPACEDADED
EDDYHNPGYLVVLPDSTPATSTAAPSAPALSTPGIRDSAFSMESIDDYVNVPESGESAEA
SLDGSREYVNVSQELHPGAARTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 303.

### An Exemplary Anti-CD22-LAT-K52R-G160E CAR

(SEQ ID NO: 304)
GSMALPVTALLLPLALLLHAARPDYKDDDDKQVQLQQSGPGMVKPSQTLSLTC
AISGDSVSSNSVAWNWIRQSPSRGLEWLGRTYYRSTWYNDYAVSMKSRITINPDTNKN
QFSLQLNSVTPEDTAVYYCAREVTGDLEDAFDIWGQGTMVTVSSGGGGSGGGGSGGG
GSDIQMIQSPSSLSASVGDRVTITCRASQTIWSYLNWYRQRPGEAPNLLIYAASSLQSGVP
SRFSGRGSGTDFTLTISSLQAEDFATYYCQQSYSIPQTFGQGTKLEIKSRIEVMYPPPYLD
NEKSNGTIIHVKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIPRSPQPLGGSHRT
PSSRRDSDGANSVASYENEGASGIRGAQAGWGVWGPSWTRLTPVSLPPEPACEDADED
EDDYHNPEYLVVLPDSTPATSTAAPSAPALSTPGIRDSAFSMESIDDYVNVPESGESAEA
SLDGSREYVNVSQELHPGAAKTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 304.

### An Exemplary Anti-CD22-LAT-K52R-K233R-G160E CAR

(SEQ ID NO: 305)
SMALPVTALLLPLALLLHAARPDYKDDDDKQVQLQQSGPGMVKPSQTLSLTC
AISGDSVSSNSVAWNWIRQSPSRGLEWLGRTYYRSTWYNDYAVSMKSRITINPDTNKN
QFSLQLNSVTPEDTAVYYCAREVTGDLEDAFDIWGQGTMVTVSSGGGGSGGGGSGGG
GSDIQMIQSPSSLSASVGDRVTITCRASQTIWSYLNWYRQRPGEAPNLLIYAASSLQSGVP
SRFSGRGSGTDFTLTISSLQAEDFATYYCQQSYSIPQTFGQGTKLEIKSRIEVMYPPPYLD
NEKSNGTIIHVKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIPRSPQPLGGSHRT
PSSRRDSDGANSVASYENEGASGIRGAQAGWGVWGPSWTRLTPVSLPPEPACEDADED
EDDYHNPEYLVVLPDSTPATSTAAPSAPALSTPGIRDSAFSMESIDDYVNVPESGESAEA
SLDGSREYVNVSQELHPGAARTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 305.

### An Exemplary Anti-CD22-HiAff-LAT CAR

(SEQ ID NO: 306)
GSMALPVTALLLPLALLLHAARPDYKDDDDKQVQLQQSGPGLVKPSQTLSLTCAISGD
SVSSNSAAWNWIRQSPSRGLEWLGRTYYRSKWYNDYAVSVKSRITINPDTSKNQFSLQLNSVTP
EDTAVYYCAREVTGDLEDAFDIWGQGTMVTVSSGGGGSDIQMTQSPSSLSASVGDRVTITCRA
SQTIWSYLNWYQQRPGKAPNLLIYAASSLQSGVPSRFSGRGSGTDFTLTISSLQAEDFATYYCQ
QSYSIPQTFGQGTKLEIKSRIEVMYPPPYLDNEKSNGTIIHVKGKHLCPSPLFPGPSKPFWV
LVVVGGVLACYSLLVTVAFIIFWVHCHRLPGSYDSTSSDSLYPRGIQFRRPHTVAPWPPA
YPPVTSYPPLSQPDLLPIPRSPQPLGGSHRTPSSRRDSDGANSVASYENEGASGIRGAQAG
WGVWGPSWTRLTPVSLPPEPACEDADEDEDDYHNPGYLVVLPDSTPATSTAAPSAPALS
TPGIRDSAFSMESIDDYVNVPESGESAEASLDGSREYVNVSQELHPGAAKTEPAALSSQE
AEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 306.

### An Exemplary Anti-CD19-LAT CAR

(SEQ ID NO: 307)
GSMEFGLSWLFLVAILKGVQCSRDYKDDDDKDIQMTQTTSSLSASLGDRVTISCR
ASQDISKYLNWYQQKPDGTVKLLIYHTSRLHSGVPSRFSGSGSGTDYSLTISNLEQEDIA
TYFCQQGNTLPYTFGGGTKLEITGSTSGSGKPGSGEGSTKGEVKLQESGPGLVAPSQSLS
VTCTVSGVSLPDYGVSWIRQPPRKGLEWLGVIWGSETTYYNSALKSRLTIIKDNSKSQVF
LKMNSLQTDDTAIYYCAKHYYYGGSYAMDYWGQGTSVTVSRIEVMYPPPYLDNEKSN
GTIIHVKGKHLCPSPLFPGPSKPFWVLVVVGGVLACYSLLVTVAFIIFWVHCHRLPGSYD
STSSDSLYPRGIQFRRPHTVAPWPPAYPPVTSYPPLSQPDLLPIPRSPQPLGGSHRTPSSRR
DSDGANSVASYENEGASGIRGAQAGWGVWGPSWTRLTPVSLPPEPACEDADEDEDDY
HNPGYLVVLPDSTPATSTAAPSAPALSTPGIRDSAFSMESIDDYVNVPESGESAEASLDGS
REYVNVSQELHPGAAKTEPAALSSQEAEEVEEEGAPDYENLQELN

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 307.

### An Exemplary Anti-CD22-SAff-LAT CAR

(SEQ ID NO: 308)
GSMALPVTALLLPLALLLHAARPDYKDDDDKQVQLQQSGPGLVKPSQTLSLTCAISGD
SVSSNSAAWNWIRQSPSRGLEWLGRTYYRSKWYNDYAVSVKSRITINPDTSKNQFSLQLNSVTP
EDTAVYYCAREVTGDLEDAFDIWGQGTMVTVSSGGGGSDIQMTQSPSSLSASVGDRVTITCRA
SQTIWSYLNWYQQRPGKAPNLLIYAASSLQSGVPSRFSGRGSGTDFTLTISSLQAEDFATYYCQ
QSYSIPQTFGQGTKLEIKSRIEVMYPPPYLDNEKSNGTIIHVKGKHLCPSPLFPGPSKPFWVLV
VVGGVLACYSLLVTVAFIIFWVHCHRLPGSYDSTSSDSLYPRGIQFRRPHTVAPWPPAYPPVTSY
PPLSQPDLLPIPRSPQPLGGSHRTPSSRRDSDGANSVASYENEGASGIRGAQAGWGVWGPSWT
RLTPVSLPPEPACEDADEDEDDYHNPGYLVVLPDSTPATSTAAPSAPALSTPGIRDSAFSMESI
DDYVNVPESGESAEASLDGSREYVNVSQELHPGAAKTEPAALSSQEAEEVEEEGAPDYENLQE
LN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 308.

### Cleavage Sequences

Cleavage sequences can be used to create linked- or co-expression of genes in the constructs provided in the present disclosure. For example, cleavage sequences could be used to co-express genes (e.g. CAR1 and CAR2) by linking open reading frames to form a single cistron (e.g bicistronic CAR). In some aspects, cleavage sequences can comprise 2A self-cleaving peptide sequence elements. Exemplary 2A self-cleaving peptide sequence elements include but are not limited to T2A, P2A, E2A and F2A. In some embodiments, the cleavage sequence comprises a P2A sequence. In some embodiments, a cleavage sequence can comprise a furin cleavage peptide. In some embodiments, a cleavage sequence can comprise a furin cleavage peptide and a P2A sequence.

In some embodiments, P2A comprises or consists of an amino acid sequence having at least 90%, 91%, 92%, 93%, 94%, 95%, 96%, 97%, 98%, 99% or 100% identity with the amino acid sequence of GSGATNFSLLKQAGDVEENPGP (SEQ ID NO: 73).

In some embodiments, P2A comprises or consists of an amino acid sequence having at least 90%, 91%, 92%, 93%, 94%, 95%, 96%, 97%, 98%, 99% or 100% identity with the amino acid sequence of GSGATNFSLLKQAGDVEENPGP (SEQ ID NO: 74).

In some embodiments, T2A comprises or consists of an amino acid sequence having at least 90%, 91%, 92%, 93%, 94%, 95%, 96%, 97%, 98%, 99% or 100% identity with the amino acid sequence of GSGEGRGSLLTCGDVEENPGP (SEQ ID NO. 75).

In some embodiments, E2A comprises or consists of an amino acid sequence having at least 90%, 91%, 92%, 93%, 94%, 95%, 96%, 97%, 98%, 99% or 100% identity with the amino acid sequence of GSGQCTNYALLKLAGDVESNPGP (SEQ ID NO: 76).

In some embodiments, F2A comprises or consists of an amino acid sequence having at least 90%, 91%, 92%, 93%, 94%, 95%, 96%, 97%, 98%, 99% or 100% identity with the amino acid sequence of GSGVKQTLNFDLLKLAGDVESNPGP (SEQ ID NO: 77).

In some embodiments, a furin cleavage peptide comprises or consists of an amino acid sequence having at least 90%, 91%, 92%, 93%, 94%, 95%, 96%, 97%, 98%, 99% or 100% identity with the amino acid sequence of RKRRGSGTPDPW (SEQ ID NO: 78).

In some embodiments, a cleavage sequence comprises or consists of an amino acid sequence having at least 90%, 91%, 92%, 93%, 94%, 95%, 96%, 97%, 98%, 99% or 100% identity with the amino acid sequence of

(SEQ ID NO: 79)
RKRRGSGTPDPWGSGATNFSLLKQAGDVEENPGP.

In some embodiments, the CARs described herein can be under the control of an inducible promoter for gene transcription. In some embodiments, the inducible promoter is an EF1a promoter. In some embodiments, the inducible promoter is a PGK promoter.

iii) Exemplary Bicistronic CAR Constructs

Exemplary sequences of constructs disclosed herein comprising an anti-CD22 CAR and an anti-CD19 CAR are shown below.

An exemplary bicistronic anti-CD22 CAR and anti-CD19 CAR “CAR1-linker-CAR2” or “LAT-CAR” amino acid sequence is shown below (CAR1; Furin/P2A linker; CAR2).

(SEQ ID NO: 80)
ASATMALPVTALLLPLALLLHAARPQVQLQQSGPGLVKPSQTLSLTCAISGDSVSSNSAAWNWI
RQSPSRGLEWLGRTYYRSKWYNDYAVSVKSRITINPDTSKNQFSLQLNSVTPEDTAVYYCAREV
TGDLEDAFDIWGQGTMVTVSSGGGGSDIQMTQSPSSLSASVGDRVTITCRASQTIWSYLNWYQQ
RPGKAPNLLIYAASSLQSGVPSRFSGRGSGTDFTLTISSLQAEDFATYYCQQSYSIPQTFGQGT
KLEIKLETTTPAPRPPTPAPTIASQPLSLRPEACRPAAGGAVHTRGLDFACDIYIWAPLAGTCG
VLLLSLVITLYCKRGRKKLLYIFKQPFMRPVQTTQEEDGCSCRFPEEEEGGCELDIRVKESRSA
DAPAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPRRKRRGSGTPDPWGSGATNFSLLKQ
AGDVEENPGPGSMEFGLSWLELVAILKGVQCSRDIQMTQTTSSLSASLGDRVTISCRASQDISK
YLNWYQQKPDGTVKLLIYHTSRLHSGVPSRFSGSGSGTDYSLTISNLEQEDIATYFCQQGNTLP
YTFGGGTKLEITGSTSGSGKPGSGEGSTKGEVKLQESGPGLVAPSQSLSVTCTVSGVSLPDYGV
SWIRQPPRKGLEWLGVIWGSETTYYNSALKSRLTIIKDNSKSQVFLKMNSLQTDDTAIYYCAKH
YYYGGSYAMDYWGQGTSVTVSRIEVMYPPPYLDNEKSNGTIIHVKGKHLCPSPLFPGPSKPFWV
LVVVGGVLACYSLLVTVAFIIFWVHCHRLPGSYDSTSSDSLYPRGIQFRRPHTVAPWPPAYPPV
TSYPPLSQPDLLPIPRSPQPLGGSHRTPSSRRDSDGANSVASYENEGASGIRGAQAGWGVWGPS
WTRLTPVSLPPEPACEDADEDEDDYHNPGYLVVLPDSTPATSTAAPSAPALSTPGIRDSAFSME
SIDDYVNVPESGESAEASLDGSREYVNVSQELHPGAAKTEPAALSSQEAEEVEEEGAPDYENLQ
ELN

In some embodiments, the bicistronic anti-CD22 CAR and anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 80.

An exemplary bicistronic anti-CD22 CAR and anti-CD19 CAR “CAR1-linker-CAR2” or “LAT-CAR” or “ALA-CART′ or “22×19 ALA-CART” or ALA-CART CD22BBz” or “CD22 2nd Gen CAR+CD19-LAT CAR” or “22×19LAT” amino acid sequence is shown below (CAR1; Furin/P2A linker; CAR2).

(SEQ ID NO: 104)
MALPVTALLLPLALLLHAARPQVQLQQSGPGLVKPSQTLSLTCAISGDSVSSNSAAWNWIRQSP
SRGLEWLGRTYYRSKWYNDYAVSVKSRITINPDTSKNQFSLQINSVTPEDTAVYYCAREVTGDL
EDAFDIWGQGTMVTVSSGGGGSDIQMTQSPSSLSASVGDRVTITCRASQTIWSYLNWYQQRPGK
APNLLIYAASSLQSGVPSRFSGRGSGTDFTLTISSLQAEDFATYYCQQSYSIPQTFGQGTKLEI
KLETTTPAPRPPTPAPTIASQPLSLRPEACRPAAGGAVHTRGLDFACDIYIWAPLAGTCGVLLL
SIVITLYCKRGRKKLLYIFKQPFMRPVQTTQEEDGCSCRFPEEEEGGCELDIRVKFSRSADAPA
YQQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPRRKRRGSGTPDPWGSGATNFSLLKQAGDV
EENPGPGSMEFGLSWLFLVAILKGVQCSRDIQMTQTTSSLSASLGDRVTISCRASQDISKYLNW
YQQKPDGTVKLLIYHTSRLHSGVPSRFSGSGSGTDYSLTISNLEQEDIATYFCQQGNTLPYTFG
GGTKLEITGSTSGSGKPGSGEGSTKGEVKLQESGPGLVAPSQSLSVTCTVSGVSLPDYGVSWIR
QPPRKGLEWLGVIWGSETTYYNSALKSRLTIIKDNSKSQVFLKMNSLQTDDTAIYYCAKHYYYG
GSYAMDYWGQGTSVTVSRIEVMYPPPYLDNEKSNGTIIHVKGKHLCPSPLFPGPSKPFWVLVVV
GGVLACYSLLVTVAFIIFWVHCHRLPGSYDSTSSDSLYPRGIQFRRPHTVAPWPPAYPPVTSYP
PLSQPDLLPIPRSPQPLGGSHRIPSSRRDSDGANSVASYENEGASGIRGAQAGWGVWGPSWTRL
TPVSLPPEPACEDADEDEDDYHNPGYLVVLPDSTPATSTAAPSAPALSTPGIRDSAFSMESIDD
YVNVPESGESAEASLDGSREYVNVSQELHPGAAKTEPAALSSQEAEEVEEEGAPDYENLQELN

In some embodiments, the bicistronic anti-CD22 CAR and anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 104.

An exemplary bicistronic anti-CD22 CAR and anti-CD19 CAR “CAR1-linker-CAR2” or “LAT-CAR” polynucleotide sequence is shown below. (CAR1; furin/P2A linker; CAR2).

(SEQ ID NO: 81)
GCTAGCGCCACCATGGCTCTGCCTGTGACAGCTCTGCTGCTGCCTCTGGCCCTGCTGCTCCATG
CTGCTAGACCTCAGGTGCAGCTCCAGCAGTCTGGCCCAGGACTGGTCAAGCCTAGCCAGACCCT
GAGCCTGACCTGCGCCATCAGCGGCGACAGCGTGTCCTCTAACAGCGCCGCCTGGAACTGGATC
AGACAGAGCCCCAGCAGAGGCCTGGAATGGCTGGGCCGGACCTACTACCGGTCCAAGTGGTACA
ACGACTACGCCGTGTCCGTGAAGTCCCGGATCACCATCAACCCCGACACCAGCAAGAACCAGTT
CTCCCTGCAGCTGAACAGCGTGACCCCTGAGGACACCGCCGTGTACTACTGCGCCAGAGAAGTG
ACCGGCGACCTGGAAGATGCCTTCGACATCTGGGGCCAGGGCACCATGGTCACCGTGTCTAGCG
GAGGCGGCGGAAGCGACATCCAGATGACCCAGAGCCCTAGCTCCCTGAGCGCCAGCGTGGGCGA
CAGAGTGACCATCACCTGTCGGGCCAGCCAGACCATCTGGTCCTACCTGAATTGGTATCAGCAG
CGGCCAGGCAAGGCCCCTAACCTGCTGATCTATGCCGCCAGCAGCCTGCAGAGCGGCGTGCCAA
GCAGATTCTCTGGCAGAGGCTCCGGCACCGACTTCACCCTGACAATCAGTTCCCTGCAGGCCGA
GGACTTCGCCACCTACTACTGCCAGCAGTCCTACAGCATCCCTCAGACCTTCGGCCAGGGGACC
AAGCTGGAAATCAAGCTCGAGACCACCACCCCCGCCCCTAGGCCTCCCACACCTGCCCCCACAA
TCGCCTCCCAGCCTCTCAGCCTGAGGCCTGAAGCTTGCAGGCCCGCTGCCGGAGGAGCTGTCCA
TACCAGGGGACTCGACTTCGCCTGCGACATTTACATTTGGGCCCCTCTGGCTGGAACCTGCGGA
GTCCTGCTGCTGTCCCTGGTGATCACACTGTACTGTAAGAGGGGCAGAAAGAAGCTGCTCTACA
TCTTCAAGCAGCCCTTTATGAGACCCGTGCAGACAACCCAGGAGGAAGACGGATGCAGCTGCAG
GTTCCCTGAGGAGGAGGAGGGCGGCTGCGAACTGGATATCAGGGTGAAGTTCAGCAGGAGCGCC
GACGCCCCCGCTTATCAACAGGGCCAGAACCAGCTGTACAACGAGCTGAACCTCGGCAGAAGAG
AGGAGTATGACGTGCTGGACAAGAGGAGGGGCAGGGACCCTGAGATGGGCGGCAAGCCTAGAAG
AAAGAACCCCCAGGAAGGCCTCTACAACGAACTGCAGAAGGACAAGATGGCCGAGGCCTACAGC
GAGATCGGCATGAAAGGCGAGAGAAGGAGGGGAAAGGGACATGACGGCCTGTACCAGGGACTCT
CCACAGCCACCAAGGACACCTACGATGCCCTGCACATGCAGGCTCTGCCCCCTAGAAGGAAGAG
AAGAGGCTCTGGTACCCCCGATCCTTGGGGAAGCGGCGCTACCAACTTCTCCCTGCTCAAGCAG
GCTGGCGATGTGGAGGAGAACCCCGGCCCCGGATCCATGGAGTTTGGCCTGAGCTGGCTGTTCC
TGGTGGCCATCCTCAAGGGCGTGCAGTGCTCCAGGGACATCCAGATGACCCAGACCACAAGCAG
CCTGAGCGCTTCCCTCGGCGACAGGGTGACCATCTCCTGTAGAGCCTCCCAAGACATCTCCAAG
TACCTGAACTGGTACCAGCAGAAACCCGACGGCACCGTGAAGCTGCTGATCTACCACACCAGCA
GGCTGCATTCCGGCGTGCCCTCCAGATTTTCCGGCAGCGGCTCTGGTACCGACTACAGCCTCAC
CATCAGCAACTTAGAACAGGAGGACATCGCCACATATTTCTGCCAACAGGGAAACACACTCCCC
TATACCTTCGGCGGCGGCACAAAGTTAGAAATCACCGGCTCCACATCCGGCAGCGGAAAACCTG
GTTCTGGCGAGGGCAGCACCAAGGGCGAAGTGAAGCTGCAGGAAAGCGGACCTGGACTGGTCGC
TCCCAGCCAGAGCCTCAGCGTGACCTGTACAGTGAGCGGCGTGAGCCTGCCTGATTACGGCGTG
AGCTGGATTAGACAGCCTCCCAGGAAGGGCTTAGAATGGCTCGGCGTGATTTGGGGCAGCGAGA
CAACCTACTATAACAGCGCCCTGAAGAGCAGGCTCACCATTATCAAGGACAACAGCAAATCCCA
GGTCTTCCTGAAGATGAACAGCCTCCAGACCGACGACACCGCCATCTACTACTGCGCCAAGCAC
TACTATTATGGCGGCTCCTACGCCATGGACTACTGGGGCCAGGGCACCAGCGTGACAGTGTCTA
GAATCGAAGTGATGTACCCTCCACCTTACCTGGACAACGAGAAGTCCAACGGCACCATCATCCA
CGTGAAGGGCAAGCACCTGTGTCCTTCTCCACTGTTCCCCGGACCTAGCAAGCCTTTCTGGGTG
CTCGTTGTTGTTGGCGGCGTGCTGGCCTGTTACAGCCTGCTGGTTACCGTGGCCTTCATCATCT
TTTGGGTGCACTGCCACAGACTGCCCGGCAGCTACGATAGCACCAGCAGCGATTCTCTGTACCC
CAGAGGCATCCAGTTCAGACGGCCTCATACAGTGGCTCCCTGGCCTCCTGCTTACCCTCCTGTG
ACAAGCTACCCACCTCTGAGCCAGCCTGACCTGCTGCCTATTCCTAGAAGCCCTCAGCCTCTCG
GCGGCAGCCATAGAACACCTAGCAGCAGAAGAGATAGCGACGGCGCCAATAGCGTGGCCAGCTA
CGAAAATGAAGGCGCCTCTGGCATTAGAGGCGCCCAAGCTGGATGGGGAGTTTGGGGACCTAGC
TGGACAAGACTGACCCCTGTGTCTCTGCCTCCTGAACCTGCCTGCGAAGATGCCGACGAGGACG
AGGATGACTATCACAACCCTGGCTACCTGGTGGTGCTGCCTGATAGCACACCAGCCACATCTAC
AGCCGCTCCTAGTGCTCCTGCTCTGAGCACACCTGGCATCAGAGACAGCGCCTTCAGCATGGAA
TCCATCGACGACTACGTGAACGTGCCCGAGTCTGGCGAATCTGCCGAAGCCTCTCTTGACGGCA
GCCGCGAGTATGTGAACGTGTCCCAAGAACTGCATCCCGGCGCTGCCAAAACAGAACCTGCTGC
TCTGTCTAGCCAAGAGGCCGAGGAAGTGGAAGAAGAAGGCGCCCCTGACTACGAGAACCTGCAA
GAGCTGAACTGATGAGTCGAC

In some embodiments, the bicistronic anti-CD22 CAR and anti-CD19 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 81.

In some embodiments, the bicistronic anti-CD22 CAR and anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 104.

An exemplary bicistronic anti-CD22 CAR and anti-CD19 CAR “CAR1-linker-CAR2” or “LAT-CAR” polynucleotide sequence is shown below. (CAR1; furin/P2A linker; CAR2).

(SEQ ID NO: 105)
ATGGCTCTGCCTGTGACAGCTCTGCTGCTGCCTCTGGCCCTGCTGCTCCATGCTGCTAGACCTC
AGGTGCAGCTCCAGCAGTCTGGCCCAGGACTGGTCAAGCCTAGCCAGACCCTGAGCCTGACCTG
CGCCATCAGCGGCGACAGCGTGTCCTCTAACAGCGCCGCCTGGAACTGGATCAGACAGAGCCCC
AGCAGAGGCCTGGAATGGCTGGGCCGGACCTACTACCGGTCCAAGTGGTACAACGACTACGCCG
TGTCCGTGAAGTCCCGGATCACCATCAACCCCGACACCAGCAAGAACCAGTTCTCCCTGCAGCT
GAACAGCGTGACCCCTGAGGACACCGCCGTGTACTACTGCGCCAGAGAAGTGACCGGCGACCTG
GAAGATGCCTTCGACATCTGGGGCCAGGGCACCATGGTCACCGTGTCTAGCGGAGGCGGCGGAA
GCGACATCCAGATGACCCAGAGCCCTAGCTCCCTGAGCGCCAGCGTGGGCGACAGAGTGACCAT
CACCTGTCGGGCCAGCCAGACCATCTGGTCCTACCTGAATTGGTATCAGCAGCGGCCAGGCAAG
GCCCCTAACCTGCTGATCTATGCCGCCAGCAGCCTGCAGAGCGGCGTGCCAAGCAGATTCTCTG
GCAGAGGCTCCGGCACCGACTTCACCCTGACAATCAGTTCCCTGCAGGCCGAGGACTTCGCCAC
CTACTACTGCCAGCAGTCCTACAGCATCCCTCAGACCTTCGGCCAGGGGACCAAGCTGGAAATC
AAGCTCGAGACCACCACCCCCGCCCCTAGGCCTCCCACACCTGCCCCCACAATCGCCTCCCAGC
CTCTCAGCCTGAGGCCTGAAGCTTGCAGGCCCGCTGCCGGAGGAGCTGTCCATACCAGGGGACT
CGACTTCGCCTGCGACATTTACATTTGGGCCCCTCTGGCTGGAACCTGCGGAGTCCTGCTGCTG
TCCCTGGTGATCACACTGTACTGTAAGAGGGGCAGAAAGAAGCTGCTCTACATCTTCAAGCAGC
CCTTTATGAGACCCGTGCAGACAACCCAGGAGGAAGACGGATGCAGCTGCAGGTTCCCTGAGGA
GGAGGAGGGCGGCTGCGAACTGGATATCAGGGTGAAGTTCAGCAGGAGCGCCGACGCCCCCGCT
TATCAACAGGGCCAGAACCAGCTGTACAACGAGCTGAACCTCGGCAGAAGAGAGGAGTATGACG
TGCTGGACAAGAGGAGGGGCAGGGACCCTGAGATGGGCGGCAAGCCTAGAAGAAAGAACCCCCA
GGAAGGCCTCTACAACGAACTGCAGAAGGACAAGATGGCCGAGGCCTACAGCGAGATCGGCATG
AAAGGCGAGAGAAGGAGGGGAAAGGGACATGACGGCCTGTACCAGGGACTCTCCACAGCCACCA
AGGACACCTACGATGCCCTGCACATGCAGGCTCTGCCCCCTAGAAGGAAGAGAAGAGGCTCTGG
TACCCCCGATCCTTGGGGAAGCGGCGCTACCAACTTCTCCCTGCTCAAGCAGGCTGGCGATGTG
GAGGAGAACCCCGGCCCCGGATCCATGGAGTTTGGCCTGAGCTGGCTGTTCCTGGTGGCCATCC
TCAAGGGCGTGCAGTGCTCCAGGGACATCCAGATGACCCAGACCACAAGCAGCCTGAGCGCTTC
CCTCGGCGACAGGGTGACCATCTCCTGTAGAGCCTCCCAAGACATCTCCAAGTACCTGAACTGG
TACCAGCAGAAACCCGACGGCACCGTGAAGCTGCTGATCTACCACACCAGCAGGCTGCATTCCG
GCGTGCCCTCCAGATTTTCCGGCAGCGGCTCTGGTACCGACTACAGCCTCACCATCAGCAACTT
AGAACAGGAGGACATCGCCACATATTTCTGCCAACAGGGAAACACACTCCCCTATACCTTCGGC
GGCGGCACAAAGTTAGAAATCACCGGCTCCACATCCGGCAGCGGAAAACCTGGTTCTGGCGAGG
GCAGCACCAAGGGCGAAGTGAAGCTGCAGGAAAGCGGACCTGGACTGGTCGCTCCCAGCCAGAG
CCTCAGCGTGACCTGTACAGTGAGCGGCGTGAGCCTGCCTGATTACGGCGTGAGCTGGATTAGA
CAGCCTCCCAGGAAGGGCTTAGAATGGCTCGGCGTGATTTGGGGCAGCGAGACAACCTACTATA
ACAGCGCCCTGAAGAGCAGGCTCACCATTATCAAGGACAACAGCAAATCCCAGGTCTTCCTGAA
GATGAACAGCCTCCAGACCGACGACACCGCCATCTACTACTGCGCCAAGCACTACTATTATGGC
GGCTCCTACGCCATGGACTACTGGGGCCAGGGCACCAGCGTGACAGTGTCTAGAATCGAAGTGA
TGTACCCTCCACCTTACCTGGACAACGAGAAGTCCAACGGCACCATCATCCACGTGAAGGGCAA
GCACCTGTGTCCTTCTCCACTGTTCCCCGGACCTAGCAAGCCTTTCTGGGTGCTCGTTGTTGTT
GGCGGCGTGCTGGCCTGTTACAGCCTGCTGGTTACCGTGGCCTTCATCATCTTTTGGGTGCACT
GCCACAGACTGCCCGGCAGCTACGATAGCACCAGCAGCGATTCTCTGTACCCCAGAGGCATCCA
GTTCAGACGGCCTCATACAGTGGCTCCCTGGCCTCCTGCTTACCCTCCTGTGACAAGCTACCCA
CCTCTGAGCCAGCCTGACCTGCTGCCTATTCCTAGAAGCCCTCAGCCTCTCGGCGGCAGCCATA
GAACACCTAGCAGCAGAAGAGATAGCGACGGCGCCAATAGCGTGGCCAGCTACGAAAATGAAGG
CGCCTCTGGCATTAGAGGCGCCCAAGCTGGATGGGGAGTTTGGGGACCTAGCTGGACAAGACTG
ACCCCTGTGTCTCTGCCTCCTGAACCTGCCTGCGAAGATGCCGACGAGGACGAGGATGACTATC
ACAACCCTGGCTACCTGGTGGTGCTGCCTGATAGCACACCAGCCACATCTACAGCCGCTCCTAG
TGCTCCTGCTCTGAGCACACCTGGCATCAGAGACAGCGCCTTCAGCATGGAATCCATCGACGAC
TACGTGAACGTGCCCGAGTCTGGCGAATCTGCCGAAGCCTCTCTTGACGGCAGCCGCGAGTATG
TGAACGTGTCCCAAGAACTGCATCCCGGCGCTGCCAAAACAGAACCTGCTGCTCTGTCTAGCCA
AGAGGCCGAGGAAGTGGAAGAAGAAGGCGCCCCTGACTACGAGAACCTGCAAGAGCTGAACTGA
TGAGTCGAC

In some embodiments, the bicistronic anti-CD22 CAR and anti-CD19 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 105.

iii) Exemplary Anti-CD22 Bicistronic Affinity CAR Constructs

An exemplary bicistronic Standard affinity anti-CD22 CAR and Standard affinity anti-CD22 LAT-CAR “CAR1-linker-CAR2” or “SAff/SAff-LAT” or “LAT-CAR” or “22ALACART1” amino acid sequence is shown below (SAff scFv CAR1; Furin/P2A linker; SAff scFv CAR2).

(SEQ ID NO: 200)
ASATMALPVTALLLPLALLLHAARPQVQLQQSGPGLVKPSQTLSLTCAISGDSVS
SNSAAWNWIRQSPSRGLEWLGRTYYRSKWYNDYAVSVKSRITINPDTSKNQFSLQLNS
VTPEDTAVYYCAREVTGDLEDAFDIWGQGTMVTVSSGGGGSDIQMTQSPSSLSASVGD
RVTITCRASQTIWSYLNWYQQRPGKAPNLLIYAASSLQSGVPSRFSGRGSGTDFTLTISSL
QAEDFATYYCQQSYSIPQTFGQGTKLEIKLETTTPAPRPPTPAPTIASQPLSLRPEACRPAA
GGAVHTRGLDFACDIYIWAPLAGTCGVLLLSLVITLYCKRGRKKLLYIFKQPFMRPVQT
TQEEDGCSCRFPEEEEGGCELDIRVKFSRSADAPAYQQGQNQLYNELNLGRREEYDVLD
KRRGRDPEMGGKPRRKNPQEGLYNELQKDKMAEAYSEIGMKGERRRGKGHDGLYQG
LSTATKDTYDALHMQALPPRRKRRGSGTPDPWGSGATNFSLLKQAGDVEENPGPGS
MALPVTALLLPLALLLHAARPDYKDDDDKQVQLQQSGPGLVKPSQTLSLTCAISGDSVSSNSA
AWNWIRQSPSRGLEWLGRTYYRSKWYNDYAVSVKSRITINPDTSKNQFSLQLNSVTPEDTAVYY
CAREVTGDLEDAFDIWGQGTMVTVSSGGGGSDIQMTQSPSSLSASVGDRVTITCRASQTIWSY
LNWYQQRPGKAPNLLIYAASSLQSGVPSRFSGRGSGTDFTLTISSLQAEDFATYYCQQSYSIPQT
FGQGTKLEIKSRIEVMYPPPYLDNEKSNGTIIHVKGKHLCPSPLFPGPSKPFWVLVVVGG
VLACYSLLVTVAFIIFWVHCHRLPGSYDSTSSDSLYPRGIQFRRPHTVAPWPPAYPPVTS
YPPLSQPDLLPIPRSPQPLGGSHRTPSSRRDSDGANSVASYENEGASGIRGAQAGWGVW
GPSWTRLTPVSLPPEPACEDADEDEDDYHNPGYLVVLPDSTPATSTAAPSAPALSTPGIR
DSAFSMESIDDYVNVPESGESAEASLDGSREYVNVSQELHPGAAKTEPAALSSQEAEEV
EEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 200.

An exemplary bicistronic Standard affinity anti-CD22 CAR and Standard affinity anti-CD22 LAT-CAR “CAR1-linker-CAR2” or “SAff/SAff-LAT” or “LAT-CAR” or “22ALACART1” polynucleotide sequence is shown below (SAff scFv CAR1; SAff scFv CAR2)

(SEQ ID NO: 201)
CTCGAGATGGCTCTGCCTGTGACAGCTCTGCTGCTGCCTCTGGCCCTGCTGCT
CCATGCTGCTAGACCTCAGGTGCAGCTCCAGCAGTCTGGCCCAGGACTGGTCAAGCC
TAGCCAGACCCTGAGCCTGACCTGCGCCATCAGCGGCGACAGCGTGTCCTCTAACA
GCGCCGCCTGGAACTGGATCAGACAGAGCCCCAGCAGAGGCCTGGAATGGCTGGGC
CGGACCTACTACCGGTCCAAGTGGTACAACGACTACGCCGTGTCCGTGAAGTCCCG
GATCACCATCAACCCCGACACCAGCAAGAACCAGTTCTCCCTGCAGCTGAACAGCG
TGACCCCTGAGGACACCGCCGTGTACTACTGCGCCAGAGAAGTGACCGGCGACCTG
GAAGATGCCTTCGACATCTGGGGCCAGGGCACCATGGTCACCGTGTCTAGCGGAGG
CGGCGGAAGCGACATCCAGATGACCCAGAGCCCTAGCTCCCTGAGCGCCAGCGTGG
GCGACAGAGTGACCATCACCTGTCGGGCCAGCCAGACCATCTGGTCCTACCTGAATT
GGTATCAGCAGCGGCCAGGCAAGGCCCCTAACCTGCTGATCTATGCCGCCAGCAGC
CTGCAGAGCGGCGTGCCAAGCAGATTCTCTGGCAGAGGCTCCGGCACCGACTTCAC
CCTGACAATCAGTTCCCTGCAGGCCGAGGACTTCGCCACCTACTACTGCCAGCAGTC
CTACAGCATCCCTCAGACCTTCGGCCAGGGGACCAAGCTGGAAATCAAGACTAGTT
CGAGACCACCACCCCCGCCCCTAGGCCTCCCACACCTGCCCCCACAATCGCCTCCCA
GCCTCTCAGCCTGAGGCCTGAAGCTTGCAGGCCCGCTGCCGGAGGAGCTGTCCATAC
CAGGGGACTCGACTTCGCCTGCGACATTTACATTTGGGCCCCTCTGGCTGGAACCTG
CGGAGTCCTGCTGCTGTCCCTGGTGATCACACTGTACTGTAAGAGGGGCAGAAAGA
AGCTGCTCTACATCTTCAAGCAGCCCTTTATGAGACCCGTGCAGACAACCCAGGAGG
AAGACGGATGCAGCTGCAGGTTCCCTGAGGAGGAGGAGGGCGGCTGCGAACTGGAT
ATCAGGGTGAAGTTCAGCAGGAGCGCCGACGCCCCCGCTTATCAACAGGGCCAGAA
CCAGCTGTACAACGAGCTGAACCTCGGCAGAAGAGAGGAGTATGACGTGCTGGACA
AGAGGAGGGGCAGGGACCCTGAGATGGGCGGCAAGCCTAGAAGAAAGAACCCCCA
GGAAGGCCTCTACAACGAACTGCAGAAGGACAAGATGGCCGAGGCCTACAGCGAG
ATCGGCATGAAAGGCGAGAGAAGGAGGGGAAAGGGACATGACGGCCTGTACCAGG
GACTCTCCACAGCCACCAAGGACACCTACGATGCCCTGCACATGCAGGCTCTGCCCC
CTAGAAGGAAGAGAAGAGGCTCTGGTACCCCCGATCCTTGGGGAAGCGGCGCTACC
AACTTCTCCCTGCTCAAGCAGGCTGGCGATGTGGAGGAGAACCCCGGCCCCGCTCGA
GATGGCTCTGCCTGTGACAGCTCTGCTGCTGCCTCTGGCCCTGCTGCTCCATGCTGCTAG
ACCTCAGGTGCAGCTCCAGCAGTCTGGCCCAGGACTGGTCAAGCCTAGCCAGACCCTGA
GCCTGACCTGCGCCATCAGCGGCGACAGCGTGTCCTCTAACAGCGCCGCCTGGAACTGG
ATCAGACAGAGCCCCAGCAGAGGCCTGGAATGGCTGGGCCGGACCTACTACCGGTCCAA
GTGGTACAACGACTACGCCGTGTCCGTGAAGTCCCGGATCACCATCAACCCCGACACCAG
CAAGAACCAGTTCTCCCTGCAGCTGAACAGCGTGACCCCTGAGGACACCGCCGTGTACTA
CTGCGCCAGAGAAGTGACCGGCGACCTGGAAGATGCCTTCGACATCTGGGGCCAGGGCA
CCATGGTCACCGTGTCTAGCGGAGGCGGCGGAAGCGACATCCAGATGACCCAGAGCCCT
AGCTCCCTGAGCGCCAGCGTGGGCGACAGAGTGACCATCACCTGTCGGGCCAGCCAGAC
CATCTGGTCCTACCTGAATTGGTATCAGCAGCGGCCAGGCAAGGCCCCTAACCTGCTGAT
CTATGCCGCCAGCAGCCTGCAGAGCGGCGTGCCAAGCAGATTCTCTGGCAGAGGCTCCG
GCACCGACTTCACCCTGACAATCAGTTCCCTGCAGGCCGAGGACTTCGCCACCTACTACT
GCCAGCAGTCCTACAGCATCCCTCAGACCTTCGGCCAGGGGACCAAGCTGGAAATCAAG
ACTAGTCTAGAATCGAAGTGATGTACCCTCCACCTTACCTGGACAACGAGAAGTCCA
ACGGCACCATCATCCACGTGAAGGGCAAGCACCTGTGTCCTTCTCCACTGTTCCCCG
GACCTAGCAAGCCTTTCTGGGTGCTCGTTGTTGTTGGCGGCGTGCTGGCCTGTTACA
GCCTGCTGGTTACCGTGGCCTTCATCATCTTTTGGGTGCACTGCCACAGACTGCCCG
GCAGCTACGATAGCACCAGCAGCGATTCTCTGTACCCCAGAGGCATCCAGTTCAGA
CGGCCTCATACAGTGGCTCCCTGGCCTCCTGCTTACCCTCCTGTGACAAGCTACCCA
CCTCTGAGCCAGCCTGACCTGCTGCCTATTCCTAGAAGCCCTCAGCCTCTCGGCGGC
AGCCATAGAACACCTAGCAGCAGAAGAGATAGCGACGGCGCCAATAGCGTGGCCA
GCTACGAAAATGAAGGCGCCTCTGGCATTAGAGGCGCCCAAGCTGGATGGGGAGTT
TGGGGACCTAGCTGGACAAGACTGACCCCTGTGTCTCTGCCTCCTGAACCTGCCTGC
GAAGATGCCGACGAGGACGAGGATGACTATCACAACCCTGGCTACCTGGTGGTGCT
GCCTGATAGCACACCAGCCACATCTACAGCCGCTCCTAGTGCTCCTGCTCTGAGCAC
ACCTGGCATCAGAGACAGCGCCTTCAGCATGGAATCCATCGACGACTACGTGAACG
TGCCCGAGTCTGGCGAATCTGCCGAAGCCTCTCTTGACGGCAGCCGCGAGTATGTGA
ACGTGTCCCAAGAACTGCATCCCGGCGCTGCCAAAACAGAACCTGCTGCTCTGTCTA
GCCAAGAGGCCGAGGAAGTGGAAGAAGAAGGCGCCCCTGACTACGAGAACCTGCA
AGAGCTGAACTGATGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 201.

An exemplary bicistronic Standard affinity anti-CD22 CAR and High affinity anti-CD22 LAT-CAR “CAR1-linker-CAR2” or “SAff/HiAff-LAT” or “LAT-CAR” or “22ALACART2” amino acid sequence is shown below (SAff scFv CAR1; Furin/P2A linker; HiAff scFv CAR2).

(SEQ ID NO: 202)
ASATMALPVTALLLPLALLLHAARPQVQLQQSGPGLVKPSQTLSLTCAISGDSVS
SNSAAWNWIRQSPSRGLEWLGRTYYRSKWYNDYAVSVKSRITINPDTSKNQFSLQLNS
VTPEDTAVYYCAREVTGDLEDAFDIWGQGTMVTVSSGGGGSDIQMTQSPSSLSASVGD
RVTITCRASQTIWSYLNWYQQRPGKAPNLLIYAASSLQSGVPSRFSGRGSGTDFTLTISSL
QAEDFATYYCQQSYSIPQTFGQGTKLEIKLETTTPAPRPPTPAPTIASQPLSLRPEACRPAA
GGAVHTRGLDFACDIYIWAPLAGTCGVLLLSLVITLYCKRGRKKLLYIFKQPFMRPVQT
TQEEDGCSCRFPEEEEGGCELDIRVKFSRSADAPAYQQGQNQLYNELNLGRREEYDVLD
KRRGRDPEMGGKPRRKNPQEGLYNELQKDKMAEAYSEIGMKGERRRGKGHDGLYQG
LSTATKDTYDALHMQALPPRRKRRGSGTPDPWGSGATNFSLLKQAGDVEENPGPGS
MALPVTALLLPLALLLHAARPDYKDDDDKQVQLQQSGPGMVKPSQTLSLTCAISGDSVSSNSV
AWNWIRQSPSRGLEWLGRTYYRSTWYNDYAVSMKSRITINPDTNKNQFSLQLNSVTPEDTAVY
YCAREVTGDLEDAFDIWGQGTMVTVSSGGGGSGGGGSGGGGSDIQMIQSPSSLSASVGDRVT
ITCRASQTIWSYLNWYRQRPGEAPNLLIYAASSLQSGVPSRESGRGSGTDFTLTISSLQAEDFAT
YYCQQSYSIPQTFGQGTKLEIKSRIEVMYPPPYLDNEKSNGTIIHVKGKHLCPSPLFPGPSKP
FWVLVVVGGVLACYSLLVTVAFIIFWVHCHRLPGSYDSTSSDSLYPRGIQFRRPHTVAP
WPPAYPPVTSYPPLSQPDLLPIPRSPQPLGGSHRTPSSRRDSDGANSVASYENEGASGIRG
AQAGWGVWGPSWTRLTPVSLPPEPACEDADEDEDDYHNPGYLVVLPDSTPATSTAAPS
APALSTPGIRDSAFSMESIDDYVNVPESGESAEASLDGSREYVNVSQELHPGAAKTEPAA
LSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 202.

An exemplary bicistronic Standard affinity anti-CD22 CAR and High affinity anti-CD22 LAT-CAR “CAR1-linker-CAR2” or “SAff/HiAff-LAT” or “LAT-CAR” or “22ALACART2” polynucleotide sequence is shown below (SAff scFv CAR1; HiAff scFv CAR2).

(SEQ ID NO: 203)
CTCGAGATGGCTCTGCCTGTGACAGCTCTGCTGCTGCCTCTGGCCCTGCT
GCTCCATGCTGCTAGACCTCAGGTGCAGCTCCAGCAGTCTGGCCCAGGAC
TGGTCAAGCCTAGCCAGACCCTGAGCCTGACCTGCGCCATCAGCGGCGAC
AGCGTGTCCTCTAACAGCGCCGCCTGGAACTGGATCAGACAGAGCCCCAG
CAGAGGCCTGGAATGGCTGGGCCGGACCTACTACCGGTCCAAGTGGTACA
ACGACTACGCCGTGTCCGTGAAGTCCCGGATCACCATCAACCCCGACACC
AGCAAGAACCAGTTCTCCCTGCAGCTGAACAGCGTGACCCCTGAGGACAC
CGCCGTGTACTACTGCGCCAGAGAAGTGACCGGCGACCTGGAAGATGCCT
TCGACATCTGGGGCCAGGGCACCATGGTCACCGTGTCTAGCGGAGGCGGC
GGAAGCGACATCCAGATGACCCAGAGCCCTAGCTCCCTGAGCGCCAGCGT
GGGCGACAGAGTGACCATCACCTGTCGGGCCAGCCAGACCATCTGGTCCT
ACCTGAATTGGTATCAGCAGCGGCCAGGCAAGGCCCCTAACCTGCTGATC
TATGCCGCCAGCAGCCTGCAGAGCGGCGTGCCAAGCAGATTCTCTGGCAG
AGGCTCCGGCACCGACTTCACCCTGACAATCAGTTCCCTGCAGGCCGAGG
ACTTCGCCACCTACTACTGCCAGCAGTCCTACAGCATCCCTCAGACCTTC
GGCCAGGGGACCAAGCTGGAAATCAAGACTAGTTCGAGACCACCACCCCC
GCCCCTAGGCCTCCCACACCTGCCCCCACAATCGCCTCCCAGCCTCTCAG
CCTGAGGCCTGAAGCTTGCAGGCCCGCTGCCGGAGGAGCTGTCCATACCA
GGGGACTCGACTTCGCCTGCGACATTTACATTTGGGCCCCTCTGGCTGGA
ACCTGCGGAGTCCTGCTGCTGTCCCTGGTGATCACACTGTACTGTAAGAG
GGGCAGAAAGAAGCTGCTCTACATCTTCAAGCAGCCCTTTATGAGACCCG
TGCAGACAACCCAGGAGGAAGACGGATGCAGCTGCAGGTTCCCTGAGGAG
GAGGAGGGCGGCTGCGAACTGGATATCAGGGTGAAGTTCAGCAGGAGCGC
CGACGCCCCCGCTTATCAACAGGGCCAGAACCAGCTGTACAACGAGCTGA
ACCTCGGCAGAAGAGAGGAGTATGACGTGCTGGACAAGAGGAGGGGCAGG
GACCCTGAGATGGGCGGCAAGCCTAGAAGAAAGAACCCCCAGGAAGGCCT
CTACAACGAACTGCAGAAGGACAAGATGGCCGAGGCCTACAGCGAGATCG
GCATGAAAGGCGAGAGAAGGAGGGGAAAGGGACATGACGGCCTGTACCAG
GGACTCTCCACAGCCACCAAGGACACCTACGATGCCCTGCACATGCAGGC
TCTGCCCCCTAGAAGGAAGAGAAGAGGCTCTGGTACCCCCGATCCTTGGG
GAAGCGGCGCTACCAACTTCTCCCTGCTCAAGCAGGCTGGCGATGTGGAG
GAGAACCCCGGCCCCGGATCCATGGCTCTGCCTGTGACAGCTCTGCTGCT
GCCTCTGGCCCTGCTGCTCCATGCTGCTAGACCTGACTACAAAGACGATG
ACGACAAGCAGGTGCAGCTCCAGCAGTCTGGCCCAGGAATGGTCAAGCCT
AGCCAGACCCTGAGCCTGACCTGCGCCATCAGCGGCGACAGCGTGTCCTC
TAACAGCGTCGCCTGGAACTGGATCAGACAGAGCCCCAGCAGAGGCCTGG
AATGGCTGGGCCGGACCTACTACCGGTCCACGTGGTACAACGACTACGCC
GTGTCCATGAAGTCCCGGATCACCATCAACCCCGACACCAACAAGAACCA
GTTCTCCCTGCAGCTGAACAGCGTGACCCCTGAGGACACCGCCGTGTACT
ACTGCGCCAGAGAAGTGACCGGCGACCTGGAAGATGCCTTCGACATCTGG
GGCCAGGGCACCATGGTCACCGTGTCTAGCGGAGGCGGCGGAAGCGGTGG
AGGCGGTAGCGGCGGTGGCGGTTCCGACATCCAGATGATCCAGAGCCCTA
GCTCCCTGAGCGCCAGCGTGGGCGACAGAGTGACCATCACCTGTCGGGCC
AGCCAGACCATCTGGTCCTACCTGAATTGGTATCGGCAGCGGCCAGGCGA
GGCCCCTAACCTGCTGATCTATGCCGCCAGCAGCCTGCAGAGCGGCGTGC
CAAGCAGATTCTCTGGCAGAGGCTCCGGCACCGACTTCACCCTGACAATC
AGTTCCCTGCAGGCCGAGGACTTCGCCACCTACTACTGCCAGCAGTCCTA
CAGCATCCCTCAGACCTTCGGCCAGGGGACCAAGCTGGAAATCAAGTCTA
GAATCGAAGTGATGTACCCTCCACCTTACCTGGACAACGAGAAGTCCAAC
GGCACCATCATCCACGTGAAGGGCAAGCACCTGTGTCCTTCTCCACTGTT
CCCCGGACCTAGCAAGCCTTTCTGGGTGCTCGTTGTTGTTGGCGGCGTGC
TGGCCTGTTACAGCCTGCTGGTTACCGTGGCCTTCATCATCTTTTGGGTG
CACTGCCACAGACTGCCCGGCAGCTACGATAGCACCAGCAGCGATTCTCT
GTACCCCAGAGGCATCCAGTTCAGACGGCCTCATACAGTGGCTCCCTGGC
CTCCTGCTTACCCTCCTGTGACAAGCTACCCACCTCTGAGCCAGCCTGAC
CTGCTGCCTATTCCTAGAAGCCCTCAGCCTCTCGGCGGCAGCCATAGAAC
ACCTAGCAGCAGAAGAGATAGCGACGGCGCCAATAGCGTGGCCAGCTACG
AAAATGAAGGCGCCTCTGGCATTAGAGGCGCCCAAGCTGGATGGGGAGTT
TGGGGACCTAGCTGGACAAGACTGACCCCTGTGTCTCTGCCTCCTGAACC
TGCCTGCGAAGATGCCGACGAGGACGAGGATGACTATCACAACCCTGGCT
ACCTGGTGGTGCTGCCTGATAGCACACCAGCCACATCTACAGCCGCTCCT
AGTGCTCCTGCTCTGAGCACACCTGGCATCAGAGACAGCGCCTTCAGCAT
GGAATCCATCGACGACTACGTGAACGTGCCCGAGTCTGGCGAATCTGCCG
AAGCCTCTCTTGACGGCAGCCGCGAGTATGTGAACGTGTCCCAAGAACTG
CATCCCGGCGCTGCCAAAACAGAACCTGCTGCTCTGTCTAGCCAAGAGGC
CGAGGAAGTGGAAGAAGAAGGCGCCCCTGACTACGAGAACCTGCAAGAGC
TGAACTGATGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 203.

An exemplary bicistronic High affinity anti-CD22 CAR and Standard affinity anti-CD22 LAT-CAR “CAR1-linker-CAR2” or “HiAff/SAff-LAT” or “LAT-CAR” or “22ALACART3” amino acid sequence is shown below (HiAff scFv CAR1; Furin/P2A linker; SAff scFv CAR2)

(SEQ ID NO: 204)
MALPVTALLLPLALLLHAARPQVQLQQSGPGMVKPSQTLSLTCAISGDSV
SSNSVAWNWIRQSPSRGLEWLGRTYYRSTWYNDYAVSMKSRITINPDTNK
NQFSLQLNSVTPEDTAVYYCAREVTGDLEDAFDIWGQGTMVTVSSGGGGS
GGGGSGGGGSDIQMIQSPSSLSASVGDRVTITCRASQTIWSYLNWYRQRP
GEAPNLLIYAASSLQSGVPSRFSGRGSGTDFTLTISSLQAEDFATYYCQQ
SYSIPQTFGQGTKLEIKLETTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELDIRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPRRKRRGS
GTPDPWGSGATNFSLLKQAGDVEENPGPGSMALPVTALLLPLALLLHAAR
PDYKDDDDKQVQLQQSGPGLVKPSQTLSLTCAISGDSVSSNSAAWNWIRQ
SPSRGLEWLGRTYYRSKWYNDYAVSVKSRITINPDTSKNQFSLQLNSVTP
EDTAVYYCAREVTGDLEDAFDIWGQGTMVTVSSGGGGSDIQMTQSPSSLS
ASVGDRVTITCRASQTIWSYLNWYQQRPGKAPNLLIYAASSLQSGVPSRF
SGRGSGTDFTLTISSLQAEDFATYYCQQSYSIPQTFGQGTKLEIKSRIEV
MYPPPYLDNEKSNGTIIHVKGKHLCPSPLFPGPSKPFWVLVVVGGVLACY
SLLVTVAFIIFWVHCHRLPGSYDSTSSDSLYPRGIQFRRPHTVAPWPPAY
PPVTSYPPLSQPDLLPIPRSPQPLGGSHRTPSSRRDSDGANSVASYENEG
ASGIRGAQAGWGVWGPSWTRLTPVSLPPEPACEDADEDEDDYHNPGYLVV
LPDSTPATSTAAPSAPALSTPGIRDSAFSMESIDDYVNVPESGESAEASL
DGSREYVNVSQELHPGAAKTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 204.

An exemplary bicistronic High affinity anti-CD22 CAR and Standard affinity anti-CD22 LAT-CAR “CAR1-linker-CAR2” or “HiAff/SAff-LAT” or “LAT-CAR” or “22ALACART3” polynucleotide sequence is shown below (HiAff scFv CAR1; SAff scFv CAR2)

(SEQ ID NO: 205)
ATGGCACTGCCAGTGACTGCATTACTCTTGCCACTCGCGCTACTGTTACA
CGCAGCACGTCCACATCACCATCACCATCACCAAGTCCAATTGCAACAAA
GCGGGCCGGGCATGGTGAAACCGAGTCAAACGTTATCTCTTACGTGTGCG
ATTTCGGGGGATAGTGTCAGCAGCAATTCAGTGGCGTGGAATTGGATTCG
CCAATCGCCGAGTCGCGGGTTGGAGTGGCTCGGGCGCACGTATTATCGCA
GCACATGGTATAATGATTATGCGGTCAGCATGAAAAGCCGCATTACGATT
AATCCGGATACGAACAAAAATCAATTTAGCTTACAATTAAATTCCGTCAC
GCCGGAAGATACAGCGGTCTATTATTGTGCGCGCGAGGTCACGGGGGATC
TCGAAGACGCGTTTGATATTTGGGGGCAAGGGACCATGGTGACTGTCAGC
TCTGGTGGAGGGGGCAGTGGAGGTGGGGGATCGGGAGGTGGTGGCAGTGA
TATTCAAATGATCCAAAGTCCATCCAGCCTATCCGCATCTGTCGGAGATC
GCGTAACGATTACGTGCCGCGCGAGTCAAACGATTTGGAGCTATCTGAAC
TGGTACCGGCAACGCCCGGGCGAAGCGCCGAATCTCTTGATTTACGCGGC
GTCCTCATTACAGTCGGGTGTCCCGAGCCGCTTTAGCGGCCGCGGAAGCG
GTACGGATTTTACGTTAACCATTAGCAGCCTCCAGGCGGAAGATTTTGCG
ACGTATTACTGTCAACAGAGCTATAGCATTCCGCAGACGTTTGGTCAGGG
CACGAAATTGGAGATTAAACTCGAGACCACCACCCCCGCCCCTAGGCCTC
CCACACCTGCCCCCACAATCGCCTCCCAGCCTCTCAGCCTGAGGCCTGAA
GCTTGCAGGCCCGCTGCCGGAGGAGCTGTCCATACCAGGGGACTCGACTT
CGCCTGCGACATTTACATTTGGGCCCCTCTGGCTGGAACCTGCGGAGTCC
TGCTGCTGTCCCTGGTGATCACACTGTACTGTAAGAGGGGCAGAAAGAAG
CTGCTCTACATCTTCAAGCAGCCCTTTATGAGACCCGTGCAGACAACCCA
GGAGGAAGACGGATGCAGCTGCAGGTTCCCTGAGGAGGAGGAGGGCGGCT
GCGAACTGGATATCAGGGTGAAGTTCAGCAGGAGCGCCGACGCCCCCGCT
TATCAACAGGGCCAGAACCAGCTGTACAACGAGCTGAACCTCGGCAGAAG
AGAGGAGTATGACGTGCTGGACAAGAGGAGGGGCAGGGACCCTGAGATGG
GCGGCAAGCCTAGAAGAAAGAACCCCCAGGAAGGCCTCTACAACGAACTG
CAGAAGGACAAGATGGCCGAGGCCTACAGCGAGATCGGCATGAAAGGCGA
GAGAAGGAGGGGAAAGGGACATGACGGCCTGTACCAGGGACTCTCCACAG
CCACCAAGGACACCTACGATGCCCTGCACATGCAGGCTCTGCCCCCTAGA
AGGAAGAGAAGAGGCTCTGGTACCCCCGATCCTTGGGGAAGCGGCGCTAC
CAACTTCTCCCTGCTCAAGCAGGCTGGCGATGTGGAGGAGAACCCCGGCC
CCGCTCGAGATGGCTCTGCCTGTGACAGCTCTGCTGCTGCCTCTGGCCCT
GCTGCTCCATGCTGCTAGACCTCAGGTGCAGCTCCAGCAGTCTGGCCCAG
GACTGGTCAAGCCTAGCCAGACCCTGAGCCTGACCTGCGCCATCAGCGGC
GACAGCGTGTCCTCTAACAGCGCCGCCTGGAACTGGATCAGACAGAGCCC
CAGCAGAGGCCTGGAATGGCTGGGCCGGACCTACTACCGGTCCAAGTGGT
ACAACGACTACGCCGTGTCCGTGAAGTCCCGGATCACCATCAACCCCGAC
ACCAGCAAGAACCAGTTCTCCCTGCAGCTGAACAGCGTGACCCCTGAGGA
CACCGCCGTGTACTACTGCGCCAGAGAAGTGACCGGCGACCTGGAAGATG
CCTTCGACATCTGGGGCCAGGGCACCATGGTCACCGTGTCTAGCGGAGGC
GGCGGAAGCGACATCCAGATGACCCAGAGCCCTAGCTCCCTGAGCGCCAG
CGTGGGCGACAGAGTGACCATCACCTGTCGGGCCAGCCAGACCATCTGGT
CCTACCTGAATTGGTATCAGCAGCGGCCAGGCAAGGCCCCTAACCTGCTG
ATCTATGCCGCCAGCAGCCTGCAGAGCGGCGTGCCAAGCAGATTCTCTGG
CAGAGGCTCCGGCACCGACTTCACCCTGACAATCAGTTCCCTGCAGGCCG
AGGACTTCGCCACCTACTACTGCCAGCAGTCCTACAGCATCCCTCAGACC
TTCGGCCAGGGGACCAAGCTGGAAATCAAGACTAGTCTAGAATCGAAGTG
ATGTACCCTCCACCTTACCTGGACAACGAGAAGTCCAACGGCACCATCAT
CCACGTGAAGGGCAAGCACCTGTGTCCTTCTCCACTGTTCCCCGGACCTA
GCAAGCCTTTCTGGGTGCTCGTTGTTGTTGGCGGCGTGCTGGCCTGTTAC
AGCCTGCTGGTTACCGTGGCCTTCATCATCTTTTGGGTGCACTGCCACAG
ACTGCCCGGCAGCTACGATAGCACCAGCAGCGATTCTCTGTACCCCAGAG
GCATCCAGTTCAGACGGCCTCATACAGTGGCTCCCTGGCCTCCTGCTTAC
CCTCCTGTGACAAGCTACCCACCTCTGAGCCAGCCTGACCTGCTGCCTAT
TCCTAGAAGCCCTCAGCCTCTCGGCGGCAGCCATAGAACACCTAGCAGCA
GAAGAGATAGCGACGGCGCCAATAGCGTGGCCAGCTACGAAAATGAAGGC
GCCTCTGGCATTAGAGGCGCCCAAGCTGGATGGGGAGTTTGGGGACCTAG
CTGGACAAGACTGACCCCTGTGTCTCTGCCTCCTGAACCTGCCTGCGAAG
ATGCCGACGAGGACGAGGATGACTATCACAACCCTGGCTACCTGGTGGTG
CTGCCTGATAGCACACCAGCCACATCTACAGCCGCTCCTAGTGCTCCTGC
TCTGAGCACACCTGGCATCAGAGACAGCGCCTTCAGCATGGAATCCATCG
ACGACTACGTGAACGTGCCCGAGTCTGGCGAATCTGCCGAAGCCTCTCTT
GACGGCAGCCGCGAGTATGTGAACGTGTCCCAAGAACTGCATCCCGGCGC
TGCCAAAACAGAACCTGCTGCTCTGTCTAGCCAAGAGGCCGAGGAAGTGG
AAGAAGAAGGCGCCCCTGACTACGAGAACCTGCAAGAGCTGAACTGATGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 205.

An exemplary bicistronic High affinity anti-CD22 CAR and High affinity anti-CD22 LAT-CAR “CAR1-linker-CAR2” or “HiAff/HiAff-LAT” or “LAT-CAR” or “22ALACART4” or “22ALA-CART” or “22ALACART” amino acid sequence is shown below (HiAff scFv CAR1; Furin/P2A linker; HiAff scFv CAR2)

(SEQ ID NO: 206)
MALPVTALLLPLALLLHAARPQVQLQQSGPGMVKPSQTLSLTCAISGDSV
SSNSVAWNWIRQSPSRGLEWLGRTYYRSTWYNDYAVSMKSRITINPDTNK
NQFSLQLNSVTPEDTAVYYCAREVTGDLEDAFDIWGQGTMVTVSSGGGGS
GGGGSGGGGSDIQMIQSPSSLSASVGDRVTITCRASQTIWSYLNWYRQRP
GEAPNLLIYAASSLQSGVPSRFSGRGSGTDFTLTISSLQAEDFATYYCQQ
SYSIPQTFGQGTKLEIKLETTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELDIRVKFSRSADAPAYQQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPRRKRRGS
GTPDPWGSGATNFSLLKQAGDVEENPGPGSMALPVTALLLPLALLLHAAR
PDYKDDDDKQVQLQQSGPGMVKPSQTLSLTCAISGDSVSSNSVAWNWIRQ
SPSRGLEWLGRTYYRSTWYNDYAVSMKSRITINPDTNKNQFSLQLNSVTP
EDTAVYYCAREVTGDLEDAFDIWGQGTMVTVSSGGGGSGGGGSGGGGSDI
QMIQSPSSLSASVGDRVTITCRASQTIWSYLNWYRQRPGEAPNLLIYAAS
SLQSGVPSRFSGRGSGTDFTLTISSLQAEDFATYYCQQSYSIPQTFGQGT
KLEIKSRIEVMYPPPYLDNEKSNGTIIHVKGKHLCPSPLFPGPSKPFWVL
VVVGGVLACYSLLVTVAFIIFWVHCHRLPGSYDSTSSDSLYPRGIQFRRP
HTVAPWPPAYPPVTSYPPLSQPDLLPIPRSPQPLGGSHRTPSSRRDSDGA
NSVASYENEGASGIRGAQAGWGVWGPSWTRLTPVSLPPEPACEDADEDED
DYHNPGYLVVLPDSTPATSTAAPSAPALSTPGIRDSAFSMESIDDYVNVP
ESGESAEASLDGSREYVNVSQELHPGAAKTEPAALSSQEAEEVEEEGAPD
YENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 206.

An exemplary bicistronic High affinity anti-CD22 CAR and High affinity anti-CD22 LAT-CAR “CAR1-linker-CAR2” or “HiAff/HiAff-LAT” or “LAT-CAR” or “22ALACART4” or “22ALACART” polynucleotide sequence is shown below (HiAff scFv CAR1; HiAff scFy CAR2)

(SEQ ID NO: 207)
ATGGCACTGCCAGTGACTGCATTACTCTTGCCACTCGCGCTACTGTTACA
CGCAGCACGTCCACATCACCATCACCATCACCAAGTCCAATTGCAACAAA
GCGGGCCGGGCATGGTGAAACCGAGTCAAACGTTATCTCTTACGTGTGCG
ATTTCGGGGGATAGTGTCAGCAGCAATTCAGTGGCGTGGAATTGGATTCG
CCAATCGCCGAGTCGCGGGTTGGAGTGGCTCGGGCGCACGTATTATCGCA
GCACATGGTATAATGATTATGCGGTCAGCATGAAAAGCCGCATTACGATT
AATCCGGATACGAACAAAAATCAATTTAGCTTACAATTAAATTCCGTCAC
GCCGGAAGATACAGCGGTCTATTATTGTGCGCGCGAGGTCACGGGGGATC
TCGAAGACGCGTTTGATATTTGGGGGCAAGGGACCATGGTGACTGTCAGC
TCTGGTGGAGGGGGCAGTGGAGGTGGGGGATCGGGAGGTGGTGGCAGTGA
TATTCAAATGATCCAAAGTCCATCCAGCCTATCCGCATCTGTCGGAGATC
GCGTAACGATTACGTGCCGCGCGAGTCAAACGATTTGGAGCTATCTGAAC
TGGTACCGGCAACGCCCGGGCGAAGCGCCGAATCTCTTGATTTACGCGGC
GTCCTCATTACAGTCGGGTGTCCCGAGCCGCTTTAGCGGCCGCGGAAGCG
GTACGGATTTTACGTTAACCATTAGCAGCCTCCAGGCGGAAGATTTTGCG
ACGTATTACTGTCAACAGAGCTATAGCATTCCGCAGACGTTTGGTCAGGG
CACGAAATTGGAGATTAAACTCGAGACCACCACCCCCGCCCCTAGGCCTC
CCACACCTGCCCCCACAATCGCCTCCCAGCCTCTCAGCCTGAGGCCTGAA
GCTTGCAGGCCCGCTGCCGGAGGAGCTGTCCATACCAGGGGACTCGACTT
CGCCTGCGACATTTACATTTGGGCCCCTCTGGCTGGAACCTGCGGAGTCC
TGCTGCTGTCCCTGGTGATCACACTGTACTGTAAGAGGGGCAGAAAGAAG
CTGCTCTACATCTTCAAGCAGCCCTTTATGAGACCCGTGCAGACAACCCA
GGAGGAAGACGGATGCAGCTGCAGGTTCCCTGAGGAGGAGGAGGGCGGCT
GCGAACTGGATATCAGGGTGAAGTTCAGCAGGAGCGCCGACGCCCCCGCT
TATCAACAGGGCCAGAACCAGCTGTACAACGAGCTGAACCTCGGCAGAAG
AGAGGAGTATGACGTGCTGGACAAGAGGAGGGGCAGGGACCCTGAGATGG
GCGGCAAGCCTAGAAGAAAGAACCCCCAGGAAGGCCTCTACAACGAACTG
CAGAAGGACAAGATGGCCGAGGCCTACAGCGAGATCGGCATGAAAGGCGA
GAGAAGGAGGGGAAAGGGACATGACGGCCTGTACCAGGGACTCTCCACAG
CCACCAAGGACACCTACGATGCCCTGCACATGCAGGCTCTGCCCCCTAGA
AGGAAGAGAAGAGGCTCTGGTACCCCCGATCCTTGGGGAAGCGGCGCTAC
CAACTTCTCCCTGCTCAAGCAGGCTGGCGATGTGGAGGAGAACCCCGGCC
CCGGATCCATGGCTCTGCCTGTGACAGCTCTGCTGCTGCCTCTGGCCCTG
CTGCTCCATGCTGCTAGACCTGACTACAAAGACGATGACGACAAGCAGGT
GCAGCTCCAGCAGTCTGGCCCAGGAATGGTCAAGCCTAGCCAGACCCTGA
GCCTGACCTGCGCCATCAGCGGCGACAGCGTGTCCTCTAACAGCGTCGCC
TGGAACTGGATCAGACAGAGCCCCAGCAGAGGCCTGGAATGGCTGGGCCG
GACCTACTACCGGTCCACGTGGTACAACGACTACGCCGTGTCCATGAAGT
CCCGGATCACCATCAACCCCGACACCAACAAGAACCAGTTCTCCCTGCAG
CTGAACAGCGTGACCCCTGAGGACACCGCCGTGTACTACTGCGCCAGAGA
AGTGACCGGCGACCTGGAAGATGCCTTCGACATCTGGGGCCAGGGCACCA
TGGTCACCGTGTCTAGCGGAGGCGGCGGAAGCGGTGGAGGCGGTAGCGGC
GGTGGCGGTTCCGACATCCAGATGATCCAGAGCCCTAGCTCCCTGAGCGC
CAGCGTGGGCGACAGAGTGACCATCACCTGTCGGGCCAGCCAGACCATCT
GGTCCTACCTGAATTGGTATCGGCAGCGGCCAGGCGAGGCCCCTAACCTG
CTGATCTATGCCGCCAGCAGCCTGCAGAGCGGCGTGCCAAGCAGATTCTC
TGGCAGAGGCTCCGGCACCGACTTCACCCTGACAATCAGTTCCCTGCAGG
CCGAGGACTTCGCCACCTACTACTGCCAGCAGTCCTACAGCATCCCTCAG
ACCTTCGGCCAGGGGACCAAGCTGGAAATCAAGTCTAGAATCGAAGTGAT
GTACCCTCCACCTTACCTGGACAACGAGAAGTCCAACGGCACCATCATCC
ACGTGAAGGGCAAGCACCTGTGTCCTTCTCCACTGTTCCCCGGACCTAGC
AAGCCTTTCTGGGTGCTCGTTGTTGTTGGCGGCGTGCTGGCCTGTTACAG
CCTGCTGGTTACCGTGGCCTTCATCATCTTTTGGGTGCACTGCCACAGAC
TGCCCGGCAGCTACGATAGCACCAGCAGCGATTCTCTGTACCCCAGAGGC
ATCCAGTTCAGACGGCCTCATACAGTGGCTCCCTGGCCTCCTGCTTACCC
TCCTGTGACAAGCTACCCACCTCTGAGCCAGCCTGACCTGCTGCCTATTC
CTAGAAGCCCTCAGCCTCTCGGCGGCAGCCATAGAACACCTAGCAGCAGA
AGAGATAGCGACGGCGCCAATAGCGTGGCCAGCTACGAAAATGAAGGCGC
CTCTGGCATTAGAGGCGCCCAAGCTGGATGGGGAGTTTGGGGACCTAGCT
GGACAAGACTGACCCCTGTGTCTCTGCCTCCTGAACCTGCCTGCGAAGAT
GCCGACGAGGACGAGGATGACTATCACAACCCTGGCTACCTGGTGGTGCT
GCCTGATAGCACACCAGCCACATCTACAGCCGCTCCTAGTGCTCCTGCTC
TGAGCACACCTGGCATCAGAGACAGCGCCTTCAGCATGGAATCCATCGAC
GACTACGTGAACGTGCCCGAGTCTGGCGAATCTGCCGAAGCCTCTCTTGA
CGGCAGCCGCGAGTATGTGAACGTGTCCCAAGAACTGCATCCCGGCGCTG
CCAAAACAGAACCTGCTGCTCTGTCTAGCCAAGAGGCCGAGGAAGTGGAA
GAAGAAGGCGCCCCTGACTACGAGAACCTGCAAGAGCTGAACTGATGA

In some embodiments, the anti-CD22 CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 207.

### iv) Exemplary First CARs

### An Exemplary Anti-CD19 CAR

(SEQ ID NO: 309)
GSMEFGLSWLFLVAILKGVQCSRDIQMTQTTSSLSASLGDRVTISCRASQ
DISKYLNWYQQKPDGTVKLLIYHTSRLHSGVPSRFSGSGSGTDYSLTISN
LEQEDIATYFCQQGNTLPYTFGGGTKLEITGSTSGSGKPGSGEGSTKGEV
KLQESGPGLVAPSQSLSVTCTVSGVSLPDYGVSWIRQPPRKGLEWLGVIW
GSETTYYNSALKSRLTIIKDNSKSQVFLKMNSLQTDDTAIYYCAKHYYYG
GSYAMDYWGQGTSVTVLETTTPAPRPPTPAPTIASQPLSLRPEACRPAAG
GAVHTRGLDFACDIYIWAPLAGTCGVLLLSLVITLYCKRGRKKLLYIFKQ
PFMRPVQTTQEEDGCSCRFPEEEEGGCELDIRVKFSRSADAPAYQQGQNQ
LYNELNLGRREEYDVLDKRRGRDPEMGGKPRRKNPQEGLYNELQKDKMAE
AYSEIGMKGERRRGKGHDGLYQGLSTATKDTYDALHMQALPPR

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 309.

### v) Exemplary Second CARs

### An Exemplary Anti-CD22-LAT CAR

(SEQ ID NO: 300)
GSMALPVTALLLPLALLLHAARPDYKDDDDKQVQLQQSGPGMVKPSQTLS
LTCAISGDSVSSNSVAWNWIRQSPSRGLEWLGRTYYRSTWYNDYAVSMKS
RITINPDTNKNQFSLQLNSVTPEDTAVYYCAREVTGDLEDAFDIWGQGTM
VTVSSGGGGSGGGGSGGGGSDIQMIQSPSSLSASVGDRVTITCRASQTIW
SYLNWYRQRPGEAPNLLIYAASSLQSGVPSRFSGRGSGTDFTLTISSLQA
EDFATYYCQQSYSIPQTFGQGTKLEIKSRIEVMYPPPYLDNEKSNGTIIH
VKGKHLCPSPLFPGPSKPFWVLVVVGGVLACYSLLVTVAFIIFWVHCHRL
PGSYDSTSSDSLYPRGIQFKRPHTVAPWPPAYPPVTSYPPLSQPDLLPIP
RSPQPLGGSHRTPSSRRDSDGANSVASYENEGASGIRGAQAGWGVWGPSW
TRLTPVSLPPEPACEDADEDEDDYHNPGYLVVLPDSTPATSTAAPSAPAL
STPGIRDSAFSMESIDDYVNVPESGESAEASLDGSREYVNVSQELHPGAA
KTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 300.

### An Exemplary Anti-CD22-LAT-K52R CAR

(SEQ ID NO: 301)
GSMALPVTALLLPLALLLHAARPDYKDDDDKQVQLQQSGPGMVKPSQTLS
LTCAISGDSVSSNSVAWNWIRQSPSRGLEWLGRTYYRSTWYNDYAVSMKS
RITINPDTNKNQFSLQLNSVTPEDTAVYYCAREVTGDLEDAFDIWGQGTM
VTVSSGGGGSGGGGSGGGGSDIQMIQSPSSLSASVGDRVTITCRASQTIW
SYLNWYRQRPGEAPNLLIYAASSLQSGVPSRFSGRGSGTDFTLTISSLQA
EDFATYYCQQSYSIPQTFGQGTKLEIKSRIEVMYPPPYLDNEKSNGTIIH
VKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIP
RSPQPLGGSHRTPSSRRDSDGANSVASYENEGASGIRGAQAGWGVWGPSW
TRLTPVSLPPEPACEDADEDEDDYHNPGYLVVLPDSTPATSTAAPSAPAL
STPGIRDSAFSMESIDDYVNVPESGESAEASLDGSREYVNVSQELHPGAA
KTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 301.

### An Exemplary Anti-CD22-LAT-K233R CAR

(SEQ ID NO: 302)
GSMALPVTALLLPLALLLHAARPDYKDDDDKQVQLQQSGPGMVKPSQTLS
LTCAISGDSVSSNSVAWNWIRQSPSRGLEWLGRTYYRSTWYNDYAVSMKS
RITINPDTNKNQFSLQLNSVTPEDTAVYYCAREVTGDLEDAFDIWGQGTM
VTVSSGGGGSGGGGSGGGGSDIQMIQSPSSLSASVGDRVTITCRASQTIW
SYLNWYRQRPGEAPNLLIYAASSLQSGVPSRESGRGSGTDFTLTISSLQA
EDFATYYCQQSYSIPQTFGQGTKLEIKSRIEVMYPPPYLDNEKSNGTIIH
VKGKHLCPSPLFPGPSKPFWVLVVVGGVLACYSLLVTVAFIIFWVHCHRL
PGSYDSTSSDSLYPRGIQFKRPHTVAPWPPAYPPVTSYPPLSQPDLLPIP
RSPQPLGGSHRTPSSRRDSDGANSVASYENEGASGIRGAQAGWGVWGPSW
TRLTPVSLPPEPACEDADEDEDDYHNPGYLVVLPDSTPATSTAAPSAPAL
STPGIRDSAFSMESIDDYVNVPESGESAEASLDGSREYVNVSQELHPGAA
RTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 302.

### An Exemplary Anti-CD22-LAT-K52R-K233R CAR

(SEQ ID NO: 303)
GSMALPVTALLLPLALLLHAARPDYKDDDDKQVQLQQSGPGMVKPSQTLS
LTCAISGDSVSSNSVAWNWIRQSPSRGLEWLGRTYYRSTWYNDYAVSMKS
RITINPDTNKNQFSLQLNSVTPEDTAVYYCAREVTGDLEDAFDIWGQGTM
VTVSSGGGGSGGGGSGGGGSDIQMIQSPSSLSASVGDRVTITCRASQTIW
SYLNWYRQRPGEAPNLLIYAASSLQSGVPSRFSGRGSGTDFTLTISSLQA
EDFATYYCQQSYSIPQTFGQGTKLEIKSRIEVMYPPPYLDNEKSNGTIIH
VKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIP
RSPQPLGGSHRTPSSRRDSDGANSVASYENEGASGIRGAQAGWGVWGPSW
TRLTPVSLPPEPACEDADEDEDDYHNPGYLVVLPDSTPATSTAAPSAPAL
STPGIRDSAFSMESIDDYVNVPESGESAEASLDGSREYVNVSQELHPGAA
RTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 303.

### An Exemplary Anti-CD22-LAT-K52R-G160E CAR

(SEQ ID NO: 304)
GSMALPVTALLLPLALLLHAARPDYKDDDDKQVQLQQSGPGMVKPSQTLS
LTCAISGDSVSSNSVAWNWIRQSPSRGLEWLGRTYYRSTWYNDYAVSMKS
RITINPDTNKNQFSLQLNSVTPEDTAVYYCAREVTGDLEDAFDIWGQGTM
VTVSSGGGGSGGGGSGGGGSDIQMIQSPSSLSASVGDRVTITCRASQTIW
SYLNWYRQRPGEAPNLLIYAASSLQSGVPSRFSGRGSGTDFTLTISSLQA
EDFATYYCQQSYSIPQTFGQGTKLEIKSRIEVMYPPPYLDNEKSNGTIIH
VKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIP
RSPQPLGGSHRTPSSRRDSDGANSVASYENEGASGIRGAQAGWGVWGPSW
TRLTPVSLPPEPACEDADEDEDDYHNPEYLVVLPDSTPATSTAAPSAPAL
STPGIRDSAFSMESIDDYVNVPESGESAEASLDGSREYVNVSQELHPGAA
KTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 304.

### An Exemplary Anti-CD22-LAT-K52R-K233R-G160E CAR

(SEQ ID NO: 305)
GSMALPVTALLLPLALLLHAARPDYKDDDDKQVQLQQSGPGMVKPSQTLS
LTCAISGDSVSSNSVAWNWIRQSPSRGLEWLGRTYYRSTWYNDYAVSMKS
RITINPDTNKNQFSLQLNSVTPEDTAVYYCAREVTGDLEDAFDIWGQGTM
VTVSSGGGGSGGGGSGGGGSDIQMIQSPSSLSASVGDRVTITCRASQTIW
SYLNWYRQRPGEAPNLLIYAASSLQSGVPSRFSGRGSGTDFTLTISSLQA
EDFATYYCQQSYSIPQTFGQGTKLEIKSRIEVMYPPPYLDNEKSNGTIIH
VKGKHLCPSPLFPGPSKPFWVLVVVGGVLACYSLLVTVAFIIFWVHCHRL
PGSYDSTSSDSLYPRGIQFRRPHTVAPWPPAYPPVTSYPPLSQPDLLPIP
RSPQPLGGSHRTPSSRRDSDGANSVASYENEGASGIRGAQAGWGVWGPSW
TRLTPVSLPPEPACEDADEDEDDYHNPEYLVVLPDSTPATSTAAPSAPAL
STPGIRDSAFSMESIDDYVNVPESGESAEASLDGSREYVNVSQELHPGAA
RTEPAALSSQEAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 305.

### An Exemplary Anti-CD22-HiAff-LAT CAR

(SEQ ID NO: 306)
GSMALPVTALLLPLALLLHAARPDYKDDDDKQVQLQQSGPGLVKPSQTLS
LTCAISGDSVSSNSAAWNWIRQSPSRGLEWLGRTYYRSKWYNDYAVSVKS
RITINPDTSKNQFSLQLNSVTPEDTAVYYCAREVTGDLEDAFDIWGQGTM
VTVSSGGGGSDIQMTQSPSSLSASVGDRVTITCRASQTIWSYLNWYQQRP
GKAPNLLIYAASSLQSGVPSRFSGRGSGTDFTLTISSLQAEDFATYYCQQ
SYSIPQTFGQGTKLEIKSRIEVMYPPPYLDNEKSNGTIIHVKGKHLCPSP
LFPGPSKPFWVLVVVGGVLACYSLLVTVAFIIFWVHCHRLPGSYDSTSSD
SLYPRGIQFRRPHTVAPWPPAYPPVTSYPPLSQPDLLPIPRSPQPLGGSH
RTPSSRRDSDGANSVASYENEGASGIRGAQAGWGVWGPSWTRLTPVSLPP
EPACEDADEDEDDYHNPGYLVVLPDSTPATSTAAPSAPALSTPGIRDSAF
SMESIDDYVNVPESGESAEASLDGSREYVNVSQELHPGAAKTEPAALSSQ
EAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 306.

### An Exemplary Anti-CD19-LAT CAR

(SEQ ID NO: 307)
GSMEFGLSWLFLVAILKGVQCSRDYKDDDDKDIQMTQTTSSLSASLGDRV
TISCRASQDISKYLNWYQQKPDGTVKLLIYHTSRLHSGVPSRFSGSGSGT
DYSLTISNLEQEDIATYFCQQGNTLPYTFGGGTKLEITGSTSGSGKPGSG
EGSTKGEVKLQESGPGLVAPSQSLSVTCTVSGVSLPDYGVSWIRQPPRKG
LEWLGVIWGSETTYYNSALKSRLTIIKDNSKSQVFLKMNSLQTDDTAIYY
CAKHYYYGGSYAMDYWGQGTSVTVSRIEVMYPPPYLDNEKSNGTIIHVKG
KHLCPSPLFPGPSKPFWVLVVVGGVLACYSLLVTVAFIIFWVHCHRLPGS
YDSTSSDSLYPRGIQFRRPHTVAPWPPAYPPVTSYPPLSQPDLLPIPRSP
QPLGGSHRTPSSRRDSDGANSVASYENEGASGIRGAQAGWGVWGPSWTRL
TPVSLPPEPACEDADEDEDDYHNPGYLVVLPDSTPATSTAAPSAPALSTP
GIRDSAFSMESIDDYVNVPESGESAEASLDGSREYVNVSQELHPGAAKTE
PAALSSQEAEEVEEEGAPDYENLQELN

In some embodiments, the anti-CD19 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 307.

### An Exemplary Anti-CD22-SAff-LAT CAR

(SEQ ID NO: 308)
GSMALPVTALLLPLALLLHAARPDYKDDDDKQVQLQQSGPGLVKPSQTLS
LTCAISGDSVSSNSAAWNWIRQSPSRGLEWLGRTYYRSKWYNDYAVSVKS
RITINPDTSKNQFSLQLNSVTPEDTAVYYCAREVTGDLEDAFDIWGQGTM
VTVSSGGGGSDIQMTQSPSSLSASVGDRVTITCRASQTIWSYLNWYQQRP
GKAPNLLIYAASSLQSGVPSRFSGRGSGTDFTLTISSLQAEDFATYYCQQ
SYSIPQTFGQGTKLEIKSRIEVMYPPPYLDNEKSNGTIIHVKGKHLCPSP
LFPGPSKPFWVLVVVGGVLACYSLLVTVAFIIFWVHCHRLPGSYDSTSSD
SLYPRGIQFRRPHTVAPWPPAYPPVTSYPPLSQPDLLPIPRSPQPLGGSH
RTPSSRRDSDGANSVASYENEGASGIRGAQAGWGVWGPSWTRLTPVSLPP
EPACEDADEDEDDYHNPGYLVVLPDSTPATSTAAPSAPALSTPGIRDSAF
SMESIDDYVNVPESGESAEASLDGSREYVNVSQELHPGAAKTEPAALSSQ
EAEEVEEEGAPDYENLQELN

In some embodiments, the anti-CD22 CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 308.

### 4. CAR Expression Levels

The present disclosure provides a population of engineered T cells, wherein a plurality of the engineered T cells of the population comprise any chimeric stimulatory receptor (CAR) disclosed herein. The present disclosure also provides a composition comprising a population of T cells, wherein a plurality of the T cells of the population comprise a non-naturally occurring CAR comprising, consisting essentially of, or consisting of: a) a first chimeric antigen receptor (CAR) comprising an antigen recognition domain that binds to a first antigen, a transmembrane domain and a intracellular signaling domain; b) a second CAR comprising an antigen recognition domain that binds to a second antigen, a transmembrane domain and a Linker for Activation of T cell (LAT) intracellular signaling domain. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the first CAR and the second CAR. In some embodiments, each CAR polypeptide is expressed at a copy number of at least 1, 2, 3, 4, 5, 6, 7, 8, 9, 10, 20, 30, 40, 50, 60, 70, 80, 90 or 100 copies per cell. In some embodiments, the nucleic acid encoding the CAR is integrated into the genome at a copy number of at least 1, 2, 3, 4, 5, 6, 7, 8, 9, 10, 20 or 30 copies per cell.

In some embodiments, the ratio of the copy number of CAR1:CAR2 is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10.

### 5. Antigens

In some embodiments, provided herein are cells (e.g., T cells) expressing a first CAR targeting a first antigen (e.g. anti-CD22) and a second CAR targeting a second antigen (e.g. anti-CD19).

Among the antigens that may be targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, glycosylated antigens, TnAntigens, auto-/self-antigens, tumor-/cancer-associated antigens, and tumor neoantigens (Linnemann et al, 2015). In particular aspects, the antigens include those listed in Table 1.

In particular aspects, the antigens for targeting by two or more antigen recognition domains include, but are not limited to CD22 and CD19 (e.g., for B cell malignancies) The sequences for these antigens are known in the art, for example, CD22 (e.g, Accession No. NM_001772.4); CD19 (e.g., Accession No. NC_000023.11).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in PCT Publication No. WO 99/40188), PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-AI, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MCIR, gp100, PSA, PSM, Tyrosinase, TRP-1, TRP-2, ART-4, CAMEL, CEA, Cyp-B, hTERT, hTRT, ICE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mber-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g, Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AMLI, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B 1, polysialic acid, MYCN, RhoC, GD3, fucosyl GM1, mesothelian, PSCA, sLe, PLACI, GM3, BORIS, Tn, GLoboH, NY-BR-1, RGSS, SART3, STn, PAX5, OY-TES 1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, ber/abl rearrangement, Her2/neu, mutated or wild-type p53, cytochrome P450 1B 1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRS A), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, pl7, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (HI, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman et al. 2006), envelope glycoprotein B (gB), gH, gN, and ppl50. As would be understood by the skilled person, CMV proteins for use as antigens described herein may be identified in public databases such as GENBANK®, SWISS-PROT®, and TREMBL® (see e.g., Bennekov et al. 2004; Loewendorf et al. 2010; Marschall et al. 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al, 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof. large protein (L), glycoprotein (G), nucleoprotein (N), phosphoprotein (P), and matrix protein (M) (see, e.g., Rieder et al, 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus El or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010). P Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis Fl and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhino sporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Tricho strongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PILSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### 6. Safety Switch Proteins

Although cellular therapies hold great promise for the treatment of human disease, significant toxicities from the cells themselves or from their transgene products have hampered clinical investigation. In some embodiments described herein, immune effector cells (e.g., T cells) comprising a CAR described herein that have been infused into a mammalian subject, e.g., a human, can be ablated in order to regulate the effect of such immune effector cells should toxicity arise from their use. In some embodiments, the immune cells of the present disclosure may comprise one or more suicide genes.

As used herein, the term “safety switch protein”, “suicide protein” or “kill switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis or DNA replication, growth arrest, transcriptional and post-transcriptional genetic regulation and/or antibody-mediated depletion. In some instances, the safety switch protein is activated by an exogenous molecule, e.g., a prodrug, that, when activated, triggers apoptosis and/or cell death of a therapeutic cell.

The term “suicide gene” or “kill switch gene” as used herein is defined as a gene which, upon administration of a prodrug, effects transition of a gene product to a compound which kills its host cell. Examples of suicide gene/prodrug combinations which may be used include, but are not limited to inducible caspase 9 (iCASP9) and rimiducid; RQR8 and rituximab; truncated version of EGFR variant III (EGFRv3) and cetuximab; Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include but are not limited to inducible caspase 9 (or caspase 3 or 7), CD20, CD52, EGFRt, or, thymidine kinase, cytosine deaminase, HER1 and any combination thereof. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-a,Y-lyase (MET), and Thymidine phosphorylase (TP).

### 7. T Cell Activity

In some embodiments, a population of genetically engineered T cells as disclosed herein exhibits T cell functions (e.g., effector functions). In some embodiments, the population is cytotoxic to CD22-expressing cells and CD19 expressing cells (e.g, CD22-positive tumor cells, CD22-low tumor cells, CD19 positive tumor cells, CD19 low tumor cells) Effector function of a T cell, for example, may be cytolytic activity or helper activity including the secretion of cytokines. In some embodiments, the population exhibits one or more T cell effector functions at a level that is least 3-4-fold higher than the functions exhibited by a population of T cells not expressing the CAR.

### III. METHODS

Chimeric antigen receptors may be readily inserted into and expressed by immune cells, (e.g., T cells). In certain embodiments, cells (e.g., immune cells such as T cells) are obtained from a donor subject. In some embodiments, the donor subject is human patient afflicted with a cancer or a tumor. In other embodiments, the donor subject is a human patient not afflicted with a cancer or a tumor. In some embodiments, an engineered cell is autologous to a subject. In some embodiments, an engineered cell is allogeneic to a subject.

The cell of the present disclosure may be obtained through any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. In certain embodiments, the cells collected by apheresis are washed to remove the plasma fraction, and placed in an appropriate buffer or media for subsequent processing. In some embodiments, the cells are washed with PBS. As will be appreciated, a washing step can be used, such as by using a semiautomated flowthrough centrifuge, e.g., the Cobe™ 2991 cell processor, the Baxter CytoMate™, or the like. In some embodiments, the washed cells are resuspended in one or more biocompatible buffers, or other saline solution with or without buffer. In certain embodiments, the undesired components of the apheresis sample are removed. Additional methods of isolating T cells for a T cell therapy are disclosed in U.S. Patent Publication No. 2013/0287748, which is herein incorporated by references in its entirety.

In certain embodiments, T cells are isolated from PBMCs by lysing the red blood cells and depleting the monocytes, e.g., by using centrifugation through a PERCOLL™ gradient. In some embodiments, a specific subpopulation of T cells, such as CD4⁺, CD8⁺, CD28⁺, CD45RA⁺, and CD45RO⁺ T cells is further isolated by positive or negative selection techniques known in the art. For example, enrichment of a T cell population by negative selection can be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected can be used. For example, to enrich for CD4⁺ cells by negative selection, a monoclonal antibody cocktail typically includes antibodies to CD8, CDI lb, CD14, CD16, CD20, and HLA-DR. In certain embodiments, flow cytometry and cell sorting are used to isolate cell populations of interest for use in the present disclosure.

In some embodiments, PBMCs are used directly for genetic modification with the immune cells (such as CARs or TCRs) using methods as described herein. In certain embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8⁺ cells are further sorted into naive, central memory, and effector cells by identifying cell surface antigens that are associated with each of these types of CD8⁺ cells. In some embodiments, the expression of phenotypic markers of central memory T cells includes CCR7, CD3, CD28, CD45RO, CD62L, and CD127 and are negative for granzyme B. In some embodiments, central memory T cells are CD8⁺, CD45RO⁺, and CD62L⁺ T cells. In some embodiments, effector T cells are negative for CCR7, CD28, CD62L, and CD127 and positive for granzyme B and perforin. In certain embodiments, CD4⁺ T cells are further sorted into subpopulations. For example, CD4⁺ T helper cells can be sorted into naive, central memory, and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

### IV. METHODS OF GENE DELIVERY AND CELL MODIFICATION

One of skill in the art would be well-equipped to construct a vector through standard recombinant techniques (see, for example, Sambrook et al., 2001 and Ausubel et al, 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

### 1. Viral Vectors

Viral vectors encoding an antigen receptor, a cytokine and/or an functional effector element may be provided in certain aspects of the methods of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

An engineered virus vector may comprise long terminal repeats (LTRs), a cargo nucleotide sequence, or a cargo cassette. A viral vector-related “cargo cassette” as used herein refers to a nucleotide sequence comprising a left LTR at the 5′ end and a right LTR at the 3′ end, and a nucleotide sequence positioned between the left and right LTRs. The nucleotide sequence flanked by the LTRs is a nucleotide sequence intended for integration into acceptor DNA. A “cargo nucleotide sequence” refers to a nucleotide sequence (e.g., a nucleotide sequence intended for integration into acceptor DNA), flanked by an LTR at each end, wherein the LTRs are heterologous to the nucleotide sequence. A cargo cassette can be artificially engineered.

In some embodiments of the methods of the disclosure, introducing a nucleic acid sequence and/or a genomic editing construct into an immune cell ex vivo, in vivo, in vitro or in situ comprises a viral vector. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), Lentiviruses, and gamma-retroviruses.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

A retroviral vector may also be, e.g., a gammaretroviral vector. A gammaretroviral vector may include, e.g., a promoter, a packaging signal (w), a primer binding site (PBS), one or more (e.g, two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., Viruses. 2011 June; 3 (6): 677-713.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

In some embodiments of the methods of the disclosure, introducing a nucleic acid sequence and/or a genomic editing construct into an immune cell ex vivo, in vivo, in vitro or in situ comprises a combination of vectors. Exemplary, non-limiting vector combinations include: viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

### A. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

### (i) Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (ere), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no X05244, nucleotide 283-341) or a mouse mammary tumor promoter (available from the ATCC, Cat. No. ATCC 45007). In certain embodiments, the promoter is EF1, EF1alpha, MND, CMV IE, dectin-1, dectin-2, human CDI lc, F4/80, SM22, RSV, SV40, Ad MLP, beta-actin, MHC class I, MHC class II promoter, U6 promoter or HI promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### (ii) Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription functional effector elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A) or a P2A (e.g. porcine teschovirus-1 2A).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### B. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### 2. Other Methods of Nucleic Acid Delivery

In addition to viral delivery of the nucleic acids encoding the antigen receptor, the following are additional methods of recombinant gene delivery to a given cell, (e.g. an NK cell) and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### A. Transposition Based Methods of Modification

Generally, the gene transfer system can include a transposon-based or a viral-based integration system.

In some embodiments, the gene transfer system comprises a transposon system. DNA transposons can translocate via a non-replicative “cut-and-paste” mechanism. This mechanism requires recognition of the two inverse terminal repeats (ITRs) by a catalytic enzyme, i.e., transposase, which can cleave its target and consequently release the DNA transposon from its donor template. Upon excision, the DNA transposons may subsequently integrate into the acceptor DNA that is cleaved by the same transposase. In some of their natural configurations, DNA transposons are flanked by two ITRs and may contain a gene encoding a transposase that catalyzes transposition.

Transposon systems offer many advantages for nucleic acid integration, e.g., as compared to viral vectors. For example, transposons can carry larger cargos, which can be advantageous for delivering one or more of the CARs, functional effector elements, and/or cytokines disclosed herein, to an immune cell (e.g., an NK cell). Further, transposons may comprise, for example, CRISPR tools (e.g., along with cargo), and thereby allow multiplex engineering of a cell.

A transposon system comprises (i) a plasmid backbone with inverse terminal repeats (ITRs) and (ii) a transposase enzyme that recognizes the ITRs. The term “inverse terminal repeats,” “inverted terminal repeats”, or “ITRs”, as used interchangeably herein, refers to short sequence repeats flanking the transposase gene in a natural transposon, or flanking a cargo polynucleotide sequence in an artificially engineered transposon. Two inverted terminal repeats are generally required for the mobilization of the transposon in the presence of a corresponding transposase. Inverted repeats as described herein may contain one or more direct repeat (DR) sequences. These DR sequences usually are embedded in the terminal inverted repeats (ITRs) of the elements. The compositions and methods of the present disclosure comprise, in various embodiments, one or more artificially engineered transposons. An engineered transposon may comprise ITRs, a cargo nucleotide sequence, or a cargo cassette. A transposon-related “cargo cassette” as used herein refers to a nucleotide sequence comprising a left ITR at the 5′ end and a right ITR at the 3′ end, and a nucleotide sequence positioned between the left and right ITRs. The nucleotide sequence flanked by the ITRs is a nucleotide sequence intended for integration into acceptor DNA. The cargo cassette can, in some embodiments, be comprised in a vector, such as plasmid. A “cargo nucleotide sequence” refers to a nucleotide sequence (e.g., a nucleotide sequence intended for integration into acceptor DNA), flanked by an ITR at each end, wherein the ITRs are heterologous to the nucleotide sequence. A cargo cassette can be artificially engineered.

### Transposons and Transposase

Exemplary transposon systems for use as described in the disclosure include, but are not limited to, piggyBac, hyperactive piggyBac, Sleeping Beauty (SB), hyperactive Sleeping Beauty (SB100x), SB11, SB110, Tn7, TcBuster, hyperactive TcBuster, Frog Prince, IS5, TnIO, Tn903, SPIN, hAT, Hermes, Hobo, AeBusterl, AeBuster2, AeBuster3, BtBusterl, BtBuster2, CfBusterl, CfBuster2, Tol2, mini-Tol2, Tc3, Mos1, MuA, Himar I, Helitron, and engineered versions of transposase family enzymes (Zhang et al. (2009) PLOS Genet. 5:e 1000689; Wilson et al. (2007) J. Microbiol. Methods 71:332-5, the entire contents of which are incorporated by reference herein). Exemplary transposons also include the transposons of the hAT transposon superfamily described in Arensburger et al. (2011) Genetics 188 (1): 45-57, the entire contents of which are incorporated by reference herein) or a SPACE INVADERS (SPIN) transposon (see, e.g., Pace et al. (2008) Proc. Natl. Acad. Sci. USA. 2008, 105 (44): 17023-17028, the entire contents of which are incorporated by reference herein).

In some embodiments, the gene transfer system can be delivered to the cell encoded in DNA, encoded in mRNA, as a protein, or as a nucleoprotein complex. Alternatively, the gene transfer system can be integrated into the genome of a host cell using, for example, a retro-transposon, random plasmid integration, recombinase-mediated integration, homologous recombination mediated integration, or non-homologous end joining mediated integration. More examples of transposition systems that can be used with certain embodiments of the compositions and methods provided herein include Staphylococcus aureus Tn552 (Colegio et al, J. BacterioL, 183:2384-8, 2001; Kirby C et al, Mol. Microbiol, 43:173-86, 2002), Tyl (Devine & Boeke, Nucleic Acids Res., 22:3765-72, 1994 and International Publication WO 95/23875), Transposon Tn7 (Craig, N L, Science. 271:1512, 1996; Craig, N L, Review in: Curr Top Microbiol Immunol, 204:27-48, 1996), Tn/O and IS10 (Kleckner N, et al, Curr Top Microbiol Immunol, 204:49-82, 1996), Mariner transposase (Lampe D J, et al, EMBO J., 15:5470-9, 1996), Tel (Plasterk R H, Curr. Topics Microbiol. Immunol, 204:125-43, 1996), P Element (Gloor, G B, Methods Mol. Biol, 260:97-114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204:1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). The entire contents of each of the foregoing references are incorporated by reference herein.

Transposition efficiency can be measured by the percent of successful transposition events occurring in a population of host cells normalized by the amount of transposon and transposase introduced into the population of host cells. In many instances, when the transposition efficiency of two or more transposases is compared, the same transposon construct is paired with each of the two or more transposases for transfection of the host cells under same or similar transfection conditions. The amount of transposition events in the host cells can be examined by various approaches. For example, the transposon construct may be designed to contain a reporter gene positioned between the inverted repeats, and transfected cells positive for the reporter gene can be counted as the cells where successful transposition events occurs, which can give an estimate of the amount of the transposition events. Another non-limiting example includes sequencing of the host cell genome to examine the insertion of the cassette cargo of the transposon. In some embodiments, when the transposition efficiency of two or more different transposons is compared, the same transposase can be paired with each of the different transposons for transfection of the host cells under same or similar transfection conditions. Similar approaches to the above, and other methods commonly known to one skilled in the art, may also be implemented for the comparison of transposition efficiency.

### Polynucleotides Encoding the Transposase System

One aspect of the present disclosure provides a polynucleotide comprising a nucleotide sequence that encodes for a transposase described herein. In some embodiments, the polynucleotide further comprises a nucleotide sequence of a transposon (e.g., an engineered transposon) recognizable by the transposase. In some embodiments, the polynucleotide is comprised in an expression vector. In some embodiments, the expression vector is a DNA plasmid. In some embodiments, the expression vector is a mini-circle vector. In some embodiments, the expression vector is a nanoplasmid.

The term “mini-circle vector” as used herein can refer to a small circular plasmid derivative that is free of most, if not all, prokaryotic vector parts (e.g., control sequences or non-functional sequences of prokaryotic origin).

For genome editing applications with transposons, in some embodiments, it may be desirable to design a transposon for use in a binary system based on two distinct plasmids, whereby the nucleic acid sequence encoding for the transposase is physically separated from the transposon nucleic acid sequence containing the gene of interest flanked by the inverted repeats. Co-delivery of the transposon and transposase-encoding plasmids into the target cells enables transposition via a conventional cut-and-paste mechanism. In some other embodiments, a transposon based system as described herein may comprise a polynucleotide comprising both a nucleic acid sequence encoding a transposase as described herein, and a nucleic acid sequence of a transposon as described herein, i.e., wherein the nucleic acid encoding for the transposase and the transposon nucleic acid are present in the same plasmid.

One of the limitations of application of plasmid vectors is that transgene expression duration from plasmid vectors is reduced due to promoter inactivation mediated by the bacterial region (i.e., the region encoding the bacterial replication origin and selectable marker) of the vector (Chen et al., 2004. Gene Ther 11:856-864; Suzuki et al., 2006. J Virol 80:3293-3300) This results in short duration transgene expression. A strategy to improve transgene expression duration is to remove the bacterial region of the plasmid. For example, minicircle vectors have been developed which do not contain a bacterial region. Removal of the bacterial region in minicircle vectors improved transgene expression duration (Chen et al., 2004). In minicircle vectors, the eukaryotic region polyadenylation signal is covalently linked to the eukaryotic region promoter through a short spacer typically less than 200 bp comprised of the recombined attachment sites. This linkage (spacer region) can tolerate a much longer spacer sequence since while long spacers >1 kb in length resulted in transgene expression silencing in vivo, shorter spacers <500 bp exhibited similar transgene expression patterns to conventional minicircle DNA vectors (Lu et al., 2012. Mol Ther. 20:2111-9).

In some embodiments, a vector useful in various aspects of the disclosure is a nanoplasmid vector. The term “nanoplasmid vector” as used herein, refers to a vector combining an RNA selectable marker with a R6K, ColE2 or ColE2 related replication origin. Nanoplasmid vectors can be selected from the nanoplasmid vectors disclosed in any of International PCT Publication No. WO2014/035457, International PCT Publication No. WO2014/077866, and International PCT Publication No. WO2019/183248, each of which is incorporated in its entirety herein by reference. For example, International PCT Publication No. WO2014/035457 discloses minimalized nanoplasmid vectors that utilize RNA-OUT antibiotic-free selection and replace the large 1000 bp pUC replication origin with a novel, 300 bp, R6K origin, which result in improved expression from the plasmid. Reduction of the spacer region linking the 5′ and 3′ ends of the transgene expression cassette to <500 bp with R6K origin-RNA-OUT backbones improved expression duration to that of conventional minicircle DNA vectors. The 1.1 kb pFAR4 vector pUC-origin tRNA antibiotic free selection spacer has improved expression duration compared to a 2.2 kb pUC origin-kanR antibiotic selection marker spacer region (Quiviger et al., 2014. Gene Therapy 21:1001-1007). This indicates that improved expression duration can be obtained with some bacterial regions up to 1.1 kb. Expression level improvement compared to plasmid vectors is also observed with some spacer regions <1.1 kb. For example, pVAXl derivatives with the 2 kb bacterial backbone reduced to 1.2, 1.1 or 0.7 kb show >2-fold improved expression compared to the parent pVAXI vector. NTC8685 derivatives with the 1.5 kb bacterial backbone reduced to 0.9 kb, 466 bp or 281 bp (nanoplasmid vectors) show >2-fold improved expression compared to the parent NTC8685 vector.

In some embodiments, the nanoplasmid vector is useful for viral and non-viral gene therapy, viral and non-viral cell therapy, and more particularly, for improving viral and non-viral vector manufacturing yield and quality, for reducing transfection associated toxicity, for improving transposition from non-viral transposon vectors, for improving packaging titers from viral vectors, for improving expression of viral and non-viral vector encoded transgenes, and for eliminating antibiotic resistance marker gene transfer by viral and non-viral vectors, as described in International PCT Publication No. WO2019/183248, which is incorporated in its entirety herein by reference.

In some embodiments, the nanoplasmid vector comprises modifications that improve the replication of the vector. In some embodiments, the nanoplasmid vector utilizes a Pol III-dependent origin of replication to replicate. In some embodiments, the nanoplasmid vector utilizes a Pol I-dependent origin of replication to replicate. In some embodiments, the nanoplasmid vector comprises an antibiotic selectable marker. In some embodiments, the nanoplasmid vector does not comprise an antibiotic selectable marker. In some embodiments, the nanoplasmid vector comprises an RNA selectable marker.

### B. Other Methods of Modification

In some embodiments of the methods of the disclosure, a modified immune cell of the disclosure may be produced by introducing a transgene into an immune cell of the disclosure. The introducing step may comprise delivery of a nucleic acid sequence and/or a genomic editing construct via a non-transposition delivery system.

In some embodiments of the methods of the disclosure, introducing a nucleic acid sequence and/or a genomic editing construct into an immune cell ex vivo, in vivo, in vitro or in situ comprises one or more of topical delivery, adsorption, absorption, electroporation, spin-fection, co-culture, transfection, mechanical delivery, sonic delivery, vibrational delivery, magnetofection or by nanoparticle-mediated delivery. In some embodiments of the methods of the disclosure, introducing a nucleic acid sequence and/or a genomic editing construct into an immune cell ex vivo, in vivo, in vitro or in situ comprises liposomal transfection, calcium phosphate transfection, fugene transfection, and dendrimer-mediated transfection. In some embodiments of the methods of the disclosure, introducing a nucleic acid sequence and/or a genomic editing construct into an immune cell ex vivo, in vivo, in vitro or in situ by mechanical transfection comprises cell squeezing, cell bombardment, or gene gun techniques. In some embodiments of the methods of the disclosure, introducing a nucleic acid sequence and/or a genomic editing construct into an immune cell ex vivo, in vivo, in vitro or in situ by nanoparticle-mediated transfection comprises liposomal delivery, delivery by micelles, and delivery by polymerosomes.

In some embodiments of the methods of the disclosure, introducing a nucleic acid sequence and/or a genomic editing construct into an immune cell ex vivo, in vivo, in vitro or in sifu comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In some embodiments, the non-viral vector comprises a transposon of the disclosure.

In some embodiments of the methods of the disclosure, enzymes may be used to create strand breaks in the host genome to facilitate delivery or integration of the transgene. In some embodiments, enzymes create single-strand breaks. In some embodiments, enzymes create double-strand breaks. In some embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) or zinc finger nucleases (ZFN). In some embodiments, break-inducing enzymes can be delivered to the cell encoded in DNA, encoded in mRNA, as a protein, as a nucleoprotein complex with a guide RNA (gRNA).

In some embodiments of the methods of the disclosure, the site-specific transgene integration is controlled by a vector-mediated integration site bias. In some embodiments vector-mediated integration site bias is controlled by the chosen lentiviral vector. In some embodiments vector-mediated integration site bias is controlled by the chosen gamma-retroviral vector.

In some embodiments of the methods of the disclosure, the site-specific transgene integration site is a non-stable chromosomal insertion. In some embodiments, the integrated transgene may become silenced, removed, excised, or further modified

In some embodiments of the methods of the disclosure, the genome modification is a non-stable integration of a transgene. In some embodiments, the non-stable integration can be a transient non-chromosomal integration, a semi-stable non chromosomal integration, a semi-persistent non-chromosomal insertion, or a non-stable chromosomal insertion. In some embodiments, the transient non-chromosomal insertion can be epi-chromosomal or cytoplasmic.

In some embodiments, the transient non-chromosomal insertion of a transgene does not integrate into a chromosome and the modified genetic material is not replicated during cell division.

In some embodiments of the methods of the disclosure, the genome modification is a semi-stable or persistent non-chromosomal integration of a transgene. In some embodiments, a DNA vector encodes a Scaffold/matrix attachment region (S-MAR) module that binds to nuclear matrix proteins for episomal retention of a non-viral vector allowing for autonomous replication in the nucleus of dividing cells.

In some embodiments of the methods of the disclosure, the genome modification is a non-stable chromosomal integration of a transgene. In some embodiments, the integrated transgene may become silenced, removed, excised, or further modified.

In some embodiments of the methods of the disclosure, the modification to the genome by transgene insertion can occur via host cell-directed double-strand breakage repair (homology-directed repair) by homologous recombination (HR), microhomology-mediated end joining

(MMEJ), nonhomologous end joining (NHEJ), transposase enzyme-mediated modification, integrase enzyme-mediated modification, endonuclease enzyme-mediated modification, or recombinant enzyme-mediated modification. In some embodiments, the modification to the genome by transgene insertion can occur via CRISPR-Cas9, CRISPR-CasX, TALEN or ZFNs.

### C. Nanoparticle Delivery

Poly (histidine) (i.e., poly (L-histidine)), is a pH-sensitive polymer due to the imidazole ring providing an electron lone pair on the unsaturated nitrogen. That is, poly (histidine) has amphoteric properties through protonation-deprotonation. The various embodiments enable intracellular delivery of gene editing tools by complexing with poly (histidine)-based micelles. In particular, the various embodiments provide triblock copolymers made of a hydrophilic block, a hydrophobic block, and a charged block. In some embodiments, the hydrophilic block may be poly (ethylene oxide) (PEO), and the charged block may be poly (L-histidine) An example tri-block copolymer that may be used in various embodiments is a PEO-b-PLA-b-PHIS, with variable numbers of repeating units in each block varying by design. The gene editing tools may be various molecules that are recognized as capable of modifying, repairing, adding and/or silencing genes in various cells. The correct and efficient repair of double-strand breaks (DSBs) in DNA is critical to maintaining genome stability in cells. Structural damage to DNA may occur randomly and unpredictably in the genome due to any of a number of intracellular factors (e.g., nucleases, reactive oxygen species, etc.) as well as external forces (e.g., ionizing radiation, ultraviolet (UV) radiation, etc.). In particular, correct and efficient repair of double-strand breaks (DSBs) in DNA is critical to maintaining genome stability. Accordingly, cells naturally possess a number of DNA repair mechanisms, which can be leveraged to alter DNA sequences through controlled DSBs at specific sites. Genetic modification tools may therefore be composed of programmable, sequence-specific DNA-binding modules associated with a nonspecific DNA nuclease, introducing DSBs into the genome. For example, CRISPR, mostly found in bacteria, are loci containing short direct repeats, and are part of the acquired prokaryotic immune system, conferring resistance to exogenous sequences such as plasmids and phages. RNA-guided endonucleases are programmable genetic engineering tools that are adapted from the CRISPR/CRISPR-associated protein 9 (Cas9) system, which is a component of prokaryotic innate immunity.

Diblock copolymers that may be used as intermediates for making triblock copolymers of the embodiment micelles may have hydrophilic biocompatible poly (ethylene oxide) (PEO), which is chemically synonymous with PEG, coupled to various hydrophobic aliphatic poly (anhydrides), poly (nucleic acids), poly (esters), poly (ortho esters), poly (peptides), poly (phosphazenes) and poly (saccharides), including but not limited by poly (lactide) (PLA), poly (glycolide) (PLGA), poly (lactic-co-glycolic acid) (PLGA), poly (ε-caprolactone) (PCL), and poly (trimethylene carbonate) (PTMC). Polymeric micelles comprised of 100% PEGylated surfaces possess improved in vitro chemical stability, augmented in vivo bioavailablity, and prolonged blood circulatory half-lives. For example, aliphatic polyesters, constituting the polymeric micelle's membrane portions, are degraded by hydrolysis of their ester linkages in physiological conditions such as in the human body Because of their biodegradable nature, aliphatic polyesters have received a great deal of attention for use as implantable biomaterials in drug delivery devices, bioresorbable sutures, adhesion barriers, and as scaffolds for injury repair via tissue engineering.

In various embodiments, molecules required for gene editing (i.e., gene editing tools) may be delivered to cells using one or more micelle formed from self-assembled triblock copolymers containing poly (histidine). The term “gene editing” as used herein refers to the insertion, deletion or replacement of nucleic acids in genomic DNA so as to add, disrupt or modify the function of the product that is encoded by a gene. Various gene editing systems require, at a minimum, the introduction of a cutting enzyme (e.g., a nuclease or recombinase) that cuts genomic DNA to disrupt or activate gene function.

Further, in gene editing systems that involve inserting new or existing nucleotides/nucleic acids, insertion tools (e.g. DNA template vectors, transposable elements (transposons or retrotransposons) must be delivered to the cell in addition to the cutting enzyme (e.g. a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. In some embodiments, an example recombinase that may be used as a cutting enzyme is the CRE recombinase. In various embodiments, example integrases that may be used in insertion tools include viral based enzymes taken from any of a number of viruses including, but not limited to, AAV, gamma retrovirus, and lentivirus. Example transposons/retrotransposons that may be used in insertion tools include, but are not limited to, the piggyBac® transposon, Sleeping Beauty transposon, TcBuster transposon and the L1 retrotransposon.

In certain embodiments of the methods of the disclosure, the transgene is delivered in vivo. In certain embodiments of the methods of the disclosure, in vivo transgene delivery can occur by: topical delivery, adsorption, absorption, electroporation, spin-fection, co-culture, transfection, mechanical delivery, sonic delivery, vibrational delivery, magnetofection or by nanoparticle-mediated delivery. In certain embodiments of the methods of the disclosure, in vivo transgene delivery by transfection can occur by liposomal transfection, calcium phosphate transfection, fugene transfection, and dendrimer-mediated transfection. In certain embodiments of the methods of the disclosure, in vivo mechanical transgene delivery can occur by cell squeezing, bombardment, and gene gun. In certain embodiments of the methods of the disclosure, in vivo nanoparticle-mediated transgene delivery can occur by liposomal delivery, delivery by micelles, and delivery by polymerosomes. In various embodiments, nucleases that may be used as cutting enzymes include, but are not limited to, Cas9, transcription activator-like effector nucleases (TALENs) and zinc finger nucleases.

In various embodiments, the gene editing systems described herein, particularly proteins and/or nucleic acids, may be complexed with nanoparticles that are poly (histidine)-based micelles. In particular, at certain pHs, poly (histidine)-containing triblock copolymers may assemble into a micelle with positively charged poly (histidine) units on the surface, thereby enabling complexing with the negatively-charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA may be enabled by delivery of a nuclease using the poly (histidine)-based micelles.

The various embodiments enable intracellular delivery of gene editing tools by complexing with poly (histidine)-based micelles. In particular, the various embodiments provide triblock copolymers made of a hydrophilic block, a hydrophobic block, and a charged block. In some embodiments, the hydrophilic block may be poly (ethylene oxide) (PEO), and the charged block may be poly (L-histidine). An example tri-block copolymer that may be used in various embodiments is a PEO-b-PLA-b-PHIS, with variable numbers of repeating units in each block varying by design. Without wishing to be bound by a particular theory, it is believed that believed that in the micelles that are formed by the various embodiment triblock copolymers, the hydrophobic blocks aggregate to form a core, leaving the hydrophilic blocks and poly (histidine) blocks on the ends to form one or more surrounding layer.

In certain embodiments of the methods of the disclosure, non-viral vectors are used for transgene delivery. In certain embodiments, the non-viral vector is a nucleic acid. In certain embodiments, the nucleic acid non-viral vector is plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In certain embodiments, the non-viral vector is a transposon. In certain embodiments, the transposon is TcBuster.

In certain embodiments of the methods of the disclosure, transgene delivery can occur via viral vector. In certain embodiments, the viral vector is a non-integrating non-chromosomal vectors. Non-integrating non-chromosomal vectors can include adeno-associated virus (AAV), adenovirus, and herpes viruses. In certain embodiments, the viral vector is an integrating chromosomal vectors. Integrating chromosomal vectors can include adeno-associated vectors (AAV), Lentiviruses, and gamma-retroviruses.

In certain embodiments of the methods of the disclosure, transgene delivery can occur by a combination of vectors. Exemplary but non-limiting vector combinations can include: viral plus non-viral vectors, more than one non-viral vector, or more than one viral vector. Exemplary but non-limiting vectors combinations can include: DNA-derived plus RNA-derived vectors, RNA plus reverse transcriptase, a transposon and a transposase, a non-viral vectors plus an endonuclease, and a viral vector plus an endonuclease.

In certain embodiments of the methods of the disclosure, the genome modification can be a stable integration of a transgene, a transient integration of a transgene, a site-specific integration of a transgene, or a biased integration of a transgene.

In certain embodiments of the methods of the disclosure, the genome modification can be a stable chromosomal integration of a transgene. In certain embodiments, the stable chromosomal integration can be a random integration, a site-specific integration, or a biased integration. In certain embodiments, the site-specific integration can be non-assisted or assisted.

In certain embodiments, the assisted site-specific integration is co-delivered with a site-directed nuclease. In certain embodiments, the site-directed nuclease comprises a transgene with 5′ and 3′ nucleotide sequence extensions that contain homology to upstream and downstream regions of the site of genomic integration. In certain embodiments, the transgene with homologous nucleotide extensions enable genomic integration by homologous recombination, microhomology-mediated end joining, or nonhomologous end-joining. In certain embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

In certain embodiments, the site-specific transgene integration occurs at a site that disrupts expression of a target gene. In certain embodiments, disruption of target gene expression occurs by site-specific integration at introns, exons, promoters, genetic elements, enhancers, suppressors, start codons, stop codons, and response elements. In certain embodiments, exemplary target genes targeted by site-specific integration include but are not limited to any immunosuppressive gene, and genes involved in allo-rejection.

In certain embodiments, the site-specific transgene integration occurs at a site that results in enhanced expression of a target gene. In certain embodiments, enhancement of target gene expression occurs by site-specific integration at introns, exons, promoters, genetic elements, enhancers, suppressors, start codons, stop codons, and response elements.

In certain embodiments of the methods of the disclosure, enzymes may be used to create strand breaks in the host genome to facilitate delivery or integration of the transgene. In certain embodiments, enzymes create single-strand breaks. In certain embodiments, enzymes create double-strand breaks. In certain embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) and zinc finger nucleases (ZFN). In certain embodiments, break-inducing enzymes can be delivered to the cell encoded in DNA, encoded in mRNA, as a protein, as a nucleoprotein complex with a guide RNA (gRNA).

In certain embodiments of the methods of the disclosure, the site-specific transgene integration is controlled by a vector-mediated integration site bias. In certain embodiments vector-mediated integration site bias is controlled by the chosen lentiviral vector. In certain embodiments vector-mediated integration site bias is controlled by the chosen gamma-retroviral vector.

In certain embodiments of the methods of the disclosure, the site-specific transgene integration site is a non-stable chromosomal insertion. In certain embodiments, the integrated transgene may become silenced, removed, excised, or further modified. In certain embodiments of the methods of the disclosure, the genome modification is a non-stable integration of a transgene. In certain embodiments, the non-stable integration can be a transient non-chromosomal integration, a semi-stable non chromosomal integration, a semi-persistent non-chromosomal insertion, or a non-stable chromosomal insertion. In certain embodiments, the transient non-chromosomal insertion can be epi-chromosomal or cytoplasmic. In certain embodiments, the transient non-chromosomal insertion of a transgene does not integrate into a chromosome and the modified genetic material is not replicated during cell division.

In certain embodiments of the methods of the disclosure, the genome modification is a semi-stable or persistent non-chromosomal integration of a transgene. In certain embodiments, a DNA vector encodes a Scaffold/matrix attachment region (S-MAR) module that binds to nuclear matrix proteins for episomal retention of a non-viral vector allowing for autonomous replication in the nucleus of dividing cells.

In certain embodiments of the methods of the disclosure, the genome modification is a non-stable chromosomal integration of a transgene. In certain embodiments, the integrated transgene may become silenced, removed, excised, or further modified.

In certain embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In certain embodiments, the modification to the genome by transgene insertion can occur via CRISPR-Cas9, CRISPR-CasX, TALEN or ZFNs.

In certain embodiments of the methods of the disclosure, a cell with an in vivo or ex vivo genomic modification can be a germline cell or a somatic cell. In certain embodiments the modified cell can be a human, non-human, mammalian, rat, mouse, or dog cell. In certain embodiments, the modified cell can be differentiated, undifferentiated, or immortalized. In certain embodiments, the modified undifferentiated cell can be a stem cell. In certain embodiments, the modified cell can be differentiated, undifferentiated, or immortalized. In certain embodiments, the modified undifferentiated cell can be an induced pluripotent stem cell. In certain embodiments, the modified cell can be a T cell, a hematopoietic stem cell, a natural killer cell, a macrophage, a dendritic cell, a monocyte, a megakaryocyte, or an osteoclast. In certain embodiments, the modified cell can be modified while the cell is quiescent, in an activated state, resting, in interphase, in prophase, in metaphase, in anaphase, or in telophase. In certain embodiments, the modified cell can be fresh, cryopreserved, bulk, sorted into sub-populations, from whole blood, from leukapheresis, or from an immortalized cell line.

### B. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZENs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al, Trends in Biotechnology, 2013, 31 (7), 397-405). In some embodiments, commercially available zinc fingers are used or are custom designed. (See, for example, Sigma-Aldrich catalog numbers CSTZEND, CSTZEN, CTil-IKT, and PZD0020).

### C. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to Gor A, and NO binds to T and non-canonical (atypical) RVDs are also known. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., 2013). A library of TALENs targeting 18,740 human protein-coding genes has been constructed (Kim et al, 2013). Custom-designed TALE arrays are commercially available through Cellectis Bioresearch (Paris, France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1, HTN222870-2, and HTN222870-3). Exemplary molecules are described, e.g., in U.S. Patent Publication Nos. US 2014/0120622, and 2013/0315884.

In some embodiments the TALEN s are introduced as trans genes encoded by one or more plasmid vectors. In some aspects, the plasmid vector can contain a selection marker which provides for identification and/or selection of cells which received said vector.

### D. Meganucleases and MegaTALs

In certain embodiments, the nuclease comprises a meganuclease (homing endonuclease) or a portion thereof that exhibits cleavage activity. In some embodiments, a “meganuclease,” also referred to as a “homing endonuclease,” refers to an endodeoxyribonuclease characterized by a large recognition site (double stranded DNA sequences of about 12 to about 40 base pairs). Naturally-occurring meganucleases recognize 15-40 base-pair cleavage sites and are commonly grouped into four families: the LAGLIDADG family, the GIY-YIG family, the His-Cyst box family and the HINH family. Exemplary homing endonucleases include I-Scel, I-Ceul, PI-PspI, PI-Sce, I-SceIV, I-CsmI, I-PanI, I-Scell, I-PpoI, I-SceIII, I-Crel, I-TevI, I-TevII and I-TevIII Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263.163-180, Argast et al. (1998) J. Mol. Biol. 280:345-353 and the New England Biolabs catalogue.

DNA-binding domains from naturally-occurring meganucleases, primarily from the LAGLIDADG family, have been used to promote site-specific genome modification in plants, yeast, Drosophila, mammalian cells and mice, but this approach has been limited to the modification of either homologous genes that conserve the meganuclease recognition sequence (Monet et al. (1999), Biochem. Biophysics. Res. Common. 255:88-93) or to pre-engineered genomes into which a recognition sequence has been introduced (Route et al. (1994), Mol. Cell. Biol. 14:8096-106; Chilton et al. (2003), Plant Physiology. 133:956-65; Puchta et al. (1996), Proc. Natl. Acad. Sci. USA 93:5055-60; Rong et al. (2002), Genes Dev. 16:1568-81; Gouble et al. (2006), J. Gene Med. 8 (5): 616-622). Accordingly, attempts have been made to engineer meganucleases to exhibit novel binding specificity at medically or biotechnologically relevant sites (Porteus et al. (2005), Nat. Biotechnol. 23:967-73; Sussman et al. (2004), J. Mol. Biol. 342:31-41; Epinat et al. (2003), Nucleic Acids Res. 31:2952-62; Chevalier et al. (2002) Molec. Cell 10:895-905: Epinat et al. (2003) Nucleic Acids Res. 31:2952-2962; Ashworth et al. (2006) Nature 441:656-659; Paques et al. (2007) Current Gene Therapy 7:49-66; U.S. Patent Publication Nos. 20070117128; 20060206949; 20060153826; 20060078552; and 20040002092). In addition, naturally-occurring or engineered DNA-binding domains from meganucleases can be operably linked with a cleavage domain from a heterologous nuclease (e.g., FokI) and/or cleavage domains from meganucleases can be operably linked with a heterologous DNA-binding domain (e.g., ZFP or TALE).

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site specific interaction with the target sequence of the user's choosing have been published (see U.S. Pat. No. 8,586,526). In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224). In addition, the nuclease domain may also exhibit DNA-binding functionality.

### E. RGENs (CRISPR/Cas Systems)

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The traer sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

The components of a CRISPR system can be implemented in any suitable manner, meaning that the components of such systems including the RNA-guided nuclease (e.g., Cas enzyme) and gRNA can be delivered, formulated or administered in any suitable form to the cells. For example, the RNA-guided nuclease may be delivered to a cell complexed with a gRNA (e.g., as a ribonucleoprotein (RNP) complex), the RNA-guided nuclease may be delivered to a cell separate (e.g., uncomplexed) to a gRNA, the RNA-guided nuclease may be delivered to a cell as a polynucleotide (e.g., DNA or RNA) encoding the nuclease that is separate from a gRNA, or both the RNA-guided nuclease and the gRNA molecule may be delivered as polynucelotides encoding each component.

One or more vectors driving expression of one or more elements of the CRISPR system can be introduced into the cell such that expression of the elements of the CRISPR system direct formation of the CRISPR complex at one or more target sites. Components can also be delivered to cells as ribonucleoprotein complexes, proteins, DNA, and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. In addition, a nucleic acid encoding the endonuclease (e.g., a Cas enzyme such as Cas8 or Cas9) may be delivered with gRNAs When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csnl and Csx12), Cas10, CasX, Csy1, Csy2, Csy3, Csel, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csxl4, CsxlO, Csxl6, CsaX, Csx3, Csxl, Csxl5, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g, from S. pyogenes or S. pneumonia) The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference.

### VII. Methods of Use

In some embodiments, the present disclosure provides methods for immunotherapy comprising administering an effective amount of the immune cells of the present disclosure. In one embodiments, a medical disease or disorder is treated by transfer of an immune cell population that elicits an immune response. In certain embodiments of the present disclosure, cancer or infection is treated by transfer of an immune cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. In some embodiments, the cancer is a CD22-positive cancer. In some embodiments, the cancer has a low expression of CD22 (e.g. a CD22 low cancer). In some embodiments, the cancer is a CD19-positive cancer. In some embodiments, the cancer has a low expression of CD19 (e.g. a CD19 low cancer).

Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include but are not limited to tumors of the bone marrow, Tor B cell malignancies, myeloid malignancies, leukemias, lymphomas, blastomas, myelomas. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma, papillary and follicular adenocarcinoma; nonencapsulating sclerosing carcinoma; adrenal cortical carcinoma; endometroid carcinoma; skin appendage carcinoma, apocrine adenocarcinoma; sebaceous adenocarcinoma; ceruminous adenocarcinoma, mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; T lymphoblastic leukemia; T lymphoblastic lymphoma; Hodgkin's disease; Hodgkin's lymphoma, paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL, bulky disease NHL; mantle cell lymphoma; AIDS-related lymphoma; Waldenstrom's macroglobulinemia; malignant histiocytosis; multiple myeloma; mast cell sarcoma, immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); chronic myeloid leukemia, acute lymphoblastic leukemia (ALL); acute lymphoblastic lymphoma; acute myeloid leukemia (AML); myelodysplastic syndrome (MDS); myeloproliferative neoplasms; chronic myeloblasts leukemia; diffuse large B-cell lymphoma (DLBCL); peripheral T-cell lymphoma (PTCL); or anaplastic large cell lymphoma (ALCL).

Particular embodiments concern methods of treatment of leukemia. Leukemia is a cancer of the blood or bone marrow and is characterized by an abnormal proliferation (production by multiplication) of blood cells, usually immature white blood cells (leukocytes). It is part of the broad group of diseases called hematological neoplasms. Leukemia is a broad term covering a spectrum of diseases. Leukemia is clinically and pathologically split into its acute and chronic forms and/or by and the cell type of origin (myeloid or lymphoid). In some embodiments, the leukemia is an antigen-low leukemia. In some embodiments, the leukemia is a CD22-low leukemia

In certain embodiments of the present disclosure, immune cells are delivered to an individual in need thereof, such as an individual that has cancer or an infection. The cells then enhance the individual's immune system to attack or directly attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Bechet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or membranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosus, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m2 fludarabine is administered for five days.

In some embodiments, the subject can be administered nonmyeloablative lymphodepleting immunotherapy prior to the immune cell therapy. The nonmyeloablative lymphodepleting immunotherapy can be any suitable such therapy, which can be administered by any suitable route. The nonmyeloablative lymphodepleting immunotherapy can comprise, for example, the administration of an anti-CD52 agent or anti-CD20 agent. In some embodiments, the lymphodepleting immunotherapy is an anti-CD52 antibody. In some embodiments, the anti-CD52 antibody is alemtuzumab. In some embodiments, the lymphodepleting immunotherapy is an anti-CD20 antibody. Exemplary anti-CD20 antibodies include, but are not limited to rituximab, ofatumumab, ocrelizumab, obinutuzumab, ibritumomab or iodine i131 tositumomab. An exemplary route of administering anti-CD52 agent or anti-CD20 agent is intravenously. Likewise, any suitable dose of anti-CD52 agent or anti-agent can be administered.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as from about 2×10⁷ cells to about 5×10⁸ cells per kg body weight, or from about 5×10⁷ cells to about 2×10⁸ cells per kg body weight, or from about 5×10⁶ cells per kg body weight to about 1×10⁷ cells per kg body weight. In some embodiments, a therapeutically effective amount of immune cells ranges from about 1×10⁵ cells per kg body weight to about 10×10⁹ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetyls alicylic acid, ibuprofen or naproxen sodium), cytokine antagonists (for example, anti-TNF and anti-IL-6), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

### 1. Pharmaceutical Compositions

Also provided herein are pharmaceutical compositions and formulations comprising immune cells (e.g., T cells) and a pharmaceutically acceptable carrier.

In some embodiments, a pharmaceutical composition comprises a dose ranging from about 1×10⁵ T cells to about 1×10⁹ T cells. In some embodiments, the dose is about 1×10⁵, 1×10⁶, 1×10⁷, 1×10⁸ or 1×10⁹ T cells. In some embodiments, a pharmaceutical composition comprises a dose ranging from about 5×10⁵ T cells to about 10×10¹² T cells.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEXR, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### 2. Combination Therapies

In some embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, decitabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide, mitoxantrone; vincristine; vinorelbine; novantrone, teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above. In some embodiments, azacitidine is administered at 75 mgs/m2 subcutaneously.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al, 1998); cytokine therapy, e.g, interferons α, β, and γ, IL-1, GM-CSF, and TNF (Bukowski et al, 1998; Davidson et al, 1998; Hellstrand et al, 1998); gene therapy, e.g., TNF, IL-1, IL-2, and p53 (Qin et al, 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al, 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12 (4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g, a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab, formerly ticilimumab), U.S. Pat. No. 6,207,156; Hurwitz et al. (1998) Proc Natl Acad Sci USA 95 (17): 10067-10071; Camacho et al. (2004)/Clin Oncology 22 (145). Abstract No. 2505 (antibody CP-675206), and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### 3. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 4. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy

### VIII. Dosage Regimens

In one embodiment, the immune effector cells (e.g., T cells) are modified by engineering/introducing chimeric antigen receptors (e.g., anti-CD22 and anti-CD19 CAR) into said immune effector cells and then infused into a subject. In some embodiments, immune effector cells are modified by engineering/introducing a chimeric receptor, and functional effector element and/or a cytokine into the immune effector cells and then infused within about 0 days, within about 1 day, within about 2 days, within about 3 days, within about 4 days, within about 5 days, within about 6 days or within about 7 days into a subject.

In some embodiments, an amount of modified effector cells is administered to a subject in need thereof and the amount is determined based on the efficacy and the potential of inducing a cytokine-associated toxicity. In another embodiment, the modified effector cells are CAR⁺ and CD56⁺ cells. In some embodiments, an amount of modified effector cells comprises about 10⁺ to about 10⁹ modified effector cells/kg. In some cases, an amount of modified effector cells comprises about 10⁴ to about 10⁵ modified effector cells/kg. In some cases, an amount of modified effector cells comprises about 10⁵ to about 10⁶ modified effector cells/kg. In some cases, an amount of modified effector cells comprises about 10⁶ to about 10⁷ modified effector cells/kg. In some cases, an amount of modified effector cells comprises about 10⁷ to about 10⁸ modified effector cells/kg. In some cases, an amount of modified effector cells comprises about 10⁸ to about 10⁹ modified effector cells/kg. In some cases, am amount of modified effector cells comprises about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹ modified effector cells/kg.

In one embodiment, the modified immune effector cells are targeted to the cancer via regional delivery directly to the tumor tissue. For example, in ovarian or renal cancer, the modified immune effector cells can be delivered intraperitoneally (IP) to the abdomen or peritoneal cavity. Such IP delivery can be performed via a port or pre-existing port placed for delivery of chemotherapy drugs. Other methods of regional delivery of modified immune effector cells can include catheter infusion into resection cavity, ultrasound guided intratumoral injection, hepatic artery infusion or intrapleural delivery.

In one embodiment, a subject in need thereof, can begin therapy with a first dose of modified immune effector cells delivered via IV followed by a second dose of modified immune effector cells delivered via IV. In one embodiment, a subject in need thereof, can begin therapy with a first dose of modified immune effector cells delivered via IP followed by a second dose of modified immune effector cells delivered via IV. In a further embodiment, the second dose of modified immune effector cells can be followed by subsequent doses which can be delivered via IV or IP. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the duration between the first and second or further subsequent dose can be about. 0, 1, 2, 3, 4, 5, 6, 7, 8, 9 or 10 years.

In another embodiment, a catheter can be placed at the tumor or metastasis site for further administration of 1, 2, 3, 4, 5, 6, 7, 8, 9, 10 doses of modified immune effector cells. In some cases, doses of modified effector cells can comprise about 10² to about 10⁹ modified effector cells/kg. In cases where toxicity is observed, doses of modified effector cells can comprise about 10² to about 10⁵ modified effector cells/kg. In some cases, doses of modified effector cells can start at about 102 modified effector cells/kg and subsequent doses can be increased to about: 10⁴, 10⁵, 10⁶, 10⁷, 10⁸ or 10⁹ modified effector cells/kg.

### IX. Articles of Manufacture or Kits

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use.

The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### X. REFERENCES

Maude, S. L. et al. Chimeric Antigen Receptor T Cells for Sustained Remissions in Leukemia. N. Engl. J. Med. 371, 1507-1517 (2014).
Lee, D. W. et al. T cells expressing CD19 chimeric antigen receptors for acute lymphoblastic leukaemiain children and young adults: a phase 1 dose-escalation trial. Lancet 385, 517-528 (2015).
Park, J. H. et al. Long-Term Follow-up of CD19 CAR Therapy in Acute Lymphoblastic Leukemia. N. Engl. J. Med. 378, 449-459 (2018).
Gardner, R. et al. Acquisition of a CD19-negative myeloid phenotype allows immune escape of MLL-rearranged B-ALL from CD19 CAR-T-cell therapy. Blood 127, 2406-2410 (2016).
Turtle, C. J. et al. CD19 CAR-T cells of defined CD4+:CD8+ composition in adult B cell ALL patients. J. Clin. Invest. 126, 2123-2138 (2016).
Gardner, R. A. et al. Intent to treat leukemia remission by CD19CAR T cells of defined formulation and dose in children and young adults. Blood blood-2017-02-769208 (2017). doi: 10.1182/blood-2017-02-769208
Maude, S. L. et al. Tisagenlecleucel in Children and Young Adults with B-Cell Lymphoblastic Leukemia. N. Engl. J. Med. 378, 439-448 (2018).
Pasquini, M. C. et al. Real-world evidence of tisagenlecleucel for pediatric acute lymphoblastic leukemia and non-Hodgkin lymphoma. Blood Adv. 4, (2020).
Wudhikarn, K. et al. Interventions and Outcomes of Adult Patients with B-ALL Progressing After CD19Chimeric Antigen Receptor T Cell Therapy. Blood (2021). doi: 10.1182/blood.2020009515
Myers, R. M. et al. Humanized CD19-Targeted Chimeric Antigen Receptor (CAR) T Cells in CAR-Naive and CAR-Exposed Children and Young Adults With Relapsed or Refractory Acute Lymphoblastic Leukemia J. Clin. Oncol. JCO.20.03458 (2021). doi: 10.1200/JCO 20.03458
Fry, T. J. et al. CD22-targeted CAR T cells induce remission in B-ALL that is naive or resistant to CD19-targeted CAR immunotherapy. Nat. Med. 24, 20-28 (2018)
Shah, N. N. et al. CD4/CD8 T-Cell Selection Affects Chimeric Antigen Receptor (CAR) T-Cell Potency and Toxicity: Updated Results From a Phase I Anti-CD22 CAR T-Cell Trial. J. Clin. Oncol. 38, 1938-1950 (2020).
Ramakrishna, S. et al. Modulation of Target Antigen Density Improves CAR T-cell Functionality and Persistence. Clin. Cancer Res. (2019). doi:10.1158/1078-0432.CCR-18-3784
Kohler, M. E., Walsh, Z., Degolier, K. & Fry, T. J. Enhancing the Antigen-Sensitivity of the CD22 CAR through Modulation of the Affinity and Linker-Length of the Single-Chain Fragment Variable. Blood 136, 41-42 (2020).
Walsh, Z., Yang, Y. & Kohler, M. E. Immunobiology of chimeric antigen receptor T cells and novel

### XI. EXAMPLES

### Example 1—Standard Second Generation CAR T Cells Targeting CD22 Cannot Effectively Target Antigen-Low Leukemia Due to Deficiencies in Signal Generated by the CAR

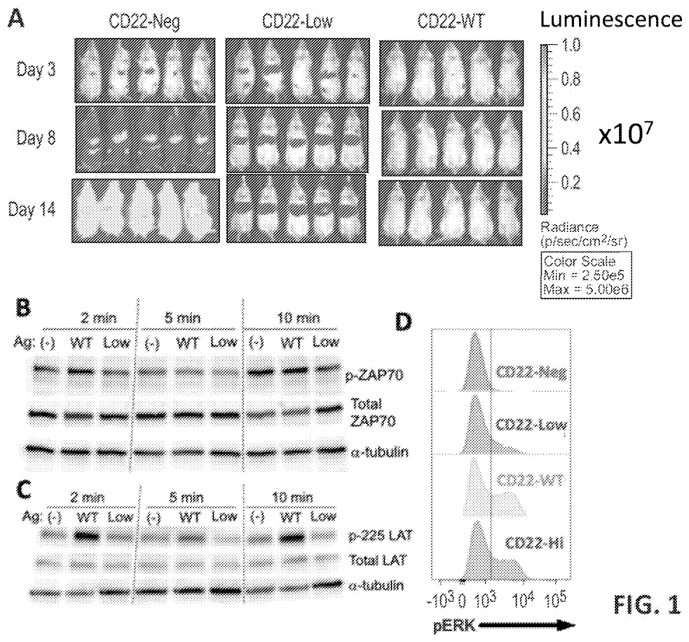

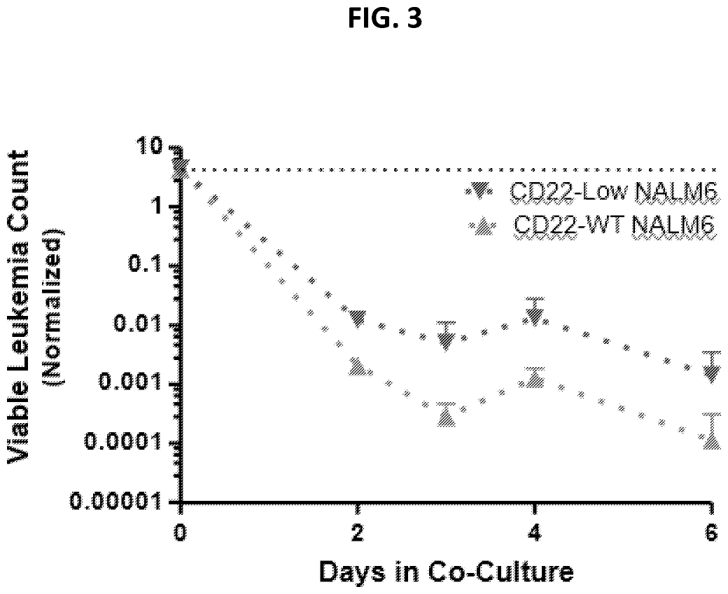

Using standard second generation CD22 CAR T cells generated from healthy donors, it was determined that in vivo efficacy was related to the amount of CD22 expressed on NALM6 (FIG. 1A). Signal transduction downstream of the CAR in response to NALM6 cells expressing no, low- or WT-levels of CD22 was tested. ZAP70 activation, which is proximal to LAT, demonstrated no relationship to the presence or amount of CD22 antigen (FIG. 1B); a finding likely related to the basal-dimerization of this CAR²⁸. However, the impact of antigen density was apparent at the level of LAT activation and further downstream, as phosphorylation of activating residues of both LAT and ERK were reduced in response to CD22-low target cells relative to targets expressing WT-levels of antigen (FIGS. 1C-1D). This correlated with decreased in vitro killing of CD22-low (upside down triangles) leukemia by standard 2nd generation CD22 CAR T cells, compared to NALM6 leukemia cells expressing wild-type (WT) levels (upright triangles) of CD22 antigen, as shown in FIG. 3. Thus, low levels of CD22 expression resulted in insufficient signaling through the LAT complex, resulting in attenuated downstream signaling events and diminished T cell killing of tumor cells.

### Example 2—A Novel Bicistronic Car Construct Incorporating the LAT Signaling Domains Overcomes Signaling Deficits from Antigen-Low Stimulation of CAR T Cells

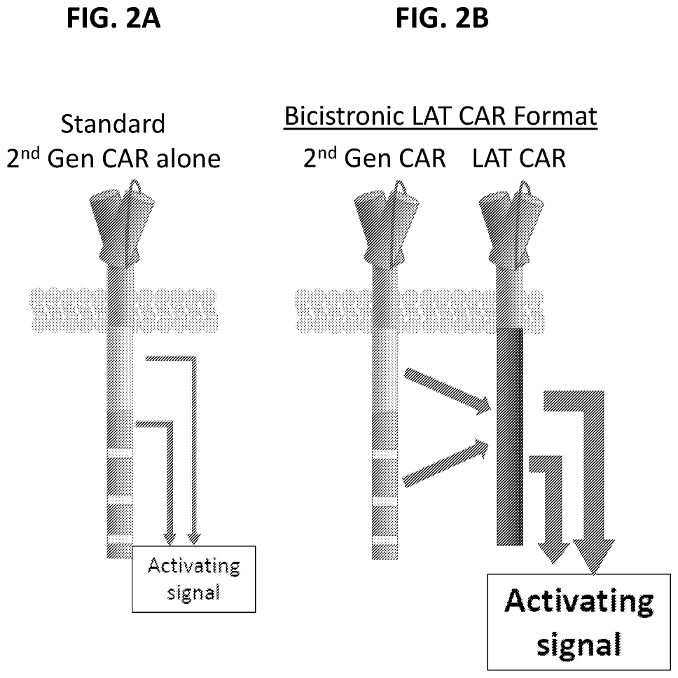

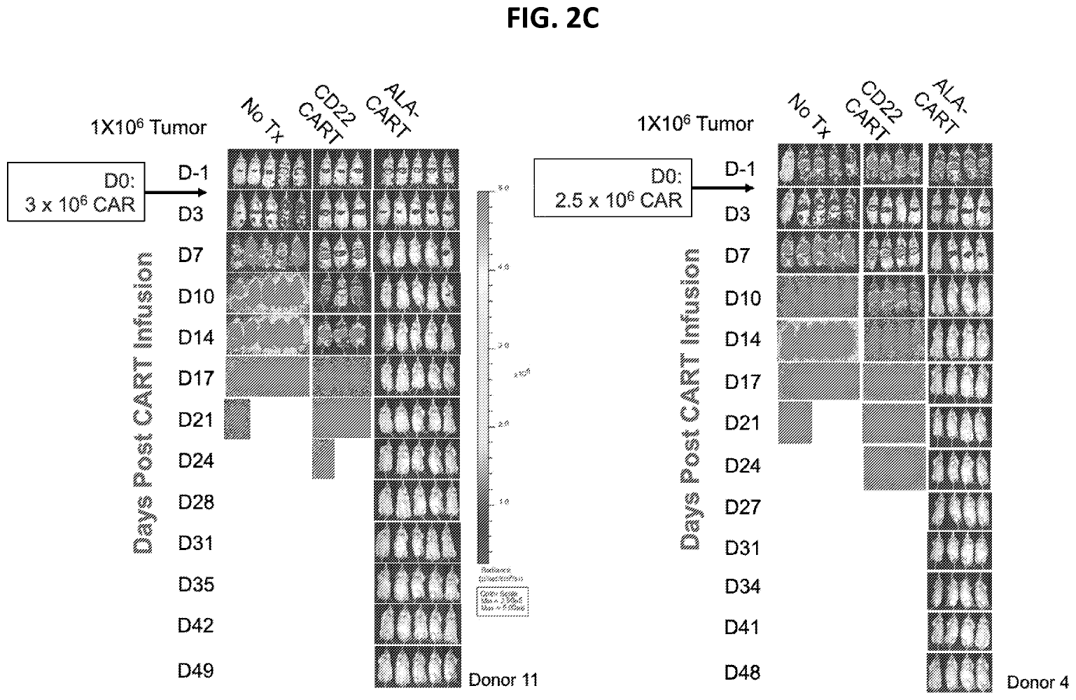

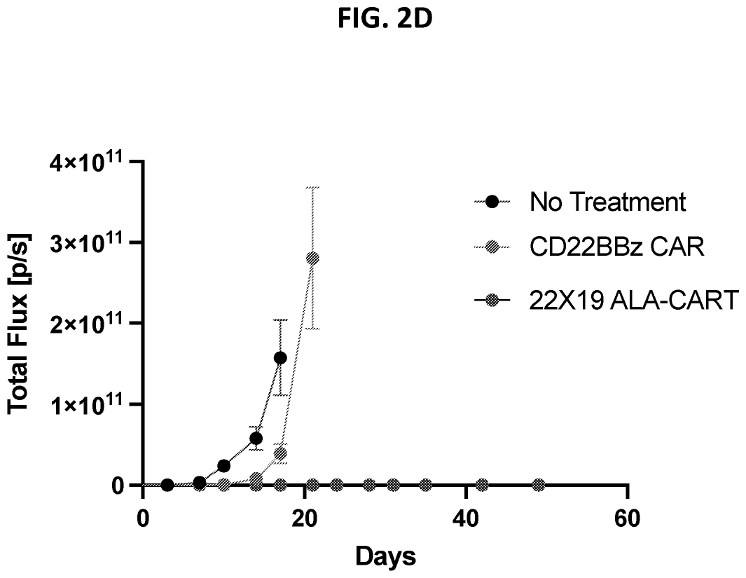

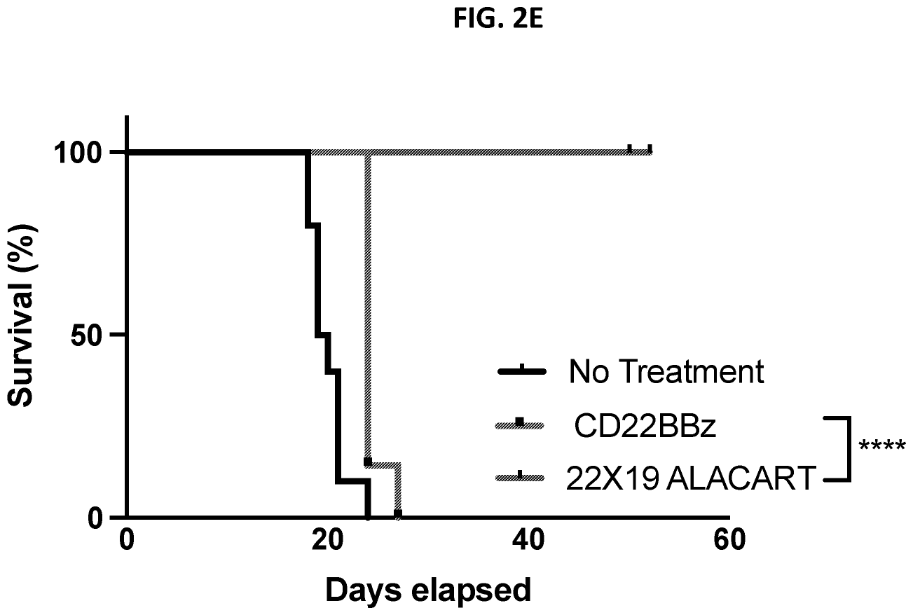

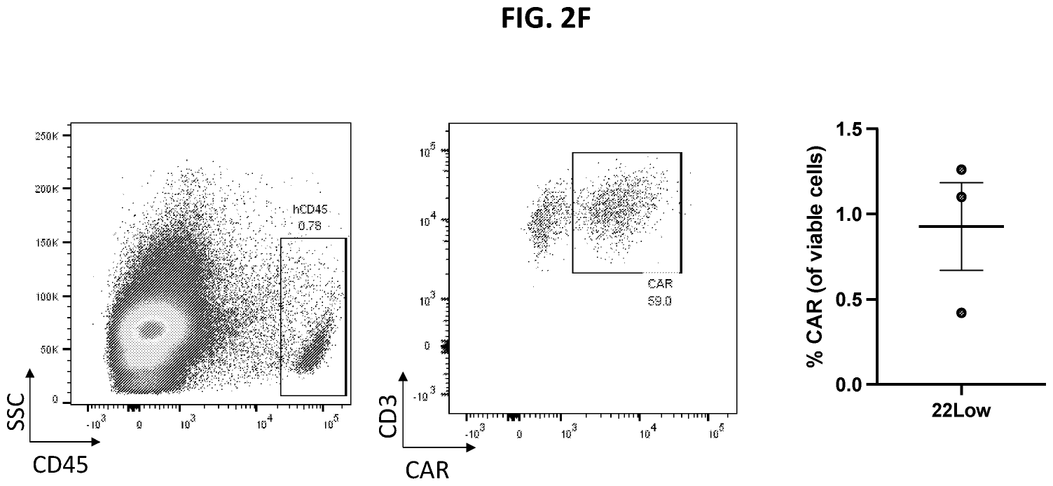

A novel CAR construct was designed to increase the efficiency of LAT activation in the setting of low antigen-density. A CD22-directed 2^{nd} generation (2G) CAR (also referred to as “2G CAR”) with m971 scFv, a 4-1BB costimulatory domain and a CD3-zeta chain was cloned into a bicistronic CAR vector (FIG. 2A). To enhance signaling through LAT, a second CAR was cloned downstream of the 2G CAR, separated by a P2A ribosomal skip sequence. The second CAR comprises a FMC63 scFv (targeting CD19) with a LAT intracellular domain (FIG. 2B). This bicistronic CAR (also referred to as “LAT-CAR” and “ALA-CART”, “22×19 ALA-CART′, “LAT-CAR”, “ALA-CART CD22BBz”, “CD22 2nd Gen CAR+CD19-LALT CAR”, or “22×19LAT”) forces localization of LAT with the 2G CAR as a means of amplifying CAR signaling, in response to low levels of CD22 expression.

### Example 3—Transduction of T Cells with ALA-CART Construct Results in Surface Expression of Each Individual CAR

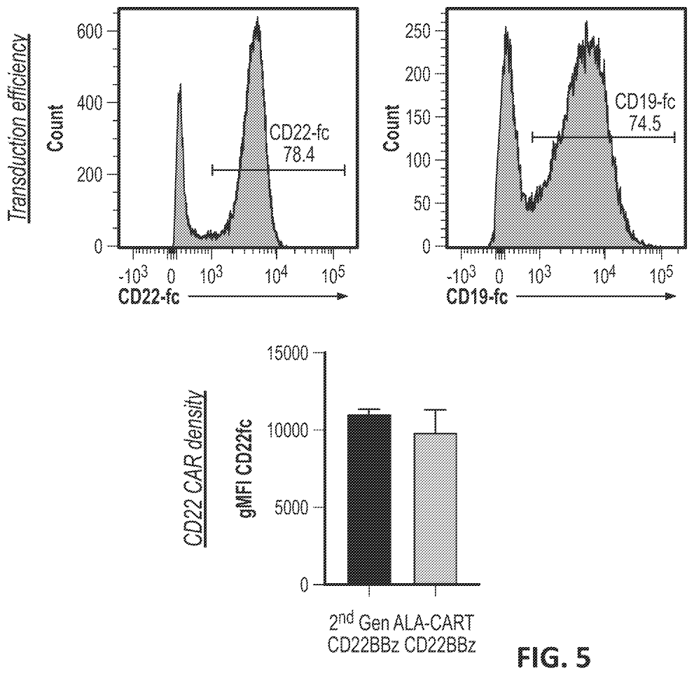

ALA-CART constructs were generated by cloning a 2^{nd} generation CAR targeting CD22 with the m971 scFv, a CD8α TM domain, a 4-1BB costimulatory domain and the signaling domain of CD3-zeta into a bicistronic construct with an Adjunctive LAT-Activating CAR targeting CD19 with the FMC63 scFv, CD28 transmembrane domain and the LAT intracellular domain. This was cloned into a 3^{rd} Generation Lentivirus transfer plasmid and replication-incompetent Lentivirus was generated by established lentivirus protocols. T cells from a healthy donor were activated and transduced with lentivirus encoding ALA-CART construct using standard transduction techniques. In parallel, T cells were transduced with a standard 2^{nd} generation CD22 CAR with a 4-1BB costimulatory domain (identical to the first CAR of the ALA-CART format) using the same transduction techniques. 3 days after transduction, surface expression of each CAR was evaluated by staining with fluorescently-labeled soluble CD22 antigen (detection of the standard 2^{nd} generation CAR) and fluorescently-labeled soluble CD19 antigen (detection of the Alternative LAT-Activating CAR). Stained cells were analyzed by flow cytometry. The results of this analysis are shown in FIG. 5. The data shown in FIG. 5 demonstrate that both CARs of the ALA-CART bicistronic construct are expressed on the surface of T cells after transduction. Furthermore, without wishing to be bound by theory, the ALA-CART construct does not lead to higher expression of the 200 Gen CD22 CAR, indicating that increases of function of ALA-CART cells is most likely due to the signaling properties of the ALA-CART design and not due to overexpression of the standard CAR.

ALA-CART constructs were generated by cloning a 2^{nd} generation CAR targeting CD22 with the m971 scFv, a CD8a domain, a 4-1BB costimulatory domain and the signaling domain of CD3-zeta into a bicistronic construct with a Adjunctive LAT-Activating CAR targeting CD19 with the FMC63 scFv connected to the LAT intracellular domain through either: a) the endogenous LAT transmembrane domain or b) the transmembrane domain of CD28 at position TM #2. These two completed constructs were cloned into a 3^{rd} Generation Lentivirus transfer plasmid and replication-incompetent Lentivirus was generated by established lab protocols. T cells from 3 separate healthy donors were activated and transduced with lentivirus encoding ALA-CART construct with either the LAT-TM or the CD28-TM at position TM #2 by established lab protocol. 3 days after transduction, surface expression of each CAR was evaluated by staining with fluorescently-labeled soluble CD22 antigen (detection of the standard 2^{nd} generation CAR) and fluorescently-labeled soluble CD19 antigen (detection of the Alternative LAT-Activating CAR). Stained cells were analyzed by flow cytometry.

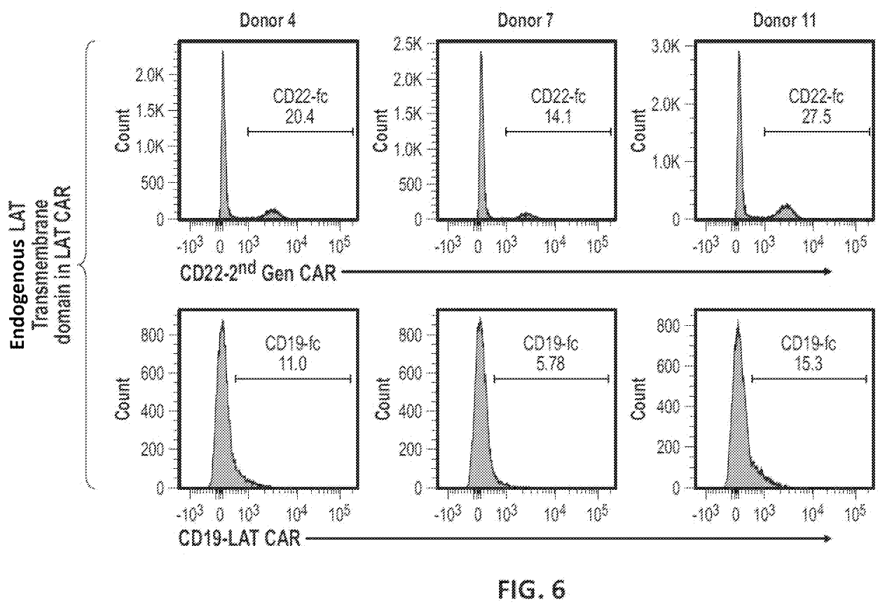

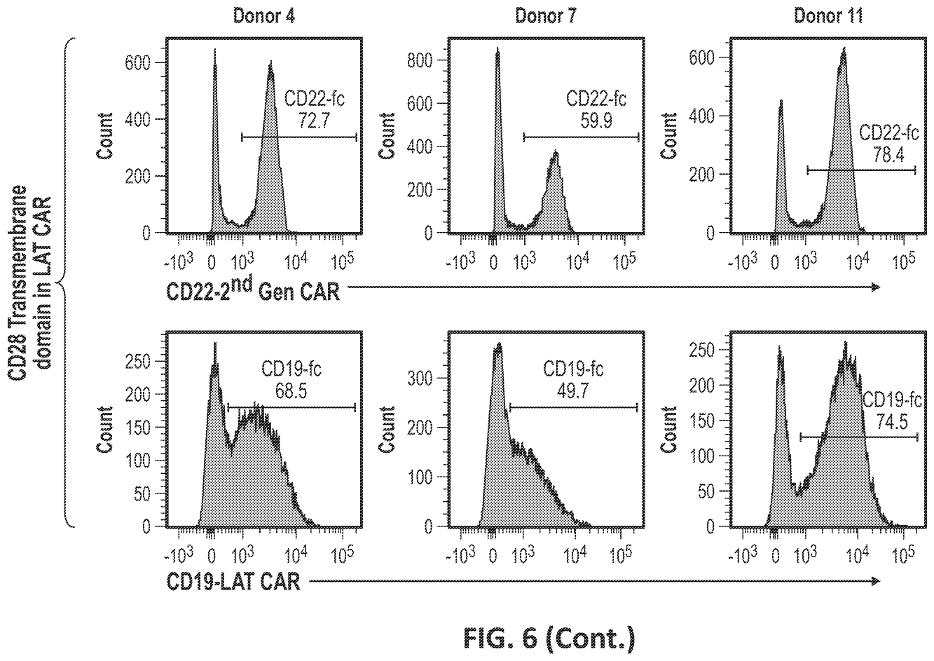

T cells from healthy donors were activated and transduced with the novel bicistronic ALA-CART construct to verify both individual CARs (e.g., a first CAR-CD22-directed 2^{nd} generation CAR and a second CAR-CD19-directed LAT CAR) were expressed upon the surface of the T cells. CAR surface expression was detected by staining each CAR with a fluorescently-labeled antigen (CD22 and CD19, respectively), followed by flow cytometric analysis. Use of a LAT-CAR construct that incorporated the endogenous LAT transmembrane domain into the LAT-containing CAR resulted in poor surface expression of the CD22-directed 2^{nd} generation CAR and no expression of the CD19-directed LAT-CAR (FIG. 6—top panels). By incorporating a transmembrane domain from the CD28 molecule into the CD19-directed LAT-CAR, transduced T cells were able to express high levels of both the CD22-directed 2^{nd} generation CAR and the CD19-directed LAT CAR (FIG. 6—bottom panels) demonstrating the important negative impact of the native transmembrane domain. LAT-CARs incorporating the CD28 transmembrane domain resulted in consistent surface expression of both CARs of the bicistronic CAR construct across T cells from multiple donors (FIG. 6). The data shown in FIG. 6 demonstrates superior surface expression of the CARs when the CD28 transmembrane is used. Importantly, the incorporation of a CD19-directed LAT-CAR in conjunction with the CD22-directed 2^{nd} generation CAR did not result in increased surface expression of the CD22-directed 2^{nd} generation CAR when compared to T cells transduced with a CD22-directed 200 generation CAR alone, establishing that changes in the function of ALA-CART-transduced T cells is due to the dual expression of the 200 generation-CAR and LAT-CAR and not simply a manipulation of expression of the previously described 2^{nd} generation CAR (FIG. 5).

### Example 4—T Cells Transduced with the ALA-CART Construct are Capable of Eliminating CD22-Low Leukemia which Cannot be Treated with Standard 2^{ND} Generation CAR T Cells

2^{nd} Gen CD22 CAR T cells and ALA-CART cells (expressing a CD22-directed 2^{nd} generation CAR and a CD19-directed LAT-CAR) were generated using T cells from healthy donors as described herein. NSG mice were engrafted with 1 million Luciferase-positive NALM6 leukemia cells expressing low-levels of CD22 antigen on Day-3. Leukemia progression was monitored by bioluminescent imaging (BLI) after intraperitoneal injection of firefly luciferin, using a Xenogen In Vitro Imaging System (IVIS). On Day 0, 2.5-3 million CAR+2^{nd} Gen CD22 CAR T cells (CD22-CART) or ALA-CART cells (“ALA-CART” or “22×19 ALA-CART”) were injected into the leukemia bearing mice. As a negative control, some mice received no T cells (No Tx). Leukemia progression/regression was monitored by BLI twice weekly (FIG. 2C) and graphed versus time (FIG. 2D). Mice were euthanized in accordance with IACUC policies when endpoint symptoms arose. Survival of cohorts is shown in FIG. 2E. Data collected in 2 independent experiments utilizing CAR T cells generated from two separate donors. These data demonstrate that ALA-CART cells are capable of completely eliminating CD22-low leukemia in vivo, whereas the standard 2^{nd} Gen CD22 CAR T cells are ineffective. The clearance of leukemia lead to significantly prolonged survival of all ALA-CART treated mice to the end of the study. These data demonstrate the ability of the ALA-CART construct to enhance the antigen sensitivity of CAR T cells in vivo.

Surviving ALA-CART-treated mice were euthanized on Days 49 and bone marrow was harvested. Bone marrow was stained with antibodies against human CD45, human CD3 and the fluorescently-tagged CD22-Fc, which binds to CAR molecules. The results of this analysis are shown in FIG. 2F. The data presented in FIG. 2F demonstrates that mice treated with ALA-CART cells had a definable population of persistent ALA-CART cells which was readily detected in all evaluated mice. This data suggests that the ALA-CART design results in CAR T cell persistence after clearance of antigen-low leukemia.

### Example 5—ALA-CART Construct Enhances CAR Signaling of a Standard 2^{ND} Generation Car in Response to Low-Levels of Antigen and can be Utilized to Enhance the Antigen Sensitivity of CAR T Cell Therapy Against CD22-Low Leukemia

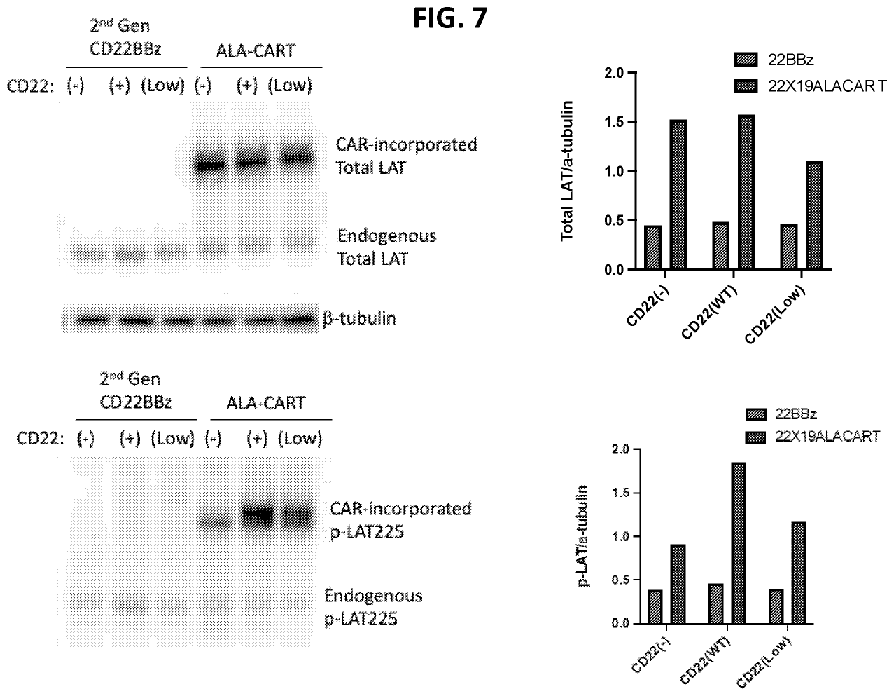

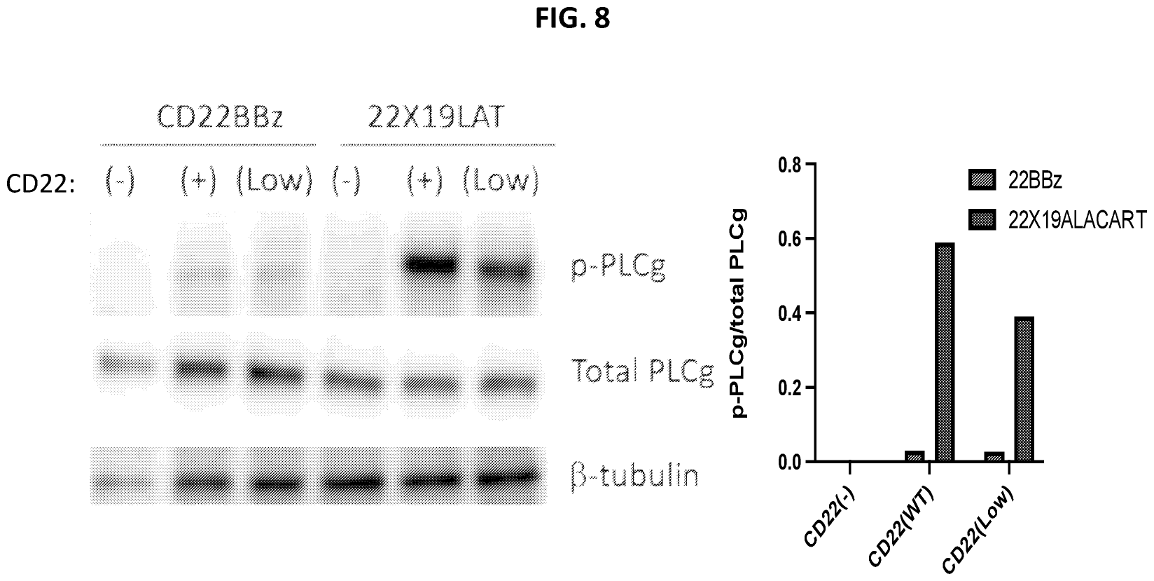

Quantitative and qualitative differences in signal transduction underlying the response of ALA-CART cells to antigen-low leukemia. ALA-CAR T cells have demonstrated superior in vivo clearance of antigen-low leukemia in a xenograft model relative to a standard 2^{nd} generation (2G) CD22 CAR with proven clinical efficacy (FIG. 2C). The design of the ALA-CART construct was hypothesized to drive antigen-directed co-localization of the intracellular LAT domain in close proximity with a 2G-CAR to amplify the signal generated downstream of the CAR in a way that leads to optimal T cell activation. As previously demonstrated, standard 2^{nd} generation CARs targeting CD22 have diminished phosphorylation of activating tyrosines when stimulated with low levels of antigen (FIG. 1C and FIG. 7), however ALA-CART cells demonstrate high amounts of activating LAT phosphorylation even in response to low levels of CD22 antigen (FIG. 7). The enhanced LAT activation seen in ALA-CART cells translated to increased downstream activation of Phospholipase C-gamma (PLCg) relative to standard 2^{nd} generation CAR T cells (FIG. 8). Given the central role PLCg plays in the subsequent activation of the NFAT and NF-kB pathways, the increased activation of this enzyme in ALA-CART cells would be expected to drive enhanced killing of antigen-low tumor cells.

2^{nd} Gen CD22 CAR T cells and ALA-CART cells were generated using T cells from healthy donors as described above. CAR+ T cells were enriched to greater than 90% purity using magnetic bead selection. 2^{nd} Gen CAR T cells and ALA-CART cells were co-incubated with NALM6 leukemia cells that expressed either: No CD22 antigen (−), wild type levels of CD22 antigen (+) or low levels of CD22 antigen (Low) for 10 minutes. Cells were lysed and lysate was separated by SDS-PAGE electrophoresis. Western blot analysis was performed after electrophoresis and the blots were probed with antibodies against: total LAT protein, LAT protein that has been phosphorylated at the activating residue, Tyrosine 225 (p-LAT225), or Beta-tubulin as a loading control. Blots were stained with an Alkaline Phosphatase-linked secondary antibody to allow visualization through luminescent chemical reaction. The results of this analysis are shown in FIG. 7 and demonstrate that ALA-CART cells express higher levels of the LAT-containing CAR than the endogenous levels of LAT. The data further demonstrates a greater amount of activated LAT (p-LAT225) in ALA-CART cells relative to 2^{nd} Gen CD22 CAR T cells. Importantly, ALA-CART cells demonstrate higher levels of phosphorylated LAT in response to CD22-low cells relative to standard 2^{nd} Gen CD22 CAR T cells in response to wild type levels of CD22.

2^{nd} Gen CD22 CAR T cells and ALA-CART cells were generated using T cells from healthy donors as described above. CAR+ T cells were enriched to greater than 90% purity using magnetic bead selection. 21d Gen CAR T cells and ALA-CART cells were co-incubated with NALM6 leukemia cells that expressed either: No CD22 antigen (−), wild type levels of CD22 antigen (+) or low levels of CD22 antigen (Low) for 10 minutes. Cells were lysed and lysate was separated by SDS-PAGE electrophoresis. Western blot analysis was performed after electrophoresis and the blots were probed with antibodies against: total phospholipase C (PLCg) protein, PLCg protein that has been phosphorylated at the activating residue, Tyrosine 738 (p-PLCg), or Beta-tubulin as a loading control. Blots were stained with an Alkaline Phosphatase-linked secondary antibody to allow visualization through luminescent chemical reaction. The results of this analysis are shown in FIG. 8. The data shown in FIG. 8 demonstrate a greater activation of PLCg (p-PLCg) in ALA-CART cells relative to 2^{nd} Gen CD22 CAR T cells. Moreover, ALA-CART cells demonstrate higher levels of phosphorylated PLCg in response to CD22-low cells than seen in standard 2^{nd} Gen CD22 CAR T cells in response to wild type levels of CD22.

### Defining Functional Responses of LAT-CAR T Versus 2^{nd} Generation CAR T Cells to CD22-Low Leukemia Cells

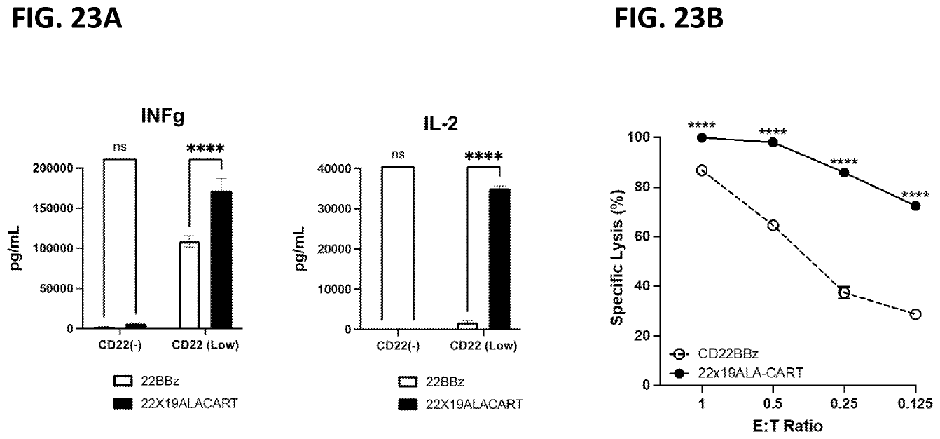

CAR T cell functional responses are negatively impacted by low antigen-density (FIG. 3), however as the signaling deficits produced in response to low levels of antigen are reversed in ALA-CART cells we expected to see enhanced function in ALA-CART cells relative to standard 2^{nd} generation CAR T cells. To test if ALA-CART induces increased cytokine production and cytotoxicity in response to low-levels of CD22 antigen relative to 2^{nd} Generation CAR alone, ALA-CAR T and CD22BBz 2^{nd} generation-CAR T cells were generated and co-cultured overnight with NALM6 cells expressing low-levels of CD22 antigen or NALM6 cells expressing no CD22 antigen (−) as a control for non-specific cytokine secretion. Supernatant was evaluated for Interferon-gamma (IFNg) and Interleukin-2 (IL-2) concentration by ELISA at the end of the co-incubation period, and ALA-CART cells were found to produce more IFNg and IL-2 in response to CD22-low leukemia than standard 200 generation CD22 CAR T cells (FIG. 23A). Importantly, the increased cytokine production of the ALA-CART cells was only seen in the presence of CD22 antigen, as there was no significant difference in background IFNg or IL-2 production between ALA-CART cells and standard 2^{nd} generation CAR T cells when co-incubated with CD22-negative cells (FIG. 23A).

To assess differences in the killing of antigen-low tumor cells by ALA-CART cells and standard 2^{nd} generation CAR T cells, a cytotoxicity assay was performed in which luciferase-positive NALM6 cells expressing low levels of CD22 were co-incubated with CAR T cells at decreasing effector-to-target (E:T) ratios overnight. As the enzymatic production of light by the luciferase enzyme is dependent upon ATP within a living cell, light production at the end of the co-incubation was used to measure the percentage of tumor cell death relative to NALM6 cells cultured in the absence of CAR T cells (Specific Lysis). ALA-CART cells demonstrated significantly higher specific lysis of CD22-low leukemia cells relative to standard 2^{nd} generation CD22 CAR T cells (CD22BBz) at every E:T ratio tested (FIG. 23B). These data demonstrate that the ALA-CART construct not only corrects the signaling deficits associated with antigen-low tumor cells, but through this correction allows the ALA-CART cells to respond to and kill antigen-low tumor cells better than current 2^{nd} generation CAR T cells.

### Example 6—ALA-CART Cells Demonstrate Enhanced In Vivo Persistance Relative to Standard 2Nd Generation CAR T Cells

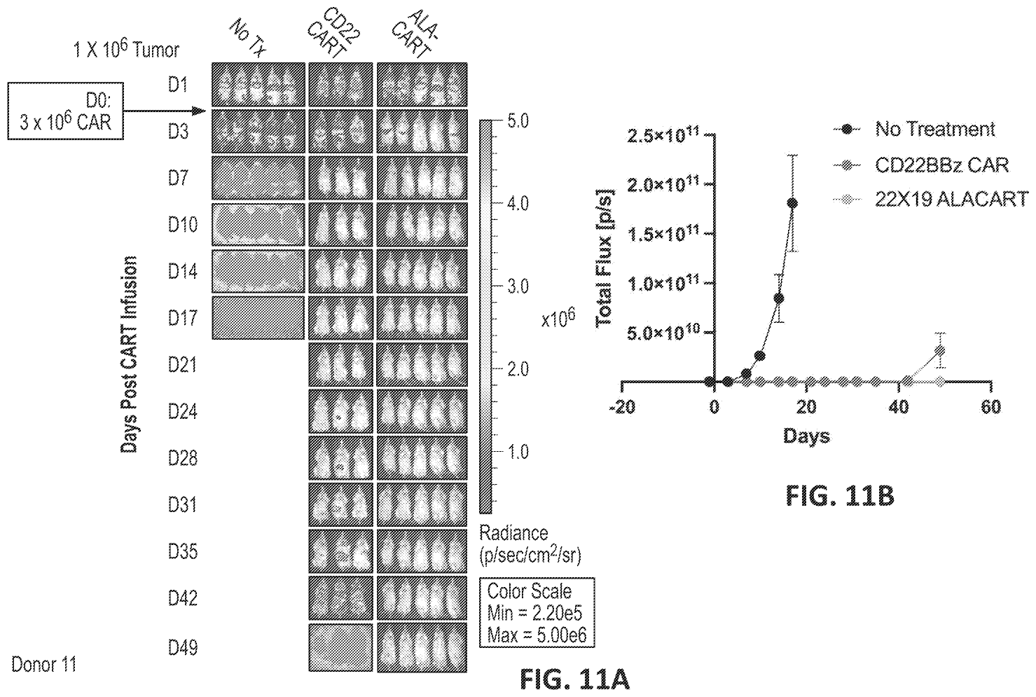

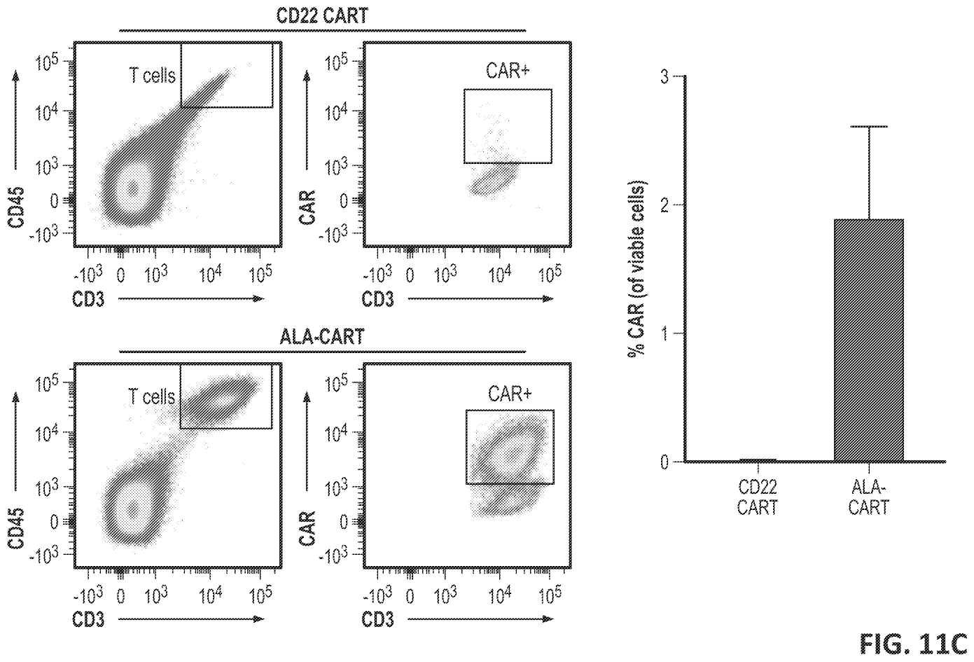

In vivo persistence of CAR T cells has been correlated with a decreased risk of relapse in patients. To determine the persistence of ALA-CART cells relative to standard 2^{nd} generation CAR T cells, NSG mice were engrafted with 1 million wild type (WT) NALM6 cells which can be cleared by standard 2^{nd} generation CD22-directed CAR T cells (FIG. 1A). NSG mice were engrafted with NALM6 leukemia cells on Day-3. On Day 0, 2.5-3 million CAR+2^{nd} Gen CD22 CAR T cells (CD22-CART) or ALA-CART cells (ALA-CART) were injected into the leukemia bearing mice. 3 days later, mice were treated with 3 million ALA-CART cells or 3 million standard 2^{nd} generation CD22 CAR T cells A group of mice received no T cell treatment (No Tx) as a negative control. 2^{nd} Gen CD22 CAR T cells and ALA-CART cells (targeting CD22 and CD19) were generated using T cells from healthy donors as described above. Leukemia progression was monitored by bioluminescent imaging (BLI) after intraperitoneal injection of firefly luciferin, using a Xenogen In Vitro Imaging System (IVIS). Leukemia progression/regression was monitored by BLI twice weekly (see FIG. 11A) and graphed versus time (see FIG. 11B). Data is representative of 2 independent experiments utilizing CAR T cells generated from two separate donors.

Mice treated with either ALA-CART or standard 2^{nd} generation CD22 CAR T cells showed upfront clearance of leukemia in vivo, relative to the progressive leukemia seen in untreated mice (FIGS. 11A-B). Recurrent leukemia was noted in mice receiving standard 2^{nd} generation CD22 CAR T cells at late time points, and this was correlated with minimal CAR T cell persistence at 49 days after CAR T cell infusion (FIGS. 11A-C). In contrast, mice treated with ALA-CART cells did not show any signs of leukemic relapse and persistent ALA-CART cells were readily identifiable in the bone marrow of these mice at Day 49 (FIGS. 11A-C). Mice were euthanized on Days 49-50 and bone marrow was harvested. Bone marrow was stained with antibodies against human CD45, human CD3 and the fluorescently-tagged CD22-Fc, which binds to CAR molecules. The results of this analysis are shown in FIG. 11C. The data demonstrates that the relapsed leukemia seen in mice treated with standard 2^{nd} Gen CD22 CAR T cells correlated with a complete loss of detectable CAR T cells in the mice, whereas mice treated with ALA-CART cells had no relapse and a definable population of persistent ALA-CART cells was readily detected in all mice treated with ALA-CART cells. This data suggests that the ALA-CART design leads to enhanced CAR T cell persistence which in turns protects against relapse.

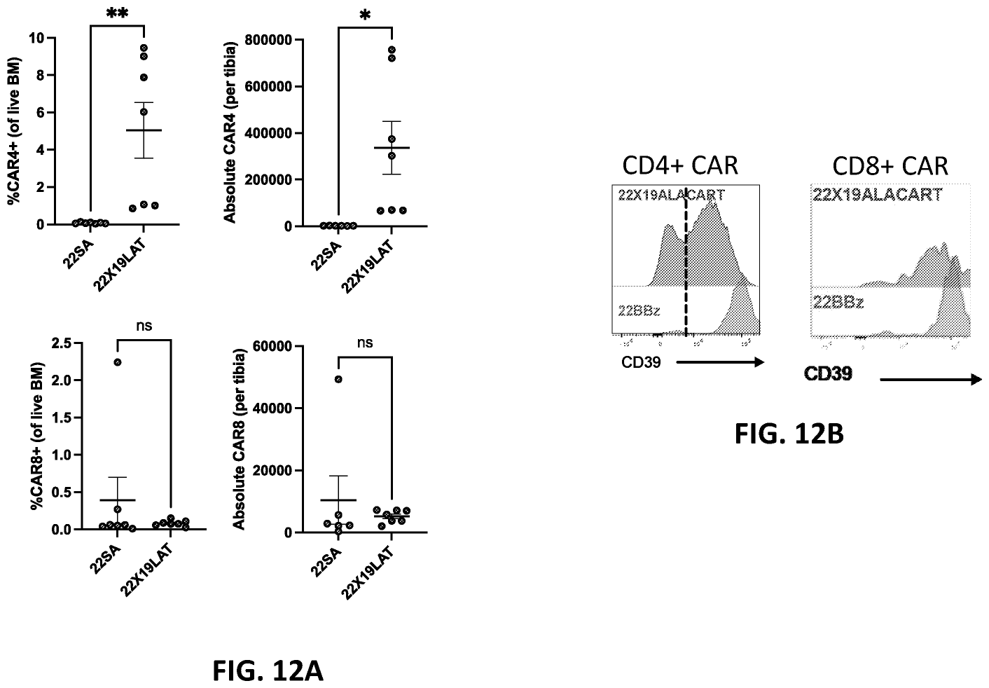

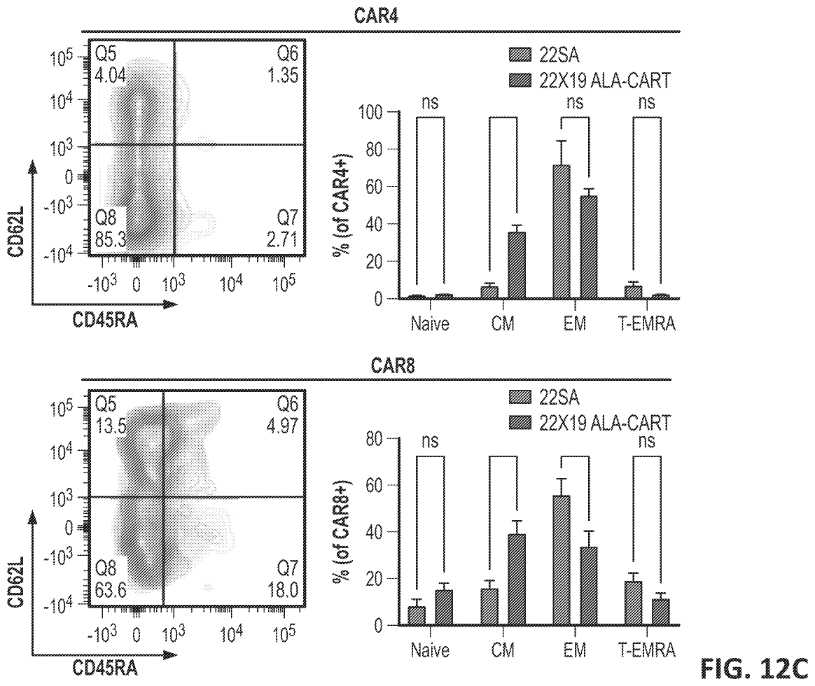

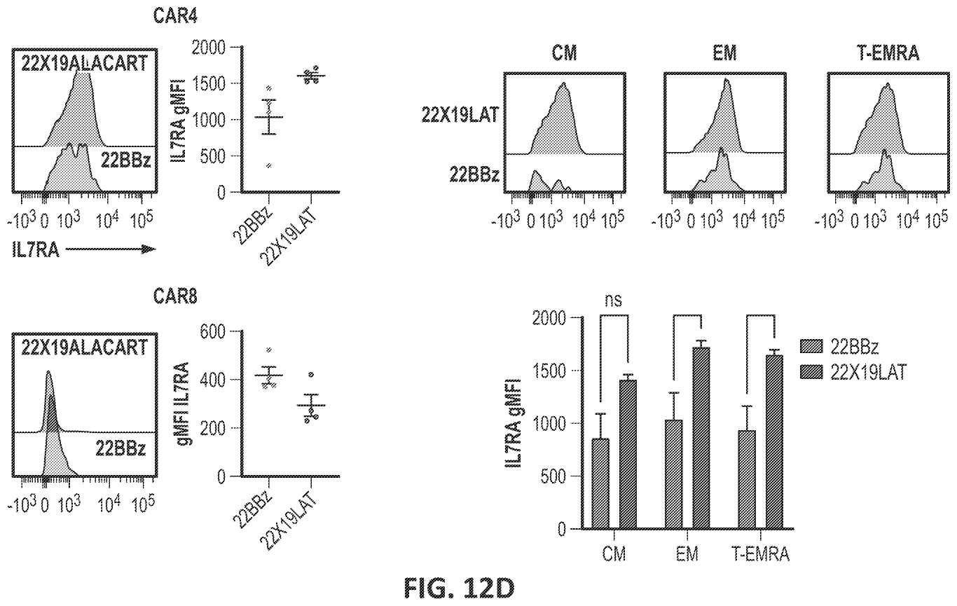

Further analysis of the persistent CAR T cell populations in mice revealed that the persistence of ALA-CART cells was primarily driven by persistence of CD4+ CAR T cells, which were virtually absent mice receiving standard 2^{nd} generation CAR T cells (FIG. 12A). Furthermore, the persistent CD4+ and CD8+ ALA-CART cells expressed lower levels of the exhaustion marker, CD39, than did standard 2^{nd} generation CAR T cells (FIG. 12B). This corresponded to significantly more persistent ALA-CART cells retaining a T-central memory (CM) phenotype, which has been associated with increased in vivo efficacy of adoptive T cell therapies (FIG. 12C). We also observed increased expression of the IL-7 receptor-alpha (IL-7RA) on the surface of persistent CD4+ ALA-CART cells regardless of their differentiation status, which corresponds with the observed increase in long-term survival of these cells relative to standard 2^{nd} generation CD22-CAR T cells (FIG. 12D). Collectively, these data demonstrate that ALA-CART cells have properties consistent with deceased T cell exhaustion and increased memory potential promoting enhanced persistence in vivo.

### Example 7—ALA-CART Cells Demonstrate Effective Responses Against Leukemia when Triggered Through Either CAR Alone

### ALA-CAR T Cells Targeting CD22 and CD19 Can Eliminate Leukemia Singularly Expressing Either CD19 or CD22 Antigen

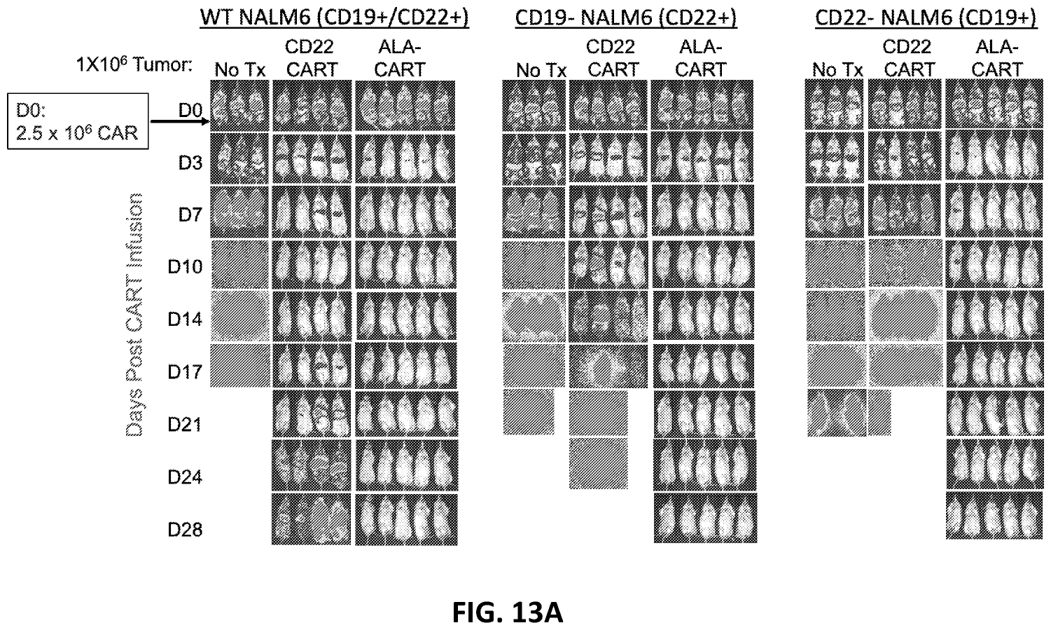

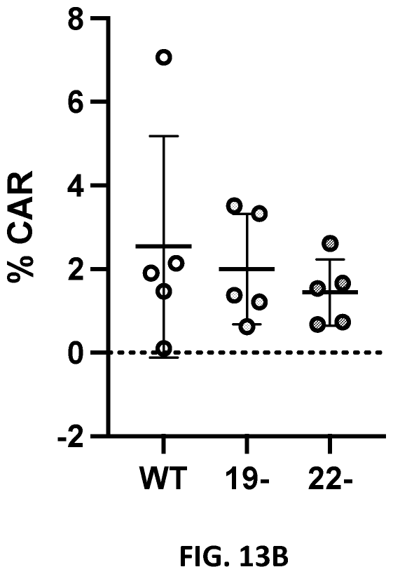

2^{nd} Gen CD22 CAR T cells and ALA-CART cells (targeting CD22 and CD19) were generated using T cells from healthy donors as described above. NSG mice were engrafted with 1 million Luciferase-positive NALM6 leukemia cells expressing either. wild type-levels of CD22 and CD19 antigen (WT NALM6), CD22 antigen only (CD19-NALM6) or CD19 antigen only (CD22-NALM6) on Day-3. Leukemia progression was monitored by bioluminescent imaging (BLI) after intraperitoneal injection of firefly luciferin, using a Xenogen In Vitro Imaging System (IVIS). On Day 0, 2.5 million CAR+2^{nd} Gen CD22 CAR T cells (CD22 CART) or ALA-CART cells (ALA-CART) were injected into the leukemia bearing mice. As a negative control, some mice received no T cells (No Tx). Leukemia progression/regression was monitored by BLI twice weekly (see FIG. 13A). These data demonstrate that both standard 2^{nd} Gen CD22 CAR T cells and ALA-CART cells initially eliminate leukemia expressing normal levels of CD22 and CD19 in vivo. 2^{nd} Gen CD22 CAR T cell treated mice relapsed with progressive leukemia, whereas ALA-CART treated mice controlled the leukemia throughout the experiment. Mice engrafted with leukemia expressing only CD22 antigen (CD19-NALM6) did not respond to treatment with 2^{nd} Gen CD22 CAR T cells, as the CD22 antigen expression is lower than in WT NALM6. This leukemia was completely eliminated by ALA-CART treatment. In mice engrafted with leukemia expressing only the CD19 antigen (CD22-NALM6), the leukemia was only eliminated by ALA-CART cells and not by standard 2^{nd} Gen CD22 CAR T cells. Bone marrow of mice treated with ALA-CART cells was analyzed by flow cytometry as described previously and there was no difference of ALA-CART cell persistence regardless of which antigen was targeted (see FIG. 13B). These data demonstrate that ALA-CART cells can effectively clear leukemia expressing only one of the two targeted antigens and can be effective in vivo regardless of which of the CARs in the ALA-CART construct are activated.

As all previous data presented was based on an ALA-CART construct targeting the CD22 antigen with a 2^{nd} generation CAR in conjunction with a LAT-CAR targeting the CD19 antigen, we tested the ability of our 22×19 ALA-CART cells to respond to stimulation of each individual CAR. NSG mice were engrafted with 1 million cells of wild-type (WT) NALM6, which expresses both CD19 and CD22 or CD19-NALM6, which only expresses CD22 or CD22-NALM6, which only expresses CD19. Mice engrafted with each variant of NALM6 cells were treated 3 days later with 2.5 million ALA-CART cells or 2.5 million standard 2^{nd} generation CD22-CAR T cells or received no treatment (No Tx) as a negative control. Leukemia burden was followed by bioluminescent imaging (BLI) twice a week, and mice were euthanized when developing symptomatic leukemia in accordance with our established animal protocols. Mice engrafted with WT NALM6 (which stimulates both individual CARs of the ALA-CART cells) demonstrated complete clearance with ALA-CART treatment (FIG. 13A). Similar to prior experiments, mice with WT NALM6 treated with standard 2^{nd} generation CAR T cells showed initial clearance of the leukemia, followed by relapse (FIG. 13A). Mice engrafted with CD19-NALM6 were also completely cleared of leukemia by ALA-CART cells, despite only triggering the 2^{nd} generation CAR of the ALA-CART construct (FIG. 13A). Mice engrafted with CD19-NALM6 and treated with the standard 2^{nd} generation CD22 CAR T cells only had a modest slowing of leukemia progression, likely attributed to the lower expression of the CD22 molecule on this cell line (FIG. 13A). Mice engrafted with CD22-NALM6 had no benefit from treatment with the standard 2^{nd} generation CD22 CAR T cells, as expected due to the lack of a cognate antigen for these CAR T cells (FIG. 13A). Surprisingly, ALA-CART cells, which could only recognize CD22-NALM6 with the CD19-directed LAT-CAR, were capable of completely eradicating the CD22-negative leukemia demonstrating that the LAT-CAR by itself is capable of driving a potent response against tumor cells (FIG. 13A). These data demonstrate that the ALA-CART format can work as an “OR” CAR, successfully targeting tumor cells through one antigen “or” a second antigen. Mice treated with ALA-CART cells were euthanized at 30 days after CAR T cell infusion and the bone marrow was evaluated for persistent ALA-CART cells. There was no significant difference in the persistence of ALA-CART cells based on which antigens were present on the leukemia, indicating that stimulation through either the 2^{nd} generation CAR or the LAT-CAR of ALA-CART cells was sufficient to drive in vivo persistence (FIG. 13B).

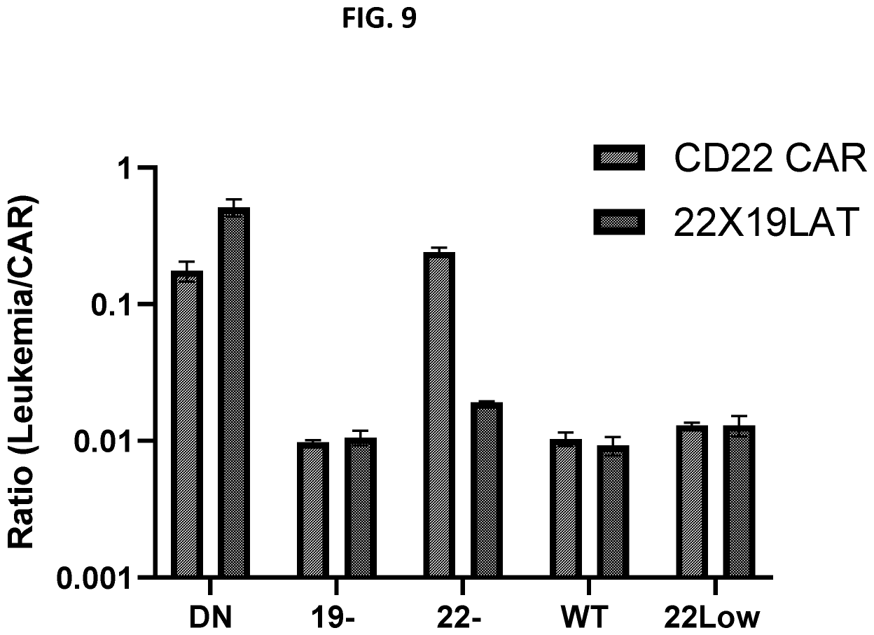

The “OR” function of the ALA-CART cells was further evaluated through in vitro testing. ALA-CART cells and standard 2^{nd} generation CD22 CAR T cells were co-cultured with NALM6 leukemia cells expressing various combinations of CD19 and CD22 antigens for 24 hours. At the end of this co-culture, NALM6 survival was assessed by flow cytometry. ALA-CART cells demonstrated the ability to kill NALM6 cells through both individual CARs, as they killed NALM6 cells expressing either CD19 or CD22, but not double negative (DN) NALM6 cells which did not express either antigen (FIG. 9).

ALA-CAR T cells Kill Target Cells Through Each Independent CAR Molecule Equivalently to 2^{nd}-Generation CD22 CAR T Cells In Vitro

2^{nd} Gen CD22 CAR T cells and ALA-CART cells (targeting CD22 and CD19) were generated using T cells from healthy donors as described above. 2^{nd} Gen CAR T cells and ALA-CART cells were co-incubated with NALM6 leukemia cells that expressed either: No CD22 or CD19 antigen (DN), No CD19 antigen (19-), No CD22 (22-), Wild type levels of CD22 and CD19 antigen (WT) or low levels of CD22 antigen with WT levels of CD19 (CD22Low) for 24 hours. Surviving cells were quantified by flow cytometry. The results of this analysis are shown in FIG. 9. The data shown in FIG. 9 demonstrates that ALA-CART eliminates leukemia cells in vitro in an antigen-dependent manner. ALA-CART demonstrates equivalent killing of CD22-expressing leukemia cells (19-, WT, 22Low) as dose standard 2^{nd} Gen CD22 CAR T cells. ALA-CART kill leukemia cells that lack CD22 and only express CD19 (22-) as well as leukemia cells that only express CD22 and lack CD19 (19-), thus demonstrating that stimulation of either CAR making up the ALA-CART construct is sufficient for cytotoxicity against target cells.

As expected, standard 2^{nd} generation CD22 CAR T cells effectively killed NALM6 cells only if the CD22 antigen was expressed (FIG. 9).

### ALA-CAR T Cells Secrete Cytokine in Response to Stimulation Through Either of the CAR Molecules In Vitro

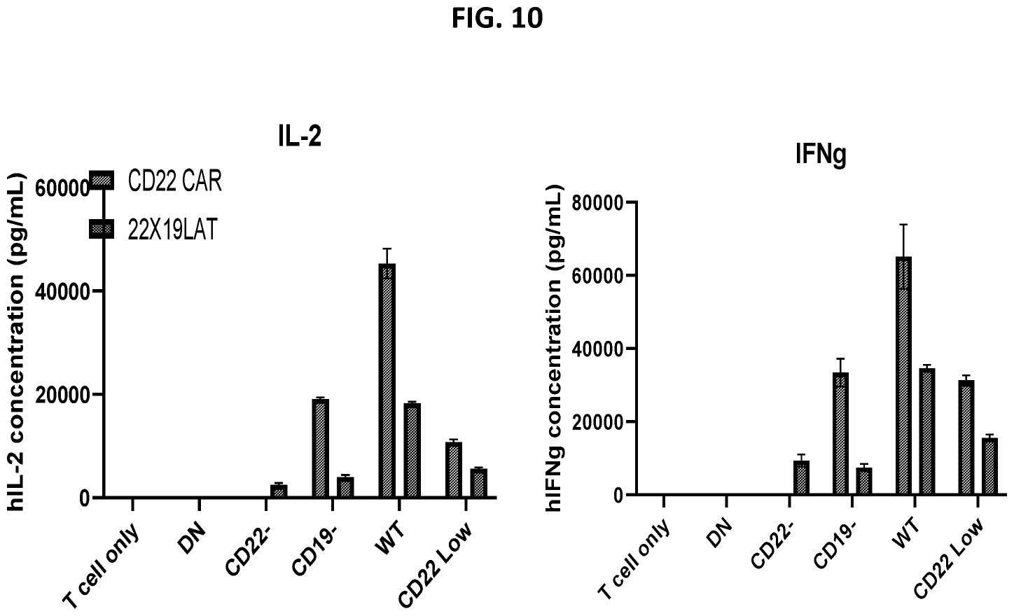

The supernatant of these co-cultures was evaluated for IL-2 and IFNg by ELISA to evaluate cytokine production in response to stimulation through each receptor. Similar to the cytotoxicity, ALA-CART cells demonstrated the ability to secrete cytokines in response to either CD19 or CD22 stimulation (FIG. 10). 2^{nd} Gen CD22 CAR T cells and ALA-CART cells (targeting CD22 and CD19) were generated using T cells from healthy donors as described above. 2^{nd} Gen CAR T cells and ALA-CART cells were co-incubated with NALM6 leukemia cells that expressed either: No CD22 or CD19 antigen (DN), No CD19 antigen (19-), No CD22 (22-), Wild type levels of CD22 and CD19 antigen (WT) or low levels of CD22 antigen with WT levels of CD19 (CD22Low) for 24 hours. IL-2 and interferon-gamma (IFNg) in the supernatant was quantified by ELISA. The results of this analysis are shown in FIG. 10. The data shown in FIG. 10 demonstrates that ALA-CART cells secrete cytokines in an antigen-dependent manner in response to stimulation of either CAR molecule independently with a synergistic increase in cytokine production when both CARs of the ALA-CART construct are simultaneously activated.

ALA-CAR T Cells Targeting CD22 and CD19 Activate PLCg, ERK, and P38 Pathways in Response to Stimulation with Either Antigen

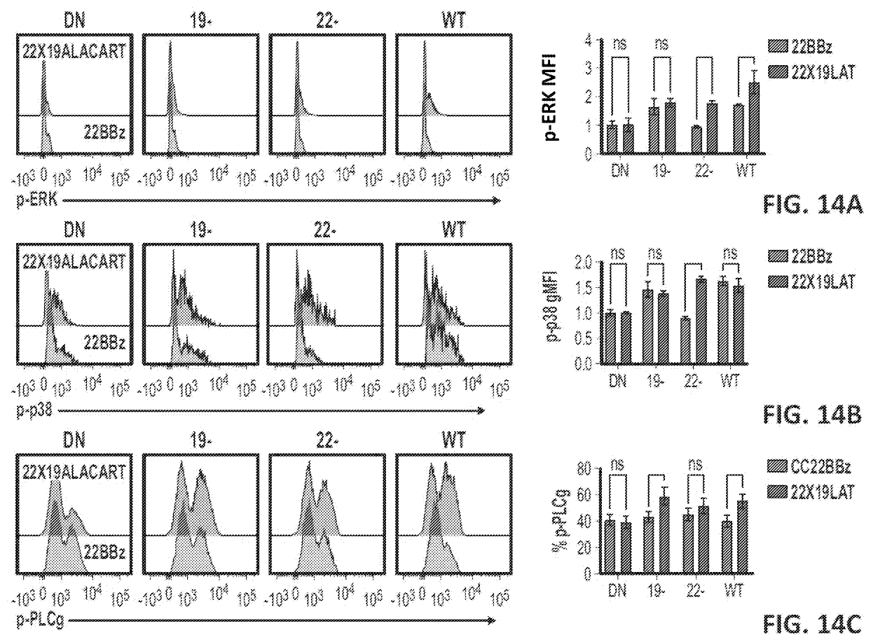

We further investigated the impact of stimulating each of the CARs expressed by ALA-CART cells by evaluating signal transduction. ALA-CART and standard 2^{nd} generation CD22 CAR T cells were co-incubated with NALM6 cells expressing either wild type-levels of CD22 and CD19 antigen (WT NALM6), CD22 antigen only (CD19-NALM6), CD19 antigen only (CD22-NALM6) or no CD22 or CD19 (double negative, DN) for 15 minutes. Cells were fixed with paraformaldehyde and permeabilized with methanol and then stained for CD3, CAR expression and either phosphorylated ERK (p-ERK, see FIG. 14A), phosphorylated p38 (p-p38, see FIG. 14B) or phosphorylated PLCg (p-PLCg, see FIG. 14C) and then analyzed by flow cytometry. As shown in FIGS. 14A-C, increased phosphorylation was observed of each signaling molecule in response to stimulation with either CD19 or CD22 or with both antigens together. Activation of the ERK and p38 pathways were seen in ALA-CART cells upon stimulation of either CAR or both CARs simultaneously, whereas standard 2^{nd} generation CD22 CARs only responded to leukemia cells expressing the CD22 antigen (FIGS. 14A-B). PLCg, however was only significantly phosphorylated in ALA-CART cells when the CD22 antigen was expressed, suggesting that stimulation through the CD19-directed LAT-CAR results in anti-leukemic activity outside of this signaling pathway (FIG. 14C). These data demonstrate that ALA-CART cells can effectively trigger signal transduction in response to leukemia expressing only one of the two targeted antigens regardless of which of the CARs in the ALA-CART construct are activated. Taken together, these data demonstrate that the ALA-CART format is effective at independently targeting multiple antigens in an “OR” manner

### Example 8—Defining Early Transcriptional Changes in Response to LAT-CAR in Comparison to 2G CAR Signaling

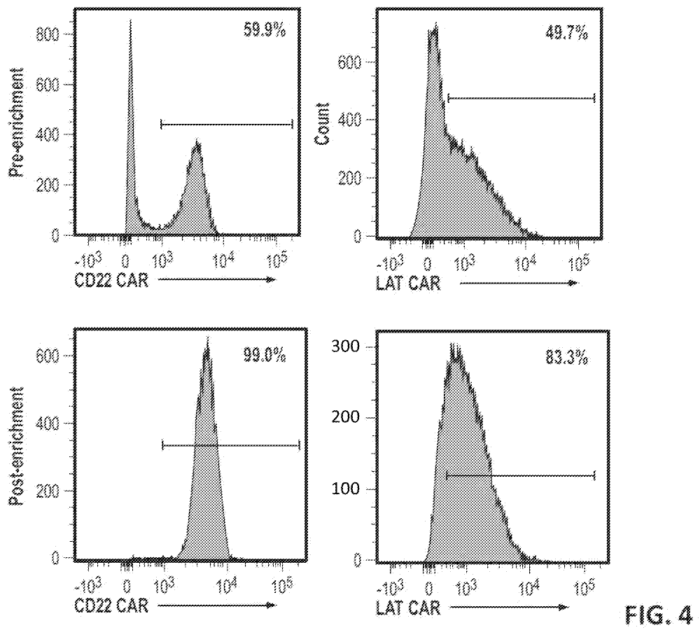

TCR stimulation leads to signal transduction events which culminate in the translocation of multiple transcription factors into the nucleus resulting in global transcriptome alterations. Differences in transcriptomic changes downstream of CAR activation were shown between CAR- and TCR-driven T cell responses. Global transcriptional changes in CAR T cells are evaluated to elucidate differences in the downstream effects of LAT-CAR and 2G-CAR activation. CAR T cells from healthy donors are obtained using methods described in examples above. To minimize the impact of non-transduced “passenger” T cells, CAR-positive cells are enriched 2 days after transduction through magnetic selection using CD22-Fc proteins which are selectively bound by the CAR. CAR T cells are expanded for another 4 days and frozen prior to use in experiments. This results in >90% CAR-positive cells in the end product with no signs of activation in the absence of stimulation with NALM6 cells (FIG. 4). LAT-CAR and 2G-CARs are stimulated by plate-bound human CD22-Fc and CD19-Fc for 30 minutes. Preliminary studies are run to optimize the concentration of antigen used for plate-coating for CAR activation utilizing CD19- and CD22-directed 2G-CAR T cells. LAT- and 2G-CAR T cells are plated in uncoated wells in parallel as unstimulated controls. Each condition are run in quadruplicate. Cells are lysed with Trizol and RNA is extracted per manufacturer's protocols and purified using RNA-Easy kit. RAN quality is assessed and libraries are prepared using Illumina TrueSeq Stranded mRNA sample prep kits. Libraries are sequenced using theNovaSeq 6000 platform with paired-end reads and approximately 40-50 million reads/sample are collected. The resulting data are analyzed using a custom computational pipeline consisting of the open-source gSNAP, Cufflinks, and R for alignment and discovery of differential gene expression. Briefly, each high-resolution sequencing read generated by each sample is mapped to the human genome (hg38) using gSNAP, Cufflinks to calculate the prevalence of transcripts from each known gene, and each gene is expressed as a transcript in fragments perkilobase of exon per million mapped reads (FPKM). From this, significant differentially expressed mRNA profiles are identified using ANOVA in R with an FDR of P<0.05. Significant differentially expressed gene lists areanalyzed using GSEA and Ingenuity Pathway analysis. Validation of identified pathways are carried out by quantitative RT-PCR and/or protein quantification by Western blot or flow cytometry studies. Transcriptional changes are analyzed to identify transcription factor activity downstream of each CAR. Identified differences in transcription factor activity are further investigated by reporter assays or by flow cytometry of isolated nuclei. Validation experiments are performed in CART cells generated from at least 3 different donors.

### Example 9—Signal Transduction of LAT-CAR T Versus 2G-CAR T Cells in Response to Low-Levels of Antigen

Antigen-density alters the efficiency of LAT-phosphorylation and ERK signaling without impacting ZAP70-phosphorylation in 2G-CD22 CAR T cells (FIGS. 1B-1D) demonstrating that antigen-density impacts selective areas of CAR T cell signaling without leading to a global decrease in all aspects of signal transduction. Accordingly, LAT-CAR T cells remain effective against CD22-low NALM6 in vivo. To determine the differences in the signal transduction of LAT-CAR and 2G-CAR T cells, LAT-CAR T cells and 2G-CAR T cells are generated as described in examples above. LAT-CAR T cells and 2G-CAR T cells are co-incubated with DN-NALM6, CD22-Low NALM6 and WT-NALM6. Co-incubation timing vary based on the signal transduction pathway to be evaluated and include a range of times to evaluate for differences in signaling kinetics as well as magnitude. Selected signaling proteins known to be involved in canonical TCR signaling are evaluated for phosphorylation of activating residues by intracellular flow cytometry or Western blot analysis. Flow cytometry (FC) approaches utilize GFP+ NALM6 cells in conjunction with staining for CD3 and surface CAR expression to determine signaling events in CAR T cells. For Western blot (WB) analysis, Jurkat cells stably expressing the CAR are used. Primary T cells are used for follow up analysis. CAR T cells from healthy donors are enriched for CAR+ cells as described in examples above (FIG. 4). NALM6 cells lines are screened by WB to verify lack of expression of protein of interest prior to co-incubations. Signaling pathways for analysis include: proximal TCR signaling (CD3z, LCK, FYN, ZAP70, SLP76, LAT, PLC1), MAPK pathways (ERK, JNK, p38, b-RAF, and RAF-c), NFkB, and the mTOR pathway (AKT, mTOR). Calcium flux will also be measured by flow cytometry to evaluate the NFAT signaling pathway. Signal transduction pathways shown to be preferentially utilized by the LAT-CAR relative to the standard 2G-CAR are further evaluated using CD19-negative and CD22-negative NALM6 cells to better define the role of each component of the bicistronic LAT-CAR on that signaling pathway. All signaling assays are repeated at least 3 times across different donors. As described throughout, negative controls including antigen-negative NALM6 cells and mock T cells are used to control for non-specific and/or allo-reactivity and multiple donors are used to verify reproducibility.

### Example 10 In Vivo Antigen-Sensitivity of the LAT-CAR Construct Against a Single Target Antigen

LAT-CAR has demonstrated superior in vivo activity against CD22-low NALM6 in preliminary experiments, overcoming one of the main limitations of CD22-directed CAR therapy observed in clinical trials. These experiments were performed in the context of simultaneously targeting CD22 and CD19. Therefore, to directly address the problem of CD22 antigen-sensitivity in a CAR that would primarily be used in settings of CD19-negative relapse, bicistronic LAT-CARs targeting CD22 at both positions are generated.

### In Vitro Function of LAT-CAR Solely Targeting the CD22 Antigen.

The 2^{nd} Generation CD22 CAR used in our experiments has demonstrated the ability to induce remission in 70-80% of patients and has demonstrated clinical utility in treating CD19-negative relapses. However, due to its inability to target CD22-low leukemia cells, responses are short-lived and consolidative hematopoietic stem cell transplantation (HSCT) is recommended. Therefore, increasing the sensitivity of CD22-directed CAR T cells could increase the efficacy of this therapy as a stand-alone treatment and provide options for patients who are not candidates for a subsequent HSCT. To address this clinical need, a series of bicistronic LAT-CARs which singularly target the CD22 antigen are generated. Using the bicistronic LAT-CAR construct, a CD22-reactive scFv is cloned into each of the individual CARs. The scFvs to be tested will include m971 and an affinity-enhanced version of m971 (HA-m971) which has 1000-fold higher affinity for CD22 and contains a (G4S)×3 linker which does not favor self-dimerization14. All four combinations of m971 and HA-m971 on either/both the CARs of the bicistronic construct are cloned, utilizing codon-wobbled sequences to prevent internal recombination. 6-His, FLAG and/or MYC tags are attached to the scFvs to differentiate and verify expression of each individual CAR. Surface expression is confirmed by flow cytometry and CAR T cells are tested for function in vitro against NALM6 expressing low- or WT-levels of CD22 by measuring cytokine production by ELISA, cytotoxicity by flow cytometry, proliferation by membrane-dye dilution as described in above examples. In vitro testing is carried out in comparison to standard 2G-CAR and CD22-negative NALM6 cells and mock-transduced T cells are used as negative controls. Experiments are carried out in triplicate and repeated with T cells from at least three different donors.

In vivo efficacy of a LAT-CAR singularly targeting CD22 against antigen-low leukemia. LAT-CAR T cells solely targeting the CD22 antigen that show in vitro are tested in xenograft models. Male and female NSG mice are inoculated with either CD22-Low or WT-NALM6 cells expressing GFP/Luciferase (10⁶ cells) by tail vein injection. Mice are randomized and engraftment will be verified by intraperitoneal injection of luciferin followed by bioluminescent imaging (BLI) on a Xenogen In Vivo Imaging System (IVIS) 3-4 days later. The following day mice are inoculated with CD22-directed 2G- or bicistronic LAT-CAR T cells at 3×10⁶ CAR+ cells per mouse (n=5/group). Mice are followed by serial BLI twice weekly until the end of experiment. Survival are monitored and mice are euthanized when they reach pre-defined humane endpoints for weight loss, decreased activity, paralysis, impaired ambulation or manifestations of progressive xGVHD. The bone marrow of mice which are euthanized for progressive leukemia or xGVHD are analyzed by flow cytometry for presence of NALM6 cells (GFP+/CD19+/CD22+) and their level of CD22 expression. Bone marrow are evaluated for the presence of CAR T cells (CD45+/CD3+/CD4+ or CD8+/CAR+) with surface CAR expression detected with fluorescently-conjugated CD22-Fc protein. This experiment is independently repeated three times using different T cell donors each time.

LAT-CAR construct can be effective against leukemia expressing low levels of CD19. To investigate whether the findings above are specific to targeting CD22, a bicistronic LAT-CAR is constructed utilizing the CD19-specific scFv, FMC63, on both individual CARs. The same cloning strategies as described in the section above will be used, including codon-wobbling and different tags for the scFv at each position. CD19-targeting LAT-CAR T cells are tested in vitro for activity against three separate NALM6 clones expressing low levels of CD19 in comparison to standard 2G CAR T cells. In vivo xenograft experiments are carried out as described above, including repeat experiments with different T cell donors.

### Example 11—a Combination of Two High-Affinity CD22-Directed SCFV's Gives the Optimal In Vivo Efficacy of ALA-CART Cells

ALA-CART cells have demonstrated superior in vivo activity against CD22-low NALM6 in preclinical experiments, overcoming the main limitation of CD22-directed CAR therapy observed in clinical trials. These experiments were performed in the context of simultaneously targeting CD22 and CD19. Therefore, to directly address the problem of CD22 antigen-sensitivity in a CAR that would primarily be used in settings of CD19-negative relapse, bicistronic LAT-CARs targeting CD22 at both positions are generated.

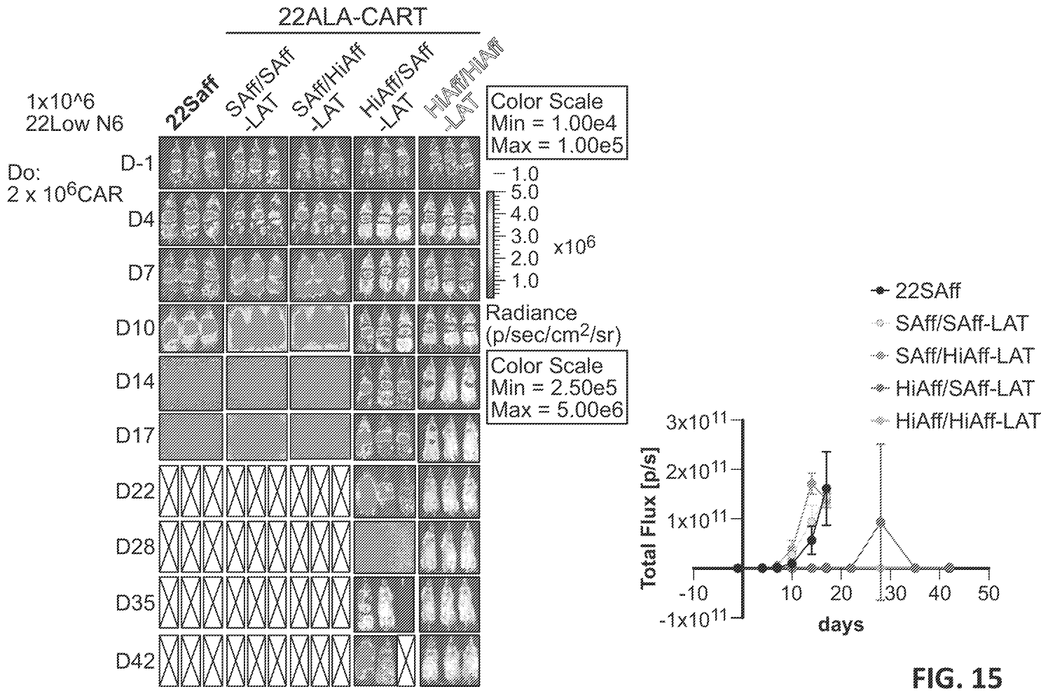

2^{nd} Gen CD22 CAR T cells and ALA-CART cells (targeting CD22 only) were generated using T cells from healthy donors as in previous experiments. Four variants of ALA-CART cells were made using combinations of the standard affinity m971 scFv (Saff) and an affinity enhanced version of m971 (HiAff) (combinations of scFv's depicted in Table A). NSG mice were engrafted with 1 million Luciferase-positive NALM6 leukemia cells expressing low levels of CD22 on Day −3. Leukemia progression was monitored by bioluminescent imaging (BLI) after intraperitoneal injection of firefly luciferin, using a Xenogen In Vitro Imaging System (IVIS). On Day 0, 2 million CAR+2^{nd} Gen CD22 CAR T cells (CD22 CART) or ALA-CART cells (ALA-CART) were injected into the leukemia bearing mice. Leukemia progression/regression was monitored by BLI twice weekly (see FIG. 15). These data demonstrate that having the SAff m971 scFv in the first CAR of the ALA-CART construct (scFv #1, FIG. 1) does not allow for effective elimination of CD22-low leukemia. Placement of the HiAff scFv in position scFv #1 improves in vivo efficacy of the CD22-directed ALA-CART cells, but optimal in vivo efficacy and clearance of CD22-low leukemia only occurred when the HiAff scFv was utilized in both scFv #1 and scFv #2 positions. These data demonstrate that the ALA-CART construct is capable of targeting low levels of a single antigen.

TABLE A

| | scFv #1 | scFv #2 |
|---|---|---|
| CAR | (2^{nd} Gen) | (LAT CAR) |
| 22SAff | m971 | n/a |
| (control) | | |
| SAff/SAff | m971 | m971 |
| Saff/HiAff | m971 | High-Aff m971 |
| HiAff/SAff | High-Aff | m971 |
| | m971 | |
| HiAff/HiAff | High-Aff | High-Aff |
| | m971 | m971 |

### Example 12—a Combination of Two High-Affinity CD22-Directed SCFV's Gives the Optimal In Vivo Efficacy of Ala-Cart Cells

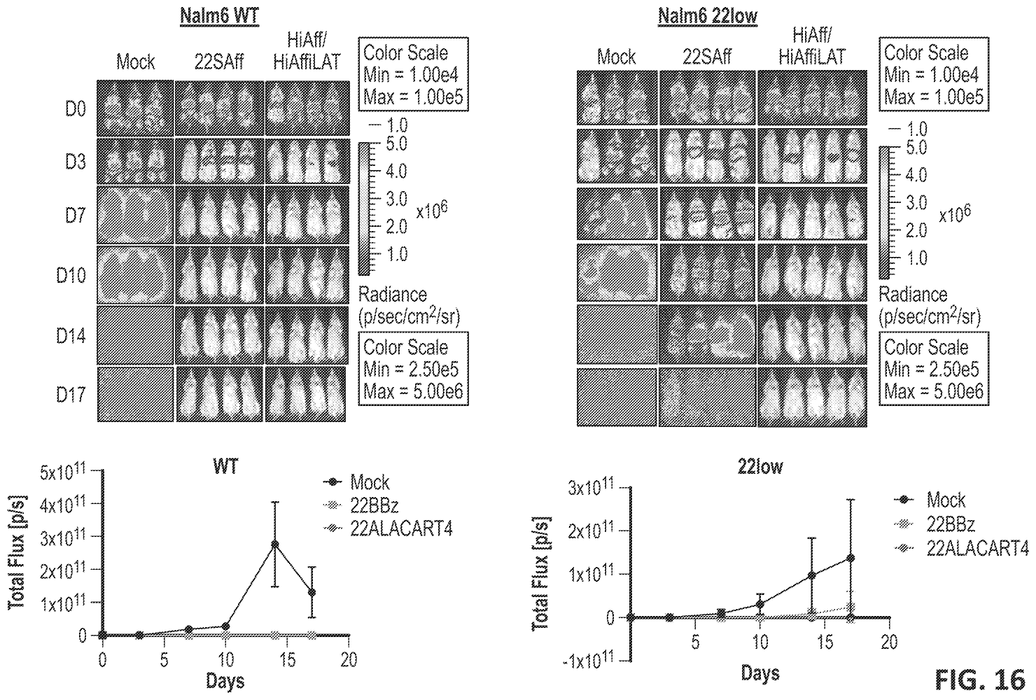

2^{nd} Gen CD22 CAR T cells and ALA-CART cells (targeting CD22 only using HiAff scFv's at both positions) were generated using T cells from healthy donors as in previous experiments. NSG mice were engrafted with 1 million Luciferase-positive NALM6 leukemia cells expressing either: WT-levels of CD22 (panel A) or low levels of CD22 (panel B) on Day-3. Leukemia progression was monitored by bioluminescent imaging (BLI) after intraperitoneal injection of firefly luciferin, using a Xenogen In Vitro Imaging System (IVIS). On Day 0, 2.5 million CAR+2^{nd} Gen CD22 CAR T cells (CD22 CART) or ALA-CART cells (ALA-CART) were injected into the leukemia bearing mice. Leukemia progression/regression was monitored by BLI twice weekly (see FIG. 16). These results were consistent across three independent experiments using three different T cell donors. These data demonstrate that the ALA-CART construct containing 2 HiAff scFv's is effective at eliminating both WT and CD22-low leukemia in vivo.

### Example 13—ALA-CART Cells Solely Targeting CD22 have Enhanced In Vivo Persistence Relative to Standard 2^{ND} Gen CD22 CAR T Cells

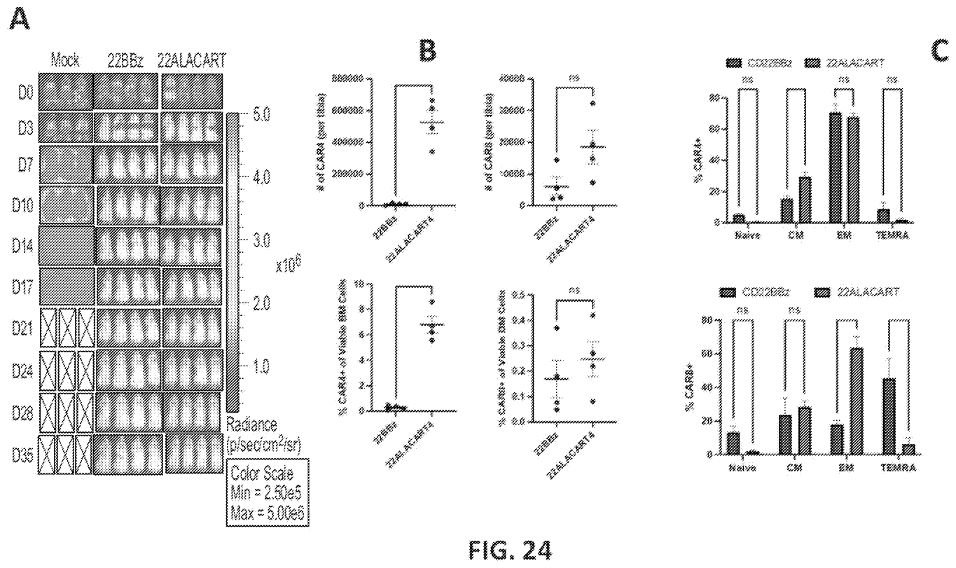

ALA-CART cells solely targeting the CD22 antigen (22ALA-CART) were tested for their in vivo persistence. NSG mice were engrafted with 1 million wild type (WT) NALM6 cells and 3 days later were treated with 3 million non-transduced T cells (Mock), 3 million standard 2^{nd} generation CD22 CAR T cells (22BBz) or 3 million 22ALA-CART cells and leukemia burden was monitored by BLI twice a week for 5 weeks. Mice treated with either standard 21d generation CAR T cells or 22ALA-CART cells cleared their leukemia, whereas Mock treated mice succumbed to progressive leukemia (FIG. 24A). Mice were euthanized on Day 40 and bone marrow was evaluated for persistent CAR T cells by flow cytometry. Mice treated with 22ALA-CART had significantly higher persistent CD4+/CAR+ T cells compared to mice treated with standard 2^{nd} generation CD22 CAR T cells (FIG. 24B) There was a similar trend for more CD8+/CAR+ T cells in the bone marrow of mice treated with 22ALA-CART cells, but the trend did not reach statistical significance (FIG. 24B). The increased persistence of CD4+ ALA-CART cells was correlated with a significantly higher percentage of cells with a central memory (CM) phenotype, whereas the persisting CD8+ ALA-CART cells predominantly expressed an effector memory (EM) phenotype at this time point (FIG. 24C). These data demonstrate that similar to the multi-antigen targeting ALA-CART, the 22ALA-CART construct drives a more persistent CAR T cell product than the standard 2^{nd} generation CAR construct.

### Example 14—22ALA-CART Cells are Enriched for T Cells with a Stem Cell Memory Phenotype

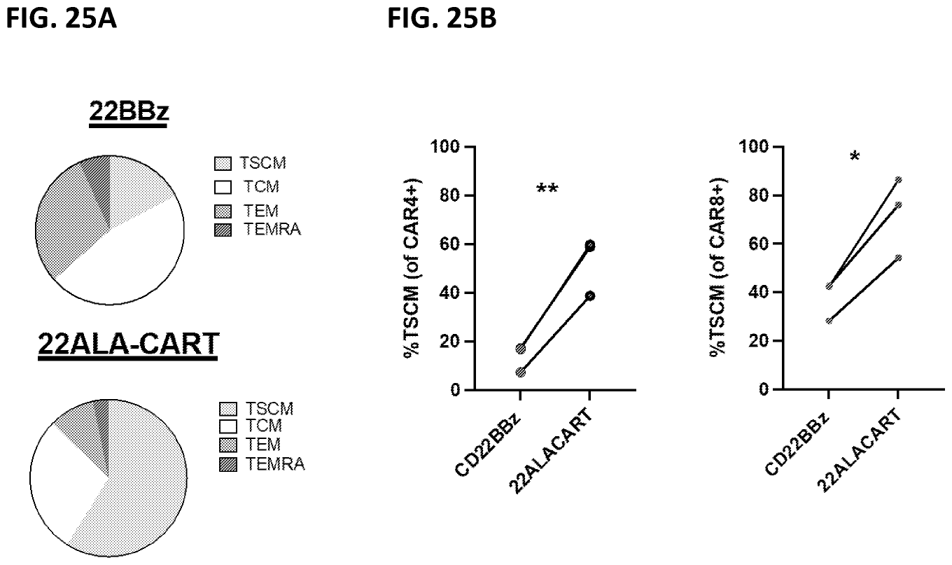

2^{nd} Generation CD22 CAR T cells and 22ALA-CART cells were generated using T cells from 3 independent, healthy donors. CAR T cells were analyzed by flow cytometry the 2 days after transduction to evaluate the expression of markers associated with effectory/memory differentiation and persistence. T cells were stained for the expression of CD45RA, CD62L, CCR7, CD95 and CD22-Fc for detection of CAR. ALA-CART cells were found to have a statistically significantly higher percentage of cells with a T Stem Cell Memory (TSCM) phenotype relative to standard 2^{nd} generation CD22 CAR T cells (FIG. 25A). Further analysis revealed that CD4+ and CD8+ ALA-CART cells consistently had increased proportions of TSCM cells across multiple donors (FIG. 25B). Given the association of the TSCM phenotype with in vivo memory formation and efficacy of adoptive T cell therapies, our data suggest that the ALA-CART cell product intrinsically has higher potential for persistence and in vivo effectiveness than standard 2^{nd} generation CAR T cells.

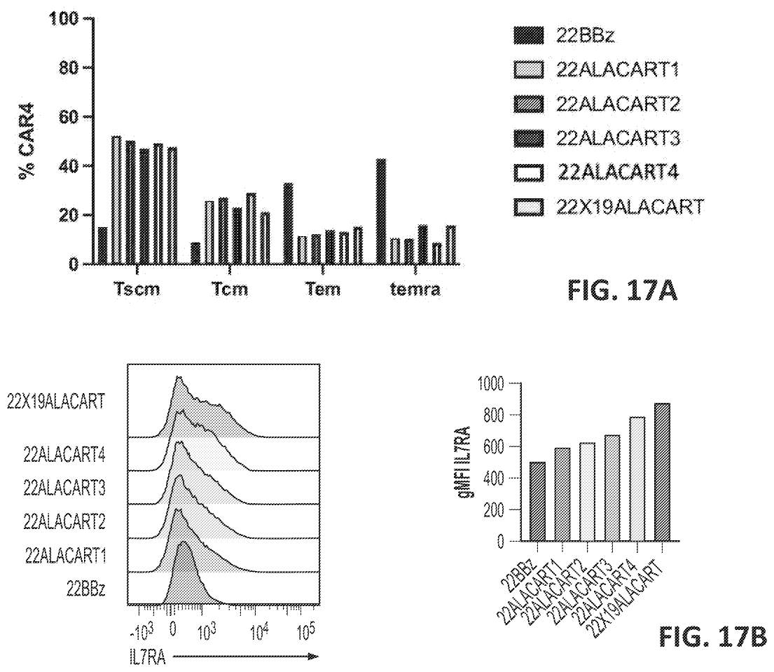

2^{nd} Gen CD22 CAR T cells and ALA-CART cells (targeting CD22 only using all 4 combinations of SAff and HiAff scFv's at both positions) were generated using T cells from healthy donors as in previous experiments. CAR T cells were analyzed by flow cytometry the 2 days after transduction to evaluate the expression of markers associated with memory formation and persistence. CD4+ T cells (see FIG. 17A and FIG. 17B) and CD8 T cells (see FIG. 17C and FIG. 17D) were analyzed for percentage of cells expressing phenotypes consistent with T stem cell memory (tscm), T central memory (Tem), T effector memory (Tem) and T effector memory re-expressing CD45RA (temra) and IL-7Ralpha expression. The data demonstrates that all ALA-CART variants had approximately 50% of CD4's and CD8's with a Tscm phenotype whereas only 10-15% of standard 2^{nd} Gen CD22 CAR T cells had this phenotype (see FIG. 17A and FIG. 17C). Furthermore, CD4+ ALA-CART cells (regardless of scFv combination) had a higher percentage of Tcm than did the 2^{nd} Gen CAR T cells (see FIG. 17A). Both CD4 and CD8 ALA-CART cells also demonstrated higher expression of IL-7Ralpha than did the 2^{nd} Gen CD22 CAR T cells (see FIG. 17B and FIG. 17D) Taken together, these data demonstrate that early after transduction, the ALA-CART construct drives T cell differentiation in a way that favors the development of persistent T cell populations.

### Example 15—ALA-CART Cells Solely Targeting CD22 have Decreased Expression of the Exhaustion Marker CD39 than Standard 2^{ND} Generation CD22 CAR T Cells

2^{nd} generation CD22 CAR T cells and 22ALA-CART cells (all 4 combinations of SAff and HiAff scFv's at both positions) were generated using T cells from healthy donors. CAR T cells were analyzed by flow cytometry the 2 days after transduction to evaluate the expression of markers associated with T cell exhaustion. CD4+ T cells and CD8 T cells were analyzed for percentage of cells expressing the exhaustion markers PD1, TIM3 and CD39. This data is shown in FIG. 18. The data shown in FIG. 18 demonstrates that all ALA-CART variants had lower levels of CD39 expression than did standard 2ªd Generation CD22 CAR T cells. These data demonstrate that early after transduction, the ALA-CART construct drives less T cell exhaustion than do standard 2^{nd} Gen CAR constructs which can lead to enhanced in vivo function and persistence.

### Example 16—ALA-CART Cells are Able to Target Tumor Cells Expressing Low Levels of Antigens Other than CD22

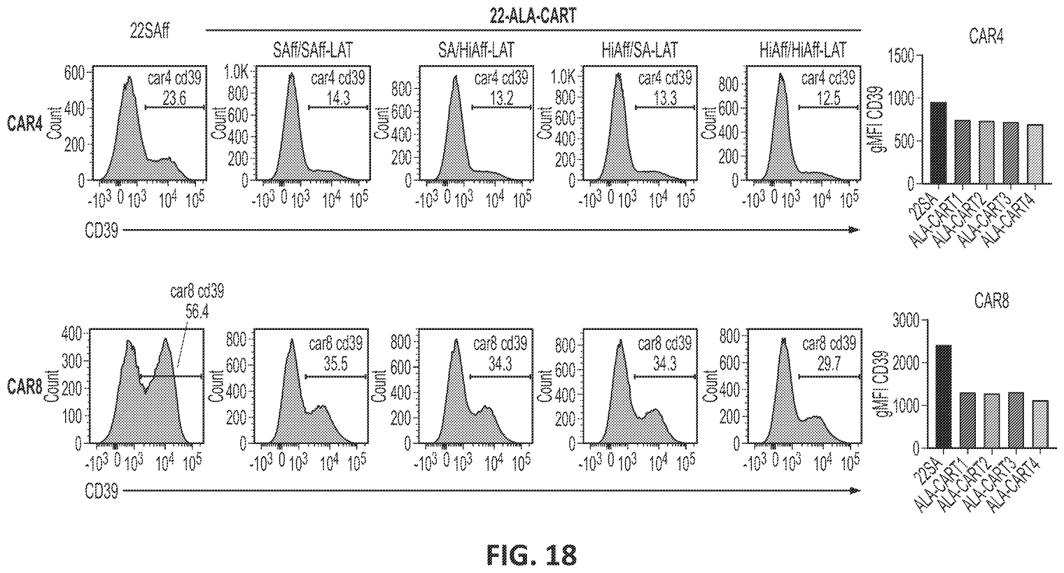

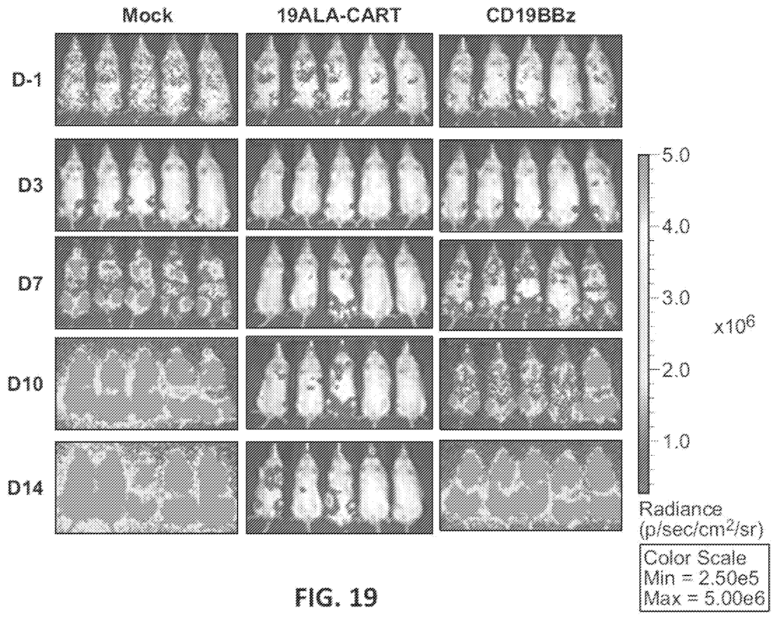

To evaluate if the ability of ALA-CART cells to effectively target low levels of antigen was restricted to CD22 or if it was generalizable to other CAR targets, the ALA-CART construct was designed to solely target the CD19 antigen by replacing the scFv's from the 22ALA-CART (also referred to as “22ALA-CART4 or “HiAff/HiAff-LATCAR”) with the CD19-directed scFv, FMC63, at both positions (19ALA-CART). ALA-CART cells expressing a CD19-BBz 2^{nd} generation CAR in conjunction with a CD19-LAT CAR (19ALA-CART) were generated from T cells from a heathy donor. T cells from the same donor were activated, but not transduced (Mock) and transduced with a standard 2^{nd} generation CD19 CAR (CD19BBz). NSG mice were engrafted with 1 million cells of a CD19-low clone of NALM6, three days prior to injection of T cells. Three days later, mice were treated with 1 million untransduced cells (Mock, negative control), 1 million standard 2^{nd} generation CD19BBz CAR T cells or 1 million 19ALA-CART cells. Leukemia burden was monitored twice weekly by bioluminescent imaging and demonstrated that mice treated with 19ALA-CART had leukemia regression whereas mice treated with the standard 200 generation CD19BBz CAR had progression of their leukemia (FIG. 19). This data demonstrates that the structure of the ALA-CART construct allows for the targeting of antigen-low tumor cells, and is not restricted to CD22-directed therapy.

### Example 17—19ALA-CART Cells Demonstrate Increased In Vivo Potency Relative to Standard 2^{ND} Generation CAR T Cells

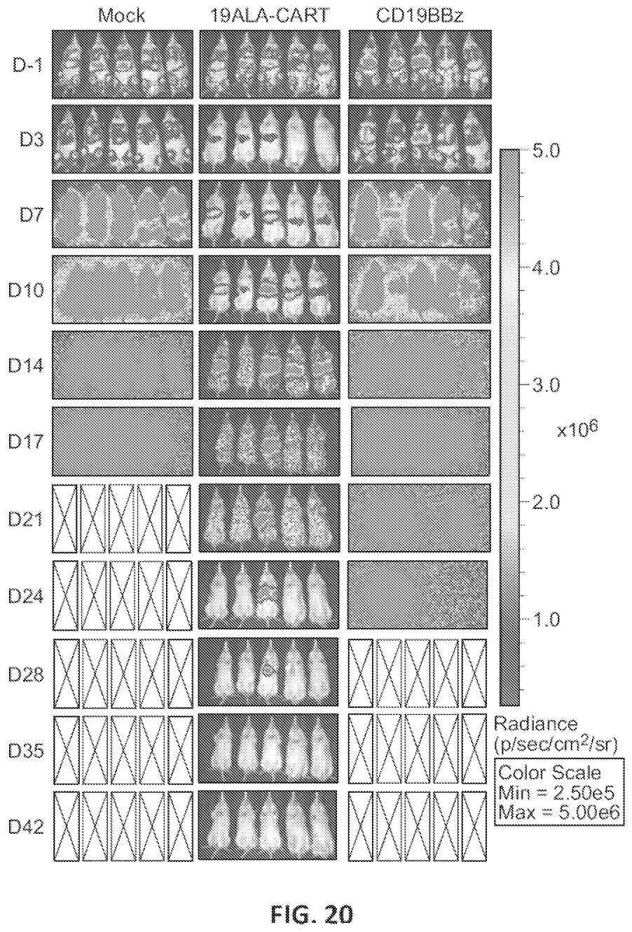

ALA-CART cells expressing a CD19-BBz 2^{nd} generation CAR in conjunction with a CD19-LAT CAR (19ALA-CART) were generated from T cells from a heathy donor. T cells from the same donor were activated, but not transduced (Mock) and transduced with a standard 2^{nd} generation CD19 CAR (CD19BBz). To assess the relative potency of the 19ALA-CART cells, NSG mice were engrafted with 1 million CD19 wild type NALM6 and three days later were treated with either 1 million untransduced T cells (Mock, negative control), 1 million standard 2^{nd} generation CD19BBz CAR T cells or 1 million 19ALA-CART cells. Leukemia burden was monitored twice weekly by bioluminescent imaging and demonstrated that mice treated with 19ALA-CART had leukemia regression whereas mice treated with the standard 21d generation CD19BBz CAR had moderate slowing of leukemic progression, but ultimately uniformly succumbed to leukemia (FIG. 20). This data demonstrates that the ALA-CART format not only increases the sensitivity of the T cells to low levels of antigen but also enhances the potency of the CAR T cell response when antigen levels are high.

### Example 18—Targeted Mutations to Prevent Ubiquitination of LAT can be Incorporated without Reducing Cytotoxicity

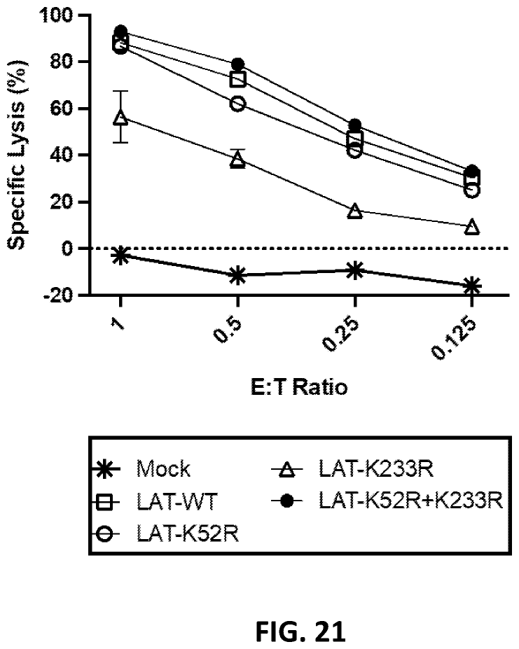

The LAT molecule is known to have 2 lysine residues (K52 and K233) that through ubiquitination target the LAT molecule for degradation thereby degrading the T cell signal. All prior experiments utilized a LAT-CAR with a mutation to the K52 residue to prevent ubiquitination at that site. To determine if this mutation, or mutation to the other ubiquitination site, impacts ALA-CART cytotoxicity, healthy donor T cells were transduced with various 22ALA-CART constructs were generated. All constructs included CD22-BBz 2^{nd} generation CAR in conjunction with a CD22-LAT CAR incorporating the signaling domain of the LAT molecule Variations of the LAT molecule were tested for their impact on the ALA-CART cells ability to kill CD22-low NALM6 cells in an overnight cytotoxicity assay. The LAT variations include: an unmodified, wild type LAT signaling domain (LAT-WT, SEQ ID NO: 26); a LAT signaling domain in which the Lysine (K) at position 52 (a target of ubiquitination) was substituted with an Arginine (K52R, SEQ ID NO: 27); a LAT signaling domain in which the Lysine at position 233 (a target of ubiquitination) was substituted with an Arginine (K233R, SEQ ID NO. 28); and a LAT signaling domain in which both Lysines at positions 52 and 233 were substituted with Arginines (K52R+K233R, SEQ ID NO: 29). Results demonstrate that mutations which specifically block ubiquitination of the K52 site preserves the cytotoxicity of 22ALA-CART cells, whereas mutating the K233 residue alone negatively impacts 22ALA-CART cytotoxicity (FIG. 21).

### Example 19—Targeted Mutations to Enhance Phosphorylation of the Phospholipase C (Plc) Binding Residue of LAT Preserves Cytotoxic Activity of 22ALA-CART

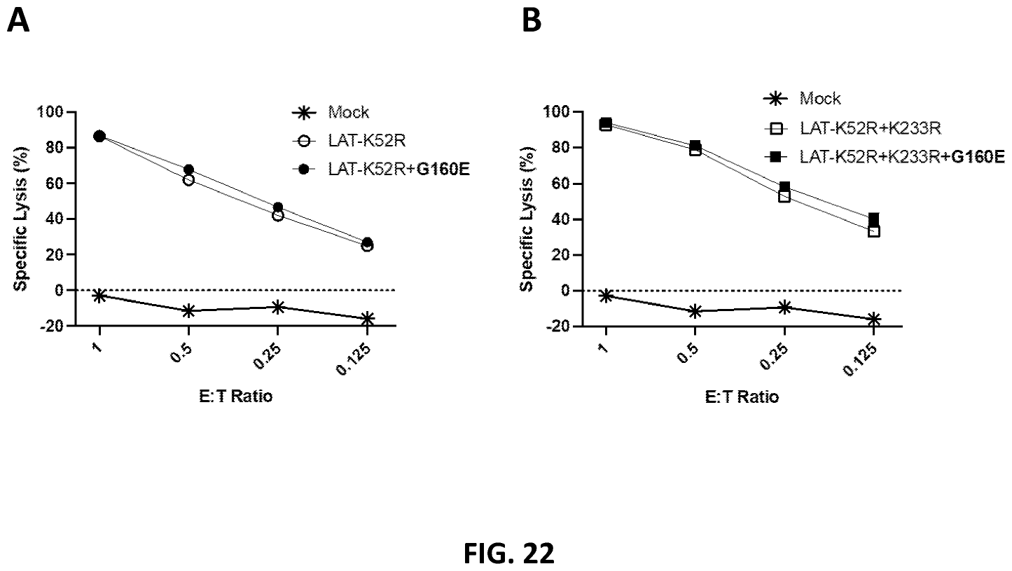

The tyrosine in LAT which is responsible for the binding of Phospholipase C (PLC), is preceded by a glycine, which has been shown to decrease the efficiency of phosphorylation of this tyrosine by ZAP70. Mutations of the preceding glycine to a glutamic acid has been shown to increase the efficiency of this phosphorylation and subsequent PLC activation. To test the impact of mutating this glycine to a glutamic acid, healthy donor T cells were transduced with various 22ALA-CART constructs. All constructs included CD22-BBz 2^{nd} generation CAR in conjunction with a CD22-LAT CAR incorporating the signaling domain of the LAT molecule. Variations of the LAT molecule were tested for their impact on the ALA-CART cells ability to kill CD22-low NALM6 cells in an overnight cytotoxicity assay. The LAT variations include: a LAT signaling domain in which the Lysine (K) at position 52 (a target of ubiquitination) was substituted with an Arginine (K52R, SEQ ID NO: 27, Panel A); a LAT signaling domain incorporating the K52R mutation as well as the substitution of the Glycine at residue 160 for a Glutamic Acid which enhances the phosphorylation of the adjacent Tyrosine responsible for binding PLC (K52R+G160E, SEQ ID NO: 30, Panel A); a LAT signaling domain incorporating the ubiquitination-blocking mutations at K52 and K233 (K52R+K233R, SEQ ID NO: 29, Panel B); and a LAT signaling domain incorporating the ubiquitination-blocking mutations K52R+K233R as well as the substitution of the Glycine at residue 160 for a Glutamic Acid which enhances the phosphorylation of the adjacent Tyrosine responsible for binding PLC (K52R+K233R+G160E, SEQ ID NO: 31, Panel B). Results demonstrate that mutations which enhance PLC-binding to LAT, preserves the cytotoxicity of 22ALA-CART cells relative to 22ALA-CART cells without the G160E mutation (FIG. 22). Furthermore, there is a trend towards increased cytotoxicity of G160E mutated ALA-CART when coupled with ubiquitination-blocking mutations (FIG. 22).

What is claimed is:

1. A genetically modified immune cell comprising:
a) a first chimeric antigen receptor (CAR) comprising an antigen recognition domain that binds to a first antigen, a transmembrane domain and an intracellular signaling domain;
b) a second CAR comprising an antigen recognition domain that binds to a second antigen, a transmembrane domain and a Linker for Activation of T cell (LAT) intracellular signaling domain.

2. The genetically modified immune cell of claim 1, wherein the first antigen and the second antigen are different.

3. The genetically modified immune cell of claim 1, wherein the first antigen and the second antigen are the same.

4. The genetically modified immune cell of any one of claims 1-3, wherein the intracellular signaling domain of the first CAR comprises a CD3zeta intracellular signaling domain.

5. The genetically modified immune cell of claim 4, wherein the CD3zeta intracellular signaling domain comprises the amino acid sequence of SEQ ID NO: 24 or SEQ ID NO: 25, preferably wherein the CD3zeta intracellular signaling domain comprises the amino acid sequence of SEQ ID NO: 24.

6. The genetically modified immune cell of any one of claims 1-5, wherein the intracellular signaling domain of the first CAR further comprises at least one additional intracellular signaling domains selected from the group consisting of a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8a intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain and any combination thereof.

7. The genetically modified immune cell of claim 6, wherein the at least one additional intracellular signaling domain is a 4-1BB intracellular signaling domain comprising the amino acid sequence of SEQ ID NO: 17.

8. The genetically modified immune cell of claim any one of claims 1-7, wherein the LAT intracellular signaling domain of the second CAR comprises the amino acid sequence of any one of SEQ ID NOs: 26-34, preferably wherein the LAT intracellular signaling domain of the second CAR comprises the amino acid sequence of SEQ ID NO: 27.

9. The genetically modified immune cell of claim any one of claims 1-7, wherein the LAT intracellular signaling domain of the second CAR comprises the amino acid sequence of SEQ ID NO: 26 having a substitution of arginine for the lysine (K25R) at position 25 of SEQ ID NO: 26, a substitution of glutamic acid for the glycine at position 133 (G133E) of SEQ ID NO: 26, a substitution of arginine for the lysine at position 206 (K206R) of SEQ ID No: 26, or any combination of the preceding substitutions.

10. The genetically modified immune cell of claim any one of claims 1-7, wherein the LAT intracellular signaling domain of the second CAR comprises the amino acid sequence of SEQ ID NO: 32 having a substitution of arginine for the lysine (K25R) at position 25 of SEQ ID NO: 32, a substitution of glutamic acid for the glycine at position 10⁴ (G10⁴E) of SEQ ID NO: 32, a substitution of arginine for the lysine at position 177 (K177R) of SEQ ID NO: 32, or any combination of the preceding substitutions.

11. The genetically modified immune cell of claim any one of claims 1-7, wherein the LAT intracellular signaling domain of the second CAR comprises the amino acid sequence of SEQ ID NO: 33 having a substitution of arginine for the lysine (K25R) at position 25 of SEQ ID NO: 33, a substitution of glutamic acid for the glycine at position 103 (G103E) of SEQ ID NO: 33, a substitution of arginine for the lysine at position 176 (K176R) of SEQ ID No: 33, or any combination of the preceding substitutions.

12. The genetically modified immune cell of claim any one of claims 1-7, wherein the LAT intracellular signaling domain of the second CAR comprises the amino acid sequence of SEQ ID NO: 34 having a substitution of arginine for the lysine (K25R) at position 25 of SEQ ID NO: 34, a substitution of glutamic acid for the glycine at position 132 (G132E) of SEQ ID NO: 34, a substitution of arginine for the lysine at position 205 (K205R) of SEQ ID No: 34, or any combination of the preceding substitutions.

13. The genetically modified immune cell of any one of claims 1-12, wherein the transmembrane domain of the first CAR and/or the second CAR is derived from a transmembrane domain selected from the group consisting of a CD8a transmembrane domain, a CD28 transmembrane domain, a CD3z transmembrane domain, a CD4 transmembrane domain, a 4-1 BB transmembrane domain, a OX40 transmembrane domain, a ICOS transmembrane domain, a PD-1 transmembrane domain, a LAG-3 transmembrane domain, a 2B4 transmembrane domain, a BTLA transmembrane domain and any combination thereof.

14. The genetically modified immune cell of claim 13, wherein the transmembrane domain of the first CAR is derived from a CD8alpha transmembrane domain comprising the amino acid sequence of SEQ ID NO: 13.

15. The genetically modified immune cell of claim 13, wherein the transmembrane domain of the second CAR is derived from a CD28 transmembrane domain comprising the amino acid sequence of SEQ ID NO: 14.

16. The genetically modified immune cell of any one of claims 1-15, wherein the antigen recognition domain of the first CAR and/or the antigen recognition domain of the second CAR is an antibody, an antibody fragment, a single chain antibody, a single domain antibody, an scFv, a VH or a VHH or antigen binding fragment thereof.

17. The genetically modified immune cell of any one of claims 1-16, wherein the antigen recognition domain of the first CAR and the antigen recognition domain of the second CAR further comprises a leader domain selected from the group consisting of a CD8alpha leader domain.

18. The genetically modified immune cell of claim 17, wherein the leader domain is a CD8alpha leader domain comprising the amino acid sequence of SEQ ID NO: 1 or SEQ ID NO: 2.

19. The genetically modified immune cell of any one of claims 1-18, wherein the first antigen and the second antigen is a tumor associated antigen.

20. The genetically modified immune cell of claim 19, wherein the tumor associated antigen is selected from a group consisting of CD19, CD22, CD20, CD138, BCMA, CD33, CD123, FLT, CLL, CD56, CD34, CD117, CD14, CD133, CD44v6, CD47, CD64, CD96, CD97, CD99, CD45, CD9, Muc1, Lewis-Y, IL1RAP, FR-beta, CD5, CD7, CD38, CD30, B7-H3, HER2, CD44v6, CEA, c-Met, EGFRvIII, Epcam, EphA2, FR-alpha, GD2, GPC3, IL13R-alpha2, IL11R-alpha, L1-CAM, mesothelin, MUC1, MUC16, NKGD2 and PSCA.

21. The genetically modified immune cell of claim 20, wherein the first antigen is CD22.

22. The genetically modified immune cell of claim 20, wherein the first antigen is CD19.

23. The genetically modified immune cell of claim 21, wherein the second antigen is CD19.

24. The genetically modified immune cell of claim 21, wherein the second antigen is CD22.

25. The genetically modified immune cell of claim 22, wherein the second antigen is CD22.

26. The genetically modified immune cell of any one of claims 1-25, wherein the immune cell is a T-cell, a Natural Killer (NK) cell, a Natural Killer (NK)-like cell, a Cytokine Induced Killer (CIK) cell, a hematopoietic progenitor cell, a peripheral blood (PB) derived T cell or an umbilical cord blood (UCB) derived T-cell.

27. The genetically modified immune cell of claim 26, wherein the immune cell is a T-cell.

28. The genetically modified immune cell of any one of the preceding claims, wherein the first CAR comprises the amino acid sequence of SEQ ID NO: 69, SEQ ID NO: 102, SEQ ID NO: 306, or SEQ ID NO: 309.

29. The genetically modified immune cell of any one of the preceding claims, wherein the second CAR comprises the amino acid sequence of SEQ ID NO: 71, SEQ ID NO: 100, SEQ ID NO: 206, or SEQ ID NO: 300-308.

30. The genetically modified immune cell of any one of claims 1-29, wherein the first CAR comprises the amino acid sequence of SEQ ID NO: 102 and the second CAR comprises SEQ ID NO: 100.

31. The genetically modified immune cell of any one of the claims 1-29, wherein the first CAR comprises the amino acid sequence of SEQ ID NO: 102 and the second CAR comprises SEQ ID NO: 306.

32. The genetically modified immune cell of any one of the claims 1-29, wherein the first CAR comprises the amino acid sequence of SEQ ID NO: 309 and the second CAR comprises SEQ ID NO: 100.

33. A composition comprising the genetically modified immune cell of any one of claims 1-32 and a pharmaceutically acceptable carrier.

34. A composition comprising a population of cells, wherein the plurality of cells of the population comprises the genetically modified immune cell of any one of claims 1-32.

35. The composition of claim 34, wherein the plurality of the cells of the population comprises at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the genetically modified immune cell of any one of claims 1-26.

36. A polynucleotide encoding the first CAR and the second CAR of the genetically modified immune cell of any one of claims 1-32.

37. The polynucleotide of claim 36, wherein a nucleic acid sequence encoding a self cleaving peptide sequence is located in between the nucleic acid sequence encoding the first CAR and the nucleic acid sequence encoding the second CAR.

38. The polynucleotide of claim 37, wherein the self cleaving peptide sequence comprises the amino acid sequence of SEQ ID NO: 79.

39. The polynucleotide of any one of claims 36-38, wherein the first CAR and the second CAR encoded on a single vector.

40. The polynucleotide of claim 39, wherein the vector is a viral vector, a lentivirus vector, a non-viral vector or a transposon.

41. The polynucleotide of claim 40, wherein the vector is a bicistronic lentiviral vector.

42. A method of producing a population of genetically modified immune cells, comprising:
a) introducing into a plurality of immune cells a composition comprising the polynucleotide sequence of any one of claims 36-41, thereby generating a population of genetically modified immune cells;
b) culturing the population of genetically modified immune cells under conditions suitable for integration of the polynucleotide sequence;
c) expanding and/or selecting at least one cell from the population of genetically modified immune cells that expresses the first CAR and the second CAR on the cell surface.

43. A method of treating cancer in a subject in need thereof comprising administrating the composition of any one of claims 33-35.

44. The method of claim 43, wherein the administration of a composition comprising a modified immune cell comprising first CAR and the second CAR increases the immune response against a target cell in comparison to the administration of a composition comprising a modified immune cell comprising a first CAR alone.

45. The method of claim 44, wherein the increased immune response at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 99% or any percentage in between greater than a composition comprising a modified immune cell comprising a first CAR alone.

46. The method of any one of claims 43-45, wherein the cancer is a solid tumor, a B cell malignancy, a myeloid malignancy, a T-cell malignancy, acute lymphoblastic leukemia, acute lymphoblastic lymphoma, Non-Hodgkin lymphoma, Hodgkin's lymphoma, chronic lymphocytic leukemia, multiple myeloma, acute myeloid leukemia, myelodysplastic syndrome, myeloproliferative neoplasms, chronic myeloid leukemia, T lymphoblastic leukemia, T lymphoblastic lymphoma or Anaplastic Large Cell Leukemia.

47. The method of any one of claims 43-46, wherein the cancer has a low cell surface expression of the first antigen and/or a low cell surface expression of the second antigen.


SEQUENCE LISTING (310 sequences)

SEQ ID NO: 1 - protein, 25 aa, synthetic construct
ASATMALPVTALLLPLALLLHAARP

SEQ ID NO: 2 - protein, 21 aa, synthetic construct
MALPVTALLLPLALLLHAARP

SEQ ID NO: 3 - protein, 23 aa, synthetic construct
GSMEFGLSWLFLVAILKGVQCSR

SEQ ID NO: 4 - protein, 21 aa, synthetic construct
MEFGLSWLFLVAILKGVQCSR

SEQ ID NO: 5 - protein, 47 aa, synthetic construct
LETTTPAPRPPTPAPTIASQPLSLRPEACRPAAGGAVHTRGLDFACD

SEQ ID NO: 6 - protein, 45 aa, synthetic construct
TTTPAPRPPTPAPTIASQPLSLRPEACRPAAGGAVHTRGLDFACD

SEQ ID NO: 7 - protein, 41 aa, synthetic construct
SRIEVMYPPPYLDNEKSNGTIIHVKGKHLCPSPLFPGPSKP

SEQ ID NO: 8 - protein, 39 aa, synthetic construct
IEVMYPPPYLDNEKSNGTIIHVKGKHLCPSPLFPGPSKP

SEQ ID NO: 9 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 12 aa, synthetic construct
EPKSCDKTHTCP

SEQ ID NO: 11 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 12 aa, synthetic construct
ESKYGPPCPSCP

SEQ ID NO: 13 - protein, 24 aa, synthetic construct
IYIWAPLAGTCGVLLLSLVITLYC

SEQ ID NO: 14 - protein, 27 aa, synthetic construct
FWVLVVVGGVLACYSLLVTVAFIIFWV

SEQ ID NO: 15 - protein, 41 aa, synthetic construct
RSKRSRGGHSDYMNMTPRRPGPTRKHYQPYAPPRDFAAYRS

SEQ ID NO: 16 - protein, 41 aa, synthetic construct
RSKRSRLLHSDYMNMTPRRPGPTRKHYQPYAPPRDFAAYRS

SEQ ID NO: 17 - protein, 42 aa, synthetic construct
KRGRKKLLYIFKQPFMRPVQTTQEEDGCSCRFPEEEEGGCEL

SEQ ID NO: 18 - protein, 24 aa, synthetic construct
LCARPRRSPAQEDGKVYINMPGRG

SEQ ID NO: 19 - protein, 52 aa, synthetic construct
YFLGRLVPRGRGAAEAATRKQRITETESPYQELQGQRSDVYSDLNTQRPYYK

SEQ ID NO: 20 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 21 - protein, 42 aa, synthetic construct
ALYLLRRDQRLPPDAHKPPGGGSFRTPIQEEQADAHSTLAKI

SEQ ID NO: 22 - protein, 49 aa, synthetic construct
HQRRKYRSNKGESPVEPAEPCHYSCPREEEGSTIPIQEDYRKPEPACSP

SEQ ID NO: 23 - protein, 48 aa, synthetic construct
QRRKYRSNKGESPVEPAEPCHYSCPREEEGSTIPIQEDYRKPEPACSP

SEQ ID NO: 24 - protein, 114 aa, synthetic construct
DIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 235 aa, synthetic construct
HCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 27 - protein, 235 aa, synthetic construct
HCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 28 - protein, 235 aa, synthetic construct
HCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 29 - protein, 235 aa, synthetic construct
HCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 30 - protein, 235 aa, synthetic construct
HCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPEYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 31 - protein, 235 aa, synthetic construct
HCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPEYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 32 - protein, 206 aa, synthetic construct
HCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 33 - protein, 205 aa, synthetic construct
HCHRLPGSYDSTSSDSLYPRGIQFKRPHTVAPWPPAYPPVTSYPPLSQPDLLPIPSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 34 - protein, 234 aa, synthetic construct
HCHRLPGSYDSTSSDSLYPRGIQFKRPHTVAPWPPAYPPVTSYPPLSQPDLLPIPSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 35 - DNA, 75 bp, synthetic construct
  source 1..75: mol_type = other DNA
gctagcgccaccatggctctgcctgtgacagctctgctgctgcctctggccctgctgctccatgctgctagacct

SEQ ID NO: 36 - DNA, 63 bp, synthetic construct
  source 1..63: mol_type = other DNA
atggctctgcctgtgacagctctgctgctgcctctggccctgctgctccatgctgctagacct

SEQ ID NO: 37 - DNA, 69 bp, synthetic construct
  source 1..69: mol_type = other DNA
ggatccatggagtttggcctgagctggctgttcctggtggccatcctcaagggcgtgcagtgctccagg

SEQ ID NO: 38 - DNA, 63 bp, synthetic construct
  source 1..63: mol_type = other DNA
atggagtttggcctgagctggctgttcctggtggccatcctcaagggcgtgcagtgctccagg

SEQ ID NO: 39 - DNA, 141 bp, synthetic construct
  source 1..141: mol_type = other DNA
ctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgac

SEQ ID NO: 40 - DNA, 135 bp, synthetic construct
  source 1..135: mol_type = other DNA
accaccacccccgcccctaggcctcccacacctgcccccacaatcgcctcccagcctctcagcctgaggcctgaagcttgcaggcccgctgccggaggagctgtccataccaggggactcgacttcgcctgcgac

SEQ ID NO: 41 - DNA, 123 bp, synthetic construct
  source 1..123: mol_type = other DNA
tctagaatcgaagtgatgtaccctccaccttacctggacaacgagaagtccaacggcaccatcatccacgtgaagggcaagcacctgtgtccttctccactgttccccggacctagcaagcct

SEQ ID NO: 42 - DNA, 117 bp, synthetic construct
  source 1..117: mol_type = other DNA
atcgaagtgatgtaccctccaccttacctggacaacgagaagtccaacggcaccatcatccacgtgaagggcaagcacctgtgtccttctccactgttccccggacctagcaagcct

SEQ ID NO: 43 - DNA, 696 bp, synthetic construct
  source 1..696: mol_type = other DNA
gagcccaagagctgcgacaagacccacacctgccccccctgccccgcccccgagctgctgggcggccccagcgtgttcctgttcccccccaagcccaaggacaccctgatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctgaccgtgctgcaccaggactggctgaacggcaaggagtacaagtgcaaggtgagcaacaaggccctgcccgcccccatcgagaagaccatcagcaaggccaagggccagccccgggagccccaggtgtacaccctgccccccagccgggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagccggtggcagcagggcaacgtgttcagctgcagcgtgatgcacgaggccctgcacaaccactacacccagaagagcctgagcctgagccccggcaag

SEQ ID NO: 44 - DNA, 36 bp, synthetic construct
  source 1..36: mol_type = other DNA
gagcccaagagctgcgacaagacccacacctgcccc

SEQ ID NO: 45 - DNA, 687 bp, synthetic construct
  source 1..687: mol_type = other DNA
gagagcaagtacggccccccctgccccagctgccccgcccccgagttcctgggcggccccagcgtgttcctgttcccccccaagcccaaggacaccctgatgatcagccggacccccgaggtgacctgcgtggtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccggctgaccgtggacaagagccggtggcaggagggcaacgtgttcagctgcagcgtgatgcacgaggccctgcacaaccactacacccagaagagcctgagcctgagcctgggcaag

SEQ ID NO: 46 - DNA, 27 bp, synthetic construct
  source 1..27: mol_type = other DNA
gagagcaagtacggccccccctgcccc

SEQ ID NO: 47 - DNA, 72 bp, synthetic construct
  source 1..72: mol_type = other DNA
atttacatttgggcccctctggctggaacctgcggagtcctgctgctgtccctggtgatcacactgtactgt

SEQ ID NO: 48 - DNA, 81 bp, synthetic construct
  source 1..81: mol_type = other DNA
ttctgggtgctcgttgttgttggcggcgtgctggcctgttacagcctgctggttaccgtggccttcatcatcttttgggtg

SEQ ID NO: 49 - DNA, 123 bp, synthetic construct
  source 1..123: mol_type = other DNA
cgaagcaagcggagccggggaggacacagcgactacatgaacatgacccctcggaggccaggccccaccagaaagcactaccagccctacgcccctccccgggactttgccgcctatcggagc

SEQ ID NO: 50 - DNA, 123 bp, synthetic construct
  source 1..123: mol_type = other DNA
cgaagcaagcggagccggctgctgcacagcgactacatgaacatgacccctcggaggccaggccccaccagaaagcactaccagccctacgcccctccccgggactttgccgcctatcggagc

SEQ ID NO: 51 - DNA, 126 bp, synthetic construct
  source 1..126: mol_type = other DNA
aagaggggcagaaagaagctgctctacatcttcaagcagccctttatgagacccgtgcagacaacccaggaggaagacggatgcagctgcaggttccctgaggaggaggagggcggctgcgaactg

SEQ ID NO: 52 - DNA, 72 bp, synthetic construct
  source 1..72: mol_type = other DNA
ctgtgcgcccggccccggcggagccccgcccaggaggacggcaaggtgtacatcaacatgcccggccggggc

SEQ ID NO: 53 - DNA, 156 bp, synthetic construct
  source 1..156: mol_type = other DNA
tacttcctgggccggctggtgccccggggccggggcgccgccgaggccgccacccggaagcagcggatcaccgagaccgagagcccctaccaggagctgcagggccagcggagcgacgtgtacagcgacctgaacacccagcggccctactacaag

SEQ ID NO: 54 - DNA, 360 bp, synthetic construct
  source 1..360: mol_type = other DNA
tggcggcggaagcggaaggagaagcagagcgagaccagccccaaggagttcctgaccatctacgaggacgtgaaggacctgaagacccggcggaaccacgagcaggagcagaccttccccggcggcggcagcaccatctacagcatgatccagagccagagcagcgcccccaccagccaggagcccgcctacaccctgtacagcctgatccagcccagccggaagagcggcagccggaagcggaaccacagccccagcttcaacagcaccatctacgaggtgatcggcaagagccagcccaaggcccagaaccccgcccggctgagccggaaggagctggagaacttcgacgtgtacagc

SEQ ID NO: 55 - DNA, 126 bp, synthetic construct
  source 1..126: mol_type = other DNA
gccctgtacctgctgcggcgggaccagcggctgccccccgacgcccacaagccccccggcggcggcagcttccggacccccatccaggaggagcaggccgacgcccacagcaccctggccaagatc

SEQ ID NO: 56 - DNA, 147 bp, synthetic construct
  source 1..147: mol_type = other DNA
caccagcggcggaagtaccggagcaacaagggcgagagccccgtggagcccgccgagccctgccactacagctgcccccgggaggaggagggcagcaccatccccatccaggaggactaccggaagcccgagcccgcctgcagcccc

SEQ ID NO: 57 - DNA, 144 bp, synthetic construct
  source 1..144: mol_type = other DNA
cagcggcggaagtaccggagcaacaagggcgagagccccgtggagcccgccgagccctgccactacagctgcccccgggaggaggagggcagcaccatccccatccaggaggactaccggaagcccgagcccgcctgcagcccc

SEQ ID NO: 58 - DNA, 342 bp, synthetic construct
  source 1..342: mol_type = other DNA
gatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 59 - DNA, 336 bp, synthetic construct
  source 1..336: mol_type = other DNA
agggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 60 - DNA, 705 bp, synthetic construct
  source 1..705: mol_type = other DNA
cactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaac

SEQ ID NO: 61 - DNA, 705 bp, synthetic construct
  source 1..705: mol_type = other DNA
cactgccacagactgcccggcagctacgatagcaccagcagcgattctctgtaccccagaggcatccagttcaaacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaac

SEQ ID NO: 62 - DNA, 705 bp, synthetic construct
  source 1..705: mol_type = other DNA
cactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccagaacagaacctgctgctctgtctagccaagaggccgaggaagtggaagaagaaggcgcccctgactacgagaacctgcaagagctgaac

SEQ ID NO: 63 - DNA, 705 bp, synthetic construct
  source 1..705: mol_type = other DNA
cactgccacagactgcccggcagctacgatagcaccagcagcgattctctgtaccccagaggcatccagttcaaacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccagaacagaacctgctgctctgtctagccaagaggccgaggaagtggaagaagaaggcgcccctgactacgagaacctgcaagagctgaac

SEQ ID NO: 64 - DNA, 705 bp, synthetic construct
  source 1..705: mol_type = other DNA
cactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctgagtacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaac

SEQ ID NO: 65 - DNA, 705 bp, synthetic construct
  source 1..705: mol_type = other DNA
cactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctgagtacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccagaacagaacctgctgctctgtctagccaagaggccgaggaagtggaagaagaaggcgcccctgactacgagaacctgcaagagctgaac

SEQ ID NO: 66 - DNA, 618 bp, synthetic construct
  source 1..618: mol_type = other DNA
cactgccaccggctgcccggcagctacgacagcaccagcagcgacagcctgtacccccggggcatccagttcaagcggccccacaccgtggccccctggccccccgcctacccccccgtgaccagctacccccccctgagccagcccgacctgctgcccatcccccggagcccccagcccctgggcggcagccaccggacccccagcagccggcgggacagcgacggcgccaacagcgtggccagctacgagaacgaggagcccgcctgcgaggacgccgacgaggacgaggacgactaccacaaccccggctacctggtggtgctgcccgacagcacccccgccaccagcaccgccgcccccagcgcccccgccctgagcacccccggcatccgggacagcgccttcagcatggagagcatcgacgactacgtgaacgtgcccgagagcggcgagagcgccgaggccagcctggacggcagccgggagtacgtgaacgtgagccaggagctgcaccccggcgccgccaagaccgagcccgccgccctgagcagccaggaggccgaggaggtggaggaggagggcgcccccgactacgagaacctgcaggagctgaac

SEQ ID NO: 67 - DNA, 615 bp, synthetic construct
  source 1..615: mol_type = other DNA
cactgccaccggctgcccggcagctacgacagcaccagcagcgacagcctgtacccccggggcatccagttcaagcggccccacaccgtggccccctggccccccgcctacccccccgtgaccagctacccccccctgagccagcccgacctgctgcccatccccagcccccagcccctgggcggcagccaccggacccccagcagccggcgggacagcgacggcgccaacagcgtggccagctacgagaacgaggagcccgcctgcgaggacgccgacgaggacgaggacgactaccacaaccccggctacctggtggtgctgcccgacagcacccccgccaccagcaccgccgcccccagcgcccccgccctgagcacccccggcatccgggacagcgccttcagcatggagagcatcgacgactacgtgaacgtgcccgagagcggcgagagcgccgaggccagcctggacggcagccgggagtacgtgaacgtgagccaggagctgcaccccggcgccgccaagaccgagcccgccgccctgagcagccaggaggccgaggaggtggaggaggagggcgcccccgactacgagaacctgcaggagctgaac

SEQ ID NO: 68 - DNA, 702 bp, synthetic construct
  source 1..702: mol_type = other DNA
cactgccaccggctgcccggcagctacgacagcaccagcagcgacagcctgtacccccggggcatccagttcaagcggccccacaccgtggccccctggccccccgcctacccccccgtgaccagctacccccccctgagccagcccgacctgctgcccatccccagcccccagcccctgggcggcagccaccggacccccagcagccggcgggacagcgacggcgccaacagcgtggccagctacgagaacgagggcgccagcggcatccggggcgcccaggccggctggggcgtgtggggccccagctggacccggctgacccccgtgagcctgccccccgagcccgcctgcgaggacgccgacgaggacgaggacgactaccacaaccccggctacctggtggtgctgcccgacagcacccccgccaccagcaccgccgcccccagcgcccccgccctgagcacccccggcatccgggacagcgccttcagcatggagagcatcgacgactacgtgaacgtgcccgagagcggcgagagcgccgaggccagcctggacggcagccgggagtacgtgaacgtgagccaggagctgcaccccggcgccgccaagaccgagcccgccgccctgagcagccaggaggccgaggaggtggaggaggagggcgcccccgactacgagaacctgcaggagctgaac

SEQ ID NO: 69 - protein, 488 aa, synthetic construct
ASATMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - DNA, 1464 bp, synthetic construct
  source 1..1464: mol_type = other DNA
gctagcgccaccatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 71 - protein, 569 aa, synthetic construct
GSMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 72 - DNA, 1719 bp, synthetic construct
  source 1..1719: mol_type = other DNA
ggatccatggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactgatgagtcgac

SEQ ID NO: 73 - protein, 22 aa, synthetic construct
GSGATNFSLLKQAGDVEENPGP

SEQ ID NO: 74 - protein, 22 aa, synthetic construct
GSGATNFSLLKQAGDVEENPGP

SEQ ID NO: 75 - protein, 21 aa, synthetic construct
GSGEGRGSLLTCGDVEENPGP

SEQ ID NO: 76 - protein, 23 aa, synthetic construct
GSGQCTNYALLKLAGDVESNPGP

SEQ ID NO: 77 - protein, 25 aa, synthetic construct
GSGVKQTLNFDLLKLAGDVESNPGP

SEQ ID NO: 78 - protein, 12 aa, synthetic construct
RKRRGSGTPDPW

SEQ ID NO: 79 - protein, 34 aa, synthetic construct
RKRRGSGTPDPWGSGATNFSLLKQAGDVEENPGP

SEQ ID NO: 80 - protein, 1091 aa, synthetic construct
ASATMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 81 - protein, 3285 aa, synthetic construct
GCTAGCGCCACCATGGCTCTGCCTGTGACAGCTCTGCTGCTGCCTCTGGCCCTGCTGCTCCATGCTGCTAGACCTCAGGTGCAGCTCCAGCAGTCTGGCCCAGGACTGGTCAAGCCTAGCCAGACCCTGAGCCTGACCTGCGCCATCAGCGGCGACAGCGTGTCCTCTAACAGCGCCGCCTGGAACTGGATCAGACAGAGCCCCAGCAGAGGCCTGGAATGGCTGGGCCGGACCTACTACCGGTCCAAGTGGTACAACGACTACGCCGTGTCCGTGAAGTCCCGGATCACCATCAACCCCGACACCAGCAAGAACCAGTTCTCCCTGCAGCTGAACAGCGTGACCCCTGAGGACACCGCCGTGTACTACTGCGCCAGAGAAGTGACCGGCGACCTGGAAGATGCCTTCGACATCTGGGGCCAGGGCACCATGGTCACCGTGTCTAGCGGAGGCGGCGGAAGCGACATCCAGATGACCCAGAGCCCTAGCTCCCTGAGCGCCAGCGTGGGCGACAGAGTGACCATCACCTGTCGGGCCAGCCAGACCATCTGGTCCTACCTGAATTGGTATCAGCAGCGGCCAGGCAAGGCCCCTAACCTGCTGATCTATGCCGCCAGCAGCCTGCAGAGCGGCGTGCCAAGCAGATTCTCTGGCAGAGGCTCCGGCACCGACTTCACCCTGACAATCAGTTCCCTGCAGGCCGAGGACTTCGCCACCTACTACTGCCAGCAGTCCTACAGCATCCCTCAGACCTTCGGCCAGGGGACCAAGCTGGAAATCAAGCTCGAGACCACCACCCCCGCCCCTAGGCCTCCCACACCTGCCCCCACAATCGCCTCCCAGCCTCTCAGCCTGAGGCCTGAAGCTTGCAGGCCCGCTGCCGGAGGAGCTGTCCATACCAGGGGACTCGACTTCGCCTGCGACATTTACATTTGGGCCCCTCTGGCTGGAACCTGCGGAGTCCTGCTGCTGTCCCTGGTGATCACACTGTACTGTAAGAGGGGCAGAAAGAAGCTGCTCTACATCTTCAAGCAGCCCTTTATGAGACCCGTGCAGACAACCCAGGAGGAAGACGGATGCAGCTGCAGGTTCCCTGAGGAGGAGGAGGGCGGCTGCGAACTGGATATCAGGGTGAAGTTCAGCAGGAGCGCCGACGCCCCCGCTTATCAACAGGGCCAGAACCAGCTGTACAACGAGCTGAACCTCGGCAGAAGAGAGGAGTATGACGTGCTGGACAAGAGGAGGGGCAGGGACCCTGAGATGGGCGGCAAGCCTAGAAGAAAGAACCCCCAGGAAGGCCTCTACAACGAACTGCAGAAGGACAAGATGGCCGAGGCCTACAGCGAGATCGGCATGAAAGGCGAGAGAAGGAGGGGAAAGGGACATGACGGCCTGTACCAGGGACTCTCCACAGCCACCAAGGACACCTACGATGCCCTGCACATGCAGGCTCTGCCCCCTAGAAGGAAGAGAAGAGGCTCTGGTACCCCCGATCCTTGGGGAAGCGGCGCTACCAACTTCTCCCTGCTCAAGCAGGCTGGCGATGTGGAGGAGAACCCCGGCCCCGGATCCATGGAGTTTGGCCTGAGCTGGCTGTTCCTGGTGGCCATCCTCAAGGGCGTGCAGTGCTCCAGGGACATCCAGATGACCCAGACCACAAGCAGCCTGAGCGCTTCCCTCGGCGACAGGGTGACCATCTCCTGTAGAGCCTCCCAAGACATCTCCAAGTACCTGAACTGGTACCAGCAGAAACCCGACGGCACCGTGAAGCTGCTGATCTACCACACCAGCAGGCTGCATTCCGGCGTGCCCTCCAGATTTTCCGGCAGCGGCTCTGGTACCGACTACAGCCTCACCATCAGCAACTTAGAACAGGAGGACATCGCCACATATTTCTGCCAACAGGGAAACACACTCCCCTATACCTTCGGCGGCGGCACAAAGTTAGAAATCACCGGCTCCACATCCGGCAGCGGAAAACCTGGTTCTGGCGAGGGCAGCACCAAGGGCGAAGTGAAGCTGCAGGAAAGCGGACCTGGACTGGTCGCTCCCAGCCAGAGCCTCAGCGTGACCTGTACAGTGAGCGGCGTGAGCCTGCCTGATTACGGCGTGAGCTGGATTAGACAGCCTCCCAGGAAGGGCTTAGAATGGCTCGGCGTGATTTGGGGCAGCGAGACAACCTACTATAACAGCGCCCTGAAGAGCAGGCTCACCATTATCAAGGACAACAGCAAATCCCAGGTCTTCCTGAAGATGAACAGCCTCCAGACCGACGACACCGCCATCTACTACTGCGCCAAGCACTACTATTATGGCGGCTCCTACGCCATGGACTACTGGGGCCAGGGCACCAGCGTGACAGTGTCTAGAATCGAAGTGATGTACCCTCCACCTTACCTGGACAACGAGAAGTCCAACGGCACCATCATCCACGTGAAGGGCAAGCACCTGTGTCCTTCTCCACTGTTCCCCGGACCTAGCAAGCCTTTCTGGGTGCTCGTTGTTGTTGGCGGCGTGCTGGCCTGTTACAGCCTGCTGGTTACCGTGGCCTTCATCATCTTTTGGGTGCACTGCCACAGACTGCCCGGCAGCTACGATAGCACCAGCAGCGATTCTCTGTACCCCAGAGGCATCCAGTTCAGACGGCCTCATACAGTGGCTCCCTGGCCTCCTGCTTACCCTCCTGTGACAAGCTACCCACCTCTGAGCCAGCCTGACCTGCTGCCTATTCCTAGAAGCCCTCAGCCTCTCGGCGGCAGCCATAGAACACCTAGCAGCAGAAGAGATAGCGACGGCGCCAATAGCGTGGCCAGCTACGAAAATGAAGGCGCCTCTGGCATTAGAGGCGCCCAAGCTGGATGGGGAGTTTGGGGACCTAGCTGGACAAGACTGACCCCTGTGTCTCTGCCTCCTGAACCTGCCTGCGAAGATGCCGACGAGGACGAGGATGACTATCACAACCCTGGCTACCTGGTGGTGCTGCCTGATAGCACACCAGCCACATCTACAGCCGCTCCTAGTGCTCCTGCTCTGAGCACACCTGGCATCAGAGACAGCGCCTTCAGCATGGAATCCATCGACGACTACGTGAACGTGCCCGAGTCTGGCGAATCTGCCGAAGCCTCTCTTGACGGCAGCCGCGAGTATGTGAACGTGTCCCAAGAACTGCATCCCGGCGCTGCCAAAACAGAACCTGCTGCTCTGTCTAGCCAAGAGGCCGAGGAAGTGGAAGAAGAAGGCGCCCCTGACTACGAGAACCTGCAAGAGCTGAACTGATGAGTCGAC

SEQ ID NO: 82 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 83 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 84 - protein, 10 aa, synthetic construct
GDSVSSNSAA

SEQ ID NO: 85 - protein, 9 aa, synthetic construct
TYYRSKWYN

SEQ ID NO: 86 - protein, 14 aa, synthetic construct
AREVTGDLEDAFDI

SEQ ID NO: 87 - protein, 6 aa, synthetic construct
QTIWSY

SEQ ID NO: 88 - skipped

SEQ ID NO: 89 - protein, 9 aa, synthetic construct
QQSYSIPQT

SEQ ID NO: 90-91 - skipped (2 entries)

SEQ ID NO: 92 - protein, 118 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTV

SEQ ID NO: 93 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 94 - protein, 8 aa, synthetic construct
GVSLPDYG

SEQ ID NO: 95 - protein, 7 aa, synthetic construct
IWGSETT

SEQ ID NO: 96 - protein, 14 aa, synthetic construct
AKHYYYGGSYAMDY

SEQ ID NO: 97 - protein, 6 aa, synthetic construct
QDISKY

SEQ ID NO: 98 - skipped

SEQ ID NO: 99 - protein, 9 aa, synthetic construct
QQGNTLPYT

SEQ ID NO: 100 - protein, 567 aa, synthetic construct
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 101 - DNA, 1713 bp, synthetic construct
  source 1..1713: mol_type = other DNA
atggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactgatgagtcgac

SEQ ID NO: 102 - protein, 484 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - DNA, 1452 bp, synthetic construct
  source 1..1452: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 104 - protein, 1087 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 105 - DNA, 3273 bp, synthetic construct
  source 1..3273: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagctcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactgatgagtcgac

SEQ ID NO: 106-199 - skipped (94 entries)

SEQ ID NO: 200 - protein, 1092 aa, synthetic construct
ASATMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 201 - DNA, 3263 bp, synthetic construct
  source 1..3263: mol_type = other DNA
ctcgagatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagactagttcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccgctcgagatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagactagtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactgatga

SEQ ID NO: 202 - protein, 1102 aa, synthetic construct
ASATMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 203 - DNA, 3311 bp, synthetic construct
  source 1..3311: mol_type = other DNA
ctcgagatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagactagttcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggctctgcctgtgacagctctgctgctgcctctggccctgctgctccatgctgctagacctgactacaaagacgatgacgacaagcaggtgcagctccagcagtctggcccaggaatggtcaagcctagccagaccctgagcctgacctgcgccatcagcggcgacagcgtgtcctctaacagcgtcgcctggaactggatcagacagagccccagcagaggcctggaatggctgggccggacctactaccggtccacgtggtacaacgactacgccgtgtccatgaagtcccggatcaccatcaaccccgacaccaacaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcggtggaggcggtagcggcggtggcggttccgacatccagatgatccagagccctagctccctgagcgccagcgtgggcgacagagtgaccatcacctgtcgggccagccagaccatctggtcctacctgaattggtatcggcagcggccaggcgaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactgatga

SEQ ID NO: 204 - protein, 1098 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 205 - DNA, 3300 bp, synthetic construct
  source 1..3300: mol_type = other DNA
atggcactgccagtgactgcattactcttgccactcgcgctactgttacacgcagcacgtccacatcaccatcaccatcaccaagtccaattgcaacaaagcgggccgggcatggtgaaaccgagtcaaacgttatctcttacgtgtgcgatttcgggggatagtgtcagcagcaattcagtggcgtggaattggattcgccaatcgccgagtcgcgggttggagtggctcgggcgcacgtattatcgcagcacatggtataatgattatgcggtcagcatgaaaagccgcattacgattaatccggatacgaacaaaaatcaatttagcttacaattaaattccgtcacgccggaagatacagcggtctattattgtgcgcgcgaggtcacgggggatctcgaagacgcgtttgatatttgggggcaagggaccatggtgactgtcagctctggtggagggggcagtggaggtgggggatcgggaggtggtggcagtgatattcaaatgatccaaagtccatccagcctatccgcatctgtcggagatcgcgtaacgattacgtgccgcgcgagtcaaacgatttggagctatctgaactggtaccggcaacgcccgggcgaagcgccgaatctcttgatttacgcggcgtcctcattacagtcgggtgtcccgagccgctttagcggccgcggaagcggtacggattttacgttaaccattagcagcctccaggcggaagattttgcgacgtattactgtcaacagagctatagcattccgcagacgtttggtcagggcacgaaattggagattaaactcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccgctcgagatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagactagtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactgatga

SEQ ID NO: 206 - protein, 1108 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 207 - DNA, 3348 bp, synthetic construct
  source 1..3348: mol_type = other DNA
atggcactgccagtgactgcattactcttgccactcgcgctactgttacacgcagcacgtccacatcaccatcaccatcaccaagtccaattgcaacaaagcgggccgggcatggtgaaaccgagtcaaacgttatctcttacgtgtgcgatttcgggggatagtgtcagcagcaattcagtggcgtggaattggattcgccaatcgccgagtcgcgggttggagtggctcgggcgcacgtattatcgcagcacatggtataatgattatgcggtcagcatgaaaagccgcattacgattaatccggatacgaacaaaaatcaatttagcttacaattaaattccgtcacgccggaagatacagcggtctattattgtgcgcgcgaggtcacgggggatctcgaagacgcgtttgatatttgggggcaagggaccatggtgactgtcagctctggtggagggggcagtggaggtgggggatcgggaggtggtggcagtgatattcaaatgatccaaagtccatccagcctatccgcatctgtcggagatcgcgtaacgattacgtgccgcgcgagtcaaacgatttggagctatctgaactggtaccggcaacgcccgggcgaagcgccgaatctcttgatttacgcggcgtcctcattacagtcgggtgtcccgagccgctttagcggccgcggaagcggtacggattttacgttaaccattagcagcctccaggcggaagattttgcgacgtattactgtcaacagagctatagcattccgcagacgtttggtcagggcacgaaattggagattaaactcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggctctgcctgtgacagctctgctgctgcctctggccctgctgctccatgctgctagacctgactacaaagacgatgacgacaagcaggtgcagctccagcagtctggcccaggaatggtcaagcctagccagaccctgagcctgacctgcgccatcagcggcgacagcgtgtcctctaacagcgtcgcctggaactggatcagacagagccccagcagaggcctggaatggctgggccggacctactaccggtccacgtggtacaacgactacgccgtgtccatgaagtcccggatcaccatcaaccccgacaccaacaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcggtggaggcggtagcggcggtggcggttccgacatccagatgatccagagccctagctccctgagcgccagcgtgggcgacagagtgaccatcacctgtcgggccagccagaccatctggtcctacctgaattggtatcggcagcggccaggcgaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactgatga

SEQ ID NO: 208 - protein, 267 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 209 - protein, 145 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 210 - protein, 107 aa, synthetic construct
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 211 - protein, 15 aa, synthetic construct
GGGGSGGGGSGGGGS

SEQ ID NO: 212 - protein, 10 aa, synthetic construct
GDSVSSNSVA

SEQ ID NO: 213 - protein, 9 aa, synthetic construct
TYYRSTWYN

SEQ ID NO: 214 - DNA, 801 bp, synthetic construct
  source 1..801: mol_type = other DNA
atggctctgcctgtgacagctctgctgctgcctctggccctgctgctccatgctgctagacctcaggtgcagctccagcagtctggcccaggaatggtcaagcctagccagaccctgagcctgacctgcgccatcagcggcgacagcgtgtcctctaacagcgtcgcctggaactggatcagacagagccccagcagaggcctggaatggctgggccggacctactaccggtccacgtggtacaacgactacgccgtgtccatgaagtcccggatcaccatcaaccccgacaccaacaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcggtggaggcggtagcggcggtggcggttccgacatccagatgatccagagccctagctccctgagcgccagcgtgggcgacagagtgaccatcacctgtcgggccagccagaccatctggtcctacctgaattggtatcggcagcggccaggcgaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaag

SEQ ID NO: 215 - protein, 5 aa, synthetic construct
GGGGS

SEQ ID NO: 216 - DNA, 783 bp, synthetic construct
  source 1..783: mol_type = other DNA
ctcgagatggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagactagt

SEQ ID NO: 217 - protein, 1108 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 218 - protein, 1108 aa, synthetic construct; identical to SEQ ID NO: 206

SEQ ID NO: 219 - protein, 1108 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 220 - protein, 1108 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 221 - protein, 1108 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPEYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 222 - protein, 1108 aa, synthetic construct
MALPVTALLLPLALLLHAARPQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPEYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 223 - protein, 1104 aa, synthetic construct
GSMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPGSMEFGLSWLFLVAILKGVQCSRDYKDDDDKDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 224 - DNA, 3339 bp, synthetic construct
  source 1..3339: mol_type = other DNA
atggagttcggattatcttggttatttttagtagcgattttgaaaggagtccaatgtagtcgagaacaaaaactcatctcagaagaggatctggatattcaaatgacacaaactacctcttctttatctgcgagtttgggagatcgagttactataagttgccgggctagtcaggatattagtaagtatctcaattggtatcaacaaaagccggatgggacagtcaaattattaatttatcatacatctcgattacacagtggagtaccaagtcggttcagtgggtctggtagcggcacggattattctttgactatatctaatcttgagcaagaagatatagctacctacttttgtcagcaaggtaataccttgccatacacgtttggaggggggaccaaactggagattacaggtagtacgagtggttctggtaagcccggcagcggagaaggttctactaaaggagaggttaaattacaagagtctggcccaggcttagtggccccttctcaatctttgtctgttacatgcacggtctctggggtatctttaccagactatggggtatcttggatacggcaacccccacgaaaagggctcgaatggttgggagtaatctggggttctgaaactacatattacaattctgcgttaaaatctcgattgacaatcataaaagataattctaagagtcaagtgttcttaaaaatgaactctttgcaaacagatgatactgcaatttattattgtgcaaaacattattactacggagggagttatgcaatggattattgggggcaagggacttctgtcaccgtactcgagaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccggatccatggagtttggcctgagctggctgttcctggtggccatcctcaagggcgtgcagtgctccagggactacaaagacgatgacgacaaggacatccagatgacccagaccacaagcagcctgagcgcttccctcggcgacagggtgaccatctcctgtagagcctcccaagacatctccaagtacctgaactggtatcagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgtctagaatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtgcactgccacagactgcccggcagctacgatagcaccagcagcgattctctgtaccccagaggcatccagttcagacggcctcatacagtggctccctggcctcctgcttaccctcctgtgacaagctacccacctctgagccagcctgacctgctgcctattcctagaagccctcagcctctcggcggcagccatagaacacctagcagcagaagagatagcgacggcgccaatagcgtggccagctacgaaaatgaaggcgcctctggcattagaggcgcccaagctggatggggagtttggggacctagctggacaagactgacccctgtgtctctgcctcctgaacctgcctgcgaagatgccgacgaggacgaggatgactatcacaaccctggctacctggtggtgctgcctgatagcacaccagccacatctacagccgctcctagtgctcctgctctgagcacacctggcatcagagacagcgccttcagcatggaatccatcgacgactacgtgaacgtgcccgagtctggcgaatctgccgaagcctctcttgacggcagccgcgagtatgtgaacgtgtcccaagaactgcatcccggcgctgccaaaacagaacctgctgctctgtctagccaagaggccgaggaagtggaagaagaaggcgcccctgactacgagaacctgcaagagctgaactga

SEQ ID NO: 225 - protein, 493 aa, synthetic construct
GSMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVLETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 226-299 - skipped (74 entries)

SEQ ID NO: 300 - protein, 580 aa, synthetic construct
GSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 301 - protein, 580 aa, synthetic construct
GSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 302 - protein, 580 aa, synthetic construct
GSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 303 - protein, 580 aa, synthetic construct
GSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 304 - protein, 580 aa, synthetic construct
GSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPEYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 305 - protein, 580 aa, synthetic construct
GSMALPVTALLLPLALLLHAARPDYKDDDDKQVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPEYLVVLPDSTPATSTAAPSAPALSTPGIRDSAFSMESIDDYVNVPESGESAEASLDGSREYVNVSQELHPGAARTEPAALSSQEAEEVEEEGAPDYENLQELN

SEQ ID NO: 306 - protein, 570 aa, synthetic construct
GSMALPVTALLLPLALLLHAARPDYKDDDDKQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 307 - protein, 577 aa, synthetic construct
GSMEFGLSWLFLVAILKGVQCSRDYKDDDDKDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSRIEVMYPPPYLDNEKSNGTIIHVKGKHLCPSPLFPGPSKPFWVLVVVGGVLACYSLLVTVAFIIFWVHCHRLPGSYDSTSSDSLYPRGIQFRRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 308 - protein, 570 aa, synthetic construct; identical to SEQ ID NO: 306

SEQ ID NO: 309 - protein, 493 aa, synthetic construct; identical to SEQ ID NO: 225

SEQ ID NO: 310 - protein, 261 aa, synthetic construct
ASATMALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK